SEQUENCE LISTING (59 sequences)

SEQ ID NO: 1 - protein, 95 aa, Ixodes scapularis
MKAAIAVLCFLVAVAYAIVVEARMANQPIDDGPLNPKCVKPKGPGDFKTVSYYDPNKGCQLIKLGENCTDNGNYPTLEDCNRHCLPPPGKQTRLS

SEQ ID NO: 2 - RNA, 291 nt, Ixodes scapularis
  source 1..291: mol_type = mRNA
atgaaggcagccattgcagttctttgctttctcgttgcagtagcgtacgccattgtggtggaggcgaggatggcgaaccaaccgattgacgacggccccctcaatccaaaatgcgtaaaaccgaaaggttgccccggagatttcaagacggtttcctattacgacccgaataaaggatgtcaacttattaaactgggagaaaactgtaccgacaatggcaattaccccacacttgaggattgcaatcggcattgtctacctcctccagggaagcaaacgagactcagctga

SEQ ID NO: 3 - protein, 135 aa, Ixodes scapularis
MESFVAMKVVCILFLVGVVAANESGPTKADASTADKDTKKNNVQLRFPNYISNHQKLALKLLKICKDSKSSHNSLSSRSSDVINDKYVDFKNCTFLCKHGNDVNVTLNLPEDTPCGPNGQTCAEKNKCVGHIPGC

SEQ ID NO: 4 - RNA, 408 nt, Ixodes scapularis
  source 1..408: mol_type = mRNA
atggaatctttcgtcgcaatgaaggtggtgtgcatactatttttggttggtgttgtcgctgcgaatgaaagcggcccaactaaagcagacgcatcaaccgctgacaaagatacgaagaaaaacaatgtgcaacttcgattccctaattatatttctaaccaccaaaagcttgccttgaaacttctgaaaatttgcaaggatagcaaatcttctcacaattcccttagttcccgttcgtccgatgtgataaacgacaaatacgtggacttcaagaactgtacatttctttgcaaacatggaaatgatgttaacgtgacattgaatttgccagaagacacgccttgtggaccgaatggacagacatgcgctgaaaagaataaatgcgttggccacattcccggatgttag

SEQ ID NO: 5 - protein, 222 aa, Ixodes scapularis
MKLVLSLAVLLCGVLVRTYGKAPQGGSYGKTSSTTPRGILYGSTTTVPPEEDPSKLREQNATRVVQMNATQWVKWRTYNVTDPLRGNYPLQCENFKVMEKRTPFNYSLQYKYRSGYSWVTRTETLVLGILGLQPLPPNDMFFQRTPIGIPTNNYVLYSNYVNCTILRIPMPGQGQNHCDLWMANMTVSQEPPKICSDKFYEYCNTTQKFTVYFPNCTEISSF

SEQ ID NO: 6 - RNA, 669 nt, Ixodes scapularis
  source 1..669: mol_type = mRNA
atgaagcttgttttaagcttggccgttcttttgtgtggagttcttgtgagaacctatggtaaagcacctcaagggggaagctacggcaaaacaagtagtactacacctcgagggatattatatggcagtacgactacagttccccctgaagaagacccgtcaaaactacgagaacaaaatgcaaccagagtggtccaaatgaacgctacacaatgggtcaagtggcgaacgtataatgtgacagatcctttacgtggtaactacccgctgcagtgtgaaaatttcaaggttatggaaaagagaacaccgttcaactactccttacagtacaaatataggagcggatatagttgggtgacacgtaccgaaaccctagttttggggattctcggattacagcctctcccaccaaatgacatgttctttcaaaggactcctattggcataccaacaaacaattacgtgttgtattccaactacgtgaactgcaccattcttcgaatcccaatgccaggacaaggacagaaccattgtgacctgtggatggcgaatatgactgtttctcaagaaccacccaaaatctgcagcgacaaattttatgaatattgcaacactacacaaaaattcaccgtttactttccaaattgtactgaaataagtagtttttag

SEQ ID NO: 7 - protein, 222 aa, Ixodes scapularis
MKLVLSLAVLVCGVLVRTYGKTHQVGSYGKPSSTTPRGILYGSTTTVPPEEDPAKLREQNATRVVQMNATQWVKWRTFNVTDPFRGNYPLQCENFKVMEKRTPFNYSLQYKYRSGYSWVTLTETLVLGILGEQRLPPNDMFFQRTPIGIPTNNYVLYSNYVNCTILRIPMPGQGRNHCDLWMANMTVSQEPPKICSDKFYEYCNTTQKFTVYFPNCTEISGF

SEQ ID NO: 8 - RNA, 669 nt, Ixodes scapularis
  source 1..669: mol_type = mRNA
atgaagcttgttttaagcttggccgttcttgtgtgtggagttcttgtgagaacctatggcaaaacacatcaagtgggaagctacggcaaaccaagtagtactacacctcgagggatattatatggcagtacgactacagttccccctgaagaagacccggcaaaactacgagaacaaaatgcaaccagagtggtccaaatgaacgctacacaatgggtcaagtggcgcacgtttaatgtgacagatcctttccgtggtaactacccgctgcagtgtgaaaacttcaaggttatggaaaagagaacaccgttcaactactccttacagtacaaatataggagcggatatagttgggtgacacttaccgaaaccctagttttggggattctcggagaacagcgtctcccaccaaatgacatgttctttcagaggactcctattggcataccaacaaacaattacgtgttgtattccaactacgtgaactgcaccattcttcgaatcccaatgccaggacaaggacggaaccattgtgacctgtggatggcgaatatgactgtttctcaagaaccacccaaaatctgcagcgacaaattttatgaatattgcaacactacacaaaaattcaccgtttactttccaaattgtactgaaataagtggcttttag

SEQ ID NO: 9 - protein, 221 aa, Ixodes scapularis
MKLVLSLAVFVCGVFWGTSGSTSTTTRRVGTYGSTGTTTRPGTRGARMIVTTAPPEEDPSKYKEQNATRVVEMNATQWVKWRTYDVTDFSGNPVQCENFRVMEKRTPTNYSFQYRYKSKNSWETIDETLILKDIGEHGFPNVMNFQRTPIGIATDNLVLYSNYVNCTVLRIPFTNQGERHCDLWMANLTLSQETPDDCLNKFFEYCNTTQIYRVYYPSCTN

SEQ ID NO: 10 - RNA, 666 nt, Ixodes scapularis
  source 1..666: mol_type = mRNA
atgaagcttgttttaagcttggccgtttttgtgtgtggagtgttttggggaaccagtggctccacaagtactactacacgtcgagtgggaacgtatggctctacaggaacgactactcgtccagggacacgcggtgccagaatgattgtgactacagctccccctgaagaagacccttcgaaatacaaagaacaaaatgccaccagagtggtcgaaatgaacgctacgcaatgggtcaagtggcgcacgtatgatgtgacagatttcagcggtaacccagtgcagtgtgaaaattttagggttatggaaaagagaactccaacaaactactcattccagtacagatataaaagtaaaaatagttgggagacaatcgacgaaaccctaatcttgaaggatataggtgagcacggtttcccgaacgtcatgaactttcagaggactcctattggcatagcaacggacaatctcgtgttgtattccaactacgtgaactgcaccgttcttcgaatcccattcacaaatcaaggagagaggcattgcgacctgtggatggccaatctgactctttcccaagaaacccccgacgactgcctgaataagttttttgaatattgcaacacgacgcaaatttaccgcgtgtactaccctagttgtacaaactag

SEQ ID NO: 11 - protein, 221 aa, Ixodes scapularis
MGPLNLGDPFPNFTCDTTEGKIDFHEWLGNSWGILFSHPADYTPVCTSELARAAQLHHVFQKKGVKLIALSCDSVESHRGWIKDINAFGELPDGPFPYPIIADEKRDIAVKLGMLDPVEKDKEGLPLTCRAVFIIGPDKKMKLSMLYPATTGRNFDEVLRATDSLLVTETRKVATPAGWQKGTPCMVLPSVTEEEILKLFPTGIKQYEVPSGKNYLRTTMD

SEQ ID NO: 12 - RNA, 666 nt, Ixodes scapularis
  source 1..666: mol_type = mRNA
atgggtcccctgaacctcggcgatcctttccccaacttcacctgcgacacgaccgagggcaagatcgacttccacgaatggctcggcaactcgtggggcatcctgttctcgcaccccgccgactacaccccggtgtgcacaagtgagctcgccagggcagcacagctgcaccacgtctttcagaagaagggtgtcaagctcatcgctctctcctgtgacagtgtggagagccaccgtggttggatcaaggacatcaacgcctttggggagctgccggacgggcccttcccgtaccccatcatcgccgatgagaagcgcgacattgccgtcaagctgggaatgttggaccccgtggagaaggacaaggaagggctgcctctcacctgcagggcggtgttcatcattggtcccgacaagaaaatgaagctctccatgctgtatcccgccacgactggaaggaactttgacgaggtcctgcgtgccaccgattccctgctggtgacggagaccaggaaggtggcgacgcctgctggttggcagaagggcaccccgtgcatggtcctgccttcggtgaccgaggaagagattctcaagctgttcccgacaggcatcaagcagtacgaagttccgtctggcaagaactacctccgaacaaccatggactga

SEQ ID NO: 13 - protein, 125 aa, Ixodes scapularis
MGLTGTMLVLVSLAFFGSAAAHNCQNGTRPASEQDREGCDYYCWNAETKSWDQFFFGNGEKCFYNSGDHGTCQNGECHLTNNSGGPNETDDYTPAPTEKPKQKKKKTKKTKKPKRKSKKDQEKNL

SEQ ID NO: 14 - RNA, 378 nt, Ixodes scapularis
  source 1..378: mol_type = mRNA
atggggttgaccggaaccatgctggtgttggtatctctggccttcttcgggagcgctgcagcccacaattgccagaacggaacgagacccgcatcggagcaagatagagaaggctgcgactattactgctggaacgctgagaccaaatcatgggaccaatttttctttggaaacggagaaaaatgcttttacaacagtggtgatcacggaacatgtcaaaacggagaatgtcatttgacaaataattcaggtgggcccaacgaaactgatgactatactcctgcgcccactgagaagccgaagcaaaaaaagaagaaaactaagaagactaagaaacctaagcgcaagtcaaagaaagatcaggagaaaaacttatga

SEQ ID NO: 15 - protein, 91 aa, Ixodes scapularis
MGLTETTLVLVSLAFFASAVAHNCQNGTRPASEEKREGCDYYCWNAETNSWDKFFFGNGERCFYNDGGEGLCQNGECHLTTDSSVPNDSDV

SEQ ID NO: 16 - RNA, 276 nt, Ixodes scapularis
  source 1..276: mol_type = mRNA
atggggttgactgagaccacgctggtgttggtgtctctggccttcttcgcaagcgctgtggcccacaattgccaaaacggaacaagacccgcctcggaggaaaaaagagaaggctgcgactattactgctggaacgctgagaccaattcatgggacaaatttttcttcgggaacggagaacgatgcttttacaacgatggtggtgagggactatgtcaaaatggagaatgccatttgacaaccgattcaagtgtgcccaatgacagtgatgtctaa

SEQ ID NO: 17 - RNA, 462 nt, Ixodes scapularis
  source 1..462: mol_type = mRNA
atggggttgactgagaccacgctggtgttggtgtctctggccttcttcgcaagcgctgtggcccacaattgccaaaacggaacaagacccgcctcggaggaaaaaagagaaggctgcgactattactgctggaacgctgagaccaattcatgggacaaatttttcttcgggaacggagaacgatgcttttacaacgatggtggtgagggactatgtcaaaatggagaatgccatttgacaaccgattcaagtgtgcccaatgacagtgatgtctaaactcctgctcccactgaaaagccgaagcaaaaaatggagaaaccgaagacgagtaagaagtctaagggcaactcaacgaaagattaggaaggaaacttttcattaaaagtctcgaaactaaaccaggctaaataaagctaaggcactgactctttaaaaaaaaaaaaaaaaaaaaaaaaaaaaaaa

SEQ ID NO: 18 - protein, 233 aa, Ixodes scapularis
MRAVIGALMYILFAFCFASNEVFEKYPKNIGSDEKLITVAFVLAGFDNADVNMKSDVGVWLQDSYDEAADKLSKELQVQLKFEITDIQHAPPALTDEIAYRTVGGQMHGPTILDAVKNTYKKHLNPDIISVITKDKFYDDKFSNKIGFSGFTTLCEDMVPILLTFNSDIEDDVDATATLLSKLIESSLDPVKWKEFGPRKDYFDGCNIRPKPKGDTYNEYYVFPIDKAPFYDF

SEQ ID NO: 19 - RNA, 702 nt, Ixodes scapularis
  source 1..702: mol_type = mRNA
atgagggctgtcattggagcgcttatgtatatattatttgccttctgctttgcaagcaatgaagtgtttgagaaatatccgaaaaacataggttctgatgagaaacttataacggttgcctttgttctggccggatttgacaatgcggatgtgaatatgaaatctgatgttggtgtttggcttcaggattcttatgatgaggctgcagataagttaagcaaagaattacaagttcaacttaaatttgagattaccgatatccaacacgcgcctcctgcccttacggacgaaattgcgtaccggacggttggtgggcaaatgcacggccctacaattctggatgccgtgaaaaatacttataaaaaacacctcaaccccgatatcatatctgttattacgaaagacaaattctacgatgacaagttcagcaataaaataggtttttcagggttcaccacactctgcgaagatatggtgcctattcttttgacgttcaattcggatattgaggacgacgttgatgcgaccgctaccctgttgtccaagctaattgagagcagtttggacccagttaaatggaaagaatttggaccgagaaaagactacttcgatggatgtaacataagacccaaacctaaaggagatacttataatgaatattacgtttttcctattgacaaagctcctttttacgatttttaa

SEQ ID NO: 20 - protein, 130 aa, Ixodes scapularis
MKKTLCAAFFFTVVVAATLAYSDTFEGQVPVFPPGVFTKPPGKVGEPCMTGADCRNGTCCRKEKGGSKTCRRLRKTGERCSDAPIKGEIYEGHCPCKLGLQCFGERVHRCYAVPQASVDPTYTDGREHVL

SEQ ID NO: 21 - RNA, 393 nt, Ixodes scapularis
  source 1..393: mol_type = mRNA
atgaaaaagacactgtgtgcagcgttttttttcaccgttgtcgtggcagcaacgctggcatacagcgatacatttgagggacaggttccagtatttccccctggcgtttttactaaacctcctggaaaagtgggcgagccttgcatgaccggcgccgattgccggaatggcacttgctgtagaaaagaaaaaggcggttcaaagacatgccggcgtctacgaaagactggcgaacgctgttcggacgctccaattaagggtgaaatctacgaaggtcactgtccttgcaagctaggtctgcaatgtttcggggaacgggtccacagatgttacgcagttcctcaagctagtgtggatcctacatacactgacggccgcgagcacgtgttgtaa

SEQ ID NO: 22 - protein, 144 aa, Ixodes scapularis
MVEDGMKNTGILSFIMRGTRLIMATFVLFAICKPIVAGVVIRGTENLAPKCEQKIKHLCENPTHGELTEVTVTARQCQATCTYRPDPTRDTVEVDGIPIKNRHYETVTLPDKMPCGFGARCNKKGECSCNSCNENINHNEPRQT

SEQ ID NO: 23 - RNA, 435 nt, Ixodes scapularis
  source 1..435: mol_type = mRNA
atggtggaggacggtatgaagaacaccggtattttatcctttattatgagaggaacaagactgatcatggcaacgtttgtgctgtttgcaatttgtaaaccaattgtcgccggcgttgtaatcagaggtacggaaaaccttgcgccgaaatgtgaacaaaagattaagcatctttgtgagaaccctacacatggtgagctaacggaggtcactgtgacggctcgccagtgccaagccacatgcacttatcgacccgaccctactagagatacggtggaagttgatggtatacctattaagaaccgtcattatgaaacagtcaccctgccagataaaatgccgtgcggttttggggcgagatgcaacaagaaaggggagtgcagttgcaactcttgcaatgaaaatataaaccacaacgagccaaggcaaacttaa

SEQ ID NO: 24 - protein, 150 aa, Ixodes scapularis
MKLAISLAVFVCGVLVGAYGKMTTTTPRGRLRGSMTTVPPEYDLSKQEEQNATRVVQMNATQWVKWRTYNVTDLVSGYPLQCENFKVMEKRTPTNYSLQYNYRSGYSWETINEDLILGFLNLRPLPPNDMFFARTPIGDQPTIWCCIPTT

SEQ ID NO: 25 - RNA, 453 nt, Ixodes scapularis
  source 1..453: mol_type = mRNA
atgaagcttgctataagcttggccgtttttgtgtgtggagttctagtgggagcctatggcaaaatgactactaccacaccacgagggagattgcgtggcagtatgactacagttccccctgaatacgacctatcaaaacaagaagaacaaaatgcaaccagagtggtccaaatgaacgctacacaatgggtcaagtggcgcacgtataatgtgacagatcttgtcagcggttaccctctgcagtgcgaaaatttcaaggttatggaaaagagaacaccgacaaactactctttacaatacaattataggagcggatatagttgggagacaatcaacgaagacctaattttgggatttctcaatctacggccccttcccccaaatgacatgttttttgcgaggactcctataggggaccaacccacaatttggtgttgtattccgactacatga

SEQ ID NO: 26 - protein, 222 aa, Ixodes scapularis
MLTVSLLTLSLAAYASAVATVTDANNFMDAVLHTRIPALITSEPILFPFATIPPFNFTVAGTNILTNRELQVNVSRGEIRGFSTEVKRVGDCMPPVLREGKTSIRCTLNFTGINATFDTHTRGDNIVASDKNIWVRASVIDTTGQFEAVAERGKQGNVHTFLVDKIHVKVKNDKALSLNDKRKKKFRQHFEDKVLTVLPQIFYGAYLHLLGAAVSSVPFPHV

SEQ ID NO: 27 - RNA, 669 nt, Ixodes scapularis
  source 1..669: mol_type = mRNA
atgctgactgtaagccttctcacgctgtctctagcggcctatgcctccgctgtggccacggtcaccgacgccaacaacttcatggacgcggtgctccacaccagaatcccggcactgatcacgtcggagcccattcttttcccattcgcgaccattcctcctttcaacttcacggtggccggaaccaacatcctgacgaaccgggaactacaggtgaacgtgtcccggggtgaaatccggggcttcagcactgaggtgaagcgcgtgggagactgcatgccgcctgtgctcagggaaggcaagacgtccatcaggtgtaccctcaacttcaccgggatcaacgctactttcgacacgcacacgcggggtgacaacattgtggcctcggataaaaacatctgggtccgcgctagcgtcatcgacactacgggacagttcgaagccgtcgccgagcgcggcaagcagggcaatgttcacaccttcctggtagacaagattcacgtgaaggtcaagaacgacaaggcactcagcctgaacgacaaacgcaagaagaagttcaggcagcattttgaggacaaggttctgaccgtgttgccgcagatattctacggcgcctatctccatctgctcggtgcagctgtgagctctgttcctttccctcatgtttga

SEQ ID NO: 28 - RNA, 1054 nt, Ixodes scapularis
  source 1..1054: mol_type = mRNA
atgctgactgtaagccttctcacgctgtctctagcggcctatgcctccgctgtggccacggtcaccgacgccaacaacttcatggacgcggtgctccacaccagaatcccggcactgatcacgtcggagcccattcttttcccattcgcgaccattcctcctttcaacttcacggtggccggaaccaacatcctgacgaaccgggaactacaggtgaacgtgtcccggggtgaaatccggggcttcagcactgaggtgaagcgcgtgggagactgcatgccgcctgtgctcagggaaggcaagacgtccatcaggtgtaccctcaacttcaccgggatcaacgctactttcgacacgcacacgcggggtgacaacattgtggcctcggataaaaacatctgggtccgcgctagcgtcatcgacactacgggacagttcgaagccgtcgccgagcgcggcaagcagggcaatgttcacaccttcctggtagacaagattcacgtgaaggtcaagaacgacaaggcactcagcctgaacgacaaacgcaagaagaagttcaggcagcattttgaggacaaggttctgaccgtgttgccgcagatattctacggcgcctatctccatctgctcggtgcagctgtgagctctgttcctttccctcatgtttgatcagaaaaccacacatttatttctgttggagcttgaggtcacgctgctatgattaaatttggcctaaaatcaaatagggtttcactttgaatctcgacgtcattcacaatcggggaaagtgatggtcccgaactttaacttaggttttaagactagcttcattcaatgtatttgcctcctgaaatatgttatgtgccacttgaagtgtatgcgtgcaaagatcgctagatattatagtactttgttttgcagtaggattctgtaaaaatcaatatacagtgaaaacaagcgctttaagttctttacgttgtatgcgatgcaaatatttcctgatatgaaataaaactgtcaaaatacatgaaaaaaaaaaaaaaaaaaaaaaaaa

SEQ ID NO: 29 - protein, 303 aa, Ixodes scapularis
MDAHRLMLCVWLWLVSSYVECLPRVRLSSRQQIEGETRITIDFYIDDSVSATEQQVKDYLHKVIFTTTADLQSYFVVDDIHLPYWIKYIGNEPALKSALQSYKNPEFIHLDGAIDALTAYFINQNPPDIICLVTNYTIHNGDNIRKAYGYSKDHTLCKSVVSMLLAYAPYTPGHAGRMLSEMIRASVNPQEMPNLYHRASRQGSFETHMKEYLSTCNKGFKRPGPPEDDMPPRPPPEIPPGPPNVPPPPPPPDTTAEPPQVKPQPPLPPVPPAPPVPPPSQPDKPPNPVPEATTPAEPQPDYC

SEQ ID NO: 30 - RNA, 912 nt, Ixodes scapularis
  source 1..912: mol_type = mRNA
atggacgcacatcgcttaatgttatgcgtatggctatggttggtatcttcttatgtagaatgtttgcccagagttcgattaagttcaagacagcaaattgaaggagagacacgtattacaatagatttttatatagatgattccgtcagtgcgactgaacaacaggtgaaggattatttgcacaaagtcatttttacgacaacggccgatctccaaagttattttgtcgtcgacgatatacatcttccgtactggataaaatacattgggaacgagcctgcactgaaatctgcgctgcagagttataaaaacccggaatttattcacctggatggagcaattgatgcattaacagcctattttataaaccagaaccctcctgacatcatttgcctggtaacaaactatacaatacacaatggagataatatacgaaaggcttatggatattctaaggatcacactctgtgcaagagtgttgtatctatgttgttagcatacgccccctacacaccgggacacgccggcagaatgttgtccgaaatgatcagagccagcgtaaatccacaggaaatgccaaatctgtatcatcgagcatcacgacaaggcagtttcgaaacccatatgaaggaatacttaagcacatgcaacaaagggttcaaacgtcccggccctcccgaagatgacatgcctccacgacccccaccagaaattcctcccggaccccccaatgtccctccaccaccaccaccgccggacacaactgccgaaccgccacaagttaaaccacagccaccattaccgcctgtgccaccagcgccaccagtgccaccaccatcccaaccagacaagcctcctaatccagtaccagaggcaacaacaccagcggagcctcaacctgattactgctaa

SEQ ID NO: 31 - RNA, 1051 nt, Ixodes scapularis
  source 1..1051: mol_type = mRNA
atggacgcacatcgcttaatgttatgcgtatggctatggttggtatcttcttatgtagaatgtttgcccagagttcgattaagttcaagacagcaaattgaaggagagacacgtattacaatagatttttatatagatgattccgtcagtgcgactgaacaacaggtgaaggattatttgcacaaagtcatttttacgacaacggccgatctccaaagttattttgtcgtcgacgatatacatcttccgtactggataaaatacattgggaacgagcctgcactgaaatctgcgctgcagagttataaaaacccggaatttattcacctggatggagcaattgatgcattaacagcctattttataaaccagaaccctcctgacatcatttgcctggtaacaaactatacaatacacaatggagataatatacgaaaggcttatggatattctaaggatcacactctgtgcaagagtgttgtatctatgttgttagcatacgccccctacacaccgggacacgccggcagaatgttgtccgaaatgatcagagccagcgtaaatccacaggaaatgccaaatctgtatcatcgagcatcacgacaaggcagtttcgaaacccatatgaaggaatacttaagcacatgcaacaaagggttcaaacgtcccggccctcccgaagatgacatgcctccacgacccccaccagaaattcctcccggaccccccaatgtccctccaccaccaccaccgccggacacaactgccgaaccgccacaagttaaaccacagccaccattaccgcctgtgccaccagcgccaccagtgccaccaccatcccaaccagacaagcctcctaatccagtaccagaggcaacaacaccagcggagcctcaacctgattactgctaaagtggatcgcacactagaaggggtcatcagtggcacgagtatttggtagcacgtggattgcatgtccgggattgagttttgtttttaataaatatattctggcttaacaaaaaaaaaaaaaaaaaaaaaaaaaaaaaaa

SEQ ID NO: 32 - protein, 115 aa, Ixodes scapularis
MNKVLVVLYFVLLVVAAWPDDDNIQQVATGPRGQDLADGTICSTGDECASKCCLKHFTVTGSDGPAQCHVKSDLGESCSDDQVKGGASVNHCPCSRGSCENNICTLENTDEDKDD

SEQ ID NO: 33 - RNA, 348 nt, Ixodes scapularis
  source 1..348: mol_type = mRNA
atgaataaggttctcgtagtcctctactttgttctcctggtggtggcggcgtggccggatgacgataacatacagcaagtggccacaggaccacgaggacaggacctggcagatggtacgatctgctccacgggggatgagtgcgccagcaagtgctgcctgaagcactttaccgttacggggagcgatggtcctgcccagtgtcacgttaagagtgatctcggggagagctgctctgacgaccaggtgaaaggtggtgcatccgtgaaccactgtccttgctccagagggtcttgcgaaaacaacatctgcactctcgaaaacaccgacgaagataaggacgactga

SEQ ID NO: 34 - protein, 500 aa, Ixodes scapularis
MLSLKFVTLLCALVAVHCEDYEKDEHVLVLKQTNFDKAVTEHKHVLVKFYAPWCGHCKAMAPEYVKAAKQLVDESSDIKLAKVDATIETQLAETYEVRGYPTLKFFRDGKPYDYKGGRTADEMVRWLKKRTGPAAEDLKSADAARTFVDASKVSVVGFFKDQASSEAMQFLEAAEAIDAHPFAITSDDAVYKELGASKDGVILFKKFDEGRSLMEGAVTSESVQSFVKTNSLPLVVEFTHESAQTVFGGQIKLHNLLFVSKKSPGFEDILKDYREAAKDFRHKVLFVTIDVDDEDHERILEFFGLKKDQVPVMRFVKLEGEMTKYKPEKDDLTPENVRSFVQDVLDGKLKQSLLSQDLPEDWDRHAVKVLVNKNFDEVVFDKEKDVLVEFYAPWCGHCKQLAPIYDELAEKYKEKRPDLVIAKFDGTANELEHTKMQGFPTIRLYKKGTNEAVEYNGERTLEGLSKFIDTDGEYGKAAPEEVTEEEEEEEDKEDKKRDEL

SEQ ID NO: 35 - RNA, 1503 nt, Ixodes scapularis
  source 1..1503: mol_type = mRNA
atgttgtcgctaaagttcgtcacgttactctgcgctctcgtcgccgtccactgtgaagactacgagaaggacgagcacgtcctggtcctgaaacagaccaacttcgacaaagcagttaccgaacacaagcatgtgctggtcaagttctatgccccctggtgcggccactgcaaggcgatggcgccagagtatgtcaaggccgccaagcagctagtggacgaaagctcggacatcaagcttgccaaggtcgacgccacaatagagactcagctcgccgagacctacgaggtgcgaggctacccgaccctcaagttcttccgcgatggcaagccctacgactacaagggtggccgtactgccgacgagatggtgcgttggctcaagaagaggactggacccgcagctgaggacctcaagtccgccgatgctgcacgtaccttcgtggatgcctccaaagtgtctgtggttggctttttcaaggaccaggcaagcagcgaagccatgcagttcctagaggctgcagaggccatcgacgcccacccctttgccatcacgtccgacgacgcggtctacaaggagctgggagcctccaaggatggagtcatactcttcaagaagtttgacgagggccgcagcctgatggagggtgctgtgacgtccgagagtgtccagtccttcgtcaagaccaacagcctgcccctggtggtagagttcacccacgagagtgcccagacggtcttcggaggacagatcaaactgcacaacctgctctttgtgagcaagaagagccccggcttcgaggacattctcaaggactaccgggaggcggccaaggacttccgccacaaggtgctcttcgtcaccatcgacgtggacgacgaggaccacgagcgcatcctcgagttctttggcctcaagaaagaccaggtgcccgtaatgcgcttcgtcaagctggagggcgagatgaccaagtacaagccggagaaggacgacctcacgcccgagaatgtgcgctcatttgtgcaagacgtgctggacggcaagctgaagcagagccttttgtctcaagacctgccggaggactgggacaggcacgccgtcaaggtcctcgtcaacaagaacttcgacgaggtggtctttgacaaggagaaggacgtcctggtcgagttctacgctccctggtgcggacactgcaagcagctggccccgatctacgacgagctggctgagaagtacaaggagaagcggccggacctggtgattgccaagttcgacggcacggccaacgagctggagcacaccaagatgcagggcttccccaccatccggttgtacaagaagggcaccaacgaggccgtggagtacaacggggagcgcaccctcgaggggctgtccaagttcatcgacacggacggggagtacggcaaggcggcccccgaggaggtcacggaagaggaggaggaagaggaagacaaggaggacaagaagcgtgacgagctctaa

SEQ ID NO: 36 - RNA, 2106 nt, Ixodes scapularis
  source 1..2106: mol_type = mRNA
gagtatggcgacccctcataaattgagcatgcgcaaacgcgccgcgctagcctctgccacgtttgtaacactcgaaggctggtcaccggtgccgtaggaggggagcaagttaaaacaaactttcttttatacacgcgacataggaaccatgttgtcgctaaagttcgtcacgttactctgcgctctcgtcgccgtccactgtgaagactacgagaaggacgagcacgtcctggtcctgaaacagaccaacttcgacaaagcagttaccgaacacaagcatgtgctggtcaagttctatgccccctggtgcggccactgcaaggcgatggcgccagagtatgtcaaggccgccaagcagctagtggacgaaagctcggacatcaagcttgccaaggtcgacgccacaatagagactcagctcgccgagacctacgaggtgcgaggctacccgaccctcaagttcttccgcgatggcaagccctacgactacaagggtggccgtactgccgacgagatggtgcgttggctcaagaagaggactggacccgcagctgaggacctcaagtccgccgatgctgcacgtaccttcgtggatgcctccaaagtgtctgtggttggctttttcaaggaccaggcaagcagcgaagccatgcagttcctagaggctgcagaggccatcgacgcccacccctttgccatcacgtccgacgacgcggtctacaaggagctgggagcctccaaggatggagtcatactcttcaagaagtttgacgagggccgcagcctgatggagggtgctgtgacgtccgagagtgtccagtccttcgtcaagaccaacagcctgcccctggtggtagagttcacccacgagagtgcccagacggtcttcggaggacagatcaaactgcacaacctgctctttgtgagcaagaagagccccggcttcgaggacattctcaaggactaccgggaggcggccaaggacttccgccacaaggtgctcttcgtcaccatcgacgtggacgacgaggaccacgagcgcatcctcgagttctttggcctcaagaaagaccaggtgcccgtaatgcgcttcgtcaagctggagggcgagatgaccaagtacaagccggagaaggacgacctcacgcccgagaatgtgcgctcatttgtgcaagacgtgctggacggcaagctgaagcagagccttttgtctcaagacctgccggaggactgggacaggcacgccgtcaaggtcctcgtcaacaagaacttcgacgaggtggtctttgacaaggagaaggacgtcctggtcgagttctacgctccctggtgcggacactgcaagcagctggccccgatctacgacgagctggctgagaagtacaaggagaagcggccggacctggtgattgccaagttcgacggcacggccaacgagctggagcacaccaagatgcagggcttccccaccatccggttgtacaagaagggcaccaacgaggccgtggagtacaacggggagcgcaccctcgaggggctgtccaagttcatcgacacggacggggagtacggcaaggcggcccccgaggaggtcacggaagaggaggaggaagaggaagacaaggaggacaagaagcgtgacgagctctaagcaactcccctgtgacccatttctcccaccgctcattttcccccctccccccgctgccctctcattggtcactcacccccgtccctgttgttgttcgcatgtggtccccctcttgcgtttgatccccttttcccgatctcttccaaggcctcacatgagggtgcaaagaagcccccgtttaaaggagggtgaccggaagtaccacgcacacatttggggggtgcggtttcggtcgtagtctgccgccacctccctgtggtcccccccggcccttgtgagggggggggagggcccttctggacttccgtgcgccgaaccccctttgcttgtggggtggacggtgctgcatttgtgtgcgcgagtgtgtgctggggggtgccgtctgggcacctttcgattaacaaagaaaagggctctgcggagcttcattaaactgtacaagctgtcacgtggaa

SEQ ID NO: 37 - protein, 557 aa, Ixodes scapularis
MLTSAGNQGQRTLSVTSQLTKAPQTNQRPTAQLARRRSAFSHVVFPTLKKPALGTAFFFRREVRVAVRSAAMRWFPLLAVLIPAALASDVLDYSADFDTKIQDHDAALVEFFAPWCGHCKRLAPEYEKAATELKTNDPPVPLIKVDCTSDGGKDTCSKHGVSGYPTLKIFRGGEFSADYNGPREAGGIVKYMKAQVGPSSKELLSVAEVEKYLSKDDVVIFGFFESKDASLHENFLKVADKQREAWTFGHSFDKDVLKKYGYKNQVVLFRPKILKNKFEESFAVYSGSDDKAELETFIKENYHGLVGHRTQDNYNMFQAPLLVAYYDVDYTKNAKGTNYWRNRILKVAQNYKGKLNFAVSNKDSFAAEMDDYGVTVKANKPAIAVRNSENEKFRMTNDFSVENLEKFLEEYLAGKVKAHLKSEPVPETNDGPVKVAVAENFKSLVTESTKDVLIEFYAPWCGHCKKLAPTYEEVGKTLADEDVLVVKMDATANDVPSAFEVSGFPTLYWLPKNDKQNPRRYEGGREHDDFIKWIAKHATDELKAYDRSGAKRAKEEL

SEQ ID NO: 38 - RNA, 1674 nt, Ixodes scapularis
  source 1..1674: mol_type = mRNA
atgctaacctctgctggcaaccaaggacagcggacgctgtcagtgacgtcacaacttacaaaggcgccccaaaccaatcagcgcccgacagcacaactcgcccggcgacgctctgcgtttagccacgttgttttcccaactttaaagaagccggcacttggcactgccttttttttccgtcgtgaggttagagtcgcggttcggtcagcagccatgaggtggtttcccttgctagcggttttaatccccgctgcgcttgccagcgatgtcctcgactactcggcggacttcgacacgaagattcaggaccacgacgcagcactagtcgaattcttcgctccatggtgcggacactgcaagaggttagcgcccgagtacgaaaaagcagctaccgaacttaaaaccaacgaccctccagttccattgataaaggtggactgtacctccgacggtggaaaagatacgtgttcaaagcatggagtcagcggctaccccaccctcaagatcttcagaggtggagagttttcggcggattacaatggcccaagggaagccggtggcatcgttaaatacatgaaggcccaggtgggaccaagctcgaaggaacttctgtctgttgctgaagtcgagaagtatctgtcgaaggacgacgttgtcatttttggtttcttcgagagcaaggatgccagcctgcacgaaaacttcctcaaggttgcagacaagcagcgggaggcctggactttcgggcactcgtttgacaaggatgtgctcaagaagtatggctacaaaaaccaagttgtgctgttccgacccaagattctcaagaacaagtttgaggagagctttgcagtttactctggctctgatgacaaggcggaactggagacattcatcaaggagaactaccatggcctggtgggccacaggactcaggacaactataacatgttccaggctccgctcctggttgcctactacgacgtagactacaccaagaacgccaagggcaccaactactggcgcaaccgcatcctcaaggtggcccagaactacaagggcaagctcaattttgctgtgagcaacaaggacagctttgctgccgagatggacgactacggcgtgacggtcaaggccaacaagcctgccatcgccgtgcgaaacagcgagaacgagaagttccgcatgaccaacgacttcagcgtcgagaacctggagaagttcctggaggagtaccttgctggcaaagtgaaggcccacctaaagtccgagcccgtgccggagaccaacgacggaccggtcaaggtcgcggtggcagagaacttcaagtccctggtcacggagagcaccaaggacgtcctcatcgagttctatgccccctggtgtggacactgcaagaagttggcccctacctacgaggaagtgggcaagacgcttgctgacgaggatgtcctagttgtgaagatggatgccacagccaatgatgtgccgtctgcctttgaggtgtctggtttccccaccctgtactggctgcccaagaacgacaagcagaacccgcggcggtacgaaggaggccgggagcacgacgacttcatcaaatggattgccaagcacgccacggacgagctcaaggcctacgaccgctccggagccaagagggccaaggaggagttatga

SEQ ID NO: 39 - RNA, 1830 nt, Ixodes scapularis
  source 1..1830: mol_type = mRNA
aggaaaaactcgcgagcgccgagcactgcacgcgatgctaacctctgctggcaaccaaggacagcggacgctgtcagtgacgtcacaacttacaaaggcgccccaaaccaatcagcgcccgacagcacaactcgcccggcgacgctctgcgtttagccacgttgttttcccaactttaaagaagccggcacttggcactgccttttttttccgtcgtgaggttagagtcgcggttcggtcagcagccatgaggtggtttcccttgctagcggttttaatccccgctgcgcttgccagcgatgtcctcgactactcggcggacttcgacacgaagattcaggaccacgacgcagcactagtcgaattcttcgctccatggtgcggacactgcaagaggttagcgcccgagtacgaaaaagcagctaccgaacttaaaaccaacgaccctccagttccattgataaaggtggactgtacctccgacggtggaaaagatacgtgttcaaagcatggagtcagcggctaccccaccctcaagatcttcagaggtggagagttttcggcggattacaatggcccaagggaagccggtggcatcgttaaatacatgaaggcccaggtgggaccaagctcgaaggaacttctgtctgttgctgaagtcgagaagtatctgtcgaaggacgacgttgtcatttttggtttcttcgagagcaaggatgccagcctgcacgaaaacttcctcaaggttgcagacaagcagcgggaggcctggactttcgggcactcgtttgacaaggatgtgctcaagaagtatggctacaaaaaccaagttgtgctgttccgacccaagattctcaagaacaagtttgaggagagctttgcagtttactctggctctgatgacaaggcggaactggagacattcatcaaggagaactaccatggcctggtgggccacaggactcaggacaactataacatgttccaggctccgctcctggttgcctactacgacgtagactacaccaagaacgccaagggcaccaactactggcgcaaccgcatcctcaaggtggcccagaactacaagggcaagctcaattttgctgtgagcaacaaggacagctttgctgccgagatggacgactacggcgtgacggtcaaggccaacaagcctgccatcgccgtgcgaaacagcgagaacgagaagttccgcatgaccaacgacttcagcgtcgagaacctggagaagttcctggaggagtaccttgctggcaaagtgaaggcccacctaaagtccgagcccgtgccggagaccaacgacggaccggtcaaggtcgcggtggcagagaacttcaagtccctggtcacggagagcaccaaggacgtcctcatcgagttctatgccccctggtgtggacactgcaagaagttggcccctacctacgaggaagtgggcaagacgcttgctgacgaggatgtcctagttgtgaagatggatgccacagccaatgatgtgccgtctgcctttgaggtgtctggtttccccaccctgtactggctgcccaagaacgacaagcagaacccgcggcggtacgaaggaggccgggagcacgacgacttcatcaaatggattgccaagcacgccacggacgagctcaaggcctacgaccgctccggagccaagagggccaaggaggagttatgagtggtctctgcttcacctgtgggggaccagttgggacaaggacattgttcaatcactttcaccgggggttctgtttttcgccatctctgtacaataaaccaagggtgtttttgtaatcgcga

SEQ ID NO: 40 - protein, 1329 aa, Ixodes scapularis
MRVPYVLPVLFLAAASAFEVGKDYVYHYNGKMQVYNPEQPLQSSGFAFRSKVVAQPRPDHTHFKIIDFEVDSFNGDHVHVGEHEFNYHSTEALKQFIERPFAGKFSEGKLEEAELSKSEPKWARNLKKGVLSIFQLDLVKGRHDHPHAKQFHVREEGLHGNCDTLYVVAEEEGHLKVTKIKNLEKCDKEHYAVYGRIKGHECVDCEAQETHPFVATSQVKYRLDGTPEHYVINHACATSENVFRPFGQGKTFVAQLNRTLDLEEVHDANTDTQLPEDLEKVHHIAQTFPESDEVESLEELKHVNRYVTTFDLSTDKDKFISGLNHLAALEYEDSDIKDVHSKESGGLNFLILFGSLASMPFEDIAHVYEQAVANAPEASKSQVRKVFLDLLSAVGNNPHAAFGLQLVKEDKLTDEEAEHFLAKLALNLKENSPALLTELAEVCEHVKPKRPVWVNCQLALSTLAGQEGCVRAKTDKEQDEGFCKPSIVSHFFNYEIKPEDKKDQPEYKRTVYMKAAGNLATRGAVHYLERYVSDTNQPEYRRSAALWAMVRAAPHHHELVRDVALPLYKNKSETAYLRIGAFVNVLMTKPDLYLLKYIGHNIIDDPSDQLASYVTSAFRSLVKSKYPCHQELAQHLRYVVPMWNDVSRFAKPLDHTKSQVILSSGYDPKYDYGGSSVFAQVRADDSYLPREVYFSAKDYFHGRSFESVSLKFESWGLDKLLNHVVGPQPGSTKNLWNVFGRRRFTRDASAKDLQEVEAALPITDRDYDHVYGRLSLDLYGHAFDTWEFDESILQELSKGEGEEAPGDKLKNLLGESKRKKSFILSKDICVMVPSELGVPVFYHILQVEYMYINPKKFAFDQTQDAKFAVDLERHYVHESRGYSMFGVALTFNKTSMGTGSDSQTIMNLPIDLKITLDPIHKKLSMKRPLSLPWNVLNHHFRPFTFVMSYDMHKDATTTETMMTQAMHPLYNKEELTEFDRNYFGEVLGYGLNVKGSLLSKGLKKGLHDYWYESDDRQKFYYATLNPEWHPRELQFTVVPAAQDATTEVEVDLGYKFLEPDDPRESHFEAHDNVGDDAEVPSTHVLNLDYTLKGTKERKVSAELRYSFTKDLLKHKVQLFYDRTPFHPKEQDHTKVEVDLGYKFLEPDDPRESHFEAHDNVGDDAEVPSTHVLNLDYALKGTKERKVSAELRYSFTKDLLKHKVQLFYDRTPFHPKEQDHTKITFKGKYTHTDDEEKQIRENAEGKPLGINRMKKYTLRRPYKKCTEHQKRGVPLNFECMKYLYYTSRLGKLTTDVEYKNLKPVSVEGLHPVNQGRAPASFRRFRTGAPR

SEQ ID NO: 41 - RNA, 3990 nt, Ixodes scapularis
  source 1..3990: mol_type = mRNA
atgagagtcccctacgttctgcccgtcctgttccttgccgccgcttcggccttcgaagtgggcaaggactatgtgtaccactacaacggaaagatgcaagtgtacaacccggagcagcccctgcagtcctcgggtttcgccttccgaagcaaggtcgtcgcccagcccaggcccgaccatacgcacttcaagataatcgactttgaggtggacagcttcaacggcgatcatgttcatgtcggtgaacacgagttcaactaccactccaccgaggcgctgaagcaattcatcgagaggcccttcgccggcaagttctccgaaggaaagctcgaagaagctgagctgagcaagagcgaacctaagtgggccaggaacctcaagaagggagtcctcagcattttccagctggacttggtcaagggtcgccacgaccacccccacgccaagcagttccacgtgagggaggaaggtctccacgggaactgcgacacactctacgttgtcgccgaggaggaaggtcacctcaaggtcaccaagatcaagaatctcgagaaatgcgacaaggaacactacgccgtttatggaaggatcaagggacatgagtgcgtcgactgcgaagcccaggagactcaccctttcgtagccacctctcaggtcaagtacagactcgacggaactccagaacactacgtcatcaaccacgcctgcgccacctcagagaatgtcttcaggcccttcggtcaagggaaaaccttcgttgcccagctcaaccgtaccttggatttggaggaggtgcacgacgccaacaccgacactcagcttccggaggacctggagaaggtgcaccacatcgcgcagacattccccgagtctgatgaggtggaaagcctggaggaactgaagcacgtcaaccgctacgtgaccacctttgatctctcgaccgacaaagacaagttcatctctggtctcaaccaccttgctgcattggagtacgaagattccgacatcaaggacgttcacagcaaggaaagcggtggactaaacttcctgattttgttcggctcgcttgcctccatgccctttgaagatatcgcccacgtgtacgagcaagccgttgccaatgcaccggaagcaagcaagagtcaagtcagaaaggtgttcctggacctgctctcagctgtcggaaacaaccctcacgcagcgttcggtctgcaactggtcaaggaagacaaactcactgacgaagaggctgagcacttcctcgccaagctggcgctgaaccttaaggagaacagccccgctcttctgaccgaacttgccgaggtgtgtgagcacgtgaagcctaagcgaccagtatgggtcaactgccaactcgctctgagtacactcgcaggccaagagggctgcgtccgtgccaagactgacaaggaacaggacgagggattctgcaagccctctattgtttcgcacttctttaactatgagattaagccagaagacaagaaggaccagccagaatacaagaggacggtgtacatgaaggctgcgggcaacttggccaccaggggcgccgtgcactacctggagcgctacgtttcggacacaaaccagccggagtaccggaggtcagccgccctctgggcaatggttcgggctgctccgcaccaccacgaactggtacgcgacgtcgcactcccactgtacaaaaacaagagcgagacggcctacttgcgcatcggggcatttgtgaacgtcctaatgaccaaacccgacctctacctgctcaagtacatcggacacaacatcatcgacgaccccagcgaccagctggcgtcctacgtgaccagcgccttccgcagccttgtgaaatccaagtacccgtgccaccaagaactggctcagcaccttcgctacgtcgtgcccatgtggaacgacgtaagccggtttgccaagccgctggaccataccaaatcgcaggtcatcctctcttccggctacgaccccaagtacgactacggtggctccagtgtcttcgcccaagtacgggccgacgacagctacctaccgagagaggtgtatttcagcgcaaaggactatttccacggccgctccttcgagtcagtttcactaaagtttgagagctggggtctggacaagcttctgaaccacgtcgtgggacctcagcccggatccaccaagaacctgtggaacgtctttgggcgacgtcgcttcaccagggacgcctcggcaaaggatctccaggaagttgaggctgctcttcccatcactgaccgggattatgaccatgtctacggccgcctgagtctcgatctctacggtcacgccttcgacacctgggaattcgacgagagcattctgcaggaactgagcaagggagagggagaagaggcaccaggcgacaagctgaagaacctgctaggcgaatcaaagcgtaagaagagcttcatcctcagcaaggacatctgcgttatggtacccagcgaacttggtgttcccgtattttaccacatcctgcaagtcgaatacatgtacatcaaccccaagaagtttgccttcgatcaaacgcaagatgccaaatttgccgtcgacctagaaaggcactacgtacacgaaagccggggctacagcatgttcggtgtcgctctcaccttcaacaagacgtccatgggaacgggctccgatagtcagaccatcatgaatctgccgatcgacctgaagattaccctggatcctattcacaagaagctgagtatgaagcgcccgctgagcctgccctggaacgtcctgaaccaccacttccgcccgtttaccttcgtcatgtcctacgacatgcacaaagatgctaccaccaccgaaaccatgatgacccaggctatgcacccactctacaacaaggaagaacttaccgagtttgatcgtaactacttcggcgaggtgttgggctatggcctgaacgtgaagggaagcctcctcagcaagggactcaagaagggactgcacgactactggtacgagagcgacgaccgccagaagttctactatgccactctgaaccctgagtggcacccacgtgaactgcagttcaccgtcgttccagctgcacaagatgccacaaccgaggtagaggtcgatctcggctacaagttcctcgagcccgatgacccacgggagagtcacttcgaagctcacgacaacgtcggcgacgatgctgaggttccgtccactcacgtgctgaacctcgactacactctgaagggcaccaaggaacgcaaggtgtccgcggaactgagatactccttcaccaaggacctcctcaagcacaaagtacagctcttctatgaccgaacaccattccacccgaaggagcaagaccacacgaaggtagaggtcgatctcggctacaagttcctcgagcccgatgacccacgggagagtcacttcgaagctcacgacaacgtcggcgacgatgctgaggttccgtccactcacgtgctgaacctcgactacgctctgaagggcaccaaggaacgcaaggtgtccgcggaactgagatactccttcaccaaggacctcctcaagcacaaagtacagctcttctacgaccgaacaccattccacccgaaggagcaagaccacaccaagattacctttaagggaaagtatacgcacacggacgacgaagagaagcagattcgagagaacgcagaaggcaagccgctgggaatcaaccgaatgaagaagtacaccttgcgtcggccgtacaagaagtgcaccgagcaccagaagcggggcgttcccctaaactttgagtgcatgaagtacctgtactacaccagtcgcctgggtaaattgacaacggacgtcgagtacaagaacctcaagccggtgagcgttgaaggccttcatccagtcaaccaaggtcgagctcctgccagtttccgccgattccggactggtgctccgcgttga

SEQ ID NO: 42 - protein, 1556 aa, Ixodes scapularis
MRVPYVLPVLFLAAASAFEVGKDYVYHYNGKLQVYNPEQPLQSSGFAFQSKVVAQPRPDHTHFKIIDFEVDTFNGEHVHLSDHQFHYHSTDALKQFIERPFAGKFSEGKLEAAELGKSEPMWSQNIKKGVLSVFQLDLVKGRHDDPHAKQFYVKEDGLHGNCDTLYVVAEKEGHLEVTKIKNLEKCDKDHFAIYGRIKAHECVDCEAQESHPLVATAQVKYRLDGTPEHYVIDHACATSETVLRPYGKGKTFVVQLNRTLDLEEVHDANTDTQLPEDLERVDHLSQTLPVGDHVETLQDLKKVNHFVDYFQLTNNKEKFVAGLNHLAALEYEDDDVKDVHGKESGGLQFLVLFNALSTLHFEDVVQVYEQAVASAPEASQSHVKRLFLDLLSAAGTNPQVAFGLQLVKEDKLSDDEAEHFFTKLALNLKENSPALLTELAEVCEHVKPKRQVWVNCQLALSILAGQEGCVRAKTDKEHDEGFCKPSLVSHFFNYSVKPEDKKDQPEYKRTAYIKAAGNLATRGAVHYLERFISDTNQPEYTRAAAFWALRRAGSKHPELVRDIALPVYKNKSESSYLRIAAFTAVMKTHPDLYLLKYIGHNIIDDPSDQVASYVTSLFRSFEKSKYPCHQSLAQHLRYVVPMWNDVYRFSKPPDYTKSHVHLSSGYDPKYDYGGATSFGMVRADDSYLPRDVYVSVKDYMSGHSLSTATLWFENWGMDKLLNHVVGPQPGSSKNLWNVFGRRRFTRDASAKELKEVEDALPVTDREYDHVYGRLNLDLFGHAIDSWEFDEDLLKEVGKEDGEGKCLESLKLLILTDRLRKHKPGTKLKNLLGQVQRKKNFYLSTDIIFVAPTEVGVPIFFDFKQAEFTYINRKKFSVDQTEDAKFTFDVQRHYVHESRGYTMVGVGLTFNKTSLGTGSDVQTIFNLPIDLHLTVDPIHKTLSLKRPLSLPWNVLNHHFRPFTFTMPYDIATDVSHAVTTLEQPQYPLYNKEEVTEIDHTYLTDVFGYGLNVKGSLLSKGLKKGLHDFWYESDCRQRYYYALMNPEWHPRELQFTVVPAEHDATTEVEVDLGYKFLEPDDARESHFPVQDNVGGDAEVPSTHVLSLDYTLKGSKERKASAELRYSFTKDNLKHKVQFFYDRTPFHPKEQVHTKVCMDVTSKFPKPEWSKLKNLAVFHEGKDLESELNLHYGSNCVDQSSITFKGKYTHTDDEERQIRENAEGKPRGINRMDKYNLRDPFNKCTEHQKRGVPLNLYCMKYLYYTSRLGKFTTDVEYKNLKPLLPMLTKYYKKVHPEGGFLSTVVSHFHGPNGKLHVVSQVPPVQDYADIVVTTEDGHSHNHSHVPLYSHLLEPRIMFSTLGYSNMAHYISYYKQKYCDLQGQSVRTFDNVVVQLPETDCYKVVAKDCSPNKRFTVLARATGNAELPKALKAFIQTTQVELLPVAEDSGLVLRVDGNRVPLTPGVPYSHTAHDAELFTVTLENKYFEVTSKPYGLNLGFNGNMLFVQTANFYRGKLCGLCGDYNYDRQHELVGPNLHHYNDTLEFAKSYVVPSSDCTPP

SEQ ID NO: 43 - RNA, 4671 nt, Ixodes scapularis
  source 1..4671: mol_type = mRNA
atgagagtcccctacgttctgcctgtcctgttccttgctgcagcttcggccttcgaagtgggcaaggactacgtgtaccactacaacggaaagctacaagtctataacccggagcagcccctacagtcttcgggattcgcgttccagagcaaggtcgtcgcccaacccaggcccgaccacacgcacttcaagataatcgatttcgaggtggacaccttcaacggcgaacatgtccatctcagtgaccaccagttccactaccactccaccgacgcactgaagcaatttattgagagaccatttgccggcaagttctccgaaggaaagctcgaggccgcggagctgggaaagagcgagcccatgtggtctcagaacatcaagaagggtgtcctcagcgtgttccaactggacctcgtcaagggacgtcatgacgatccccacgccaagcagttctacgtgaaggaggacggccttcacggaaactgcgacaccctctacgttgtcgccgagaaagagggtcacctcgaggttaccaagatcaagaatcttgagaaatgcgacaaagaccacttcgccatctacgggaggattaaggcgcacgagtgcgtcgattgtgaagcccaggagtcgcaccccctcgtggccaccgcccaggtcaagtacaggctggatggaactccagagcactacgtcattgaccacgcctgcgccacctcggagactgtcttgaggccttacggcaaaggaaaaaccttcgttgtccagctcaaccgcaccttggatttggaggaggtgcacgacgccaacaccgacacccagctcccagaggacctggaaagggtggaccacctgagtcagacgcttccagtaggtgatcatgtcgaaacactccaagatctgaagaaggtcaaccacttcgtcgactactttcaactcacaaacaacaaagaaaagttcgtcgcgggtctgaaccacctcgctgcccttgagtacgaagatgacgacgtcaaggatgttcacggcaaggaaagcggaggcctccagttcctggtcttgttcaacgctctttccactctgcatttcgaagatgtcgtccaggtgtacgagcaagccgtcgccagcgcaccggaagcaagccaatctcacgttaagagactgttcctggacctgctgtcagctgctggaacgaaccctcaagttgcgttcggcctgcaactggtcaaggaagataagctctcggacgatgaggctgagcacttcttcaccaagttggcgctcaaccttaaggagaacagccctgctcttctgactgaacttgccgaggtgtgtgagcacgtgaagccgaaacgacaagtgtgggtcaactgtcagctcgccctaagtatcctcgcaggccaagagggttgcgttcgcgcaaagaccgacaaggaacacgacgagggattctgcaagccctctctggtgtcgcacttctttaactattcggtcaagcctgaagacaagaaggaccagccagaatataagaggaccgcgtacatcaaggctgctggcaacctagccaccaggggcgccgtgcattacctggaacgcttcatctccgataccaaccagccagagtacacgagggccgccgccttctgggcactgaggcgagctggatcgaagcaccccgaactggtgcgcgatatcgcacttcccgtgtacaagaacaagagcgagtcatcgtatctgcgtatcgcggcgttcacagccgtcatgaagacacatcccgacctctacctgctcaagtacatcggccacaacatcatcgacgaccctagcgaccaagtggcatcttacgtgaccagcctgttccgtagtttcgagaaatccaagtatccctgccaccaatcactagctcagcaccttcgctacgtcgtgccaatgtggaacgacgtgtaccggttctccaagcccccggactacaccaaatcgcatgtgcatctttcctctggttacgaccccaagtacgactacggtggcgccacgtccttcggtatggtccgggctgacgacagctacctcccacgagacgtctacgttagcgtcaaggactacatgagcggtcactccctcagcacggctaccttgtggtttgagaattggggcatggacaagctgttgaaccacgtcgtgggacctcagcccggatcctcaaagaacctgtggaacgtctttgggcgacgtcggtttaccagggatgcgtcggcgaaggagctcaaggaagtcgaggatgcgcttcccgtcaccgatcgggagtacgaccacgtctacggcagactgaacctcgaccttttcggtcacgccatcgacagctgggagttcgacgaggatcttctcaaagaagtcggcaaggaagacggagaaggcaagtgcttagaatctttgaagcttttaatattaactgacaggctaaggaaacacaaaccaggaaccaagctgaagaacctcttgggccaagtgcagcgcaagaaaaacttctacctgagcactgacataatctttgttgcgcccaccgaggttggtgttccaatcttcttcgacttcaagcaagccgaatttacgtacatcaaccgcaagaagttcagcgtggaccagactgaagacgccaagttcacttttgatgttcagaggcactacgtgcacgagagccgtggctacaccatggtcggcgtgggcctgacctttaacaagacgtccttgggaacggggtccgacgtccagaccatcttcaacctgccgatcgacctacacctcactgtggatcccattcacaagacgctgagcctgaagcgcccgctgagcctgccctggaacgtcctgaaccaccacttccgtccgttcacctttaccatgccctatgacatcgccacagatgtgtcccacgctgtcaccacactggagcagcctcagtaccccctctacaacaaggaagaagtaactgagattgatcatacctacctcaccgacgtcttcggctatggcttgaacgtgaagggtagcctcctcagcaaaggactcaagaaaggactgcacgacttctggtacgagagcgattgccgacagaggtactactatgccctcatgaatccagagtggcacccccgtgaactgcagttcaccgtcgttccagctgagcatgacgccacgactgaggtagaggtcgatctcggctacaagttcctcgaacccgatgacgcaagagagagccacttccccgttcaggacaacgtcggcggagatgctgaggttccttccacccatgtgctcagcctcgactacactctgaagggctccaaggagcgcaaggcgtccgcagaactgaggtactcgtttaccaaggacaacctcaagcacaaagtgcagttcttctatgaccggacgcccttccacccaaaggagcaagtccacactaaggtctgcatggatgtcacgtctaaattcccaaagcccgagtggtccaagttgaagaacctcgccgttttccacgagggcaaggatctggaatctgagctgaaccttcattacggaagcaactgcgtagaccagtctagcatcacattcaagggaaaatatactcacacggatgacgaagaaaggcagattcgagagaacgctgaaggcaagccgcggggaatcaaccgaatggacaagtataacctccgagatccgttcaacaaatgcaccgagcatcagaagcgtggcgttccactaaacctttactgcatgaagtacctgtactacaccagccgcctgggtaaattcacaacagacgtcgagtacaagaacctgaagccgttgctccccatgctaacgaagtactacaagaaggtccaccccgagggaggtttcctgagcaccgtagtgtcccacttccatggacccaacggaaagctccacgtcgtctctcaagtgcctcctgttcaggactacgcggacatcgttgtaaccacggaggacggtcactcccacaaccactcccacgtgccgctctacagccacttgctcgagcccaggatcatgttctccacgctagggtactcgaacatggctcactacatctcctactacaaacagaaatactgcgatctgcaaggacagagcgtcaggactttcgacaacgtcgtcgtgcagctgccggagaccgactgctacaaggtggtcgccaaggactgctcgcccaacaagaggttcacggtcctggcacgggctaccggaaacgccgaactccccaaggcgctgaaggcattcatccagaccacccaggtcgagctcctgccagttgccgaagactccggtcttgtgctccgcgttgacggaaacagagttccactgaccccgggagtaccttacagtcacaccgcgcacgacgccgagctcttcaccgtcacgctggaaaacaaatacttcgaagtaacgtcgaagccatacggtctcaatctaggcttcaacggaaacatgctcttcgtacagaccgccaacttctaccgcggcaagctgtgcggcctttgcggcgactacaactacgaccggcagcacgagctcgtgggccccaacctccaccactacaacgacaccctggagttcgccaagagctacgtcgttccctcttccgactgcaccccgccctga

SEQ ID NO: 44 - RNA, 4934 nt, Ixodes scapularis
  source 1..4934: mol_type = mRNA
gtaaagaactgcatcgtcatgagagtcccctacgttctgcctgtcctgttccttgctgcagcttcggccttcgaagtgggcaaggactacgtgtaccactacaacggaaagctacaagtctataacccggagcagcccctacagtcttcgggattcgcgttccagagcaaggtcgtcgcccaacccaggcccgaccacacgcacttcaagataatcgatttcgaggtggacaccttcaacggcgaacatgtccatctcagtgaccaccagttccactaccactccaccgacgcactgaagcaatttattgagagaccatttgccggcaagttctccgaaggaaagctcgaggccgcggagctgggaaagagcgagcccatgtggtctcagaacatcaagaagggtgtcctcagcgtgttccaactggacctcgtcaagggacgtcatgacgatccccacgccaagcagttctacgtgaaggaggacggccttcacggaaactgcgacaccctctacgttgtcgccgagaaagagggtcacctcgaggttaccaagatcaagaatcttgagaaatgcgacaaagaccacttcgccatctacgggaggattaaggcgcacgagtgcgtcgattgtgaagcccaggagtcgcaccccctcgtggccaccgcccaggtcaagtacaggctggatggaactccagagcactacgtcattgaccacgcctgcgccacctcggagactgtcttgaggccttacggcaaaggaaaaaccttcgttgtccagctcaaccgcaccttggatttggaggaggtgcacgacgccaacaccgacacccagctcccagaggacctggaaagggtggaccacctgagtcagacgcttccagtaggtgatcatgtcgaaacactccaagatctgaagaaggtcaaccacttcgtcgactactttcaactcacaaacaacaaagaaaagttcgtcgcgggtctgaaccacctcgctgcccttgagtacgaagatgacgacgtcaaggatgttcacggcaaggaaagcggaggcctccagttcctggtcttgttcaacgctctttccactctgcatttcgaagatgtcgtccaggtgtacgagcaagccgtcgccagcgcaccggaagcaagccaatctcacgttaagagactgttcctggacctgctgtcagctgctggaacgaaccctcaagttgcgttcggcctgcaactggtcaaggaagataagctctcggacgatgaggctgagcacttcttcaccaagttggcgctcaaccttaaggagaacagccctgctcttctgactgaacttgccgaggtgtgtgagcacgtgaagccgaaacgacaagtgtgggtcaactgtcagctcgccctaagtatcctcgcaggccaagagggttgcgttcgcgcaaagaccgacaaggaacacgacgagggattctgcaagccctctctggtgtcgcacttctttaactattcggtcaagcctgaagacaagaaggaccagccagaatataagaggaccgcgtacatcaaggctgctggcaacctagccaccaggggcgccgtgcattacctggaacgcttcatctccgataccaaccagccagagtacacgagggccgccgccttctgggcactgaggcgagctggatcgaagcaccccgaactggtgcgcgatatcgcacttcccgtgtacaagaacaagagcgagtcatcgtatctgcgtatcgcggcgttcacagccgtcatgaagacacatcccgacctctacctgctcaagtacatcggccacaacatcatcgacgaccctagcgaccaagtggcatcttacgtgaccagcctgttccgtagtttcgagaaatccaagtatccctgccaccaatcactagctcagcaccttcgctacgtcgtgccaatgtggaacgacgtgtaccggttctccaagcccccggactacaccaaatcgcatgtgcatctttcctctggttacgaccccaagtacgactacggtggcgccacgtccttcggtatggtccgggctgacgacagctacctcccacgagacgtctacgttagcgtcaaggactacatgagcggtcactccctcagcacggctaccttgtggtttgagaattggggcatggacaagctgttgaaccacgtcgtgggacctcagcccggatcctcaaagaacctgtggaacgtctttgggcgacgtcggtttaccagggatgcgtcggcgaaggagctcaaggaagtcgaggatgcgcttcccgtcaccgatcgggagtacgaccacgtctacggcagactgaacctcgaccttttcggtcacgccatcgacagctgggagttcgacgaggatcttctcaaagaagtcggcaaggaagacggagaaggcaagtgcttagaatctttgaagcttttaatattaactgacaggctaaggaaacacaaaccaggaaccaagctgaagaacctcttgggccaagtgcagcgcaagaaaaacttctacctgagcactgacataatctttgttgcgcccaccgaggttggtgttccaatcttcttcgacttcaagcaagccgaatttacgtacatcaaccgcaagaagttcagcgtggaccagactgaagacgccaagttcacttttgatgttcagaggcactacgtgcacgagagccgtggctacaccatggtcggcgtgggcctgacctttaacaagacgtccttgggaacggggtccgacgtccagaccatcttcaacctgccgatcgacctacacctcactgtggatcccattcacaagacgctgagcctgaagcgcccgctgagcctgccctggaacgtcctgaaccaccacttccgtccgttcacctttaccatgccctatgacatcgccacagatgtgtcccacgctgtcaccacactggagcagcctcagtaccccctctacaacaaggaagaagtaactgagattgatcatacctacctcaccgacgtcttcggctatggcttgaacgtgaagggtagcctcctcagcaaaggactcaagaaaggactgcacgacttctggtacgagagcgattgccgacagaggtactactatgccctcatgaatccagagtggcacccccgtgaactgcagttcaccgtcgttccagctgagcatgacgccacgactgaggtagaggtcgatctcggctacaagttcctcgaacccgatgacgcaagagagagccacttccccgttcaggacaacgtcggcggagatgctgaggttccttccacccatgtgctcagcctcgactacactctgaagggctccaaggagcgcaaggcgtccgcagaactgaggtactcgtttaccaaggacaacctcaagcacaaagtgcagttcttctatgaccggacgcccttccacccaaaggagcaagtccacactaaggtctgcatggatgtcacgtctaaattcccaaagcccgagtggtccaagttgaagaacctcgccgttttccacgagggcaaggatctggaatctgagctgaaccttcattacggaagcaactgcgtagaccagtctagcatcacattcaagggaaaatatactcacacggatgacgaagaaaggcagattcgagagaacgctgaaggcaagccgcggggaatcaaccgaatggacaagtataacctccgagatccgttcaacaaatgcaccgagcatcagaagcgtggcgttccactaaacctttactgcatgaagtacctgtactacaccagccgcctgggtaaattcacaacagacgtcgagtacaagaacctgaagccgttgctccccatgctaacgaagtactacaagaaggtccaccccgagggaggtttcctgagcaccgtagtgtcccacttccatggacccaacggaaagctccacgtcgtctctcaagtgcctcctgttcaggactacgcggacatcgttgtaaccacggaggacggtcactcccacaaccactcccacgtgccgctctacagccacttgctcgagcccaggatcatgttctccacgctagggtactcgaacatggctcactacatctcctactacaaacagaaatactgcgatctgcaaggacagagcgtcaggactttcgacaacgtcgtcgtgcagctgccggagaccgactgctacaaggtggtcgccaaggactgctcgcccaacaagaggttcacggtcctggcacgggctaccggaaacgccgaactccccaaggcgctgaaggcattcatccagaccacccaggtcgagctcctgccagttgccgaagactccggtcttgtgctccgcgttgacggaaacagagttccactgaccccgggagtaccttacagtcacaccgcgcacgacgccgagctcttcaccgtcacgctggaaaacaaatacttcgaagtaacgtcgaagccatacggtctcaatctaggcttcaacggaaacatgctcttcgtacagaccgccaacttctaccgcggcaagctgtgcggcctttgcggcgactacaactacgaccggcagcacgagctcgtgggccccaacctccaccactacaacgacaccctggagttcgccaagagctacgtcgttccctcttccgactgcaccccgccctgagcatcattttccgtcgacatcatcgttcaaccctatttcggccgctcttcgtgagcaggcattttcttcgataagtactgacacgaagggacagtgcgagagttgagcagcaactgtaacctctagaacgaacgtaacgccgcggaagcctcaagacaacgtgataaaggctttattggacgtgttcactgccatggacgttttagcgaagtaaaaaaagctaataaatgcattgaaaaaattgt

SEQ ID NO: 45 - protein, 1537 aa, Ixodes scapularis
MRVPYVLPVLFLAAASAFEVGKDYVYHYNGKMQVYNPEQPLQSSGFAFRSKVVAQPRPDHTHFKIIDFEVDSFNGDHVHVGEHEFNYHSTEALKQFIERPFAGKFSEGKLEEAELSKSEPKWARNLKKGVLSIFQLDLVKGRHDNPHAKQFHVREEGLHGNCDTLYVVAEEEGHLKVTKIKNLEKCDKEHYAVYGRIKGHECVDCEAQETHPFVATSQVKYRLDGTPEHYVINHACATSENVFRPFGQGKTFVAQLNRTLDLEEVHDANTDTQLPEDLEKVHHIAQAFPESDEVESLEELKHVNRYVTTFELSTDKDKFISGLNHLAALEYEDSDIKDVHSKESGGLNFLILFGSLASMPFEDIAHVYEQAVANAPEASKSQVRKVFLDLLSAVGNNPHAAFGLQLVKEDKLTDEEAEHFLAKLALNLKENSPALLTELAEVCEHVKPKRPVWVNCQLALSSLAGQEGCVRAKTDKEHDEGFCKPSIVSHFFNYEIKPEDKKDQPEYKRTVYMKAAGNLATRGAVHYLERYVSDTNQPEYRRSAALWAMVRAAPHHHELVRDVALPVYKNKSETAYLRIGAFVNVLMTKPDLYLLKYIGHNIIDDPSDQLASYVTSAFRSLVKSKYPCHQELAQHLRYVVPMWNDVSRFAKPLDHTKSQVILSSGYDPKFDYGGSSVFAQVRADDSYLPREVYFSAKDYFHGRSFESVSLKFESWGLDKLLNHVVGPQPGSTKNLWNVFGRRRFTRDASAKDLQEVEAALPITDRDYDHVYGRLSLDLYGHAFDTWEFDESILQELSKGEGEEAPGDKLKNLLGESKRKKAFILSKDICIIVPSELGVPVFYDIKQVEYMYINPKKFAFDQTQDAKFAMDVERHYVHESRGFSMFGVALTFNKTSLGTGSDSQTIINLPIDLKITLDPIHKKLSMKRPLSLPWNVLNHHFRPFTFVMSYDINEDVTTAETKMTRAMYPLYNKEELTEFDRNYFGEVLGYGLNVKGSLLSKGLKKGLHDYWYESDDRQKFYYATLNPEWHPRELQFTVVPAAQDATTEVEVDLGYKFLEPDDPRESHFEAHDNVGDDAEVPSTHVLNLDYTLKGTKERKVSAELRYSFTKDLLKHKVQLFYDRTPFHPKEQDHTKVCLDVISKFPEPDWERLNNLAVFHEGKDIESQLNLHYGSNCVDQSSITFKGKYTHTDDEEKQIRENAEGKPLGINRMKKYTLRRPYKKCTEHQKRGVPLNFECMKYLYYTSRLGKLTTDVEYKNLKPLFPMLLKYYKKVHKEGGFLSTLASHVHGPTGKLHVVSQVPPVEKYSDIVVTTEDGHSFHHDHVPIYSHLLEPRIFSVLGYSNMVNYISYYKQKHCDLQGKSLRTFDNVVVNLPETDCFKVVAKDCSPNKRFTVLARATGNAALPKALKAFIQSTKVELLPVSADSGLVLRVDGNKVDLTAGVPYSHTAHDVELFTVMQHNKYFEVMSQPYGVFIGFDGNVLFVQTANFYRGKLCGLCGDYNYDRQHELVGPNLHHYNDTLEFAKSYVVPASDCTAP

SEQ ID NO: 46 - RNA, 4614 nt, Ixodes scapularis
  source 1..4614: mol_type = mRNA
atgagagtcccctacgttctgcccgtcctgttccttgccgccgcttcggccttcgaagtgggcaaggactatgtgtaccactacaacggaaagatgcaagtgtacaaccccgagcagcccctgcagtcctcgggtttcgccttccgaagcaaggtcgtcgcccagcccaggcccgaccatacgcacttcaagataatcgactttgaggtggacagcttcaacggtgatcatgttcatgtcggtgaacacgagttcaactaccactccacagaggcgctgaagcaattcatcgagaggcccttcgccggcaagttctccgaaggaaagctcgaagaagctgagctgagcaagagcgaacctaagtgggccaggaacctcaagaagggagtcctcagcattttccagctggacttggtcaagggtcgccatgacaacccccacgccaagcagttccacgtgagagaggagggtctccacgggaactgcgacacactctacgttgtcgccgaggaggaaggtcacctcaaggtcaccaagatcaagaatctcgagaaatgcgacaaggaacactacgccgtttatggaaggatcaagggacatgagtgcgtcgactgcgaagcccaggagactcaccctttcgtagctacctcccaggtcaagtacagactcgacggaacgccagaacactacgtcatcaaccacgcctgcgccacctcagagaatgtcttcaggcccttcggtcaagggaaaaccttcgttgcccagctcaaccgcaccttggatttggaggaggtgcacgacgccaacaccgacactcagcttccagaggacctggagaaggtgcaccacatcgcgcaggcattccccgagtcggatgaggtggaaagcctggaggaactgaagcacgtcaaccgctacgtgaccacctttgagctctcgaccgacaaagacaagttcatctctggtctcaaccaccttgctgcattggagtacgaagattccgacatcaaggacgttcacagcaaggaaagcggtggactcaacttcctgattttgttcggatcgcttgcctccatgccctttgaagatatcgcccacgtgtacgagcaagccgttgccaatgcaccggaagcaagcaagagtcaagtcagaaaggtgttcctggacctgctctcagctgtcggaaacaaccctcacgcagcgttcggtctgcaattggtcaaggaagacaaactcactgacgaggaggccgagcacttcctcgccaagctggcgctgaacctaaaggagaacagccccgctcttctgaccgaacttgccgaggtgtgtgagcacgtgaagcctaaacgaccagtgtgggtcaactgccaactcgctctgagtagcctcgctggccaagagggctgcgtccgtgccaagactgacaaggaacacgacgagggattctgcaagccctctattgtttcgcacttctttaactatgagattaagccagaagacaagaaggaccagccagaatacaagaggacggtgtacatgaaggctgcgggcaacttggccaccaggggcgccgtgcactacctggagcgctacgtctcggacacaaaccagccggagtaccggaggtcagccgccctctgggcaatggttcgagctgctccgcaccaccacgaactggtacgcgacgtcgcactcccagtgtacaaaaacaagagcgagacggcctacctgcgtatcggggcatttgtgaacgtcctaatgaccaaacccgacctctacctgctcaagtacatcggacacaacatcatcgacgaccccagcgaccagctggcgtcctacgtgaccagcgccttccgcagccttgtgaaatccaagtacccgtgccaccaagaactggctcagcaccttcgctacgtcgtgcccatgtggaatgacgtaagccggtttgccaagccgctggaccataccaaatcgcaagtcatcctctcttccggctacgatcccaagttcgactacggcggctccagtgtcttcgcccaagtacgggccgacgacagctacctaccgagagaagtgtatttcagcgcaaaggactatttccacggccgctccttcgagtcagtttcactaaagtttgagagctggggtctggacaagcttctgaaccacgtcgtgggacctcagcccggatccaccaagaacctgtggaacgtctttgggcgacgtcgctttaccagggacgcctcggcaaaggatctccaggaagttgaggctgctcttcccatcaccgaccgggattatgaccatgtctacggccgcctgagtctcgatctctacggtcacgccttcgacacctgggaattcgacgagagcattctgcaggaactgagcaagggagagggagaagaggcaccaggcgacaagctgaagaacctgctaggcgaatcaaagcgtaagaaggccttcatcctcagcaaggacatctgcattattgtacccagcgaacttggtgttcccgtattctacgacatcaagcaagtcgaatacatgtacatcaaccccaagaagtttgccttcgatcaaacgcaagatgccaaatttgccatggacgtagaaaggcactacgtacacgaaagccgtggcttcagcatgttcggtgttgctctcaccttcaacaagacgtccttgggaacgggctccgatagtcagaccatcataaatctgccgatcgacctgaagattaccctggatcctattcacaagaagctgagtatgaagcgcccgctgagcctgccctggaacgtcctgaaccaccacttccgcccgtttacctttgtcatgtcctacgacataaacgaagatgttaccacagccgaaaccaagatgacccgggctatgtaccctctctacaacaaggaagaacttaccgagtttgatcgtaactacttcggcgaggtcttgggctatggcctgaacgtgaagggaagcctcctcagcaaggggctcaagaagggactgcacgactactggtacgagagcgatgatcgccagaagttctactatgccactctaaaccctgagtggcacccacgtgaactgcagttcaccgtcgttccagctgcacaagatgccacaactgaggtagaggtcgatctcggctacaagttcctcgagcccgatgacccacgggagagtcacttcgaagctcacgacaacgtcggcgacgatgctgaggttccgtccactcacgtgctgaacctcgactacactctgaagggcaccaaggaacgcaaggtgtccgcggaactgagatactccttcaccaaggacctcctcaagcacaaagtacagctcttctatgaccgaacaccattccacccgaaggagcaagaccacacgaaggtctgcctggatgtcatatcgaagttcccagagcccgactgggaaaggctgaataacctcgccgttttccatgagggcaaggacattgaatctcagctgaacctccactacggaagcaactgtgttgaccagtctagcattacttttaagggaaagtatacgcacacggacgacgaggagaagcagattcgagagaacgcagaaggcaagccgctgggaatcaaccgaatgaagaagtacaccctgcgtcggccgtacaaaaagtgcaccgagcaccagaagcggggcgttcccctaaactttgagtgcatgaagtacctgtactacaccagtcgcctgggtaaattgacaacggacgtcgagtacaagaacctcaagccgctgttccccatgctgctgaagtactacaagaaggtccacaaggagggcggtttcctaagcaccctcgcgtcccacgtccacggacccaccggaaagctccacgtcgtctctcaagtgcctccggttgagaagtactcggacattgttgtgaccaccgaggacggtcactccttccaccatgaccacgttcccatctacagccacttgctcgagcctaggattttctctgtgctgggatactcgaacatggttaactacatctcctactacaaacagaaacattgcgatcttcaaggcaagagcctccgaaccttcgacaacgtcgtcgtgaaccttcccgagactgactgcttcaaggtggttgccaaggactgctctcccaacaagaggttcacggttctggcacgagctaccggaaacgccgcactccccaaggcgttgaaggccttcatccagtcaaccaaggtcgagctcctgccagtttccgccgattctggactggtgctccgcgtcgacggcaacaaggtggatctgacggcgggagttccttacagccacaccgcgcacgatgtcgagctcttcacggtcatgcagcacaacaagtactttgaagtgatgtcgcagccttacggggtcttcattggcttcgacgggaacgtgctctttgtgcagaccgccaacttctaccgcggcaagctgtgcggtctttgcggcgactacaactacgaccggcagcacgagctcgtgggccccaacctccaccactacaacgacaccctggagttcgccaagagctacgtcgttcccgcgtccgactgcaccgcgccttga

SEQ ID NO: 47 - RNA, 4915 nt, Ixodes scapularis
  source 1..4915: mol_type = mRNA
atgccttcagtgctcactcctcggggaatgggagcgagttgctaaggaactgcatcgtcatgagagtcccctacgttctgcccgtcctgttccttgccgccgcttcggccttcgaagtgggcaaggactatgtgtaccactacaacggaaagatgcaagtgtacaaccccgagcagcccctgcagtcctcgggtttcgccttccgaagcaaggtcgtcgcccagcccaggcccgaccatacgcacttcaagataatcgactttgaggtggacagcttcaacggtgatcatgttcatgtcggtgaacacgagttcaactaccactccacagaggcgctgaagcaattcatcgagaggcccttcgccggcaagttctccgaaggaaagctcgaagaagctgagctgagcaagagcgaacctaagtgggccaggaacctcaagaagggagtcctcagcattttccagctggacttggtcaagggtcgccatgacaacccccacgccaagcagttccacgtgagagaggagggtctccacgggaactgcgacacactctacgttgtcgccgaggaggaaggtcacctcaaggtcaccaagatcaagaatctcgagaaatgcgacaaggaacactacgccgtttatggaaggatcaagggacatgagtgcgtcgactgcgaagcccaggagactcaccctttcgtagctacctcccaggtcaagtacagactcgacggaacgccagaacactacgtcatcaaccacgcctgcgccacctcagagaatgtcttcaggcccttcggtcaagggaaaaccttcgttgcccagctcaaccgcaccttggatttggaggaggtgcacgacgccaacaccgacactcagcttccagaggacctggagaaggtgcaccacatcgcgcaggcattccccgagtcggatgaggtggaaagcctggaggaactgaagcacgtcaaccgctacgtgaccacctttgagctctcgaccgacaaagacaagttcatctctggtctcaaccaccttgctgcattggagtacgaagattccgacatcaaggacgttcacagcaaggaaagcggtggactcaacttcctgattttgttcggatcgcttgcctccatgccctttgaagatatcgcccacgtgtacgagcaagccgttgccaatgcaccggaagcaagcaagagtcaagtcagaaaggtgttcctggacctgctctcagctgtcggaaacaaccctcacgcagcgttcggtctgcaattggtcaaggaagacaaactcactgacgaggaggccgagcacttcctcgccaagctggcgctgaacctaaaggagaacagccccgctcttctgaccgaacttgccgaggtgtgtgagcacgtgaagcctaaacgaccagtgtgggtcaactgccaactcgctctgagtagcctcgctggccaagagggctgcgtccgtgccaagactgacaaggaacacgacgagggattctgcaagccctctattgtttcgcacttctttaactatgagattaagccagaagacaagaaggaccagccagaatacaagaggacggtgtacatgaaggctgcgggcaacttggccaccaggggcgccgtgcactacctggagcgctacgtctcggacacaaaccagccggagtaccggaggtcagccgccctctgggcaatggttcgagctgctccgcaccaccacgaactggtacgcgacgtcgcactcccagtgtacaaaaacaagagcgagacggcctacctgcgtatcggggcatttgtgaacgtcctaatgaccaaacccgacctctacctgctcaagtacatcggacacaacatcatcgacgaccccagcgaccagctggcgtcctacgtgaccagcgccttccgcagccttgtgaaatccaagtacccgtgccaccaagaactggctcagcaccttcgctacgtcgtgcccatgtggaatgacgtaagccggtttgccaagccgctggaccataccaaatcgcaagtcatcctctcttccggctacgatcccaagttcgactacggcggctccagtgtcttcgcccaagtacgggccgacgacagctacctaccgagagaagtgtatttcagcgcaaaggactatttccacggccgctccttcgagtcagtttcactaaagtttgagagctggggtctggacaagcttctgaaccacgtcgtgggacctcagcccggatccaccaagaacctgtggaacgtctttgggcgacgtcgctttaccagggacgcctcggcaaaggatctccaggaagttgaggctgctcttcccatcaccgaccgggattatgaccatgtctacggccgcctgagtctcgatctctacggtcacgccttcgacacctgggaattcgacgagagcattctgcaggaactgagcaagggagagggagaagaggcaccaggcgacaagctgaagaacctgctaggcgaatcaaagcgtaagaaggccttcatcctcagcaaggacatctgcattattgtacccagcgaacttggtgttcccgtattctacgacatcaagcaagtcgaatacatgtacatcaaccccaagaagtttgccttcgatcaaacgcaagatgccaaatttgccatggacgtagaaaggcactacgtacacgaaagccgtggcttcagcatgttcggtgttgctctcaccttcaacaagacgtccttgggaacgggctccgatagtcagaccatcataaatctgccgatcgacctgaagattaccctggatcctattcacaagaagctgagtatgaagcgcccgctgagcctgccctggaacgtcctgaaccaccacttccgcccgtttacctttgtcatgtcctacgacataaacgaagatgttaccacagccgaaaccaagatgacccgggctatgtaccctctctacaacaaggaagaacttaccgagtttgatcgtaactacttcggcgaggtcttgggctatggcctgaacgtgaagggaagcctcctcagcaaggggctcaagaagggactgcacgactactggtacgagagcgatgatcgccagaagttctactatgccactctaaaccctgagtggcacccacgtgaactgcagttcaccgtcgttccagctgcacaagatgccacaactgaggtagaggtcgatctcggctacaagttcctcgagcccgatgacccacgggagagtcacttcgaagctcacgacaacgtcggcgacgatgctgaggttccgtccactcacgtgctgaacctcgactacactctgaagggcaccaaggaacgcaaggtgtccgcggaactgagatactccttcaccaaggacctcctcaagcacaaagtacagctcttctatgaccgaacaccattccacccgaaggagcaagaccacacgaaggtctgcctggatgtcatatcgaagttcccagagcccgactgggaaaggctgaataacctcgccgttttccatgagggcaaggacattgaatctcagctgaacctccactacggaagcaactgtgttgaccagtctagcattacttttaagggaaagtatacgcacacggacgacgaggagaagcagattcgagagaacgcagaaggcaagccgctgggaatcaaccgaatgaagaagtacaccctgcgtcggccgtacaaaaagtgcaccgagcaccagaagcggggcgttcccctaaactttgagtgcatgaagtacctgtactacaccagtcgcctgggtaaattgacaacggacgtcgagtacaagaacctcaagccgctgttccccatgctgctgaagtactacaagaaggtccacaaggagggcggtttcctaagcaccctcgcgtcccacgtccacggacccaccggaaagctccacgtcgtctctcaagtgcctccggttgagaagtactcggacattgttgtgaccaccgaggacggtcactccttccaccatgaccacgttcccatctacagccacttgctcgagcctaggattttctctgtgctgggatactcgaacatggttaactacatctcctactacaaacagaaacattgcgatcttcaaggcaagagcctccgaaccttcgacaacgtcgtcgtgaaccttcccgagactgactgcttcaaggtggttgccaaggactgctctcccaacaagaggttcacggttctggcacgagctaccggaaacgccgcactccccaaggcgttgaaggccttcatccagtcaaccaaggtcgagctcctgccagtttccgccgattctggactggtgctccgcgtcgacggcaacaaggtggatctgacggcgggagttccttacagccacaccgcgcacgatgtcgagctcttcacggtcatgcagcacaacaagtactttgaagtgatgtcgcagccttacggggtcttcattggcttcgacgggaacgtgctctttgtgcagaccgccaacttctaccgcggcaagctgtgcggtctttgcggcgactacaactacgaccggcagcacgagctcgtgggccccaacctccaccactacaacgacaccctggagttcgccaagagctacgtcgttcccgcgtccgactgcaccgcgccttgagcaccgtcttgcgtcaacatcgtcgttcaaccttatttctgccgctccttgcgagccggtattttcattcattagtaacgagatgatgtgaaagtgcaagtgtcgtgtcaagactgtagttgttagaataatggcaaagccccgagagcctcgagacaacgggatgacggtttgtgtgaacgtttgcaatgccatgggcgcttgttcgacatgagaaaaacaaataaatacatgaagcagaa

SEQ ID NO: 48 - protein, 812 aa, Ixodes scapularis
MRLSYWNVAAALACFLLLVSIGQAQRRGNGGRGRGQGRSRGQSQRQQAAPAAAPAANKPASDCTLSTGEPGICQPLSHCVFQFNAIDDLALSKCGATTKVCCPLNTPAATGTLEFSRPVTVTARRVSIPKISKDDLDIAGRASLSIVRGIEDAERTLLNDGKFAKKRTTASLHAAFFGSKPIVQSLGRDGLIGLEATRELARKFNLDSTQGRDGLQRFNIQNTVISDACPPVPRCPSTKYRSPDGRCNNLQNREWGKSLTAFERFMPPNYADGLTLPRVSLDGNPLPNPRTISVAADPDGDFPNERNTLMLMQFAQFVDHDLTLTGVTRFRNGSAITCCDEEFLTNPTKRHFACMPIDLDANDHFYSEFNLRCIEFVRSVPAPRPQCTFGPREQLNQLTAYMDSSNIYGSTEEEAKSLRSFRDGRLASTFFSRDELLPRQTDGTQECNEQGTDFVCFRAGDERVNEQVSLTAMHTLWLREHNRVAGELHRLNPGWKDEILYQEARRIVAAEFQHICFNEFLPLLLGRKVMEQFDLLLTPYGYSHSYDPNLNAGIGNVFATAAYRYGHTLVQGNIELINEDGSVHKRIPLAAQFFNPNEIYHPGNLDRFFRGLITQPAQTYDRFVTQQLTNHLYEPIGQGFGMDLVALNIQRGRDHGIPSYNDWREHCGMSRITDFAQLADIMTPESAKAFAQVYKYPDDIDLFPAGVNEKSVPGGTLGPTFACLVAEQFRRMKNGDRFWYENGGLESSFNEVQIEEIRKASLARVICDNSNLNYVQPLVMIREAQWNPKVDCNGEDIPRVSLDNWQNEPVWT

SEQ ID NO: 49 - RNA, 2439 nt, Ixodes scapularis
  source 1..2439: mol_type = mRNA
atgcggctctcctactggaacgtggccgctgctctggcctgcttcctgctcctggtcagcatcggtcaggcacagcgaagaggaaatggcggccgaggtcgcggccagggccgtagtcggggccagagccagcgccagcaggctgcgccggcggcagcccccgcggccaacaagccggcgtccgactgcacgctgtccacgggcgagcccgggatctgccagcccctgagccactgcgtattccagttcaacgccatcgatgacctggcgctgtccaagtgcggcgccacgaccaaagtttgctgcccgctgaacacgccagctgccaccggaacactggagttcagtcgcccggtgacggtgacggcgaggagggtgtccatccccaagatcagcaaggacgacctggacatcgccggcagagccagtctcagcatcgtccgcggaattgaagacgccgagaggacccttctcaatgatggcaagttcgcgaaaaaacgaacaacagcgtcgctgcacgccgctttcttcgggtccaagcccatcgtgcagtctcttggaagggacggcctcatcggactggaagccaccagggaactggcaagaaagttcaacctggactcgacgcagggccgcgatggcctgcagcgcttcaacatccagaacacggtgatctcggacgcctgtcctcccgtgccgcgctgcccctcgaccaagtaccggtcccccgacggacgctgcaacaacctccagaacagggagtggggcaagtccctcaccgccttcgagaggttcatgccgcccaactacgccgacggcctgaccctgccccgagtgagcttggacggcaacccgctgcccaacccccgcaccatctcggtggccgccgatcccgacggagacttccccaacgagcggaacacgctgatgctcatgcagttcgctcagttcgtcgaccacgacctcacgctcaccggagtcaccagattccgcaacggctcggccatcacctgctgcgacgaggagttccttacgaacccgaccaaaaggcacttcgcctgcatgcccatcgacctggatgccaacgatcacttctactcagagttcaacctcaggtgcatcgagttcgtacgctccgtgccggcgccccgaccacagtgcacgttcggacctcgtgaacagttgaaccagctgactgcttacatggactcctccaacatctacggctcgacggaagaagaagccaagtccctgcggagcttcagggacggtcggctggcgtccacgttcttcagcagggacgagctgctgcctcgtcaaacagatggcacgcaggagtgcaacgaacaagggaccgacttcgtttgcttcagggcaggagacgagcgcgtgaacgagcaggtgagcctgacggcgatgcacacgctgtggctacgagagcacaaccgggtcgccggagagctgcaccgcctcaacccgggctggaaggacgagatcctgtaccaggaggccaggaggatcgttgccgcagagttccagcacatctgcttcaacgagtttctgccactgctcctgggtcgcaaagtgatggaacagttcgacctgctgcttacgccgtacggatacagccatagctacgatcccaatctgaacgccggcattggcaacgtgttcgccaccgctgcatacagatacgggcacaccctcgtgcagggcaacatcgagctgatcaacgaggacggatccgtgcacaagcggataccgctggcggcccagttcttcaatccgaacgagatctatcacccgggcaacctggatcgcttcttcaggggcctgatcacgcagcctgcacagacctacgatcgcttcgtcacgcaacaactgacgaaccacctgtacgagcccatcggccagggcttcggtatggacctggtggcgttgaacatccagcggggccgagaccacggcatccccagctacaacgactggcgggaacactgcggcatgtccaggattacggacttcgcccagctggccgacatcatgacgccggagtccgccaaggccttcgcacaagtctacaagtaccccgacgacatcgacttgttcccggctggcgtgaacgagaaatctgtgcccggcggcacgctaggacccaccttcgcctgtctcgtggccgagcagttccgacggatgaagaacggcgaccgcttctggtacgagaacggagggctcgagtcgtccttcaacgaagttcaaatcgaggaaattcgaaaggccagcctggcgcgggtcatctgcgacaactccaacctgaactacgttcagcctctggtcatgatcagagaagcgcagtggaacccgaaggtggattgcaatggcgaagacattccgagagtgagcttggacaactggcagaacgagccggtctggacgtag

SEQ ID NO: 50 - RNA, 2638 nt, Ixodes scapularis
  source 1..2638: mol_type = mRNA
atgcggctctcctactggaacgtggccgctgctctggcctgcttcctgctcctggtcagcatcggtcaggcacagcgaagaggaaatggcggccgaggtcgcggccagggccgtagtcggggccagagccagcgccagcaggctgcgccggcggcagcccccgcggccaacaagccggcgtccgactgcacgctgtccacgggcgagcccgggatctgccagcccctgagccactgcgtattccagttcaacgccatcgatgacctggcgctgtccaagtgcggcgccacgaccaaagtttgctgcccgctgaacacgccagctgccaccggaacactggagttcagtcgcccggtgacggtgacggcgaggagggtgtccatccccaagatcagcaaggacgacctggacatcgccggcagagccagtctcagcatcgtccgcggaattgaagacgccgagaggacccttctcaatgatggcaagttcgcgaaaaaacgaacaacagcgtcgctgcacgccgctttcttcgggtccaagcccatcgtgcagtctcttggaagggacggcctcatcggactggaagccaccagggaactggcaagaaagttcaacctggactcgacgcagggccgcgatggcctgcagcgcttcaacatccagaacacggtgatctcggacgcctgtcctcccgtgccgcgctgcccctcgaccaagtaccggtcccccgacggacgctgcaacaacctccagaacagggagtggggcaagtccctcaccgccttcgagaggttcatgccgcccaactacgccgacggcctgaccctgccccgagtgagcttggacggcaacccgctgcccaacccccgcaccatctcggtggccgccgatcccgacggagacttccccaacgagcggaacacgctgatgctcatgcagttcgctcagttcgtcgaccacgacctcacgctcaccggagtcaccagattccgcaacggctcggccatcacctgctgcgacgaggagttccttacgaacccgaccaaaaggcacttcgcctgcatgcccatcgacctggatgccaacgatcacttctactcagagttcaacctcaggtgcatcgagttcgtacgctccgtgccggcgccccgaccacagtgcacgttcggacctcgtgaacagttgaaccagctgactgcttacatggactcctccaacatctacggctcgacggaagaagaagccaagtccctgcggagcttcagggacggtcggctggcgtccacgttcttcagcagggacgagctgctgcctcgtcaaacagatggcacgcaggagtgcaacgaacaagggaccgacttcgtttgcttcagggcaggagacgagcgcgtgaacgagcaggtgagcctgacggcgatgcacacgctgtggctacgagagcacaaccgggtcgccggagagctgcaccgcctcaacccgggctggaaggacgagatcctgtaccaggaggccaggaggatcgttgccgcagagttccagcacatctgcttcaacgagtttctgccactgctcctgggtcgcaaagtgatggaacagttcgacctgctgcttacgccgtacggatacagccatagctacgatcccaatctgaacgccggcattggcaacgtgttcgccaccgctgcatacagatacgggcacaccctcgtgcagggcaacatcgagctgatcaacgaggacggatccgtgcacaagcggataccgctggcggcccagttcttcaatccgaacgagatctatcacccgggcaacctggatcgcttcttcaggggcctgatcacgcagcctgcacagacctacgatcgcttcgtcacgcaacaactgacgaaccacctgtacgagcccatcggccagggcttcggtatggacctggtggcgttgaacatccagcggggccgagaccacggcatccccagctacaacgactggcgggaacactgcggcatgtccaggattacggacttcgcccagctggccgacatcatgacgccggagtccgccaaggccttcgcacaagtctacaagtaccccgacgacatcgacttgttcccggctggcgtgaacgagaaatctgtgcccggcggcacgctaggacccaccttcgcctgtctcgtggccgagcagttccgacggatgaagaacggcgaccgcttctggtacgagaacggagggctcgagtcgtccttcaacgaagttcaaatcgaggaaattcgaaaggccagcctggcgcgggtcatctgcgacaactccaacctgaactacgttcagcctctggtcatgatcagagaagcgcagtggaacccgaaggtggattgcaatggcgaagacattccgagagtgagcttggacaactggcagaacgagccggtctggacgtagggccaccgactctcgcggggggaacttttatttgttgtgttccccaatattccctggcattactcccctaccccacgatgttgttcacttgccgcgttttttcctcgttgctttcttgttgcacgcatgtgacgatgccccgcgatattcgtttcattaaaggtcacacgagcgcacgtgcgcttcattctttcatcgt

SEQ ID NO: 51 - protein, 218 aa, Ixodes scapularis
MLSALSLALLCAVGAGVGPVAQAAAPLQIDCVQDESTERLQNFTFKDVLEKDTIPLSRFKGYVALVVNVATYUGLTPTYLQLNALQARFGERNFTVLGFPCNQFGKQEPGTRQEILNGIRYVRPGNNYVPNFPMFQKIEVNGENQHPLYTFLKGRCTSPNPVFSAKDKLFYSPQNNNDIRWNFEKFLVDRRGVPVKRYEPRYSPDEVARDIEVLTRSS

SEQ ID NO: 52 - RNA, 657 nt, Ixodes scapularis
  source 1..657: mol_type = mRNA
atgttgtcggcgctctcgctagcgctgctctgcgcggtcggggccggcgtggggccggtcgcacaggccgcggcaccgctccagatcgactgcgtccaagacgagtccaccgagcgtctgcaaaacttcaccttcaaggatgtcctggagaaggacaccatccccctgagccggttcaagggctacgtcgctctggtggtgaacgtggccacctactgaggcctgaccccgacctacctgcaactgaatgcactgcaggccaggttcggggaaaggaacttcaccgtgctcgggtttccctgcaaccagttcggaaagcaagagcccggtacccgccaggagattctgaacgggatcaggtacgtgcgtccgggcaacaactacgtgcccaactttcccatgttccagaagatcgaggtgaacggggaaaaccagcacccgctctacactttcctcaagggtcgctgcacctcgccgaatccggttttcagcgccaaggataaactcttctactcgccccagaacaacaacgacatccgttggaacttcgagaagtttctggttgacaggcgaggagtgccggtgaaacgctacgagccccgctactcgcccgacgaggtcgctcgcgacatcgaggtcctcacaagatcgagctag

SEQ ID NO: 53 - protein, 96 aa, Ixodes scapularis
MKATLAVLCFLVAVACAIVVEEKMVSHPIDEEALDPRCVKPVDCPGNEKEVSFYDPRRGCQLIRLGENCTHNDNYPTLDLCNEHCPPAPGTHPRRA

SEQ ID NO: 54 - RNA, 291 nt, Ixodes scapularis
  source 1..291: mol_type = mRNA
atgaaggcaacccttgcagttctttgctttcttgtagcagtagcgtgcgccattgtggtggaggagaagatggtgtcccatcccattgacgaagaggctctcgatccaagatgcgtaaagccggtggactgccccggaaatgaaaaggaggtttccttttacgacccacgaaggggatgtcaactaattagactgggggaaaactgtacccacaatgacaattacccaacacttgatctttgcaatgaacattgcccacctgctccagggacgcacccgagacgcgcctga

SEQ ID NO: 55 - DNA, 57 bp, synthetic construct
  source 1..57: mol_type = other DNA
atgcgcatgcagctgctgctgctgatcgccctgtccctggccctggtgaccaactcc

SEQ ID NO: 56 - DNA, 20 bp, synthetic construct
  source 1..20: mol_type = other DNA
ttcaatcaggtaacggcaca

SEQ ID NO: 57 - DNA, 20 bp, synthetic construct
  source 1..20: mol_type = other DNA
gacgcttgagaccctgaaag

SEQ ID NO: 58 - DNA, 18 bp, synthetic construct
  source 1..18: mol_type = other DNA
agcgggaaatcgtgcgtg

SEQ ID NO: 59 - DNA, 19 bp, synthetic construct
  source 1..19: mol_type = other DNA
cagggtacatggtggtgcc

(19) United States
(12) Patent Application Publication
(10) Pub. No.: US 2025/0114439 A1
(43) Pub. Date: Apr. 10, 2025

(54) mRNA VACCINES AGAINST TICK SALIVARY PROTEINS, AND METHODS OF USING SAME

(71) Applicants: The Trustees of The University of Pennsylvania, Philadelphia, PA (US); Yale University, New Haven, CT (US)

(72) Inventors: Drew Weissman, Wynnewood, PA (US); Erol Fikrig, New Haven, CT (US); Sukanya Narasimhan, New Haven, CT (US)

(21) Appl. No.: 18/684,458

(22) PCT Filed: Aug. 18, 2022
(86) PCT No.: PCT/US2022/075128

§ 371 Date: Feb. 16, 2024

Related U.S. Application Data
(60) Provisional application No. 63/234,508, filed on Aug. 18, 2021.

(51) Int. Cl. A61K 39/00; A61P 37/04

(52) U.S. Cl. CPC A61K 39/0003; A61P 37/04; A61K 2039/53; A61K 2039/545

(57) ABSTRACT

Provided are nucleoside-modified mRNA molecules encoding tick salivary antigens, and LNP vaccines comprising the mRNA molecules and methods of use thereof to treat or prevent diseases or disorders associated with a tick-borne pathogen including Lyme disease.

### CROSS REFERENCE TO RELATED APPLICATIONS

This application claims priority to U.S. Provisional Patent Application No. 63/234,508, filed Aug. 18, 2021, which is hereby incorporated by reference herein in its entirety.

### BACKGROUND OF THE INVENTION

Tick-borne diseases are currently increasing in North America and Europe. The black-legged tick, Ixodes scapularis, transmits diverse pathogens, including Borrelia burgdorferi (the Lyme disease agent), Babesia microti, Anaplasma phagocytophilum, Borrelia miyamotoi, and Powassan virus, among other infectious agents (Eisen et al., 2018, Trends Parasitol, 34:295-309; Nelder et al., 2016, Parasit Vectors, 9:265; Nelson et al., 2015, Emerg Infect Dis, 21:1625-1631). As one example, the most common I. scapularis-borne human illness in the United States, Lyme disease, results in almost 40,000 cases reported annually, and the CDC estimates that the real number of infections may be 10 times greater (Nelson et al., 2015, Emerg Infect Dis, 21:1625-1631; CDC, 2018, cdc.gov/lyme/datasurveillance/tables-recent.html; Rosenberg et al., 2018, MMWR Morb Mortal Wkly Rep, 67:496-501). Given the identification of new types of tick-borne pathogens along with their remarkable diversity and generally unsuccessful efforts towards the development of single, pathogen-specific vaccines, the development of a broad anti-tick vaccine strategy is highly desirable. A possible approach to the prevention of one or more I. scapularis-borne diseases involves developing new methods to detect the ticks early-on and preventing the arthropod from taking a successful blood meal.

The ability of animals to develop acquired resistance to tick bites following repeated exposure to ticks—so-called “acquired tick resistance” or “tick immunity”—was first described by Trager in 1939 (Trager, 1939, The Journal of Parasitology, 25:57-81). Tick immunity is associated with the recruitment of inflammatory cells to the tick bite site, including basophils which degranulate to secrete histamine, that alter tick feeding (Willadsen et al., 1999, Parasitol Today, 15:258-262; Tabakawa, et al., 2018, Front Immunol, 9:1540; Narasimhan, et al., 2019, Ticks Tick Borne Dis, 10:386-397; Brown, et al., 1981, J Immunol, 127:2163-2167; Karasuyama, et al., 2018, Front Physiol, 9:1769. Interestingly, the phenomenon of naturally acquired resistance to I. scapularis has most substantially been observed in animals that are not important in the natural life cycle of this tick, including guinea pigs, rabbits, and cows, among other animals (Wikel, 1996, Annu Rev Entomol, 41:1-22; Willadsen, in Advances in Parasitology, W. H. R. Lumsden, R. Muller, J. R. Baker, Eds. (Academic Press, 1980), vol. 18, pp. 293-313). Dermal hypersensitivity following repeated tick exposure in humans has been described, suggesting an association with acquired resistance to I. scapularis (Burke, et al., 2005, Emerg Infect Dis, 11:36-41; Krause, et al., 2009, Vector Borne Zoonotic Dis, 9:603-610). Tick immunity in guinea pigs also provides protection against I. scapularis-transmitted B. burgdorferi infection, indicating that understanding this process further can lead to new vaccine strategies to prevent Lyme disease, and possibly other tick-borne infections (Nazario, et al., 1998, Am J Trop Med Hyg, 58:780-785; Narasimhan, et al., 2007, PLoS One, 2:e451).

In contrast to guinea pigs, some animals that serve as a natural reservoir for I. scapularis, such as mice, do not readily develop robust resistance upon repeated exposure to I. scapularis (Tabakawa, et al., 2018, Front Immunol, 9:1540; Narasimhan, et al., 2019, Ticks Tick Borne Dis, 10:386-397; Wikel, 1996, Annu Rev Entomol, 41:1-22; Anderson, et al., 2017, Front Immunol, 8:1784). Mice, however, are not a natural reservoir for Haemaphysalis longicornis, and it has been demonstrated that laboratory mice can acquire resistance to these arthropods, suggesting that evolutionary pressure may lead, at least in part, to ticks feeding on vertebrate hosts on which they can readily take a blood meal without developing resistance (Tabakawa, et al., 2018, Front Immunol, 9:1540; Karasuyama et al., 2014, Curr Opin Immunol, 31:1-7; Wada, et al., 2010, J Clin Invest, 120:2867-2875). These disparities largely influence the choice of an animal model for studying tick immunity, but also provide a platform for understanding how diverse host responses contribute to resistance (Narasimhan, et al., 2019, Ticks Tick Borne Dis, 10:386-397; Anderson, et al., 2017, Front Immunol, 8:1784; Kurokawa, et al., 2020, Nat Rev Microbiol). Guinea pigs acquire robust tick immunity following repeated tick infestations at the larval, nymphal and adult stages (Nazario, et al., 1998, Am J Trop Med Hyg, 58:780-785; Narasimhan, et al., 2007, PLoS One, 2:e451; Whelen, et al., 1993, J Parasitol, 79:908-912; Brown, et al., 1986, Exp Parasitol, 62:40-50).

Naturally acquired tick resistance is generally considered to be associated with host immune responses to tick antigens that are secreted into the bite site, and present in saliva and cement (Narasimhan, et al., 2007, PLoS One, 2:e451; Simo et al., 2017, Front Cell Infect Microbiol, 7:281). Indeed, specific I. scapularis salivary proteins have functional properties that can influence host immune responses, inflammation, and coagulation, but it is unclear whether these antigens are directly involved in the genesis of tick immunity (Titus et al., 2006, Parasite Immunol, 28:131-141; Ribeiro, et al., 1985, J Exp Med, 161:332-344; Ferreira, 1998, Vet Immunol Immunopathol, 64:279-293). Several tick antigens have been shown to generate a host response, either following tick bite or upon specific immunization but robust tick immunity has not been replicated (Kotsyfakis, et al., 2008, J Immunol, 181:5209-5212; Narasimhan, et al., 2020, Ticks Tick Borne Dis, 11:101369; Schuijt, et al., 2013, Circulation, 128:254-266; Tyson, et al., 2007, Insect Mol Biol, 16:469-479; Mulenga, et al., 1999, Infect Immun, 67:1652-1658; Leboulle, et al., 2002, J Biol Chem, 277:10083-10089; Dai, et al., 2010, PLoS Pathog, 6:e1001205; de la Fuente, et al., 2006, Vaccine, 24:4082-4095; Narasimhan, et al., 2007, Cell Host Microbe, 2:7-18; Dai, et al., 2009, Cell Host Microbe, 6:482-492; Hovius, et al., 2008, PLoS Med, 5:e43; Olds, et al., 2016, Parasit Vectors, 9:484; Diaz-Martin, et al., 2015, Vet Parasitol, 212:336-349). For example, vaccination of guinea pigs with sialostatin L2, an I. scapularis immunomodulator, led to some degree of diminished tick feeding and detachment by 72 hours (Kotsyfakis, et al., 2008, J Immunol, 181:5209-5212), and immunization with a combination of two salivary proteins—Salivary protein of 14 kDa (Salp14) and tick lectin pathway inhibitor (TSLPI)—provided modest tick-rejection at 72-96 hours after tick attachment (Narasimhan, et al., 2020, Ticks Tick Borne Dis, 11:101369). Direct immunization with tick saliva alone afforded a more substantial degree of tick rejection by 48-72 hours, indicating that multiple salivary components or a specific manner of immunization are required for eliciting an optimal host response against tick bites (Narasimhan, et al., 2020, Ticks Tick Borne Dis, 11:101369). An approach that more closely replicates the response observed in naturally acquired tick immunity is needed to definitively interfere with pathogen transmission (Nazario, et al., 1998, Am J Trop Med Hyg, 58:780-785; Narasimhan, et al., 2007, PLoS One, 2:e451).

Thus, there is a need in the art for improved compositions and methods for inducing an immune response to tick antigens to treat or prevent tick-borne infection and disease. This invention satisfies this unmet need.

### SUMMARY OF THE INVENTION

In one embodiment the invention relates to a composition for inducing an immune response against at least one tick salivary antigen in a subject. In one embodiment, the composition comprises at least one nucleoside-modified RNA molecule encoding at least one tick salivary antigen. In one embodiment, at least one nucleoside-modified RNA molecule comprises pseudouridine. In one embodiment, at least one nucleoside-modified RNA molecule comprises 1-methyl-pseudouridine.

In one embodiment, at least one tick salivary antigen is Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, or IS-6-12L-5-58 putative secreted protein. In one embodiment, the composition comprises a plurality of nucleoside-modified RNA molecules encoding at least two tick salivary antigens. In one embodiment the at least two tick salivary antigens are selected from Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, or IS-6-12L-5-58 putative secreted protein.

In one embodiment, at least one tick salivary antigen comprises an amino acid sequence of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:18, SEQ ID NO:20, SEQ ID NO:22, SEQ ID NO:24, SEQ ID NO:26, SEQ ID NO:29, SEQ ID NO:32, SEQ ID NO:34, SEQ ID NO:37, SEQ ID NO:40, SEQ ID NO:42, SEQ ID NO:45, SEQ ID NO:48, SEQ ID NO:51, or SEQ ID NO:53.

In one embodiment, at least one nucleoside-modified RNA molecule comprises a nucleotide sequence encoded by a DNA sequence comprising at least one nucleotide sequence of: SEQ ID NO:2, SEQ ID NO:4, SEQ ID NO:6, SEQ ID NO:8, SEQ ID NO:10, SEQ ID NO:12, SEQ ID NO: 14, SEQ ID NO:16, SEQ ID NO:17, SEQ ID NO:19, SEQ ID NO:21, SEQ ID NO:23, SEQ ID NO:25, SEQ ID NO:27, SEQ ID NO:28, SEQ ID NO:30, SEQ ID NO:31, SEQ ID NO:33, SEQ ID NO:35, SEQ ID NO:36, SEQ ID NO:38, SEQ ID NO:39, SEQ ID NO:41, SEQ ID NO:43, SEQ ID NO:44, SEQ ID NO:46, SEQ ID NO:47, SEQ ID NO:49, SEQ ID NO:50, SEQ ID NO:52 and SEQ ID NO: 54.

In one embodiment, the composition further comprises an adjuvant. In one embodiment, the at least one nucleoside-modified RNA further encodes at least one adjuvant.

In one embodiment, the composition further comprising a lipid nanoparticle (LNP). In one embodiment, the at least one nucleoside-modified RNA is encapsulated within the LNP.

In one embodiment, the composition is a vaccine.

In one embodiment, the invention relates to a method of inducing an immune response against one or more tick salivary antigen in a subject comprising administering to the subject an effective amount of a composition comprising at least one nucleoside-modified RNA molecule encoding at least one tick salivary antigen. In one embodiment, at least one nucleoside-modified RNA molecule comprises pseudouridine. In one embodiment, at least one nucleoside-modified RNA molecule comprises 1-methyl-pseudouridine.

In one embodiment, the at least one tick salivary antigen is Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein.

In one embodiment, the method comprises administering to the subject an effective amount of a composition comprising a plurality of nucleoside-modified RNA molecules encoding at least two tick salivary antigens. In one embodiment, the two tick salivary antigens are selected from Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein.

In one embodiment, at least one tick salivary antigen comprises an amino acid sequence selected from SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:18, SEQ ID NO:20, SEQ ID NO:22, SEQ ID NO:24, SEQ ID NO:26, SEQ ID NO:29, SEQ ID NO:32, SEQ ID NO:34, SEQ ID NO:37, SEQ ID NO:40, SEQ ID NO:42, SEQ ID NO:45, SEQ ID NO:48, SEQ ID NO:51, and SEQ ID NO:53.

In one embodiment, the at least one nucleoside-modified RNA molecule comprises a nucleotide sequence encoded by a DNA molecule comprising at least one nucleotide sequence selected from the group consisting of: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:17, SEQ ID NO:19, SEQ ID NO:21, SEQ ID NO:23, SEQ ID NO:25, SEQ ID NO:27, SEQ ID NO:28, SEQ ID NO:30, SEQ ID NO:31, SEQ ID NO:33, SEQ ID NO:35, SEQ ID NO:36, SEQ ID NO:38, SEQ ID NO:39, SEQ ID NO 41, SEQ ID NO:43, SEQ ID NO:44, SEQ ID NO:46, SEQ ID NO:47, SEQ ID NO:49, SEQ ID NO:50, SEQ ID NO:52 and SEQ ID NO:54.

In one embodiment, the method further comprises administering to the subject an effective amount of an adjuvant. In one embodiment, the at least one nucleoside-modified RNA further encodes at least one adjuvant.

In one embodiment, the composition further comprises a lipid nanoparticle (LNP). In one embodiment, the at least one nucleoside-modified RNA is encapsulated within the LNP.

In one embodiment, the composition is a vaccine.

In one embodiment, the composition is administered by an intradermal, subcutaneous, inhalation, intranasal, or intramuscular delivery route.

In one embodiment, the method comprises a single administration of the composition. In one embodiment, the method comprises multiple administrations of the composition.

In one embodiment, the method treats or prevents a disease or disorder associated with a tick-borne pathogen in the subject. In one embodiment, the disease or disorder associated with a tick-borne pathogen is Lyme disease.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts the results of example experiments demonstrating antibody responses to specific antigens in guinea pigs: ELISA of guinea pig sera using specific recombinant proteins corresponding to the mRNAs in 19ISP. Sera was isolated from animals after vaccination. ELISA was performed with 19ISP antigens using serum dilutions of 1:500, 1:5000, and 1:50000. Control sera were tested at 1:500 and 1:5000 dilutions. Antibodies were detected against 10 of the tested proteins—Salp14, Salp15, Salp25D, Salp26A, TSLPI, IsPDIA3, TIX5, P32, SG10, and SG27, with the OD at 450 nm in the 19ISP-immunized groups being higher than the controls. The data represents mean±SEM of at least 6 values.

FIG. 2A and FIG. 2B depict the results of example experiments demonstrating the results of tick challenge of 19ISP mRNA-LNP immunized guinea pigs: Guinea pigs were immunized with 19ISP or control (IL21) mRNA and 25 I. scapularis nymphs were allowed to engorge on their shaved backs. All animals were monitored for the development of erythema as a cardinal initial sign of acquired tick resistance over a period of six days or until all ticks detached. The images show representative (FIG. 2A) 19ISP-immunized or (FIG. 2B) control animals. As observed in the images, 19ISP-immunized guinea pigs showed significant erythema and nymphs were fed poorly with earlier detachment, in comparison with controls.

FIG. 3A through FIG. 3F depicts the results of example experiments demonstrating that 19ISP mRNA vaccination elicits protective responses against tick challenge and B. burgdorferi transmission: Immunized animals (19ISP n=6, IL21 n=5) were challenged with I. scapularis nymphs and assessed for—(FIG. 3A) Erythema: calculated as the percent of nymphs showing redness on each animal, each symbol represents one animal challenged with approximately 25 nymphs, (FIG. 3B) Tick-detachment: The graph represents the percent of ticks remaining attached at a given time point in all animals of a group, (FIG. 3C) Recovery: The graph shows the percent of total ticks recovered from each animal after rejection or detachment. (FIG. 3D) Engorgement: Weights of ticks recovered from each animal after rejection or detachment, represented by each symbol. The error bars represent mean±SD and the significance calculated using the Mann-Whitney test. (FIG. 3E) B. burgdorferi transmission: qPCR data from skin biopsies from Guinea pigs (immunized with 19ISP, n=16 or Luc-mRNA control, n=13), challenged with B. burgdorferi-infected ticks. Data shows relative levels of B. burgdorferi flaB, normalized with guinea pig actin. The data from three biological replicates shows 6/13 B. burgdorferi infected samples in control versus 0/16 infected samples in the 19ISP group. Error bars show mean±SEM and significance was calculated using Welch's t-test (95% confidence interval). (FIG. 3F) Table shows total number of B. burgdorferi culture positive and negative samples in each group, also corroborated by qPCR shown in (FIG. 3E).

FIG. 4A and FIG. 4B depicts the results of example experiments depicting gene expression analyses by RNAseq. RNA-seq analyses were performed using Partek Genomics Flow software. (FIG. 4A) Heatmap showing that 125 differentially expressed genes were identified (p<0.05) and fold change greater than or equal to 2.0. 113 genes were upregulated and 12 genes were downregulated in the 19ISP-immunized group as compared to the control group. (FIG. 4B) Signaling pathways involved in response to 19ISP-mRNA vaccination were identified by KEGG pathway enrichment analysis. The top immune pathways enriched are T cell receptor, B-cell receptor signaling, chemokine signaling, IL-17 signaling, Natural killer cell-mediated cytotoxicity, Fc epsilon RI mediated signaling, and C type lectin receptor signaling.

FIG. 5A through FIG. 5F depicts the results of example experiments demonstrating cytokine expression in 19ISP-immunized guinea pigs: Cytokine expression in RNA from PBMCs isolated from 19ISP and control mRNA immunized guinea pigs, 2-weeks after the second boost and stimulated with I. scapularis saliva. qRT-PCR shows relative expression calculated using the deltaCq method and normalized with guinea pig gapdh.- (FIG. 5A) IFNγ, (FIG. 5B) TNFα, (FIG. 5C) CXCL10 (FIG. 5D) IL2, (FIG. 5E) IL4 and (FIG. 5F) IL8. The figure shows data from two independent experiments and each circle represents a guinea pig (n=6). The p-value was calculated using unpaired t-test, between saliva stimulated 19ISP and mRNA control and error bars show mean±SEM.

FIG. 6 depicts the results of example experiments: Immunized guinea pigs (19ISP n=16, Luc n=13) were challenged with 3 B. burgdorferi infected I. scapularis nymphs and assessed for redness. Ticks were removed from the animals at the time of redness. The graph represents the percent of ticks remaining attached at a given time point in all animals of a group.

FIG. 7A and FIG. 7B depicts the results of example experiments depicting RNAseq analyses for gene expression changes in 19ISP-immunized guinea pigs: RNA was isolated from the blood of 19ISP or control mRNA immunized guinea pigs for gene expression analysis two weeks after the final immunization. (FIG. 7A) Principal component analysis (PCA) revealed that the 19ISP-vaccinated animal group formed a separate cluster from the control animal groups, indicating a specific gene expression pattern. (FIG. 7B) To identify signaling pathways involved in response to 19ISP-mRNA immunization, we utilized KEGG pathway enrichment analysis. The data represents the top 20 enriched pathways following immunization in the 19ISP group as compared to controls.

FIG. 8A and FIG. 8B depicts the results of example experiments depicting gene expression analysis at the bite site: The changes in gene expression were assessed at the erythematous tick bite site in the 19ISP immunized guinea pigs. (FIG. 8A) Heatmap showing that differentially expressed genes (p<0.05) at erythematic site (red) and compared with non-erythematic site (non-red) in the same guinea pig. The data shows analysis from two different guinea pigs. (FIG. 8B) Table showing the major immune pathways that were found to be upregulated at the erythematic bite site.

FIG. 9A through FIG. 9C depict the results of example experiments examining 19ISP immunization in mice: Mice were immunized with 19ISP mRNA-LNPs and monitored for evidence of tick immunity. (FIG. 9A) Ticks feeding on 19ISP-immunized mice did not show a difference in attachment when compared to that on control animals. (FIG. 9B) Immunization with 19ISP mRNA-LNPs did not impact tick feeding as assessed by engorgement weights. (FIG. 9C) Upon tick challenge, some redness (approximately 20% ticks) was observed in 19ISP immunized mice at 24-48 hours post-tick challenge, which was insignificant as compared to that observed in guinea pigs.

### DETAILED DESCRIPTION

The present invention relates to compositions and methods for inducing an immune response against one or more tick salivary antigen in a subject. In some embodiments, the invention provides a composition comprising at least one nucleoside-modified RNA encoding at least one tick salivary antigen. For example, in one embodiment, the composition is a vaccine comprising at least one nucleoside-modified RNA encoding at least one tick salivary antigen, where the vaccine induces an immune response in the subject to the at least one tick salivary antigen, and therefore functions to treat or prevent a tick-borne virus or pathology associated with a tick-borne pathogen. In some embodiments, the tick-borne virus or pathogen is Borrelia burgdorferi, Babesia microti, Anaplasma phagocytophilum, Borrelia miyamotoi, Powassan virus, Rocky Mountain Spotted Fever or Ehrlichiosis. In one embodiment, the pathology associated with Borrelia burgdorferi is Lyme disease.

In some embodiments, the tick salivary protein is a protein or antigen that contributes to tick rejection. In some embodiments, the tick salivary protein is Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein or a paralog in I. scapularis with significant homology to Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, or SG09. In some embodiments, at least one nucleoside-modified RNA encodes at least one of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein. In some embodiments, the at least one nucleoside-modified RNA is encapsulated in a lipid nanoparticle (LNP).

In some embodiments, the invention relates to a vaccine comprising a combination of mRNA molecules, wherein the combination of mRNA molecules encodes at least 2, 3, 4, 5, 6, 7, 8, 9, 10, 11, 12, 13, 14, 15, 16, 17, 18, 19 or more than 19 antigens from one or more tick salivary antigen. In one embodiment, the vaccine comprises a combination of mRNA molecules, wherein the combination of mRNA molecules encodes each of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein. In some embodiments, each nucleoside-modified RNA is encapsulated in a lipid nanoparticle (LNP). Therefore, in some embodiments, the invention provides a combination of at least 2, 3, 4, 5, 6, 7, 8, 9, 10, 11, 12, 13, 14, 15, 16, 17, 18, 19 or more than 19 LNPs, wherein each LNP comprises a nucleoside-modified RNA, and further wherein the combination of LNPs comprises nucleoside-modified RNA molecules encoding a combination of at least 2, 3, 4, 5, 6, 7, 8, 9, 10, 11, 12, 13, 14, 15, 16, 17, 18, 19 or more than 19 tick salivary antigens. In one embodiment, the combination of LNPs comprises a combination of nucleoside-modified mRNA molecules encoding each of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody, which is generated using recombinant DNA technology. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned), synthesizing the RNA, or other technology, which is available and well known in the art.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “immunogen” as used herein, is intended to denote a substance of matter, which is capable of inducing an adaptive immune response in an individual, where said adaptive immune response is capable of inducing an immune response, which significantly engages pathogenic agents, which share immunological features with the immunogen. “Immunogen” refers to any substance introduced into the body in order to generate an immune response. That substance can a physical molecule, such as a protein, or can be encoded by a vector, such as DNA, mRNA, or a virus.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequence or a partial nucleotide sequence encoding a protein that elicits an adaptive immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“Immune response,” as the term is used herein, means a process involving the activation and/or induction of an effector function in, by way of non-limiting examples, a T cell, B cell, natural killer (NK) cell, and/or an antigen-presenting cell (APC). Thus, an immune response, as would be understood by the skilled artisan, includes, but is not limited to, any detectable antigen-specific activation and/or induction of a helper T cell or cytotoxic T cell activity or response, production of antibodies, antigen presenting cell activity or infiltration, macrophage activity or infiltration, neutrophil activity or infiltration, and the like.

As used herein, an “immunogenic composition” may comprise an antigen (e.g., a peptide or polypeptide), a nucleic acid encoding an antigen, a cell expressing or presenting an antigen or cellular component, a virus expressing or presenting an antigen or cellular component, or a combination thereof. In particular embodiments, the composition comprises or encodes all or part of any peptide antigen described herein, or an immunogenically functional equivalent thereof. In other embodiments, the composition is in a mixture that comprises an additional immunostimulatory agent or nucleic acids encoding such an agent. Immunostimulatory agents include but are not limited to an additional antigen, an immunomodulator, an antigen presenting cell, lipid nanoparticle, or an adjuvant. In other embodiments, one or more of the additional agent(s) is covalently bonded to the antigen or an immunostimulatory agent, in any combination.

As used herein, the term “vaccine” refers to a composition that induces an immune response upon inoculation into a subject. In some embodiments, the induced immune response provides protective immunity.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

As used herein, a nucleotide sequence is “substantially homologous” to any of the nucleotide sequences described herein when its nucleotide sequence has a degree of identity with respect to the original nucleotide sequence at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

As used herein, an amino acid sequence is “substantially homologous” to any of the amino acid sequences described herein when its amino acid sequence has a degree of identity with respect to the original amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. The identity between two amino acid sequences can be determined by using the BLASTN algorithm (BLAST Manual, Altschul, S., et al., NCBI NLM NIH Bethesda, Md. 20894, Altschul, S., et al., J. Mol. Biol. 215: 403-410 (1990)).

The term “variant” as used herein with respect to a nucleic acid refers (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto. A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof.

The term “variant” as used with respect to a peptide or polypeptide refers to a peptide or polypeptide that differs in amino acid sequence by the insertion, deletion, or conservative substitution of amino acids, but retain at least one biological activity. Variant may also refer to a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., 1982, J. Mol. Biol. 157:105-132). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

As used herein, the terms “fragment” or “functional fragment” refer to a fragment of a tick salivary antigen or a nucleic acid sequence encoding a tick salivary antigen that, when administered to a subject, provides an increased immune response. Fragments are generally 10 or more amino acids or nucleic acids in length. “Fragment” may mean a polypeptide fragment of an antigen that is capable of eliciting an immune response in a subject. A fragment of an antigen may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antigen, excluding any heterologous signal peptide added. The fragment may comprise a fragment of a polypeptide that is 95% or more, 96% or more, 97% or more, 98% or more or 99% or more identical to the antigen and additionally comprise an N terminal methionine or heterologous signal peptide which is not included when calculating percent identity.

A fragment of a nucleic acid sequence that encodes an antigen may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antigen and additionally optionally comprise sequence encoding an N terminal methionine or heterologous signal peptide which is not included when calculating percent identity.

“Isolated” means altered or removed from the natural state. For example, a nucleic acid or a peptide naturally present in a living subject is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleosides (nucleobase bound to ribose or deoxyribose sugar via N-glycosidic linkage) are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. The phrase nucleotide sequence that encodes a protein or an RNA may also include introns to the extent that the nucleotide sequence encoding the protein may in some version contain an intron(s). In addition, the nucleotide sequence may contain modified nucleosides that are capable of being translated by translational machinery in a cell. Exemplary modified nucleosides are described elsewhere herein. For example, an mRNA where some or all of the uridines have been replaced with pseudouridine, 1-methyl pseudouridine, 5-methyl-uridine, or another modified nucleoside, such as those described elsewhere herein. In some embodiments, the nucleotide sequence may contain a sequence where some or all cytodines are replaced with methylated cytidine, or another modified nucleoside, such as those described elsewhere herein.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In some instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27: 196-197).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence. By way of one non-limiting example, a promoter that is recognized by bacteriophage RNA polymerase and is used to generate the mRNA by in vitro transcription.

The term “adjuvant” as used herein is defined as any molecule to enhance an antigen-specific adaptive immune response.

In some embodiments, “pseudouridine” refers to m¹acp³Ψ (1-methyl-3-(3-amino-3-carboxypropyl) pseudouridine). In another embodiment, the term refers to m¹Ψ (1-methylpseudouridine). In another embodiment, the term refers to Ψm (2′-O-methylpseudouridine. In another embodiment, the term refers to m⁵D (5-methyldihydrouridine). In another embodiment, the term refers to m³Ψ (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

The term “lipid nanoparticle” refers to a particle having at least one dimension on the order of nanometers (e.g., 1-1,000 nm), which includes one or more lipids.

The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In some embodiments, the cationic lipid comprises a zwitterionic lipid that assumes a positive charge on pH decrease.

The term “neutral lipid” refers to any one of a number of lipid species that exist in either an uncharged or neutral zwitterionic form at physiological pH.

Representative neutral lipids include diacylphosphatidylcholines, diacylphosphatidylethanolamines, ceramides, sphingomyelins, dihydro sphingomyelins, cephalins, and cerebrosides.

The term “anionic lipid” refers to any lipid that is negatively charged at physiological pH.

The term “polymer conjugated lipid” refers to a molecule comprising both a lipid portion and a polymer portion. An example of a polymer conjugated lipid is a pegylated lipid.

The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

The terms “subject,” “patient,” “individual,” and the like are used interchangeably herein, and refer to any animal, or cells thereof whether in vitro or in situ, amenable to the methods described herein. In some non-limiting embodiments, the patient, subject or individual is a mammal, bird, poultry, cattle, pig, horse, sheep, ferret, primate, dog, cat, guinea pig, rabbit, bat, or human.

A “disease” is a state of health of a subject wherein the subject cannot maintain homeostasis, and wherein if the disease is not ameliorated then the subject's health continues to deteriorate.

In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as a human.

To “treat” a disease as the term is used herein, means to reduce the frequency or severity of at least one sign or symptom of a disease or disorder experienced by a subject.

An “effective amount” as used herein, means an amount which provides a therapeutic or prophylactic benefit.

The term “therapeutic” as used herein means a treatment and/or prophylaxis. A therapeutic effect is obtained by suppression, diminution, remission, prevention, or eradication of at least one sign or symptom of a disease or disorder.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to compositions and methods for inducing an immune response against a tick salivary antigen in a subject. In some embodiments, the method treats or prevents one or more disease or disorder associated with a tick-borne pathogen. Tick-borne pathogens include, but are not limited to, Borrelia burgdorferi, Babesia microti, Anaplasma phagocytophilum, Borrelia miyamotoi, Powassan virus, Rocky Mountain Spotted Fever or Ehrlichiosis, or a combination thereof. In some embodiments, the invention provides a composition comprising one or more lipid nanoparticles comprising one or more nucleoside-modified RNA molecules encoding at least one tick salivary antigen. In some embodiments, the invention provides a composition comprising one or more lipid nanoparticles comprising one or more nucleoside-modified RNA molecules encoding at least tick salivary antigen. In some embodiments, the tick salivary protein is a protein or antigen that contributes to tick rejection. In some embodiments, the tick salivary protein is Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein or a paralog in I. scapularis with significant homology to Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, or IS-6-12L-5-58 putative secreted protein. Therefore, in some embodiments, the invention relates to a vaccine comprising at least one nucleoside-modified RNA molecule encoding at least one of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, or IS-6-12L-5-58 putative secreted protein.

In some embodiments, the at least one nucleoside-modified RNA is encapsulated in a lipid nanoparticle (LNP). Therefore, in some embodiments, the invention relates to a vaccine comprising at least one LNP comprising at least one nucleoside-modified RNA molecules encoding at least one of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, or IS-6-12L-5-58 putative secreted protein.

For example, in one embodiment, the composition is a vaccine comprising a combination of lipid nanoparticles comprising nucleoside-modified RNA molecules encoding a combination of at least two tick salivary antigens. In one embodiment, the nucleoside-modified RNA molecules encoding a combination of at least two of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein, wherein the vaccine induces an immune response in the subject to multiple tick salivary antigens. In some embodiments, the invention relates to a vaccine comprising a combination of mRNA molecules, wherein the combination of mRNA molecules encodes at least 2, 3, 4, 5, 6, 7, 8, 9, 10, 11, 12, 13, 14, 15, 16, 17, 18, 19 or more than 19 tick salivary antigens. In one embodiment, the vaccine comprises a combination of mRNA molecules, wherein the combination of mRNA molecules encodes each of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein. In some embodiments, the at least one nucleoside-modified RNA molecule is encapsulated in an LNP. In some embodiments, the composition comprises a combination of at least two LNPs comprising nucleoside-modified RNA molecules encoding the combination of at least two tick salivary antigen. In some embodiments, the invention provides a combination of at least 2, 3, 4, 5, 6, 7, 8, 9, 10, 11, 12, 13, 14, 15, 16, 17, 18, 19 or more than 19 LNPs, wherein each LNP comprises a nucleoside-modified RNA, and further wherein the combination of LNPs comprises nucleoside-modified RNA molecules encoding a combination of at least 2, 3, 4, 5, 6, 7, 8, 9, 10, 11, 12, 13, 14, 15, 16, 17, 18, 19 or more than 19 tick salivary antigens. In one embodiment, the combination of LNPs comprises a combination of nucleoside-modified mRNA molecules encoding each of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein.

In some embodiments, the composition further comprises at least 1, 2, 3, 4, 5, 6, 7, 8, 9, 10, 11, 12, 13, 14, 15, 16, 17, 18, 19, 20, or more than twenty additional antigens. In some embodiments, one or more additional antigens may be from human immunodeficiency virus (HIV), Chikungunya virus (CHIKV), dengue fever virus, papilloma viruses, for example, human papillomoa virus (HPV), polio virus, hepatitis viruses, for example, hepatitis A virus (HAV), hepatitis B virus (HBV), hepatitis C virus (HCV), hepatitis D virus (HDV), and hepatitis E virus (HEV), smallpox virus (Variola major and minor), vaccinia virus, rhinoviruses, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, lassa virus, arenavirus, Merkel cell polyoma virus (MCV), influenza virus, severe acute respiratory syndrome-coronavirus 2, a cancer causing virus, or any combination thereof.

### Vaccine

In one embodiment, the present invention provides an immunogenic composition for inducing an immune response against at least one tick salivary antigen in a subject. For example, in one embodiment, the immunogenic composition is a vaccine. For a composition to be useful as a vaccine, the composition must induce an immune response against the tick salivary antigen in a cell, tissue or subject. In some embodiments, the composition induces a broad immune response against multiple tick salivary antigens in a cell, tissue or subject. In some instances, the vaccine induces a protective immune response in the subject.

A vaccine of the present invention may vary in its composition of nucleic acid and/or cellular components. In one embodiment, the vaccine comprises at least one nucleic acid molecule encoding at least one tick salivary antigen. In some embodiments, the vaccine further comprises one or more additional nucleic acid molecules encoding at least 1, 2, 3, 4, 5, 6, 7, 8, 9, 10, 11, 12, 13, 14, 15, 16, 17, 18, 19, or more than 19 tick salivary antigens.

In a non-limiting example, one or more nucleic acid molecule encoding a tick salivary antigen might also be formulated with an adjuvant. In another non-limiting example, the LNP vaccine may comprise one or more adjuvants. A vaccine of the present invention, and its various components, may be prepared and/or administered by any method disclosed herein or as would be known to one of ordinary skill in the art, in light of the present disclosure.

In various embodiments, the induction of immunity by the expression of one or more tick salivary antigen can be detected by observing in vivo or in vitro the response of all or any part of the immune system in the host against tick saliva.

For example, a method for detecting the induction of cytotoxic T lymphocytes is well known. A foreign substance that enters the living body is presented to T cells and B cells by the action of antigen presenting cells (APCs). Some T cells that respond to the antigen presented by APC in an antigen specific manner differentiate into cytotoxic T cells (also referred to as cytotoxic T lymphocytes or CTLs) due to stimulation by the antigen. These antigen-stimulated cells then proliferate. This process is referred to herein as “activation” of T cells. Therefore, CTL induction by an epitope of a polypeptide or peptide or combinations thereof can be evaluated by presenting an epitope of a polypeptide or peptide or combinations thereof to a T cell by APC, and detecting the induction of CTL. Furthermore, APCs have the effect of activating B cells, CD4+ T cells, CD8+ T cells, macrophages, eosinophils and NK cells.

A method for evaluating the inducing action of CTL using dendritic cells (DCs) as APC is well known in the art. DC is a representative APC having a robust CTL inducing action among APCs. In the methods of the invention, the epitope of a polypeptide or peptide or combinations thereof is initially expressed by the DC and then this DC is contacted with T cells. Detection of T cells having cytotoxic effects against the cells of interest after the contact with DC shows that the epitope of a polypeptide or peptide or combinations thereof has an activity of inducing the cytotoxic T cells. Furthermore, the induced immune response can also be examined by measuring IFN-gamma produced and released by CTL in the presence of antigen-presenting cells that carry immobilized peptide or a combination of peptides by visualizing using anti-IFN-gamma antibodies, such as an ELISPOT assay.

Apart from DC, peripheral blood mononuclear cells (PBMCs) may also be used as the APC. The induction of CTL is reported to be enhanced by culturing PBMC in the presence of GM-CSF and IL-4. Similarly, CTL has been shown to be induced by culturing PBMC in the presence of keyhole limpet hemocyanin (KLH) and IL-7.

The antigens confirmed to possess CTL-inducing activity by these methods are antigens having DC activation effect and subsequent CTL-inducing activity. Furthermore, CTLs that have acquired cytotoxicity due to presentation of the antigen by APC can be also used as vaccines against antigen-associated disorders.

The induction of immunity by expression of one or more tick salivary antigen can be further confirmed by observing the induction of antibody production against the tick salivary antigen. For example, when antibodies against an antigen are induced in a laboratory subject immunized with the composition encoding the antigens, and when antigen-associated pathology is suppressed by those antibodies, the composition is determined to induce immunity.

The specificity of the antibody response induced in a subject can include binding to many regions of the delivered antigen, as well as, the induction of neutralization capable antibodies that that prevent infection or reduce disease severity.

The induction of immunity by expression of one or more tick salivary antigen can be further confirmed by observing the induction of T cells, such as CD4+ T cells, CD8+ T cells, or a combination thereof. For example, CD4+ T cells can also lyse target cells, but mainly supply help in the induction of other types of immune responses, including CTL and antibody generation. The type of CD4+ T cell help can be characterized, as Th1, Th2, Th9, Th17, Tregulatory (Treg), or T follicular helper (Tfh) cells. Each subtype of CD4+ T cell supplies help to certain types of immune responses. In one embodiment, the composition selectively induces T follicular helper cells, which drive potent antibody responses.

The therapeutic compounds or compositions of the invention may be administered prophylactically (i.e., to prevent a disease or disorder) or therapeutically (i.e., to treat a disease or disorder) to subjects suffering from, or at risk of (or susceptible to) developing a disease or disorder. Such subjects may be identified using standard clinical methods. In the context of the present invention, prophylactic administration occurs prior to the manifestation of overt clinical symptoms of disease, such that a disease or disorder is prevented or alternatively delayed in its progression. In the context of the field of medicine, the term “prevent” encompasses any activity, which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

### Antigen

The present invention provides a composition that induces an immune response against one or more tick salivary antigen in a subject. In one embodiment, the composition comprises one or more nucleic acid molecules encoding one or more tick salivary antigen.

Exemplary tick salivary antigen that can be included in the vaccine of the invention include, but are not limited to, Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein or the I. scapularis paralogs of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein. In one embodiment, the mRNA molecule encoding the tick salivary antigen encodes one or more amino acid sequence set forth in: SEQ ID NO:1, SEQ ID NO:3, SEQ ID NO:5, SEQ ID NO:7, SEQ ID NO:9, SEQ ID NO:11, SEQ ID NO: 13, SEQ ID NO:15, SEQ ID NO:18, SEQ ID NO:20, SEQ ID NO:22, SEQ ID NO:24, SEQ ID NO:26, SEQ ID NO:29, SEQ ID NO:32, SEQ ID NO:34, SEQ ID NO:37, SEQ ID NO:40, SEQ ID NO:42, SEQ ID NO:45, SEQ ID NO:48, SEQ ID NO:51, and SEQ ID NO:53, or a fragment or variant thereof. In one embodiment, the mRNA molecule encoding the tick salivary antigen correlates to one or more nucleotide sequence set forth in: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:17, SEQ ID NO: 19, SEQ ID NO:21, SEQ ID NO:23, SEQ ID NO:25, SEQ ID NO:27, SEQ ID NO:28, SEQ ID NO:30, SEQ ID NO:31, SEQ ID NO:33, SEQ ID NO:35, SEQ ID NO:36, SEQ ID NO:38, SEQ ID NO:39, SEQ ID NO:41, SEQ ID NO:43, SEQ ID NO:44, SEQ ID NO:46, SEQ ID NO:47, SEQ ID NO:49, SEQ ID NO:50, SEQ ID NO:52 and SEQ ID NO:54, or a fragment or variant thereof.

In one embodiment, the mRNA molecule encoding the tick salivary antigen is encoded by a DNA molecule comprising a nucleotide sequence as set forth in: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:17, SEQ ID NO:19, SEQ ID NO:21, SEQ ID NO:23, SEQ ID NO:25, SEQ ID NO:27, SEQ ID NO:28, SEQ ID NO:30, SEQ ID NO:31, SEQ ID NO:33, SEQ ID NO:35, SEQ ID NO:36, SEQ ID NO:38, SEQ ID NO:39, SEQ ID NO:41, SEQ ID NO:43, SEQ ID NO:44, SEQ ID NO:46, SEQ ID NO:47, SEQ ID NO:49, SEQ ID NO:50, SEQ ID NO:52 and SEQ ID NO:54, or a fragment or variant thereof.

In one embodiment, the composition comprises a plurality of mRNA molecules encoding at least 2, 3, 4, 5, 6, 7, 8, 9, 10, 11, 12, 13, 14, 15, 16, 17, 18, 19 or more than 19 tick salivary antigens. In one embodiment, the plurality of mRNA molecules encodes two or more of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein. In one embodiment, the plurality of mRNA molecules encodes each of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein.

In one embodiment, the composition comprises a plurality of LNPs, wherein each LNP comprises at least one mRNA molecule encoding at least one tick salivary antigen. In one embodiment, the plurality of LNPs together comprise a plurality of mRNA molecules encoding a combination of at least two tick salivary antigens. In one embodiment, the combination of mRNA molecules encodes at least 2, 3, 4, 5, 6, 7, 8, 9, 10, 11, 12, 13, 14, 15, 16, 17, 18, 19 or more than 19 tick salivary antigens. In one embodiment, the vaccine comprises a plurality of LNPs comprising a combination of mRNA molecules, wherein the combination of mRNA molecules encodes two or more of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein. In one embodiment, the vaccine comprises a plurality of LNPs comprising a combination of mRNA molecules, wherein the combination of mRNA molecules encodes each of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein.

In one embodiment, the mRNA molecules encoding the tick salivary antigens encode at least 2, 3, 4, 5, 6, 7, 8, 9, 10, 11, 12, 13, 14, 15, 16, 17, 18, or each amino acid sequence set forth in: SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:18, SEQ ID NO:20, SEQ ID NO:22, SEQ ID NO:24, SEQ ID NO:26, SEQ ID NO:29, SEQ ID NO:32, SEQ ID NO:34, SEQ ID NO:37, SEQ ID NO:40, SEQ ID NO:42, SEQ ID NO:45, SEQ ID NO:48, SEQ ID NO:51, and SEQ ID NO:53 or fragments or variants thereof.

In one embodiment, the mRNA molecules encoding the tick salivary antigens comprise nucleotide sequences corresponding to at least 2, 3, 4, 5, 6, 7, 8, 9, 10, 11, 12, 13, 14, 15, 16, 17, 18, 19 or more of: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:17, SEQ ID NO:19, SEQ ID NO:21, SEQ ID NO:23, SEQ ID NO:25, SEQ ID NO:27, SEQ ID NO:28, SEQ ID NO:30, SEQ ID NO:31, SEQ ID NO:33, SEQ ID NO:35, SEQ ID NO:36, SEQ ID NO:38, SEQ ID NO:39, SEQ ID NO:41, SEQ ID NO 43, SEQ ID NO:44, SEQ ID NO:46, SEQ ID NO:47, SEQ ID NO:49, SEQ ID NO:50, SEQ ID NO:52 and SEQ ID NO:54, or fragments or variants thereof.

In one embodiment, the mRNA molecules encoding the tick salivary antigens are encoded by at least 1, 2, 3, 4, 5, 6, 7, 8, 9, 10, 11, 12, 13, 14, 15, 16, 17, 18, 19 or more than 19 DNA molecules comprising nucleotide sequences as set forth in at least 2, 3, 4, 5, 6, 7, 8, 9, 10, 11, 12, 13, 14, 15, 16, 17, 18, 19 or more than 19 of: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:17, SEQ ID NO:19, SEQ ID NO:21, SEQ ID NO:23, SEQ ID NO:25, SEQ ID NO:27, SEQ ID NO:28, SEQ ID NO:30, SEQ ID NO:31, SEQ ID NO:33, SEQ ID NO:35, SEQ ID NO:36, SEQ ID NO:38, SEQ ID NO:39, SEQ ID NO:41, SEQ ID NO:43, SEQ ID NO:44, SEQ ID NO:46, SEQ ID NO:47, SEQ ID NO:49, SEQ ID NO:50, SEQ ID NO:52 and SEQ ID NO:54, or fragments or variants thereof.

In one embodiment, the nucleic acid molecule encoding the tick salivary antigen comprises a sequence encoding a tag or signal peptide (SP). Other signal peptides that may be used include, but are not limited to, signal sequences derived from IL-2, tPA, mouse and human IgG, and synthetic optimized signal sequences. In some instances, the nucleic acid sequence comprises include additional sequences that encode linker or tag sequences that are linked to the antigen by a peptide bond. In one embodiment, the sequences encoding the tick salivary antigen are operably linked to a sequence encoding an optimized IL-2 leader sequence comprising the nucleotide sequence atgcgcatgcagctgctgctgctgatcgccctgtccctggccctggtgaccaactcc (SEQ ID NO:55).

In some embodiments, the tick salivary antigen comprises an amino acid sequence that is substantially homologous or substantially identical to the amino acid sequence of a tick salivary antigen described herein and retains the immunogenic function of the original amino acid sequence. For example, in some embodiments, the amino acid sequence of the encoded tick salivary antigen has a degree of identity with respect to the original amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the nucleotide sequence of the nucleic acid molecule encoding the tick salivary antigen has a degree of identity with respect to the original nucleotide sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

### Adjuvant

In one embodiment, the composition comprises an adjuvant. In one embodiment, the composition comprises a nucleic acid molecule encoding an adjuvant. In one embodiment, the adjuvant-encoding nucleic acid molecule is IVT RNA. In one embodiment, the adjuvant-encoding nucleic acid molecule is nucleoside-modified RNA. In one embodiment, the adjuvant-encoding nucleic acid molecule is nucleoside-modified mRNA.

Exemplary adjuvants include, but are not limited to, alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHIC, CD80, CD86. Other genes which may be useful adjuvants include those encoding: MCP-I, MIP-Ia, MIP-Ip, IL-8, RANTES, L-selectin, P-selectin, E-selectin, CD34, GlyCAM-1, MadCAM-1, LFA-I, VLA-I, Mac-1, pl50.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R2, TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, INK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig, and functional fragments thereof.

In some embodiments, the composition comprises an LNP, where the LNP acts as an adjuvant.

### Nucleic Acids

In one embodiment, the invention includes one or more nucleic acid molecules encoding one or more tick salivary antigen, as described elsewhere herein. The nucleic acid molecule can be made using any methodology in the art, including, but not limited to, in vitro transcription, chemical synthesis, or the like.

The nucleotide sequences encoding one or more tick salivary antigen as described herein, can alternatively comprise sequence variations with respect to the original nucleotide sequences, for example, substitutions, insertions and/or deletions of one or more nucleotides, with the condition that the resulting polynucleotide encodes a polypeptide according to the invention. Therefore, the scope of the present invention includes nucleotide sequences that are substantially homologous or substantially identical to the nucleotide sequences recited herein and encode one or more tick salivary antigen of the invention.

A nucleotide sequence that is substantially homologous to a nucleotide sequence encoding an antigen can typically be isolated from a producer organism of the antigen based on the information contained in the nucleotide sequence by means of introducing conservative or non-conservative substitutions, for example. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art.

Further, the scope of the invention includes nucleotide sequences that encode amino acid sequences that are substantially homologous to the amino acid sequences recited herein and preserve the immunogenic function of the original amino acid sequence.

In one embodiment, the invention relates to a construct, comprising a nucleotide sequence encoding a tick salivary antigen. In one embodiment, the construct comprises a plurality of nucleotide sequences encoding a plurality of t tick salivary antigens. For example, in some embodiments, the construct encodes 1 or more, 2 or more, 3 or more, or more than 3 tick salivary antigens. In one embodiment, the invention relates to a construct, comprising a nucleotide sequence encoding an adjuvant. In one embodiment, the construct comprises a first nucleotide sequence encoding at least one tick salivary antigen and a second nucleotide sequence encoding an adjuvant.

In one embodiment, the composition comprises a plurality of constructs, each construct encoding two or more tick salivary antigens. In one embodiment, the composition comprises a first construct, comprising a nucleotide sequence encoding a first tick salivary antigen; and one or more additional construct, comprising a nucleotide sequence encoding one or more additional tick salivary antigen.

In another particular embodiment, one or more construct is operatively bound to a translational control element. The construct can incorporate an operatively bound regulatory sequence for the expression of the nucleotide sequence of the invention, thus forming an expression cassette.

### Vectors

The nucleic acid sequences coding for the tick salivary antigen of the invention can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically.

The nucleic acid molecules can be cloned into a number of types of vectors. For example, the nucleic acid can be cloned into a vector including, but not limited to a plasmid, a phagemid, a phage derivative, an animal virus, a PCR-generated linear DNA sequence, and a cosmid. Vectors of particular interest include expression vectors, replication vectors, probe generation vectors, sequencing vectors and vectors optimized for in vitro transcription.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, carbohydrates, peptides, cationic polymers, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/RNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as it is more readily evaporated than methanol.

Regardless of the method used to introduce exogenous nucleic acids into a host cell or otherwise expose a cell to a composition of the present invention, in order to confirm the presence of the mRNA sequence in the host cell, a variety of assays may be performed. Such assays include, for example, “molecular biological” assays well known to those of skill in the art, such as Northern blotting and RT-PCR; “biochemical” assays, such as detecting the presence or absence of a particular peptide, e.g., by immunogenic means (ELISAs and Western blots) or by assays described herein to identify agents falling within the scope of the invention.

### In Vitro Transcribed RNA

In one embodiment, the composition of the invention comprises at least one in vitro transcribed (IVT) RNA molecule encoding one or more tick salivary antigen of the invention. In one embodiment, an IVT RNA can be introduced to a cell as a form of transient transfection. The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. In one embodiment, the desired template for in vitro transcription is a tick salivary antigen capable of inducing an adaptive immune response. In one embodiment, the desired template for in vitro transcription is an adjuvant capable of enhancing an adaptive immune response.

In one embodiment, the DNA to be used for PCR contains an open reading frame. The DNA can be from a naturally occurring DNA sequence from the genome of an organism. In one embodiment, the DNA is a full-length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. In another embodiment, the DNA to be used for PCR is a gene from a pathogenic or commensal organism, including bacteria, viruses, parasites, and fungi. In another embodiment, the DNA to be used for PCR is from a pathogenic or commensal organism, including bacteria, viruses, parasites, and fungi, including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

Genes that can be used as sources of DNA for PCR include genes that encode polypeptides that induce or enhance an adaptive immune response in an organism. In some instances, the genes are useful for a short term treatment. In some instances, the genes have limited safety concerns regarding dosage of the expressed gene.

In various embodiments, a plasmid is used to generate a template for in vitro transcription of mRNA, which is used for transfection.

Chemical structures with the ability to promote stability and/or translation efficiency may also be used. In some embodiments, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 RNA polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability of mRNA in the cell. On a circular DNA template, for instance, plasmid DNA, RNA polymerase produces a long concatameric product, which is not suitable for expression in eukaryotic cells. The transcription of plasmid DNA linearized at the end of the 3′ UTR results in normal sized mRNA, which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003)).

The conventional method of integration of polyA/T stretches into a DNA template is molecular cloning. However, polyA/T sequence integrated into plasmid DNA can cause plasmid instability, which can be ameliorated through the use of recombination incompetent bacterial cells for plasmid propagation.

Poly(A) tails of RNAs can be further extended following in vitro transcription with the use of a poly(A) polymerase, such as E. coli polyA polymerase (E-PAP) or yeast polyA polymerase. In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to mRNA molecules. In one embodiment, RNAs produced by the methods to include a 5′ cap1 structure. Such cap1 structure can be generated using Vaccinia capping enzyme and 2′-O-methyltransferase enzymes (CellScript, Madison, WI). Alternatively, 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001)). In some embodiments RNA of the invention is introduced to a cell with a method comprising the use of TransIT®-mRNA transfection Kit (Mirus, Madison WI), which, in some instances, provides high efficiency, low toxicity, transfection.

### Nucleoside-Modified RNA

In one embodiment, the composition of the present invention comprises a nucleoside-modified nucleic acid encoding at least one tick salivary antigen as described herein. In one embodiment, the composition of the present invention comprises a nucleoside-modified nucleic acid encoding a plurality of antigens, including one or more tick salivary antigen. In one embodiment, the composition of the present invention comprises a nucleoside-modified nucleic acid encoding an adjuvant as described herein. In one embodiment, the composition of the present invention comprises a nucleoside-modified nucleic acid encoding one or more tick salivary antigen and one or more adjuvants.

In one embodiment, the composition comprises a nucleoside-modified RNA. In one embodiment, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present invention is further described in U.S. Pat. Nos. 8,278,036, 8,691,966, and 8,835,108, each of which is incorporated by reference herein in its entirety.

In some embodiments, nucleoside-modified mRNA does not activate any pathophysiologic pathways, translates very efficiently and almost immediately following delivery, and serve as templates for continuous protein production in vivo lasting for several days to weeks (Karikó et al., 2008, Mol Ther 16:1833-1840; Karikó et al., 2012, Mol Ther 20:948-953). The amount of mRNA required to exert a physiological effect is small, making it applicable for human therapy. For example, as described herein, nucleoside-modified mRNA encoding an tick salivary antigen has demonstrated the ability to induce antigen-specific antibody production. For example, in some instances, antigen encoded by nucleoside-modified mRNA induces greater production of antigen-specific antibody production as compared to antigen encoded by non-modified mRNA.

In some instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In some embodiments, using mRNA rather than the protein also has many advantages. Half-lives of proteins in the circulation or in tissues are often short, thus protein treatment would need frequent dosing, while mRNA provides a template for continuous protein production for several days to weeks. Purification of proteins is problematic and they can contain aggregates and other impurities that cause adverse effects (Kromminga and Schellekens, 2005, Ann NY Acad Sci 1050:257-265).

In some embodiments, the nucleoside-modified RNA comprises the naturally occurring modified-nucleoside pseudouridine. In some embodiments, inclusion of pseudouridine makes the mRNA more stable, non-immunogenic, and highly translatable (Karikó et al., 2008, Mol Ther 16:1833-1840; Anderson et al., 2010, Nucleic Acids Res 38:5884-5892; Anderson et al., 2011, Nucleic Acids Research 39:9329-9338; Karikó et al., 2011, Nucleic Acids Research 39:e142; Karikó et al., 2012, Mol Ther 20:948-953; Karikó et al., 2005, Immunity 23:165-175).

It has been demonstrated that the presence of modified nucleosides, including pseudouridines in RNA suppress their innate immunogenicity (Karikó et al., 2005, Immunity 23:165-175). Further, protein-encoding, in vitro-transcribed RNA containing pseudouridine can be translated more efficiently than RNA containing no or other modified nucleosides (Karikó et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892).

Similar effects as described for pseudouridine have also been observed for RNA containing 1-methyl-pseudouridine.

In some embodiments, the nucleoside-modified nucleic acid molecule is a purified nucleoside-modified nucleic acid molecule. For example, in some embodiments, the composition is purified to remove double-stranded contaminants. In some instances, a preparative high-performance liquid chromatography (HPLC) purification procedure is used to obtain pseudouridine-containing RNA that has superior translational potential and no innate immunogenicity (Karikó et al., 2011, Nucleic Acids Research 39:e142). Administering HPLC-purified, pseudouridine-containing RNA coding for erythropoietin into mice and macaques resulted in a significant increase of serum EPO levels (Karikó et al., 2012, Mol Ther 20:948-953), thus confirming that pseudouridine-containing mRNA is suitable for in vivo protein therapy. In some embodiments, the nucleoside-modified nucleic acid molecule is purified using non-HPLC methods. In some instances, the nucleoside-modified nucleic acid molecule is purified using chromatography methods, including but not limited to HPLC and fast protein liquid chromatography (FPLC). An exemplary FPLC-based purification procedure is described in Weissman et al., 2013, Methods Mol Biol, 969: 43-54. Exemplary purification procedures are also described in U.S. Patent Application Publication No. US2016/0032316, which is hereby incorporated by reference in its entirety.

The present invention encompasses RNA, oligoribonucleotide, and polyribonucleotide molecules comprising pseudouridine or a modified nucleoside. In some embodiments, the composition comprises an isolated nucleic acid encoding an antigen, wherein the nucleic acid comprises a pseudouridine or a modified nucleoside. In some embodiments, the composition comprises a vector, comprising an isolated nucleic acid encoding an antigen, adjuvant, or combination thereof, wherein the nucleic acid comprises a pseudouridine or a modified nucleoside.

In one embodiment, the nucleoside-modified RNA of the invention is IVT RNA, as described elsewhere herein. For example, in some embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In one embodiment, the modified nucleoside is m¹acp³Ψ (1-methyl-3-(3-amino-3-carboxypropyl) pseudouridine. In another embodiment, the modified nucleoside is m¹Ψ (1-methylpseudouridine). In another embodiment, the modified nucleoside is Ψm (2′-O-methylpseudouridine). In another embodiment, the modified nucleoside is m⁵D (5-methyldihydrouridine). In another embodiment, the modified nucleoside is m¹Ψ (3-methylpseudouridine). In another embodiment, the modified nucleoside is a pseudouridine moiety that is not further modified. In another embodiment, the modified nucleoside is a monophosphate, diphosphate, or triphosphate of any of the above pseudouridines. In another embodiment, the modified nucleoside is any other pseudouridine-like nucleoside known in the art.

In another embodiment, the nucleoside that is modified in the nucleoside-modified RNA the present invention is uridine (U). In another embodiment, the modified nucleoside is cytidine (C). In another embodiment, the modified nucleoside is adenosine (A). In another embodiment, the modified nucleoside is guanosine (G).

In another embodiment, the modified nucleoside of the present invention is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine).

In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i6A (2-methylthio-N⁶isopentenyladenosine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t6A (2-methylthio-N⁶-threonyl carbamoyladenosine); m⁶t6A (N⁶-methyl-N⁶-threonylcarbamoyladenosine); hn⁶A (N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); frC (5-formylcytidine); msCm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²2G (N²,N²-dimethylguanosine); m²Gm (N²,2′-O-dimethylguanosine); m²2Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s2U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶2Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In another embodiment, a nucleoside-modified RNA of the present invention comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In various embodiments, between 0.1% and 100% of the residues in the nucleoside-modified RNA of the present invention are modified (e.g., either by the presence of pseudouridine, 1-methyl-pseudouridine, or another modified nucleoside base). In one embodiment, the fraction of modified residues is 0.1%. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.7%. In another embodiment, the fraction is 0.8%. In another embodiment, the fraction is 0.9%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 7%. In another embodiment, the fraction is 8%. In another embodiment, the fraction is 9%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 55%. In another embodiment, the fraction is 60%. In another embodiment, the fraction is 65%. In another embodiment, the fraction is 70%. In another embodiment, the fraction is 75%. In another embodiment, the fraction is 80%. In another embodiment, the fraction is 85%. In another embodiment, the fraction is 90%. In another embodiment, the fraction is 91%. In another embodiment, the fraction is 92%. In another embodiment, the fraction is 93%. In another embodiment, the fraction is 94%. In another embodiment, the fraction is 95%. In another embodiment, the fraction is 96%. In another embodiment, the fraction is 97%. In another embodiment, the fraction is 98%. In another embodiment, the fraction is 99%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.7%. In another embodiment, the fraction is 0.8%. In another embodiment, the fraction is 0.9%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 7%. In another embodiment, the fraction is 8%. In another embodiment, the fraction is 9%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 55%. In another embodiment, the fraction is 60%. In another embodiment, the fraction is 65%. In another embodiment, the fraction is 70%. In another embodiment, the fraction is 75%. In another embodiment, the fraction is 80%. In another embodiment, the fraction is 85%. In another embodiment, the fraction is 90%. In another embodiment, the fraction is 91%. In another embodiment, the fraction is 92%. In another embodiment, the fraction is 93%. In another embodiment, the fraction is 94%. In another embodiment, the fraction is 95%. In another embodiment, the fraction is 96%. In another embodiment, the fraction is 97%. In another embodiment, the fraction is 98%. In another embodiment, the fraction is 99%. In another embodiment, the fraction is 100%. In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In some embodiments, the composition comprises a purified preparation of single-stranded nucleoside modified RNA. For example, in some embodiments, the purified preparation of single-stranded nucleoside modified RNA is substantially free of double stranded RNA (dsRNA). In some embodiments, the purified preparation is at least 90%, or at least 91%, or at least 92%, or at least 93% or at least 94%, or at least 95%, or at least 96%, or at least 97%, or at least 98%, or at least 99%, or at least 99.5%, or at least 99.9% single stranded nucleoside modified RNA, relative to all other nucleic acid molecules (DNA, dsRNA, etc.).

In another embodiment, a nucleoside-modified RNA of the present invention is translated in the cell more efficiently than an unmodified RNA molecule with the same sequence. In another embodiment, the nucleoside-modified RNA exhibits enhanced ability to be translated by a target cell. In another embodiment, translation is enhanced by a factor of 2-fold relative to its unmodified counterpart. In another embodiment, translation is enhanced by a 3-fold factor. In another embodiment, translation is enhanced by a 4-fold factor. In another embodiment, translation is enhanced by a 5-fold factor. In another embodiment, translation is enhanced by a 6-fold factor. In another embodiment, translation is enhanced by a 7-fold factor. In another embodiment, translation is enhanced by an 8-fold factor. In another embodiment, translation is enhanced by a 9-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts.

In another embodiment, the nucleoside-modified antigen-encoding RNA of the present invention induces a significantly more robust adaptive immune response as compared with an unmodified in vitro-synthesized RNA molecule of the same sequence. In another embodiment, the modified RNA molecule induces an adaptive immune response that is 2-fold greater than its unmodified counterpart. In another embodiment, the adaptive immune response is increased by a 3-fold factor. In another embodiment, the adaptive immune response is increased by a 4-fold factor. In another embodiment, the adaptive immune response is increased by a 5-fold factor. In another embodiment, the adaptive immune response is increased by a 6-fold factor. In another embodiment, the adaptive immune response is increased by a 7-fold factor. In another embodiment, the adaptive immune response is increased by an 8-fold factor. In another embodiment, the adaptive immune response is increased by a 9-fold factor. In another embodiment, the adaptive immune response is increased by a 10-fold factor. In another embodiment, the adaptive immune response is increased by a 15-fold factor. In another embodiment, the adaptive immune response is increased by a 20-fold factor. In another embodiment, the adaptive immune response is increased by a 50-fold factor. In another embodiment, the adaptive immune response is increased by a 100-fold factor. In another embodiment, the adaptive immune response is increased by a 200-fold factor. In another embodiment, the adaptive immune response is increased by a 500-fold factor. In another embodiment, the adaptive immune response is increased by a 1000-fold factor. In another embodiment, the adaptive immune response is increased by a 2000-fold factor. In another embodiment, the adaptive immune response is increased by another fold difference.

In another embodiment, “induces significantly more robust adaptive immune response” refers to a detectable increase in an adaptive immune response. In another embodiment, the term refers to a fold increase in the adaptive immune response (e.g., 1 of the fold increases enumerated above). In another embodiment, the term refers to an increase such that the nucleoside-modified RNA can be administered at a lower dose or frequency than an unmodified RNA molecule while still inducing a similarly effective adaptive immune response. In another embodiment, the increase is such that the nucleoside-modified RNA can be administered using a single dose to induce an effective adaptive immune response.

In another embodiment, the nucleoside-modified RNA of the present invention exhibits significantly less innate immunogenicity than an unmodified in vitro-synthesized RNA molecule of the same sequence. In another embodiment, the modified RNA molecule exhibits an innate immune response that is 2-fold less than its unmodified counterpart. In another embodiment, innate immunogenicity is reduced by a 3-fold factor. In another embodiment, innate immunogenicity is reduced by a 4-fold factor. In another embodiment, innate immunogenicity is reduced by a 5-fold factor. In another embodiment, innate immunogenicity is reduced by a 6-fold factor. In another embodiment, innate immunogenicity is reduced by a 7-fold factor. In another embodiment, innate immunogenicity is reduced by a 8-fold factor. In another embodiment, innate immunogenicity is reduced by a 9-fold factor. In another embodiment, innate immunogenicity is reduced by a 10-fold factor. In another embodiment, innate immunogenicity is reduced by a 15-fold factor. In another embodiment, innate immunogenicity is reduced by a 20-fold factor. In another embodiment, innate immunogenicity is reduced by a 50-fold factor. In another embodiment, innate immunogenicity is reduced by a 100-fold factor. In another embodiment, innate immunogenicity is reduced by a 200-fold factor. In another embodiment, innate immunogenicity is reduced by a 500-fold factor. In another embodiment, innate immunogenicity is reduced by a 1000-fold factor. In another embodiment, innate immunogenicity is reduced by a 2000-fold factor. In another embodiment, innate immunogenicity is reduced by another fold difference.

In another embodiment, “exhibits significantly less innate immunogenicity” refers to a detectable decrease in innate immunogenicity. In another embodiment, the term refers to a fold decrease in innate immunogenicity (e.g., 1 of the fold decreases enumerated above). In another embodiment, the term refers to a decrease such that an effective amount of the nucleoside-modified RNA can be administered without triggering a detectable innate immune response. In another embodiment, the term refers to a decrease such that the nucleoside-modified RNA can be repeatedly administered without eliciting an innate immune response sufficient to detectably reduce production of the protein encoded by the modified RNA. In another embodiment, the decrease is such that the nucleoside-modified RNA can be repeatedly administered without eliciting an innate immune response sufficient to eliminate detectable production of the protein encoded by the modified RNA.

### Lipid Nanoparticle

In one embodiment, delivery of nucleoside-modified RNA comprises any suitable delivery method, including exemplary RNA transfection methods described elsewhere herein. In some embodiments, delivery of a nucleoside-modified RNA to a subject comprises mixing the nucleoside-modified RNA with a transfection reagent prior to the step of contacting. In another embodiment, a method of present invention further comprises administering nucleoside-modified RNA together with the transfection reagent. In another embodiment, the transfection reagent is a cationic lipid reagent. In another embodiment, the transfection reagent is a cationic polymer reagent.

In another embodiment, the transfection reagent is a lipid-based transfection reagent. In another embodiment, the transfection reagent is a protein-based transfection reagent. In another embodiment, the transfection reagent is a carbohydrate-based transfection reagent. In another embodiment, the transfection reagent is a cationic lipid-based transfection reagent. In another embodiment, the transfection reagent is a cationic polymer-based transfection reagent. In another embodiment, the transfection reagent is a polyethyleneimine based transfection reagent. In another embodiment, the transfection reagent is calcium phosphate. In another embodiment, the transfection reagent is Lipofectin®, Lipofectamine®, or TransIT®. In another embodiment, the transfection reagent is any other transfection reagent known in the art.

In another embodiment, the transfection reagent forms a liposome. Liposomes, in another embodiment, increase intracellular stability, increase uptake efficiency and improve biological activity. In another embodiment, liposomes are hollow spherical vesicles composed of lipids arranged in a similar fashion as those lipids, which make up the cell membrane. They have, in another embodiment, an internal aqueous space for entrapping water-soluble compounds and range in size from 0.05 to several microns in diameter. In another embodiment, liposomes can deliver RNA to cells in a biologically active form.

In one embodiment, the composition comprises a lipid nanoparticle (LNP) and one or more nucleic acid molecules described herein. For example, in one embodiment, the composition comprises an LNP and one or more nucleoside-modified RNA molecules encoding one or more antigens, adjuvants, or a combination thereof.

In some embodiments, the lipid nanoparticle is a particle having at least one dimension on the order of nanometers (e.g., 1-1,000 nm). In some embodiments, the lipid nanoparticle comprises one or more lipids. For example, in some embodiments, the lipid comprises a lipid of Formula (I), (II) or (III).

In some embodiments, lipid nanoparticles are included in a formulation comprising a nucleoside-modified RNA as described herein. In some embodiments, such lipid nanoparticles comprise a cationic lipid (e.g., a lipid of Formula (I), (II) or (III)) and one or more excipient selected from neutral lipids, charged lipids, steroids and polymer conjugated lipids (e.g., a pegylated lipid such as a pegylated lipid of structure (IV). In some embodiments, the nucleoside-modified RNA is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In some embodiments, the nucleoside-modified RNA, when present in the lipid nanoparticles, is resistant in aqueous solution to degradation with a nuclease.

The LNP may comprise any lipid capable of forming a particle to which the one or more nucleic acid molecules are attached, or in which the one or more nucleic acid molecules are encapsulated.

In one embodiment, the LNP comprises one or more cationic lipids, and one or more stabilizing lipids. Stabilizing lipids include neutral lipids and pegylated lipids.

In one embodiment, the LNP comprises a cationic lipid. In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA·Cl), 1,2-dilinoleoyl-3-trimethylaminopropane chloride salt (DLin-TAP·Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

Suitable amino lipids include those having the formula:

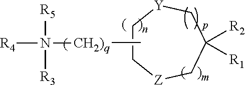

wherein R₁ and R₂ are either the same or different and independently optionally substituted C₁₀-C₂₄ alkyl, optionally substituted C₁₀-C₂₄ alkenyl, optionally substituted C₁₀-C₂₄ alkynyl, or optionally substituted C₁₀-C₂₄ acyl;
R₃ and R₄ are either the same or different and independently optionally substituted C₁-C₆ alkyl, optionally substituted C₂-C₆ alkenyl, or optionally substituted C₂-C₆ alkynyl or R₃ and R₄ may join to form an optionally substituted heterocyclic ring of 4 to 6 carbon atoms and 1 or 2 heteroatoms chosen from nitrogen and oxygen;
R₅ is either absent or present and when present is hydrogen or C₁-C₆ alkyl;
m, n, and p are either the same or different and independently either 0 or 1 with the proviso that m, n, and p are not simultaneously 0;
q is 0, 1, 2, 3, or 4; and
Y and Z are either the same or different and independently O, S, or NH.

In one embodiment, R₁ and R₂ are each linoleyl, and the amino lipid is a dilinoleyl amino lipid. In one embodiment, the amino lipid is a dilinoleyl amino lipid.

A representative useful dilinoleyl amino lipid has the formula:

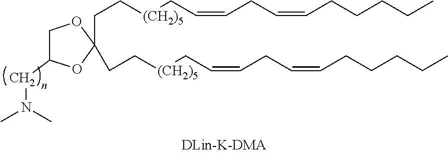

wherein n is 0, 1, 2, 3, or 4.

In one embodiment, the cationic lipid is a DLin-K-DMA. In one embodiment, the cationic lipid is DLin-KC2-DMA (DLin-K-DMA above, wherein n is 2).

In one embodiment, the cationic lipid component of the LNPs has the structure of Formula (I):
or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl;
R⁸ and R⁹ are each independently C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24;
b and c are each independently an integer from 1 to 24; and
e is 1 or 2.

In some embodiments of Formula (I), at least one of R^{1a}, R^{2a}, R^{3a} or R^{4a} is C₁-C₁₂ alkyl, or at least one of L¹ or L² is —O(C═O)— or —(C═O)O—. In other embodiments, R^{1a} and R^{1b} are not isopropyl when a is 6 or n-butyl when a is 8.

In still further embodiments of Formula (I), at least one of R¹, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

In other embodiments of Formula (I), R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;

In some embodiments of Formula (I), any one of L¹ or L² may be —O(C═O)— or a carbon-carbon double bond. L¹ and L² may each be —O(C═O)— or may each be a carbon-carbon double bond.

In some embodiments of Formula (I), one of L¹ or L² is —O(C═O)—. In other embodiments, both L¹ and L² are —O(C═O)—.

In some embodiments of Formula (I), one of L¹ or L² is —(C═O)O—. In other embodiments, both L¹ and L² are —(C═O)O—.

In some other embodiments of Formula (I), one of L¹ or L² is a carbon-carbon double bond. In other embodiments, both L¹ and L² are a carbon-carbon double bond.

In still other embodiments of Formula (I), one of L¹ or L² is —O(C═O)— and the other of L¹ or L² is —(C═O)O—. In more embodiments, one of L¹ or L² is —O(C═O)— and the other of L¹ or L² is a carbon-carbon double bond. In yet more embodiments, one of L¹ or L² is —(C═O)O— and the other of L¹ or L² is a carbon-carbon double bond.

It is understood that “carbon-carbon” double bond, as used throughout the specification, refers to one of the following structures:

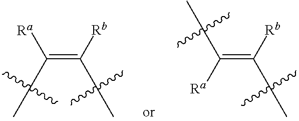

wherein R^{a} and R^{b} are, at each occurrence, independently H or a substituent. For example, in some embodiments R^{a} and R^{b} are, at each occurrence, independently H, C₁-C₁₂ alkyl or cycloalkyl, for example H or C₁-C₁₂ alkyl.

In other embodiments, the lipid compounds of Formula (I) have the following structure (Ia):

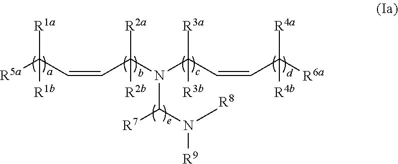

In other embodiments, the lipid compounds of Formula (I) have the following structure (Ib):

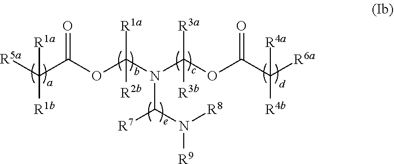

In yet other embodiments, the lipid compounds of Formula (I) have the following structure (Ic):

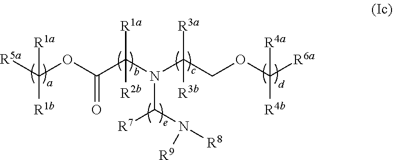

In some embodiments of the lipid compound of Formula (I), a, b, c and d are each independently an integer from 2 to 12 or an integer from 4 to 12. In other embodiments, a, b, c and d are each independently an integer from 8 to 12 or 5 to 9. In some embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some other embodiments of Formula (I), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some more embodiments of Formula (I), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some other embodiments of Formula (I), d is 0. In some embodiments, d is 1. In other embodiments, d is 2. In more embodiments, d is 3. In yet other embodiments, d is 4. In some embodiments, d is 5. In other embodiments, d is 6. In more embodiments, d is 7. In yet other embodiments, d is 8. In some embodiments, d is 9. In other embodiments, d is 10. In more embodiments, d is 11. In yet other embodiments, d is 12. In some embodiments, d is 13. In other embodiments, d is 14. In more embodiments, d is 15. In yet other embodiments, d is 16.

In some other various embodiments of Formula (I), a and d are the same. In some other embodiments, b and c are the same. In some other specific embodiments, a and d are the same and b and c are the same.

The sum of a and b and the sum of c and d in Formula (I) are factors which may be varied to obtain a lipid of Formula (I) having the desired properties. In one embodiment, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a. b, c and d are selected such the sum of a and b and the sum of c and d is 12 or greater.

In some embodiments of Formula (I), e is 1. In other embodiments, e is 2.

The substituents at R^{1a}, R^{2a}, R^{3a} and R^{4a} of Formula (I) are not particularly limited. In some embodiments R^{1a}, R^{2a}, R^{3a} and R^{4a} are H at each occurrence. In some other embodiments at least one of R^{1a}, R^{2a}, R^{3a} and R^{4a} is C₁-C₁₂ alkyl. In some other embodiments at least one of R^{1a}, R^{2a}, R^{3a} and R^{4a} is C₁-C₈ alkyl. In some other embodiments at least one of R^{1a}, R^{2a}, R^{3a} and R^{4a} is C₁-C₆ alkyl. In some of the foregoing embodiments, the C₁-C₈ alkyl is methyl, ethyl, n-propyl, iso-propyl, n-butyl, iso-butyl, tert-butyl, n-hexyl or n-octyl.

In some embodiments of Formula (I), R^{1a}, R^{1b}, R^{4a} and R^{4b} are C₁-C₁₂ alkyl at each occurrence.

In further embodiments of Formula (I), at least one of R^{1b}, R^{2b}, R^{3b} and R^{4b} is H or R^{1b}, R^{2b}, R^{3b} and R^{4b} are H at each occurrence.

In some embodiments of Formula (I), R^{1b} together with the carbon atom to which it is bound is taken together with an adjacent R^{1b} and the carbon atom to which it is bound to form a carbon-carbon double bond. In other embodiments of the foregoing R^{4b} together with the carbon atom to which it is bound is taken together with an adjacent R^{4b} and the carbon atom to which it is bound to form a carbon-carbon double bond.

The substituents at R⁵ and R⁶ of Formula (I) are not particularly limited in the foregoing embodiments. In some embodiments one or both of R⁵ or R⁶ is methyl. In some other embodiments one or both of R⁵ or R⁶ is cycloalkyl for example cyclohexyl. In these embodiments the cycloalkyl may be substituted or not substituted. In some other embodiments the cycloalkyl is substituted with C₁-C₁₂ alkyl, for example tert-butyl.

The substituents at R⁷ are not particularly limited in the foregoing embodiments of Formula (I). In some embodiments at least one R⁷ is H. In some other embodiments, R⁷ is H at each occurrence. In some other embodiments R⁷ is C₁-C₁₂ alkyl.

In some other of the foregoing embodiments of Formula (I), one of R⁸ or R⁹ is methyl. In other embodiments, both R⁸ and R⁹ are methyl.

In some different embodiments of Formula (I), R⁸ and R⁹, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring.

In various different embodiments, the lipid of Formula (I) has one of the structures set forth in Table 1 below.

TABLE 1
Representative Lipids of Formula (I).

| | | Prep. |
|---|---|---|
| No. | Structure | Method |
| I-1 | 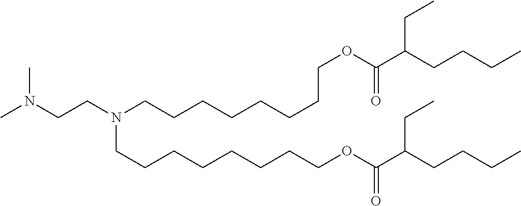 [SMILES: CCCCC(CC)C(=O)OCCCCCCCCN(CCCCCCCCOC(=O)C(CC)CCCC)CCN(C)C] | B |
| I-2 | 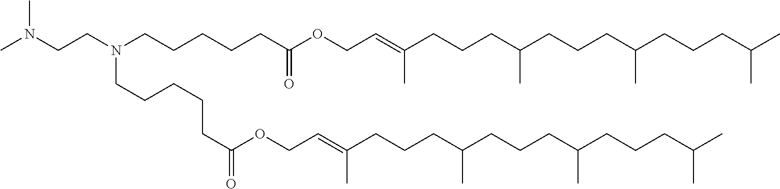 [SMILES: C/C(=C\COC(=O)CCCCCN(CCCCCC(=O)OC/C=C(\C)CCCC(C)CCCC(C)CCCC(C)C)CCN(C)C)CCCC(C)CCCC(C)CCCC(C)C] | A |
| I-3 | 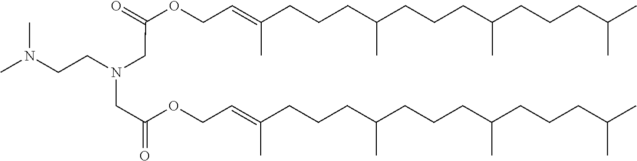 [SMILES: C/C(=C\COC(=O)CN(CCN(C)C)CC(=O)OC/C=C(\C)CCCC(C)CCCC(C)CCCC(C)C)CCCC(C)CCCC(C)CCCC(C)C] | A |
| I-4 | 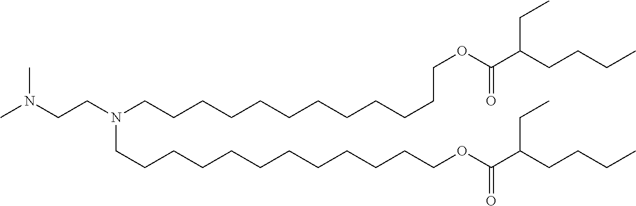 [SMILES: CCCCC(CC)C(=O)OCCCCCCCCCCCCN(CCCCCCCCCCCCOC(=O)C(CC)CCCC)CCN(C)C] | B |
| I-5 | 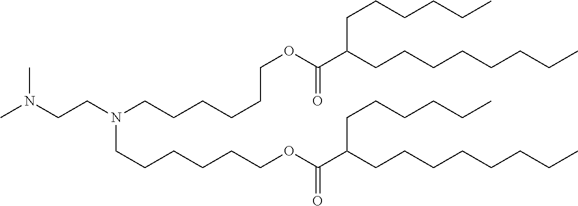 [SMILES: CCCCCCCCC(CCCCCC)C(=O)OCCCCCCN(CCCCCCOC(=O)C(CCCCCC)CCCCCCCC)CCN(C)C] | B |
| I-6 | 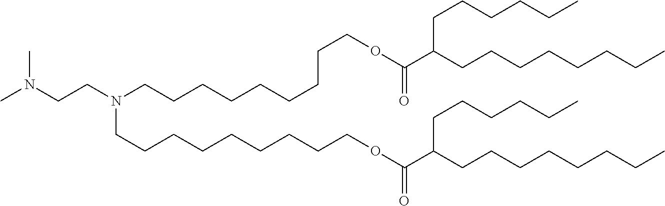 [SMILES: CCCCCCCCC(CCCCCC)C(=O)OCCCCCCCCCN(CCCCCCCCCOC(=O)C(CCCCCC)CCCCCCCC)CCN(C)C] | B |
| I-7 | 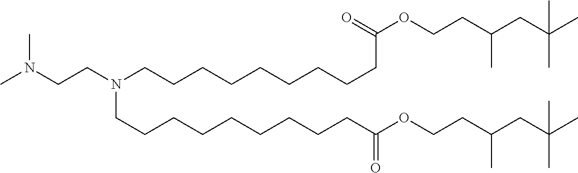 [SMILES: CC(CCOC(=O)CCCCCCCCCN(CCCCCCCCCC(=O)OCCC(C)CC(C)(C)C)CCN(C)C)CC(C)(C)C] | A |
| I-8 | 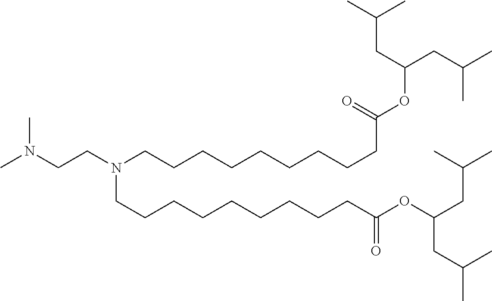 [SMILES: CC(C)CC(CC(C)C)OC(=O)CCCCCCCCCN(CCCCCCCCCC(=O)OC(CC(C)C)CC(C)C)CCN(C)C] | A |
| I-9 | 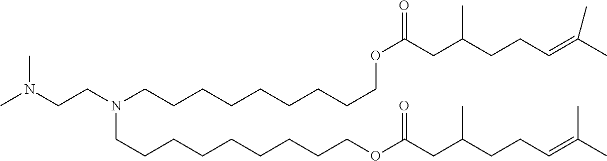 [SMILES: CC(C)=CCCC(C)CC(=O)OCCCCCCCCCN(CCCCCCCCCOC(=O)CC(C)CCC=C(C)C)CCN(C)C] | B |
| I-10 | 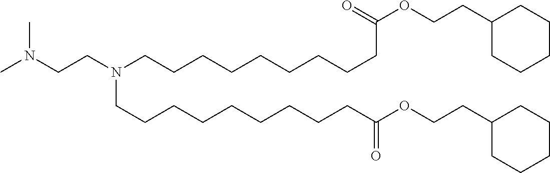 [SMILES: CN(C)CCN(CCCCCCCCCC(=O)OCCC1CCCCC1)CCCCCCCCCC(=O)OCCC1CCCCC1] | A |
| I-11 | 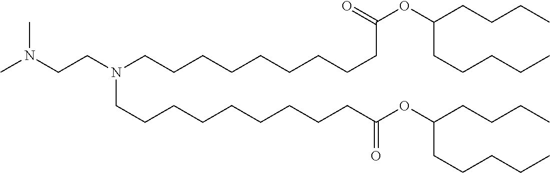 [SMILES: CCCCCC(CCCC)OC(=O)CCCCCCCCCN(CCCCCCCCCC(=O)OC(CCCC)CCCCC)CCN(C)C] | A |
| I-12 | 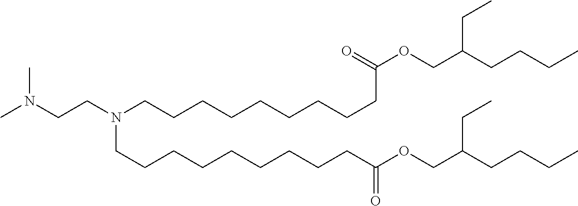 [SMILES: CCCCC(CC)COC(=O)CCCCCCCCCN(CCCCCCCCCC(=O)OCC(CC)CCCC)CCN(C)C] | A |
| I-13 | 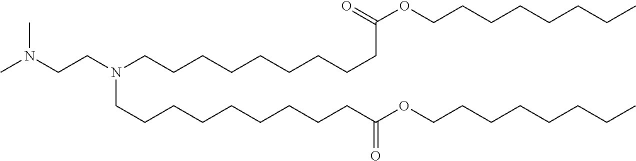 [SMILES: CCCCCCCCOC(=O)CCCCCCCCCN(CCCCCCCCCC(=O)OCCCCCCCC)CCN(C)C] | A |
| I-14 | 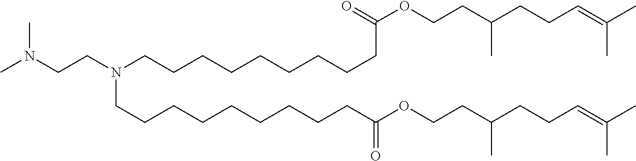 [SMILES: CC(C)=CCCC(C)CCOC(=O)CCCCCCCCCN(CCCCCCCCCC(=O)OCCC(C)CCC=C(C)C)CCN(C)C] | A |
| I-15 | 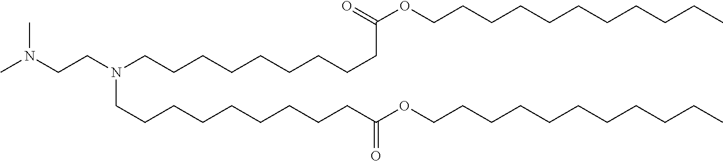 [SMILES: CCCCCCCCCCCOC(=O)CCCCCCCCCN(CCCCCCCCCC(=O)OCCCCCCCCCCC)CCN(C)C] | A |
| I-16 | 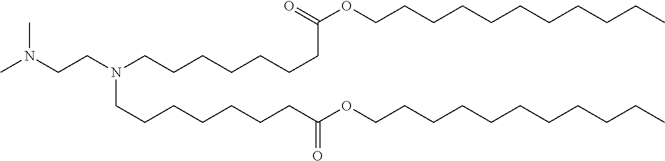 [SMILES: CCCCCCCCCCCOC(=O)CCCCCCCN(CCCCCCCC(=O)OCCCCCCCCCCC)CCN(C)C] | A |
| I-17 | 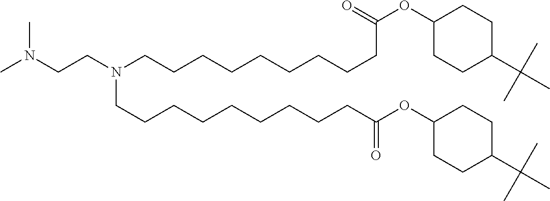 [SMILES: CN(C)CCN(CCCCCCCCCC(=O)OC1CCC(C(C)(C)C)CC1)CCCCCCCCCC(=O)OC1CCC(C(C)(C)C)CC1] | A |
| I-18 | 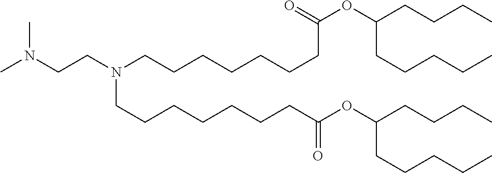 [SMILES: CCCCCC(CCCC)OC(=O)CCCCCCCN(CCCCCCCC(=O)OC(CCCC)CCCCC)CCN(C)C] | A |
| I-19 | 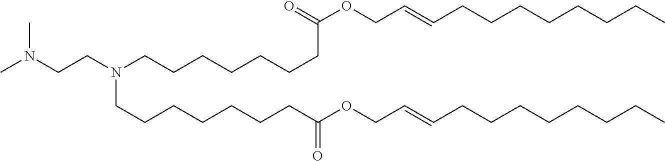 [SMILES: CCCCCCCC/C=C/COC(=O)CCCCCCCN(CCCCCCCC(=O)OC/C=C/CCCCCCCC)CCN(C)C] | A |
| I-20 | 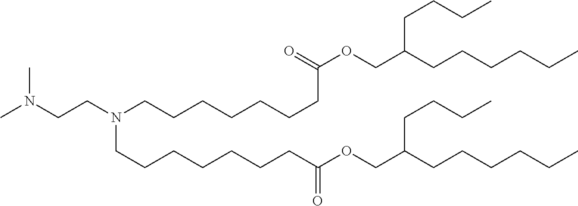 [SMILES: CCCCCCC(CCCC)COC(=O)CCCCCCCN(CCCCCCCC(=O)OCC(CCCC)CCCCCC)CCN(C)C] | A |
| I-21 | 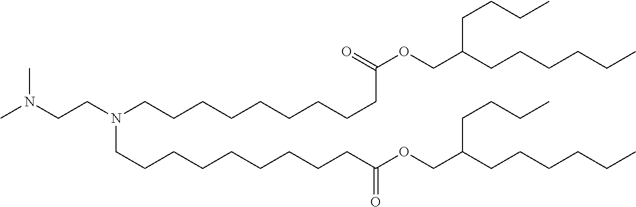 [SMILES: CCCCCCC(CCCC)COC(=O)CCCCCCCCCN(CCCCCCCCCC(=O)OCC(CCCC)CCCCCC)CCN(C)C] | A |
| I-22 | 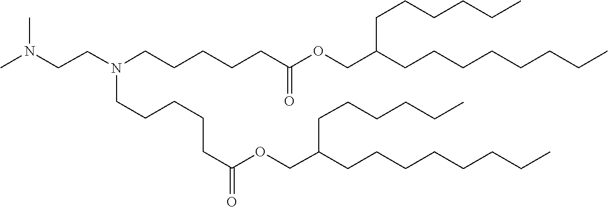 [SMILES: CCCCCCCCC(CCCCCC)COC(=O)CCCCCN(CCCCCC(=O)OCC(CCCCCC)CCCCCCCC)CCN(C)C] | A |
| I-23 | 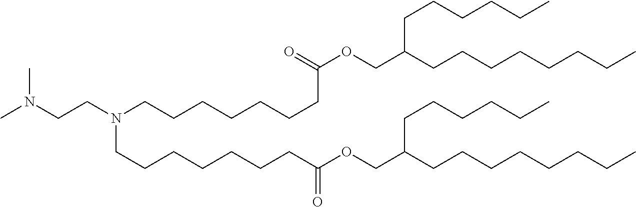 [SMILES: CCCCCCCCC(CCCCCC)COC(=O)CCCCCCCN(CCCCCCCC(=O)OCC(CCCCCC)CCCCCCCC)CCN(C)C] | A |
| I-24 | 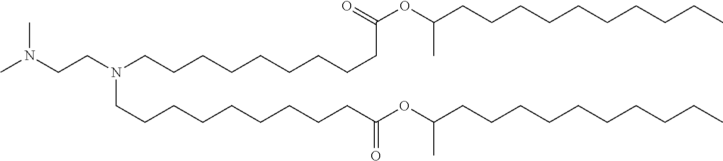 [SMILES: CCCCCCCCCCC(C)OC(=O)CCCCCCCCCN(CCCCCCCCCC(=O)OC(C)CCCCCCCCCC)CCN(C)C] | A |
| I-25 | 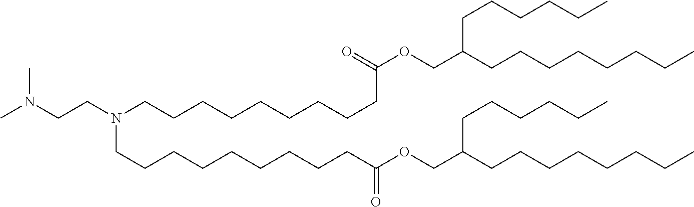 [SMILES: CCCCCCCCC(CCCCCC)COC(=O)CCCCCCCCCN(CCCCCCCCCC(=O)OCC(CCCCCC)CCCCCCCC)CCN(C)C] | A |
| I-26 | 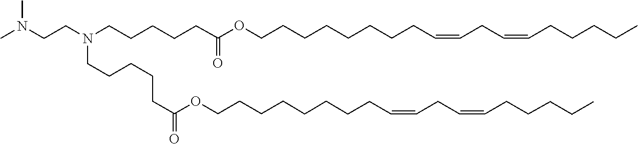 [SMILES: CCCCC/C=C\C/C=C\CCCCCCCCOC(=O)CCCCCN(CCCCCC(=O)OCCCCCCCC/C=C\C/C=C\CCCCC)CCN(C)C] | A |
| I-27 | 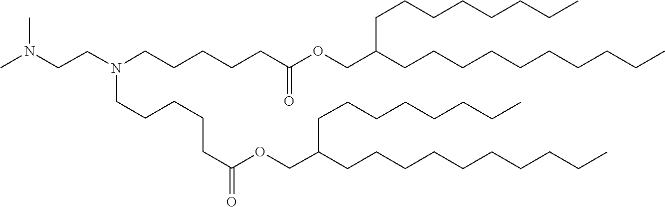 [SMILES: CCCCCCCCCCC(CCCCCCCC)COC(=O)CCCCCN(CCCCCC(=O)OCC(CCCCCCCC)CCCCCCCCCC)CCN(C)C] | A |
| I-28 | 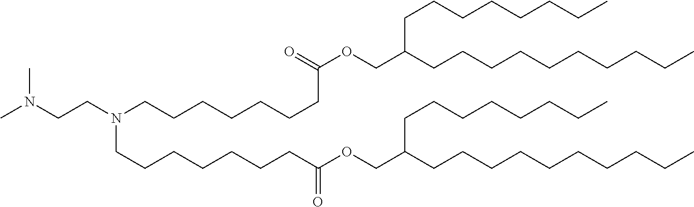 [SMILES: CCCCCCCCCCC(CCCCCCCC)COC(=O)CCCCCCCN(CCCCCCCC(=O)OCC(CCCCCCCC)CCCCCCCCCC)CCN(C)C] | A |
| I-29 | 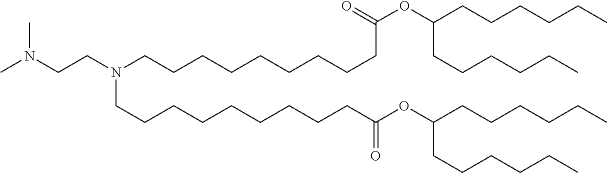 [SMILES: CCCCCCC(CCCCCC)OC(=O)CCCCCCCCCN(CCCCCCCCCC(=O)OC(CCCCCC)CCCCCC)CCN(C)C] | A |
| I-30 | 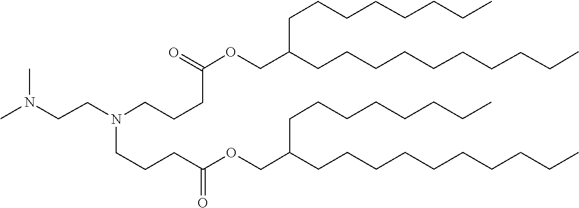 [SMILES: CCCCCCCCCCC(CCCCCCCC)COC(=O)CCCN(CCCC(=O)OCC(CCCCCCCC)CCCCCCCCCC)CCN(C)C] | A |
| I-31 | 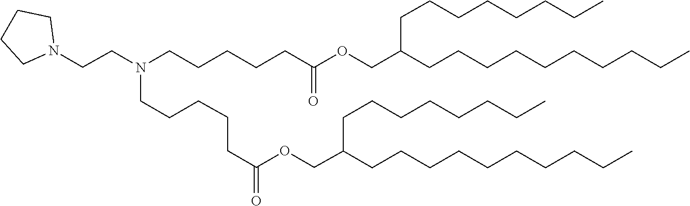 [SMILES: CCCCCCCCCCC(CCCCCCCC)COC(=O)CCCCCN(CCCCCC(=O)OCC(CCCCCCCC)CCCCCCCCCC)CCN1CCCC1] | C |
| I-32 | 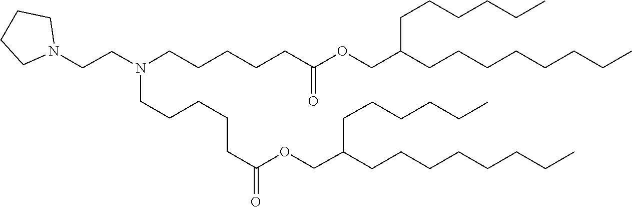 [SMILES: CCCCCCCCC(CCCCCC)COC(=O)CCCCCN(CCCCCC(=O)OCC(CCCCCC)CCCCCCCC)CCN1CCCC1] | C |
| I-33 | 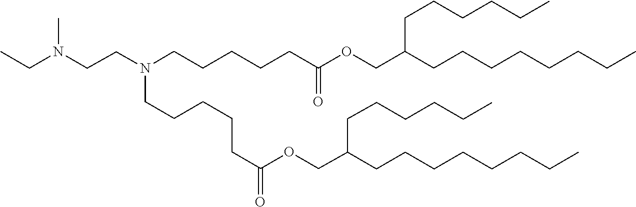 [SMILES: CCCCCCCCC(CCCCCC)COC(=O)CCCCCN(CCCCCC(=O)OCC(CCCCCC)CCCCCCCC)CCN(C)CC] | C |
| I-34 | 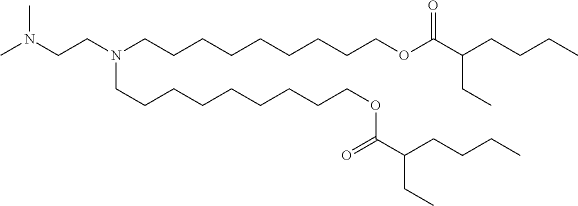 [SMILES: CCCCC(CC)C(=O)OCCCCCCCCCN(CCCCCCCCCOC(=O)C(CC)CCCC)CCN(C)C] | B |
| I-35 | 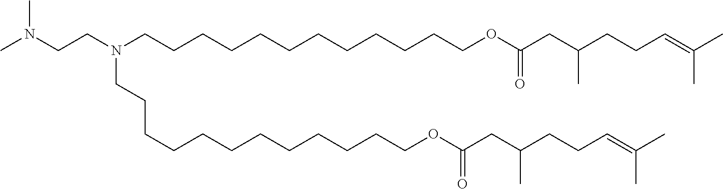 [SMILES: CC(C)=CCCC(C)CC(=O)OCCCCCCCCCCCCN(CCCCCCCCCCCCOC(=O)CC(C)CCC=C(C)C)CCN(C)C] | B |
| I-36 | 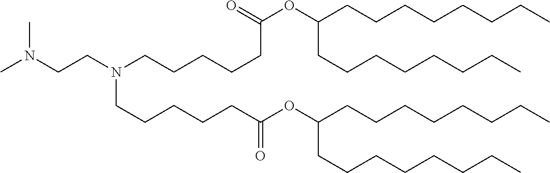 [SMILES: CCCCCCCCC(CCCCCCCC)OC(=O)CCCCCN(CCCCCC(=O)OC(CCCCCCCC)CCCCCCCC)CCN(C)C] | C |
| I-37 | 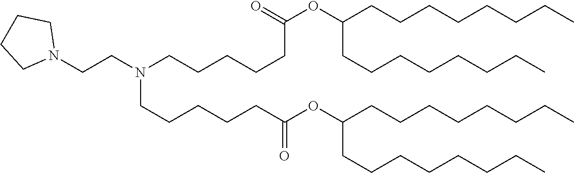 [SMILES: CCCCCCCCC(CCCCCCCC)OC(=O)CCCCCN(CCCCCC(=O)OC(CCCCCCCC)CCCCCCCC)CCN1CCCC1] | C |
| I-38 | 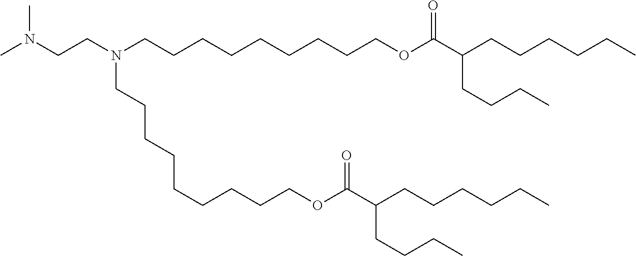 [SMILES: CCCCCCC(CCCC)C(=O)OCCCCCCCCCN(CCCCCCCCCOC(=O)C(CCCC)CCCCCC)CCN(C)C] | B |
| I-39 | 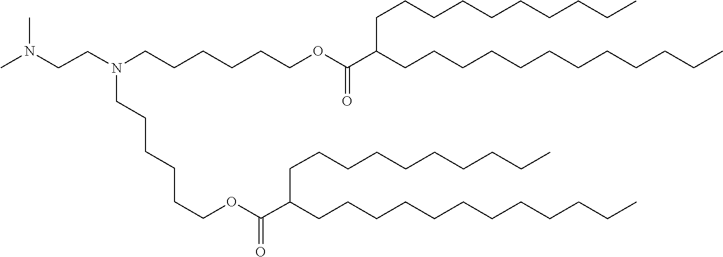 [SMILES: CCCCCCCCCCCCC(CCCCCCCCCC)C(=O)OCCCCCCN(CCCCCCOC(=O)C(CCCCCCCCCC)CCCCCCCCCCCC)CCN(C)C] | B |
| I-40 | 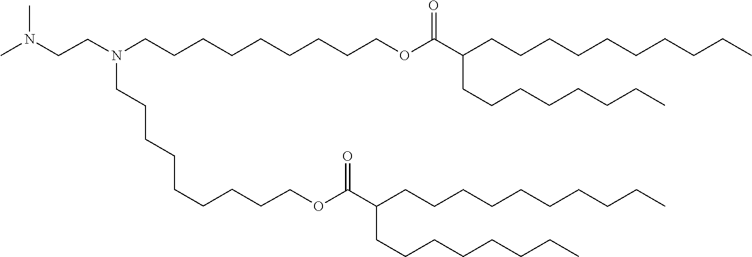 [SMILES: CCCCCCCCCCC(CCCCCCCC)C(=O)OCCCCCCCCCN(CCCCCCCCCOC(=O)C(CCCCCCCC)CCCCCCCCCC)CCN(C)C] | B |
| I-41 | 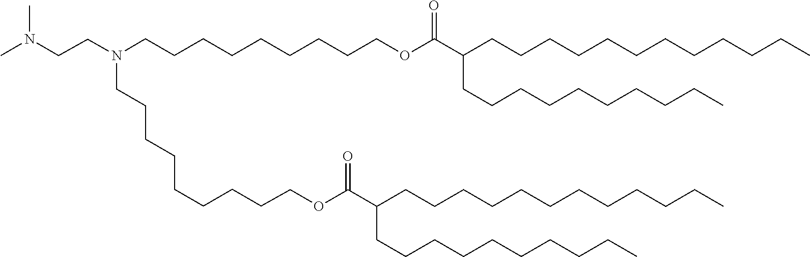 [SMILES: CCCCCCCCCCCCC(CCCCCCCCCC)C(=O)OCCCCCCCCCN(CCCCCCCCCOC(=O)C(CCCCCCCCCC)CCCCCCCCCCCC)CCN(C)C] | B |

In some embodiments, the LNPs comprise a lipid of Formula (I), a nucleoside-modified RNA and one or more excipients selected from neutral lipids, steroids and pegylated lipids. In some embodiments the lipid of Formula (I) is compound 1-5. In some embodiments the lipid of Formula (I) is compound 1-6.

In some other embodiments, the cationic lipid component of the LNPs has the structure of Formula (II):

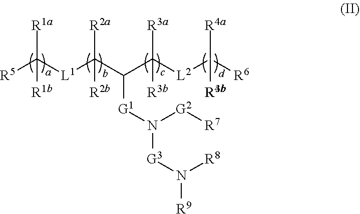

or a pharmaceutically acceptable salt, tautomer, prodrug or stereoisomer thereof, wherein:
L¹ and L² are each independently —O(C═O)—, —(C═O)O—, —C(═O)—, —O—,
—S(O)ₓ—, —S—S—, —C(═O)S—, —SC(═O)—, —NR^{a}C(═O)—, —C(═O)NR^{a}—, —NR^{a}C(═O)NR^{a},
—OC(═O)NR^{a}—, —NR^{a}C(═O)O—, or a direct bond;
G¹ is C₁-C₂ alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a} or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either: (a) H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and
x is 0, 1 or 2.

In some embodiments of Formula (II), L¹ and L² are each independently
O(C═O)—, —(C═O)O— or a direct bond. In other embodiments, G¹ and G² are each independently —(C═O)— or a direct bond. In some different embodiments, L¹ and L² are each independently —O(C═O)—, —(C═O)O— or a direct bond; and G¹ and G² are each independently —(C═O)— or a direct bond.

In some different embodiments of Formula (II), L¹ and L² are each independently —C(═O)—, —O—, —S(O)ₓ—, —S—S—, —C(═O)S—, —SC(═O)—, —NR^{a}—, —NR^{a}C(═O)—, —C(═O)NR^{a}—, —NR^{a}C(═O)NR^{a}, —OC(═O)NR^{a}—, —NR^{a}C(═O)O—, —NR^{a}S(O)ₓNR^{a}—, —NR^{a}S(O)ₓ— or —S(O)ₓNR^{a}—.

In other of the foregoing embodiments of Formula (II), the lipid compound has one of the following structures (IIA) or (IIB):

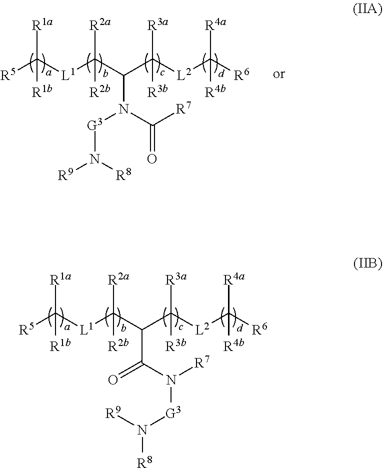

In some embodiments of Formula (II), the lipid compound has structure (IIA). In other embodiments, the lipid compound has structure (IIB).

In any of the foregoing embodiments of Formula (II), one of L¹ or L² is —O(C═O)—. For example, in some embodiments each of L¹ and L² are —O(C═O)—.

In some different embodiments of Formula (II), one of L¹ or L² is —(C═O)O—. For example, in some embodiments each of L¹ and L² is —(C═O)O—.

In different embodiments of Formula (II), one of L¹ or L² is a direct bond. As used herein, a “direct bond” means the group (e.g., L¹ or L²) is absent. For example, in some embodiments each of L¹ and L² is a direct bond.

In other different embodiments of Formula (II), for at least one occurrence of Ria and R^{1b}, R^{1a} is H or C₁-C₁₂ alkyl, and R^{1b} together with the carbon atom to which it is bound is taken together with an adjacent R^{1b} and the carbon atom to which it is bound to form a carbon-carbon double bond.

In still other different embodiments of Formula (II), for at least one occurrence of R^{4a} and R^{4b}, R^{4a} is H or C₁-C₁₂ alkyl, and R^{4b} together with the carbon atom to which it is bound is taken together with an adjacent R^{4b} and the carbon atom to which it is bound to form a carbon-carbon double bond.

In more embodiments of Formula (II), for at least one occurrence of R^{2a} and R^{2b}, R^{2a} is H or C₁-C₁₂ alkyl, and R^{2b} together with the carbon atom to which it is bound is taken together with an adjacent R^{2b} and the carbon atom to which it is bound to form a carbon-carbon double bond.

In other different embodiments of Formula (II), for at least one occurrence of R^{3a} and R^{3b}, R^{3a} is H or C₁-C₁₂ alkyl, and R^{3b} together with the carbon atom to which it is bound is taken together with an adjacent R^{3b} and the carbon atom to which it is bound to form a carbon-carbon double bond.

In various other embodiments of Formula (II), the lipid compound has one of the following structures (IIC) or (IID):

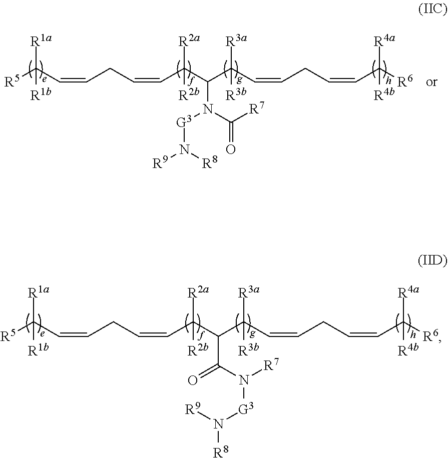

wherein e, f, g and h are each independently an integer from 1 to 12.

In some embodiments of Formula (II), the lipid compound has structure (IIC). In other embodiments, the lipid compound has structure (IID).

In various embodiments of structures (IIC) or (IID), e, f, g and h are each independently an integer from 4 to 10.

In some embodiments of Formula (II), a, b, c and d are each independently an integer from 2 to 12 or an integer from 4 to 12. In other embodiments, a, b, c and d are each independently an integer from 8 to 12 or 5 to 9. In some embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some embodiments of Formula (II), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some embodiments of Formula (II), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some embodiments of Formula (II), d is 0. In some embodiments, d is 1. In other embodiments, d is 2. In more embodiments, d is 3. In yet other embodiments, d is 4. In some embodiments, d is 5. In other embodiments, d is 6. In more embodiments, d is 7. In yet other embodiments, d is 8. In some embodiments, d is 9. In other embodiments, d is 10. In more embodiments, d is 11. In yet other embodiments, d is 12. In some embodiments, d is 13. In other embodiments, d is 14. In more embodiments, d is 15. In yet other embodiments, d is 16.

In some embodiments of Formula (II), e is 1. In other embodiments, e is 2. In more embodiments, e is 3. In yet other embodiments, e is 4. In some embodiments, e is 5. In other embodiments, e is 6. In more embodiments, e is 7. In yet other embodiments, e is 8. In some embodiments, e is 9. In other embodiments, e is 10. In more embodiments, e is 11. In yet other embodiments, e is 12.

In some embodiments of Formula (II), f is 1. In other embodiments, f is 2. In more embodiments, f is 3. In yet other embodiments, f is 4. In some embodiments, f is 5. In other embodiments, f is 6. In more embodiments, f is 7. In yet other embodiments, f is 8. In some embodiments, f is 9. In other embodiments, f is 10. In more embodiments, f is 11. In yet other embodiments, f is 12.

In some embodiments of Formula (II), g is 1. In other embodiments, g is 2. In more embodiments, g is 3. In yet other embodiments, g is 4. In some embodiments, g is 5. In other embodiments, g is 6. In more embodiments, g is 7. In yet other embodiments, g is 8. In some embodiments, g is 9. In other embodiments, g is 10. In more embodiments, g is 11. In yet other embodiments, g is 12.

In some embodiments of Formula (II), h is 1. In other embodiments, e is 2. In more embodiments, h is 3. In yet other embodiments, h is 4. In some embodiments, e is 5. In other embodiments, h is 6. In more embodiments, h is 7. In yet other embodiments, h is 8. In some embodiments, h is 9. In other embodiments, h is 10. In more embodiments, h is 11. In yet other embodiments, h is 12.

In some other various embodiments of Formula (II), a and d are the same. In some other embodiments, b and c are the same. In some other specific embodiments and a and d are the same and b and c are the same.

The sum of a and b and the sum of c and d of Formula (II) are factors which may be varied to obtain a lipid having the desired properties. In one embodiment, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a. b, c and d are selected such that the sum of a and b and the sum of c and d is 12 or greater.

The substituents at R^{1a}, R^{2a}, R^{3a} and R^{4a} of Formula (II) are not particularly limited. In some embodiments, at least one of R¹, R^{2a}, R^{3a} and R^{4a} is H. In some embodiments R^{1a}, R^{2a}, R^{3a} and R^{4a} are H at each occurrence. In some other embodiments at least one of Ria, R^{2a}, R^{3a} and R^{4a} is C₁-C₁₂ alkyl. In some other embodiments at least one of R¹, R^{2a}, R^{3a} and R^{4a} is C₁-C₈ alkyl. In some other embodiments at least one of Ria R^{2a}, R^{3a} and R^{4a} is C₁-C₆ alkyl. In some of the foregoing embodiments, the C₁-C₈ alkyl is methyl, ethyl, n-propyl, iso-propyl, n-butyl, iso-butyl, tert-butyl, n-hexyl or n-octyl.

In some embodiments of Formula (II), R^{1a}, R^{1b}, R^{4a} and R^{4b} are C₁-C₁₂ alkyl at each occurrence.

In further embodiments of Formula (II), at least one of R^{1b}, R^{2b}, R^{3b} and R^{4b} is H or R^{1b}, R^{2b}, R^{3b} and R^{4b} are H at each occurrence.

In some embodiments of Formula (II), R^{1b} together with the carbon atom to which it is bound is taken together with an adjacent R^{1b} and the carbon atom to which it is bound to form a carbon-carbon double bond. In other embodiments of the foregoing R^{4b}together with the carbon atom to which it is bound is taken together with an adjacent R^{4b} and the carbon atom to which it is bound to form a carbon-carbon double bond.

The substituents at R⁵ and R⁶ of Formula (II) are not particularly limited in the foregoing embodiments. In some embodiments one of R⁵ or R⁶ is methyl. In other embodiments each of R⁵ or R⁶ is methyl.

The substituents at R⁷ of Formula (II) are not particularly limited in the foregoing embodiments. In some embodiments R⁷ is C₆-C₁₆ alkyl. In some other embodiments, R⁷ is C₆-C₉ alkyl. In some of these embodiments, R⁷ is substituted with —(C═O)OR^{b}, —O(C═O)R^{b}, —C(═O)R^{b}, —OR^{b}, —S(O)ₓR^{b}, —S—SR^{b}, —C(═O)SR^{b},
—SC(═O)R^{b}, —NR^{a}R^{b}, —NR^{a}C(═O)R^{b}, —C(═O)NR^{a}R^{b}, —NR^{a}C(═O)NR^{a}R^{b},
—OC(═O)NR^{a}R^{b}, —NR^{a}C(═O)OR^{b}, —NR^{a}S(O)ₓNR^{a}R^{b}, —NR^{a}S(O)ₓR^{b} or —S(O)ₓNR^{a}R^{b}, wherein: R^{a} is H or C₁-C₁₂ alkyl; R^{b} is C₁-C₁₅ alkyl; and x is 0, 1 or 2. For example, in some embodiments R⁷ is substituted with —(C═O)OR^{b} or —O(C═O)R^{b}.

In various of the foregoing embodiments of Formula (II), R^{b} is branched C₁-C₁₅ alkyl. For example, in some embodiments R^{b} has one of the following structures:

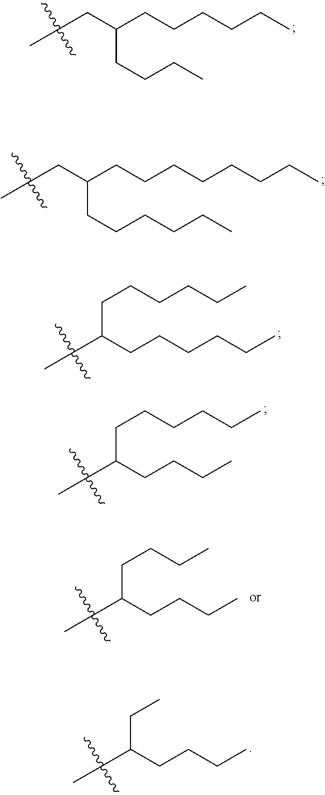

In some other of the foregoing embodiments of Formula (II), one of R⁸ or R⁹ is methyl. In other embodiments, both R⁸ and R⁹ are methyl.

In some different embodiments of Formula (II), R⁸ and R⁹, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring. In some different embodiments of the foregoing, R⁸ and R⁹, together with the nitrogen atom to which they are attached, form a 6-membered heterocyclic ring, for example a piperazinyl ring.

In still other embodiments of the foregoing lipids of Formula (II), G³ is C₂-C₄ alkylene, for example C₃ alkylene.

In various different embodiments, the lipid compound has one of the structures set forth in Table 2 below.

TABLE 2
Representative Lipids of Formula (II).

| | | Prep. |
|---|---|---|
| No. | Structure | Method |
| II-1 | 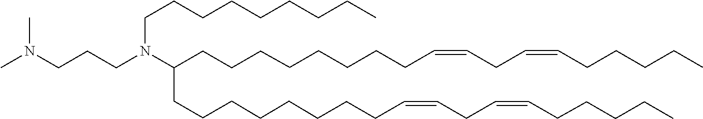 [SMILES: CCCCC/C=C\C/C=C\CCCCCCCCC(CCCCCCCC/C=C\C/C=C\CCCCC)N(CCCCCCCCC)CCCN(C)C] | D |
| II-2 | 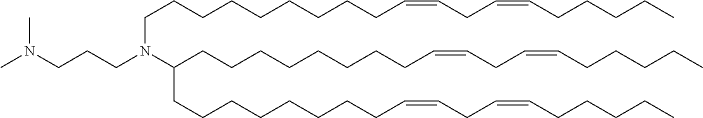 [SMILES: CCCCC/C=C\C/C=C\CCCCCCCCCN(CCCN(C)C)C(CCCCCCCC/C=C\C/C=C\CCCCC)CCCCCCCC/C=C\C/C=C\CCCCC] | D |
| II-3 | 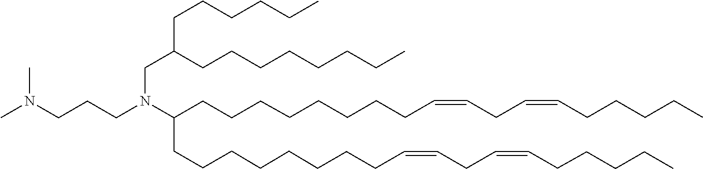 [SMILES: CCCCC/C=C\C/C=C\CCCCCCCCC(CCCCCCCC/C=C\C/C=C\CCCCC)N(CCCN(C)C)CC(CCCCCC)CCCCCCCC] | D |
| II-4 | 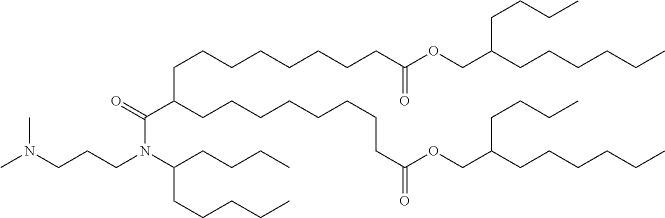 [SMILES: CCCCCCC(CCCC)COC(=O)CCCCCCCCC(CCCCCCCCC(=O)OCC(CCCC)CCCCCC)C(=O)N(CCCN(C)C)C(CCCC)CCCCC] | E |
| II-5 | 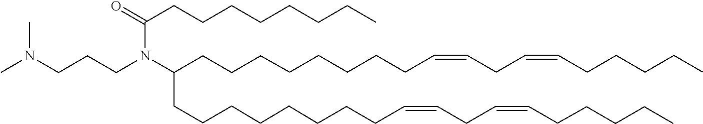 [SMILES: CCCCC/C=C\C/C=C\CCCCCCCCC(CCCCCCCC/C=C\C/C=C\CCCCC)N(CCCN(C)C)C(=O)CCCCCCCC] | D |
| II-6 | 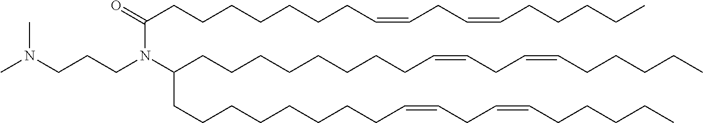 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC] | D |
| II-7 | 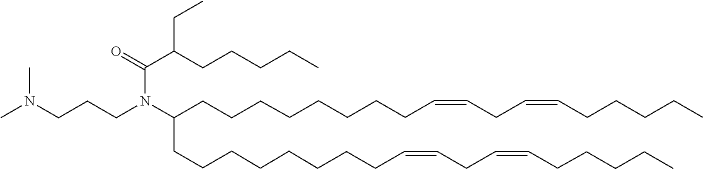 [SMILES: CCCCC/C=C\C/C=C\CCCCCCCCC(CCCCCCCC/C=C\C/C=C\CCCCC)N(CCCN(C)C)C(=O)C(CC)CCCCC] | D |
| II-8 | 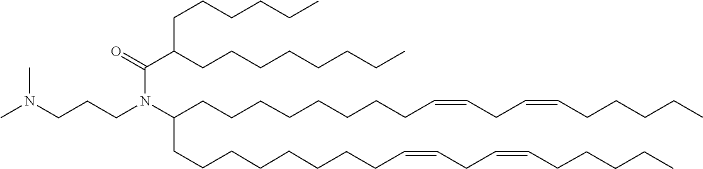 [SMILES: CCCCC/C=C\C/C=C\CCCCCCCCC(CCCCCCCC/C=C\C/C=C\CCCCC)N(CCCN(C)C)C(=O)C(CCCCCC)CCCCCCCC] | D |
| II-9 | 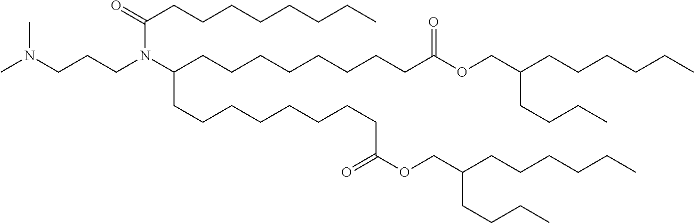 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC] | D |
| II-10 | 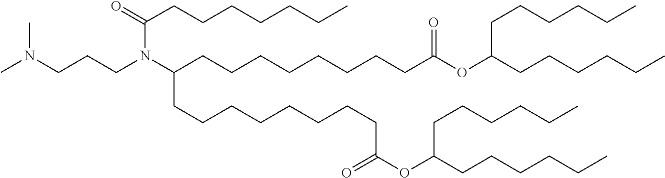 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] | D |
| II-11 | 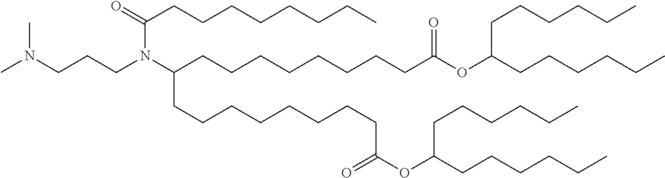 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC] | D |
| II-12 | 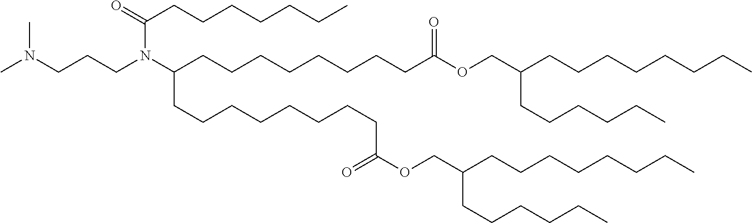 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC] | D |
| II-13 | 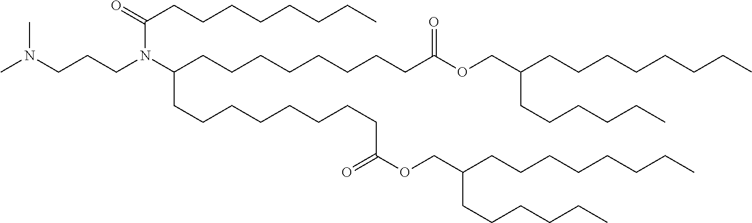 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC] | D |
| II-14 | 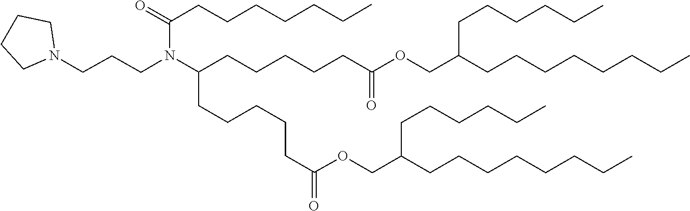 [SMILES: CCCCCCCCC(CCCCCC)COC(=O)CCCCCC(CCCCCC(=O)OCC(CCCCCC)CCCCCCCC)N(CCCN1CCCC1)C(=O)CCCCCCC] | D |
| II-15 | 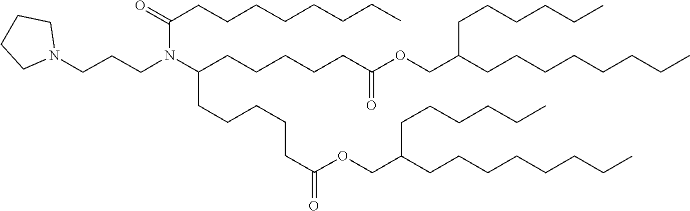 [SMILES: CCCCCCCCC(=O)N(CCCN1CCCC1)C(CCCCCC(=O)OCC(CCCCCC)CCCCCCCC)CCCCCC(=O)OCC(CCCCCC)CCCCCCCC] | D |
| II-16 | 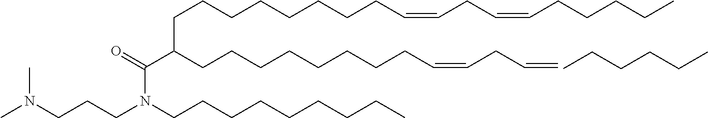 | E |
| II-17 | 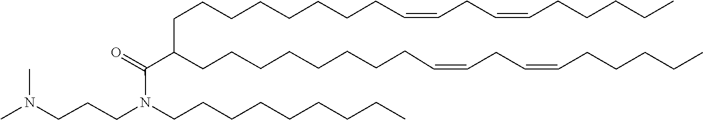 [SMILES: CCCCC/C=C\C/C=C\CCCCCCCCC(CCCCCCCC/C=C\C/C=C\CCCCC)C(=O)N(CCCCCCCCC)CCCN(C)C] | D |
| II-18 | 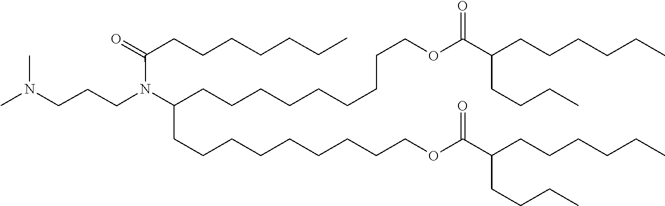 [SMILES: CCCCCCCC(=O)N(CCCN(C)C)C(CCCCCCCCCOC(=O)C(CCCC)CCCCCC)CCCCCCCCCOC(=O)C(CCCC)CCCCCC] | D |
| II-19 | 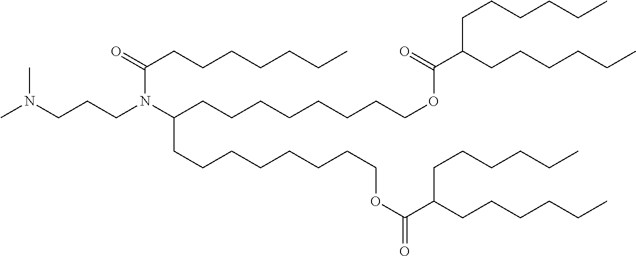 [SMILES: CCCCCCCC(=O)N(CCCN(C)C)C(CCCCCCCCOC(=O)C(CCCCCC)CCCCCC)CCCCCCCCOC(=O)C(CCCCCC)CCCCCC] | D |
| II-20 | 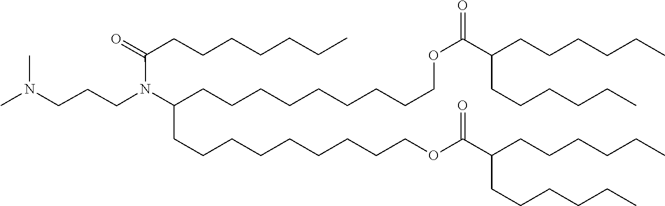 [SMILES: CCCCCCCC(=O)N(CCCN(C)C)C(CCCCCCCCCOC(=O)C(CCCCCC)CCCCCC)CCCCCCCCCOC(=O)C(CCCCCC)CCCCCC] | D |
| II-21 | 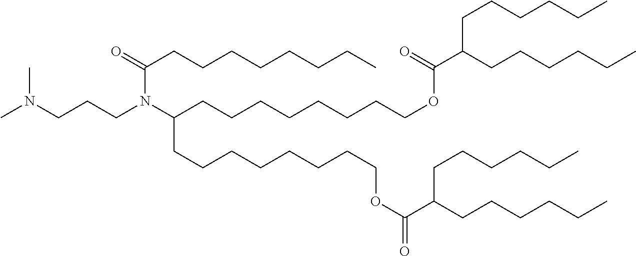 [SMILES: CCCCCCCCC(=O)N(CCCN(C)C)C(CCCCCCCCOC(=O)C(CCCCCC)CCCCCC)CCCCCCCCOC(=O)C(CCCCCC)CCCCCC] | D |
| II-22 | 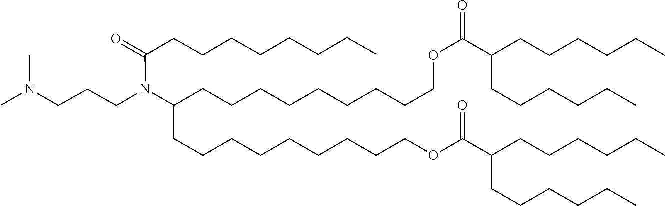 [SMILES: CCCCCCCCC(=O)N(CCCN(C)C)C(CCCCCCCCCOC(=O)C(CCCCCC)CCCCCC)CCCCCCCCCOC(=O)C(CCCCCC)CCCCCC] | D |
| II-23 | 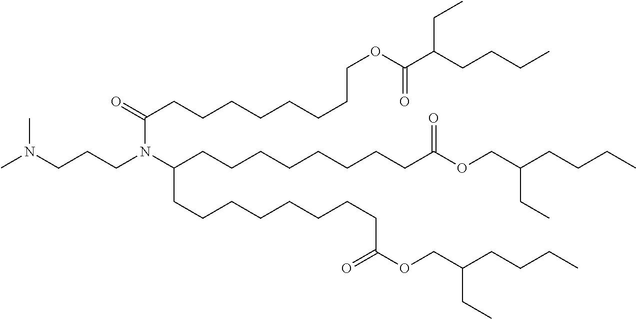 [SMILES: CCCCC(CC)COC(=O)CCCCCCCCC(CCCCCCCCC(=O)OCC(CC)CCCC)N(CCCN(C)C)C(=O)CCCCCCCCOC(=O)C(CC)CCCC] | D |
| II-24 | 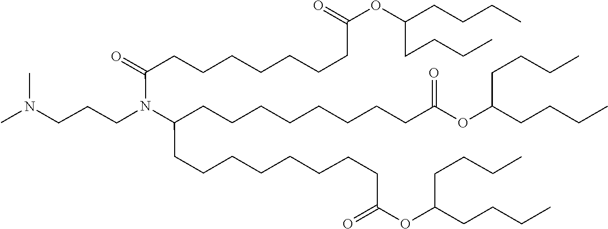 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC] | D |
| II-25 | 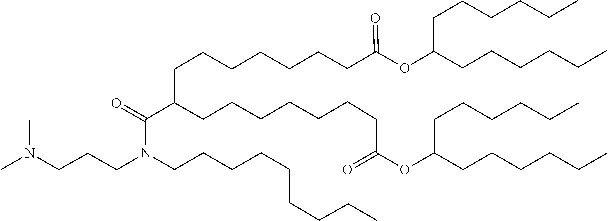 [SMILES: CCCCCCCCCN(CCCN(C)C)C(=O)C(CCCCCCCC(=O)OC(CCCCCC)CCCCCC)CCCCCCCC(=O)OC(CCCCCC)CCCCCC] | E |
| II-26 | 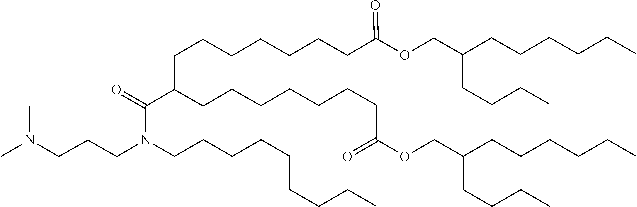 [SMILES: CCCCCCCCCN(CCCN(C)C)C(=O)C(CCCCCCCC(=O)OCC(CCCC)CCCCCC)CCCCCCCC(=O)OCC(CCCC)CCCCCC] | E |
| II-27 | 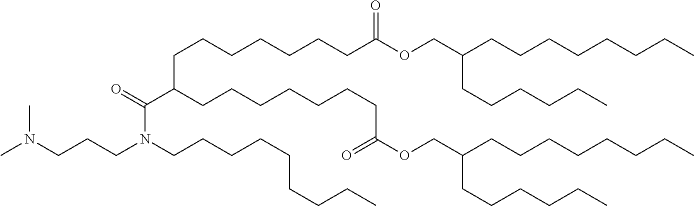 [SMILES: CCCCCCCCCN(CCCN(C)C)C(=O)C(CCCCCCCC(=O)OCC(CCCCCC)CCCCCCCC)CCCCCCCC(=O)OCC(CCCCCC)CCCCCCCC] | E |
| II-28 | 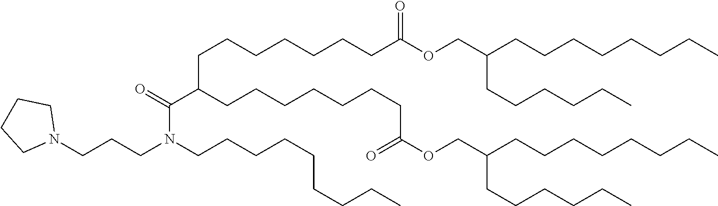 [SMILES: CCCCCCCCCN(CCCN1CCCC1)C(=O)C(CCCCCCCC(=O)OCC(CCCCCC)CCCCCCCC)CCCCCCCC(=O)OCC(CCCCCC)CCCCCCCC] | E |
| II-29 | 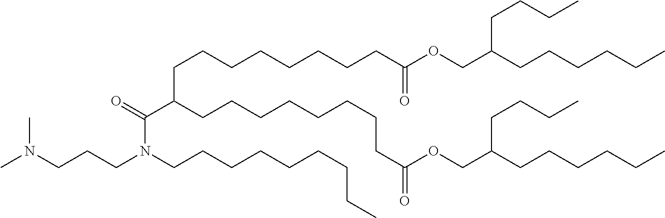 [SMILES: CCCCCCCCCN(CCCN(C)C)C(=O)C(CCCCCCCCC(=O)OCC(CCCC)CCCCCC)CCCCCCCCC(=O)OCC(CCCC)CCCCCC] | E |
| II-30 | 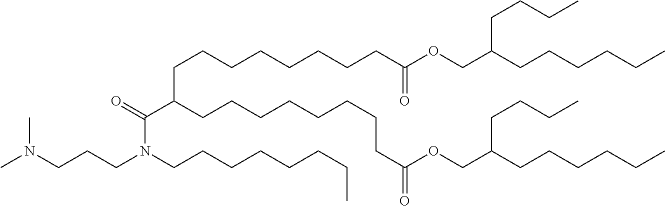 [SMILES: CCCCCCCCN(CCCN(C)C)C(=O)C(CCCCCCCCC(=O)OCC(CCCC)CCCCCC)CCCCCCCCC(=O)OCC(CCCC)CCCCCC] | E |
| II-31 | 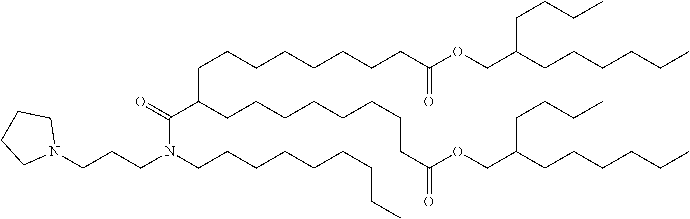 [SMILES: CCCCCCCCCN(CCCN1CCCC1)C(=O)C(CCCCCCCCC(=O)OCC(CCCC)CCCCCC)CCCCCCCCC(=O)OCC(CCCC)CCCCCC] | E |
| II-32 | 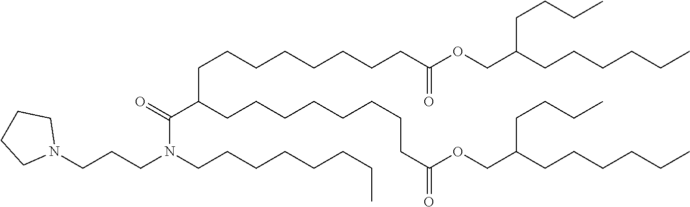 [SMILES: CCCCCCCCN(CCCN1CCCC1)C(=O)C(CCCCCCCCC(=O)OCC(CCCC)CCCCCC)CCCCCCCCC(=O)OCC(CCCC)CCCCCC] | E |
| II-33 | 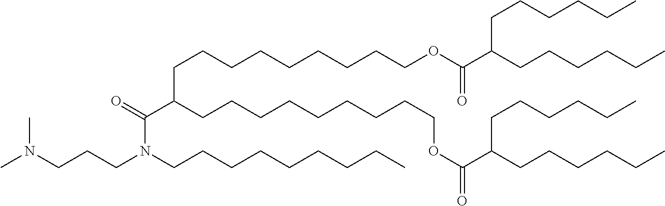 [SMILES: CCCCCCCCCN(CCCN(C)C)C(=O)C(CCCCCCCCCOC(=O)C(CCCCCC)CCCCCC)CCCCCCCCCOC(=O)C(CCCCCC)CCCCCC] | E |
| II-34 | 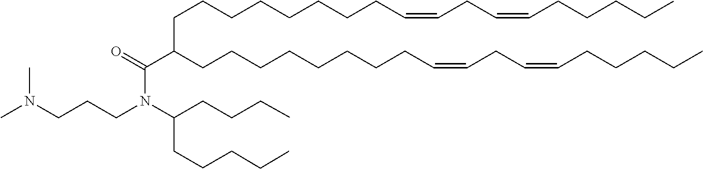 [SMILES: CCCCC/C=C\C/C=C\CCCCCCCCC(CCCCCCCC/C=C\C/C=C\CCCCC)C(=O)N(CCCN(C)C)C(CCCC)CCCCC] | E |
| II-35 | 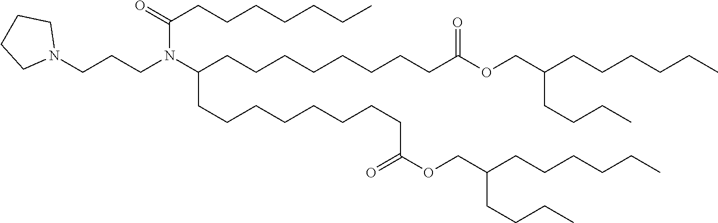 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC] | D |
| II-36 | 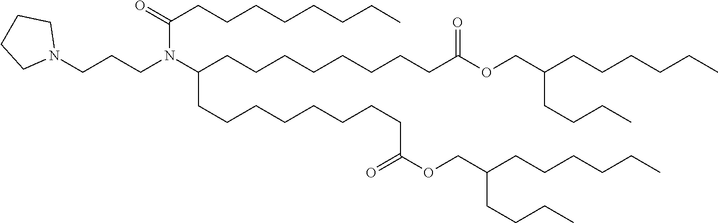 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC] | D |

In some embodiments, the LNPs comprise a lipid of Formula (II), a nucleoside-modified RNA and one or more excipient selected from neutral lipids, steroids and pegylated lipids. In some embodiments the lipid of Formula (II) is compound II-9. In some embodiments the lipid of Formula (II) is compound II-10. In some embodiments the lipid of Formula (II) is compound II-11. In some embodiments the lipid of Formula (II) is compound II-12. In some embodiments the lipid of Formula (II) is compound II-32.

In some other embodiments, the cationic lipid component of the LNPs has the structure of Formula (III):

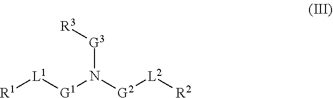

or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—,
—C(═O)S—, SC(═O)—, —NR^{a}C(═O)—, —C(═O)NR^{a}—, NR^{a}C(═O)NR^{a}—, —OC(═O)NR^{a}— or
—NR^{a}C(═O)O—, and the other of L¹ or L² is —O(C═O)—, —(C═O)O—, —C(═O)—, —O—, —S(O)ₓ—,
—S—S—, —C(═O)S—, SC(═O)—, —NR^{a}C(═O)—, —C(═O)NR^{a}—, NR^{a}C(═O)NR^{a}, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

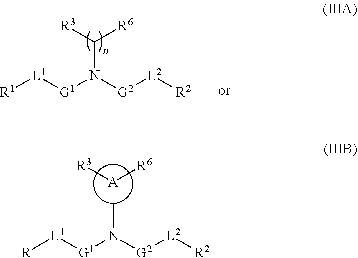

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or IIID):

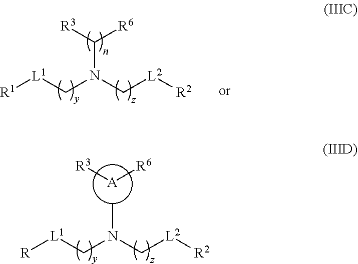

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

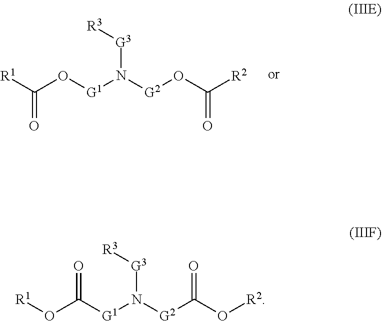

In some of the foregoing embodiments of Formula (III), the lipid has one of the following structures (IIIG), (IIIH), (IIII), or (IIIJ):

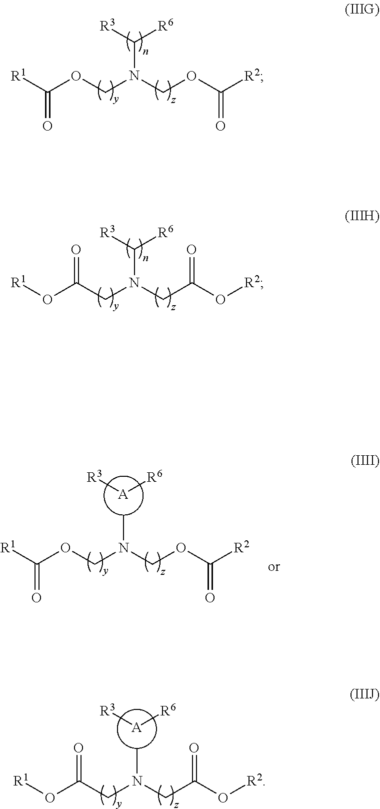

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G3 is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

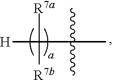

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

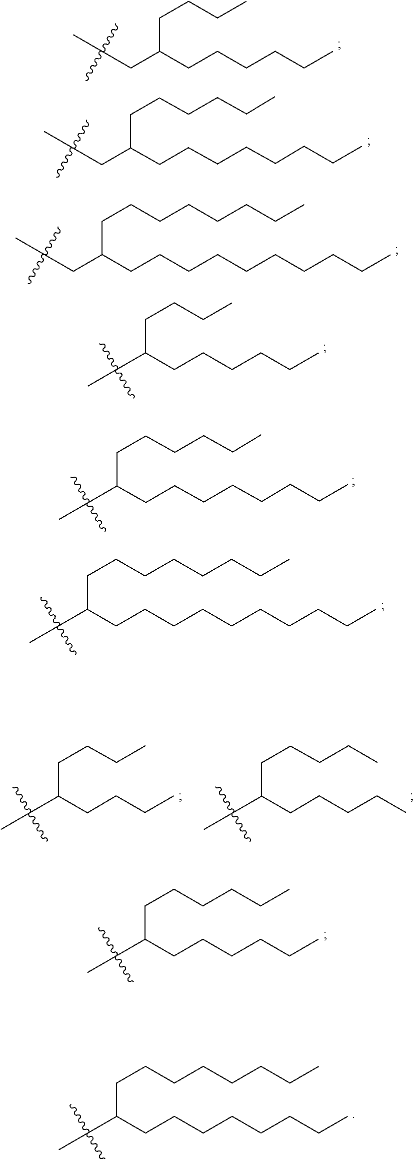

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in Table 3 below.

TABLE 3
Representative Compounds of Formula (III).

| | | Prep. |
|---|---|---|
| No. | Structure | Method |
| III-1 | 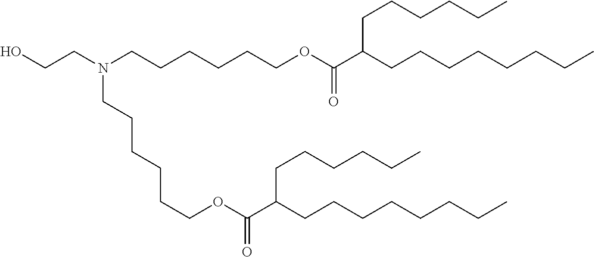 [SMILES: CCCCCCCCC(CCCCCC)C(=O)OCCCCCCN(CCO)CCCCCCOC(=O)C(CCCCCC)CCCCCCCC] | F |
| III-2 | 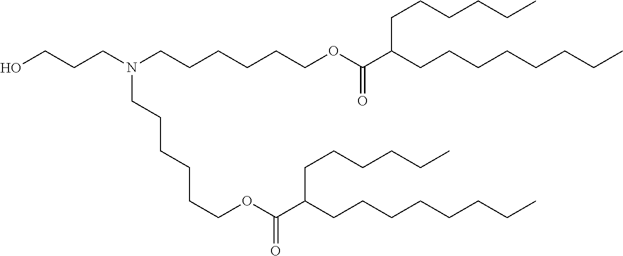 [SMILES: CCCCCCCCC(CCCCCC)C(=O)OCCCCCCN(CCCO)CCCCCCOC(=O)C(CCCCCC)CCCCCCCC] | F |
| III-3 | 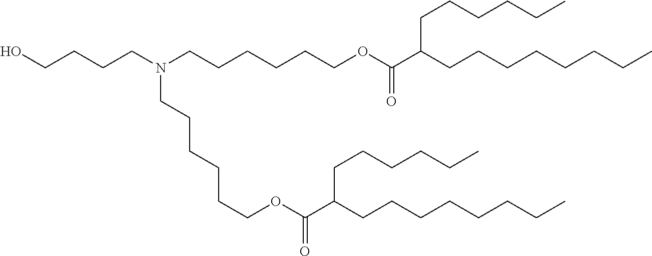 [SMILES: CCCCCCCCC(CCCCCC)C(=O)OCCCCCCN(CCCCO)CCCCCCOC(=O)C(CCCCCC)CCCCCCCC] | F |
| III-4 | 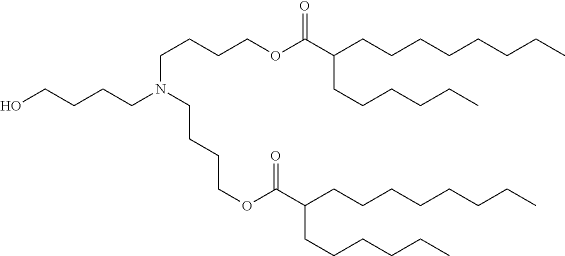 [SMILES: CCCCCCCCC(CCCCCC)C(=O)OCCCCN(CCCCO)CCCCOC(=O)C(CCCCCC)CCCCCCCC] | F |
| III-5 | 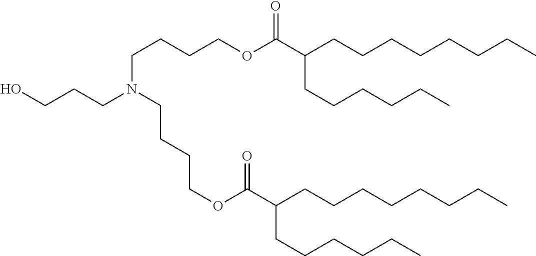 [SMILES: CCCCCCCCC(CCCCCC)C(=O)OCCCCN(CCCO)CCCCOC(=O)C(CCCCCC)CCCCCCCC] | F |
| III-6 | 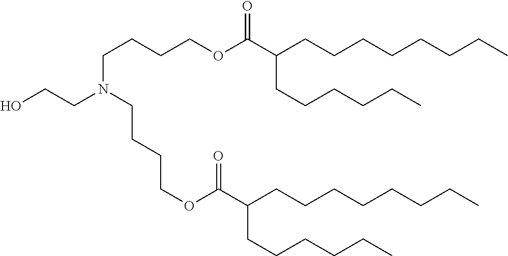 [SMILES: CCCCCCCCC(CCCCCC)C(=O)OCCCCN(CCO)CCCCOC(=O)C(CCCCCC)CCCCCCCC] | F |
| III-7 | 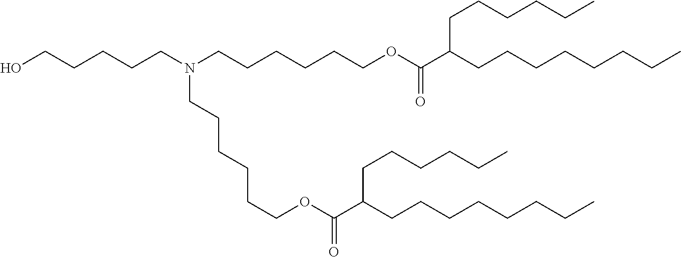 [SMILES: CCCCCCCCC(CCCCCC)C(=O)OCCCCCCN(CCCCCO)CCCCCCOC(=O)C(CCCCCC)CCCCCCCC] | F |
| III-8 | 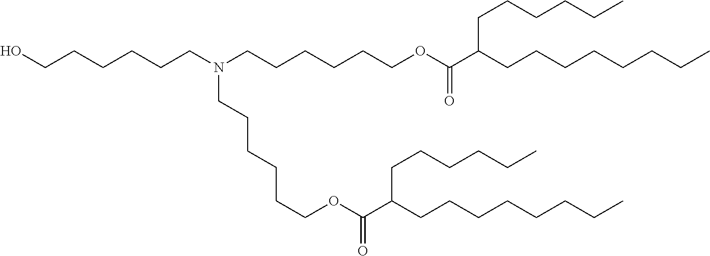 [SMILES: CCCCCCCCC(CCCCCC)C(=O)OCCCCCCN(CCCCCCO)CCCCCCOC(=O)C(CCCCCC)CCCCCCCC] | F |
| III-9 | 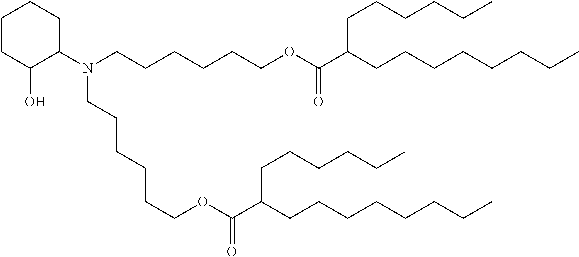 [SMILES: CCCCCCCCC(CCCCCC)C(=O)OCCCCCCN(CCCCCCOC(=O)C(CCCCCC)CCCCCCCC)C1CCCCC1O] | F |
| III-10 | 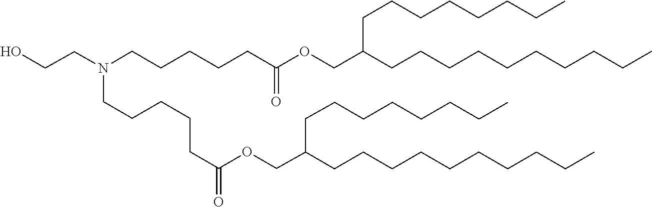 [SMILES: CCCCCCCCCCC(CCCCCCCC)COC(=O)CCCCCN(CCO)CCCCCC(=O)OCC(CCCCCCCC)CCCCCCCCCC] | F |
| III-11 | 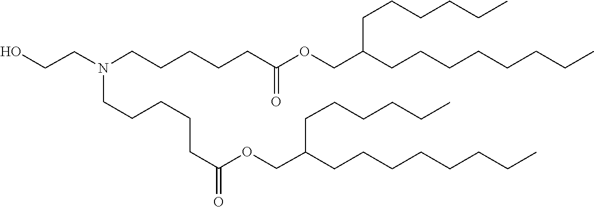 [SMILES: CCCCCCCCC(CCCCCC)COC(=O)CCCCCN(CCO)CCCCCC(=O)OCC(CCCCCC)CCCCCCCC] | F |
| III-12 | 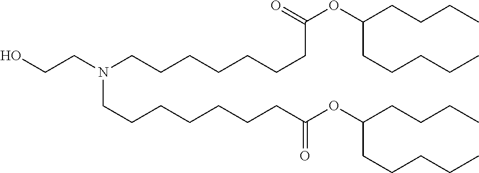 [SMILES: CCCCCC(CCCC)OC(=O)CCCCCCCN(CCO)CCCCCCCC(=O)OC(CCCC)CCCCC] | F |
| III-13 | 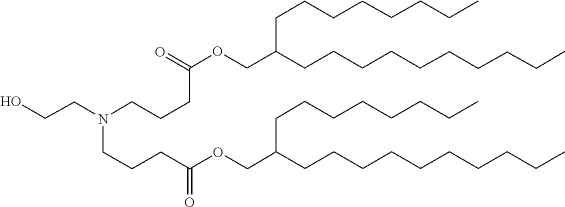 [SMILES: CCCCCCCCCCC(CCCCCCCC)COC(=O)CCCN(CCO)CCCC(=O)OCC(CCCCCCCC)CCCCCCCCCC] | F |
| III-14 | 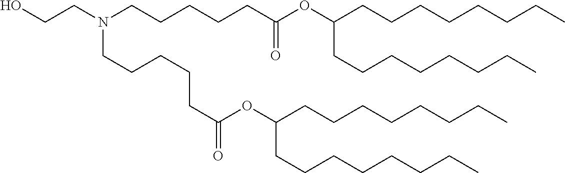 [SMILES: CCCCCCCCC(CCCCCCCC)OC(=O)CCCCCN(CCO)CCCCCC(=O)OC(CCCCCCCC)CCCCCCCC] | F |
| III-15 | 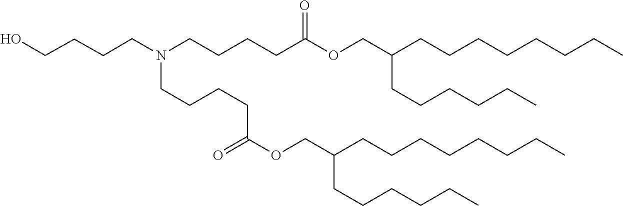 [SMILES: CCCCCCCCC(CCCCCC)COC(=O)CCCCN(CCCCO)CCCCC(=O)OCC(CCCCCC)CCCCCCCC] | F |
| III-16 | 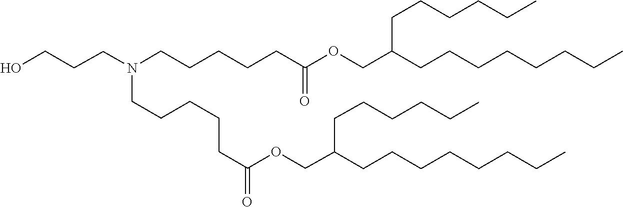 [SMILES: CCCCCCCCC(CCCCCC)COC(=O)CCCCCN(CCCO)CCCCCC(=O)OCC(CCCCCC)CCCCCCCC] | F |
| III-17 | 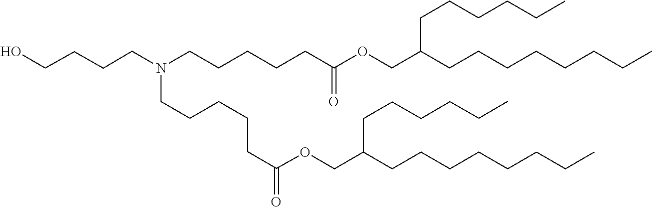 [SMILES: CCCCCCCCC(CCCCCC)COC(=O)CCCCCN(CCCCO)CCCCCC(=O)OCC(CCCCCC)CCCCCCCC] | F |
| III-18 | 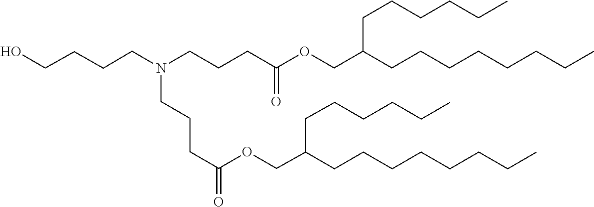 [SMILES: CCCCCCCCC(CCCCCC)COC(=O)CCCN(CCCCO)CCCC(=O)OCC(CCCCCC)CCCCCCCC] | F |
| III-19 | 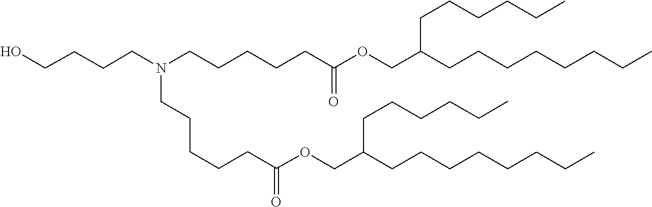 [SMILES: CCCCCCCCC(CCCCCC)COC(=O)CCCCCN(CCCCO)CCCCCC(=O)OCC(CCCCCC)CCCCCCCC] | F |
| III-20 | 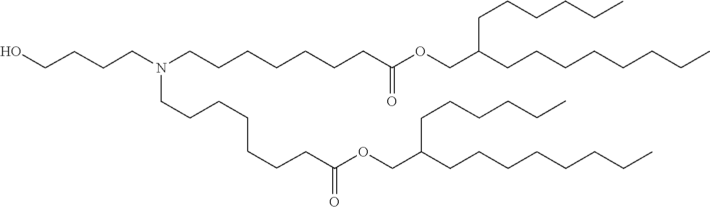 [SMILES: CCCCCCCCC(CCCCCC)COC(=O)CCCCCCCN(CCCCO)CCCCCCCC(=O)OCC(CCCCCC)CCCCCCCC] | F |
| III-21 | 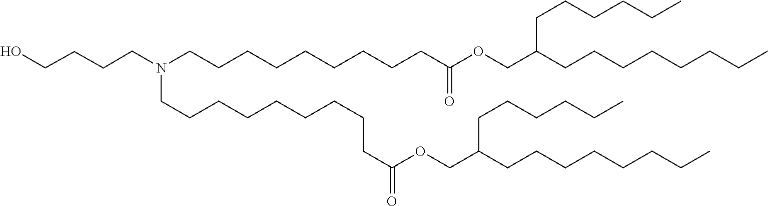 [SMILES: CCCCCCCCC(CCCCCC)COC(=O)CCCCCCCCCN(CCCCO)CCCCCCCCCC(=O)OCC(CCCCCC)CCCCCCCC] | F |
| III-22 | 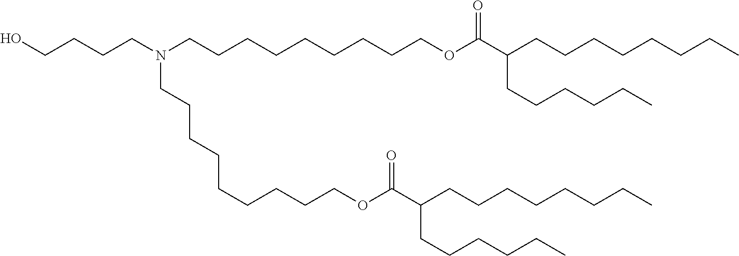 [SMILES: CCCCCCCCC(CCCCCC)C(=O)OCCCCCCCCCN(CCCCO)CCCCCCCCCOC(=O)C(CCCCCC)CCCCCCCC] | F |
| III-23 | 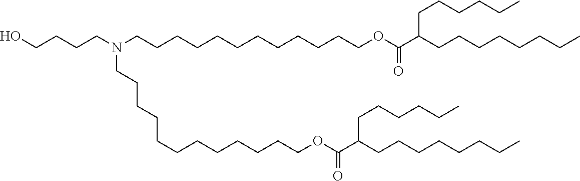 [SMILES: CCCCCCCCC(CCCCCC)C(=O)OCCCCCCCCCCCCN(CCCCO)CCCCCCCCCCCCOC(=O)C(CCCCCC)CCCCCCCC] | F |
| III-24 | 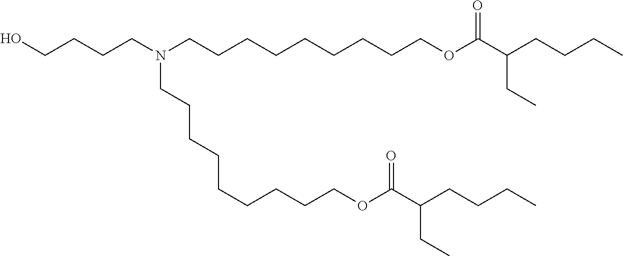 [SMILES: CCCCC(CC)C(=O)OCCCCCCCCCN(CCCCO)CCCCCCCCCOC(=O)C(CC)CCCC] | F |
| III-25 | 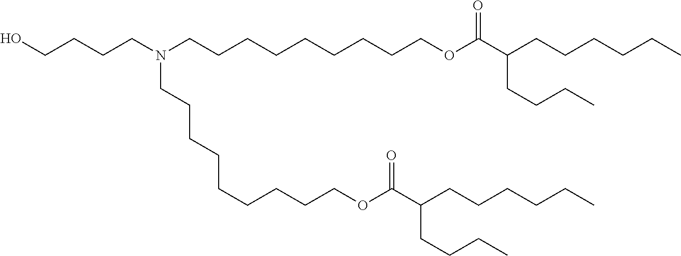 [SMILES: CCCCCCC(CCCC)C(=O)OCCCCCCCCCN(CCCCO)CCCCCCCCCOC(=O)C(CCCC)CCCCCC] | F |
| III-26 | 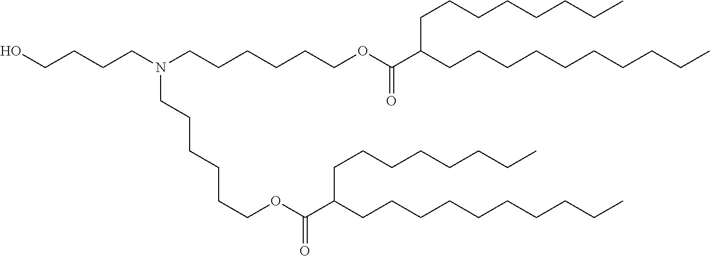 [SMILES: CCCCCCCCCCC(CCCCCCCC)C(=O)OCCCCCCN(CCCCO)CCCCCCOC(=O)C(CCCCCCCC)CCCCCCCCCC] | F |
| III-27 | 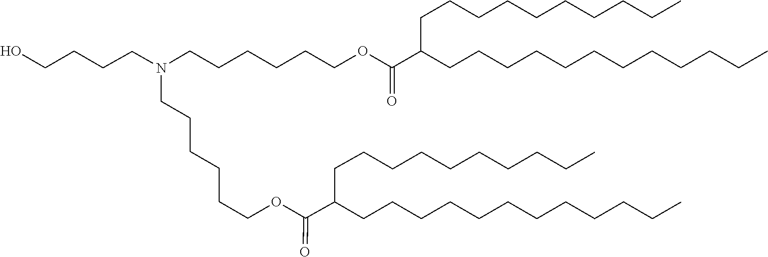 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] | F |
| III-28 | 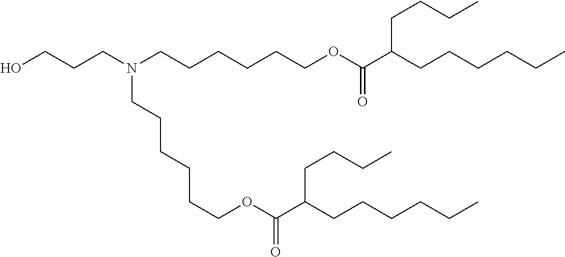 [SMILES: CCCCCCC(CCCC)C(=O)OCCCCCCN(CCCO)CCCCCCOC(=O)C(CCCC)CCCCCC] | F |
| III-29 | 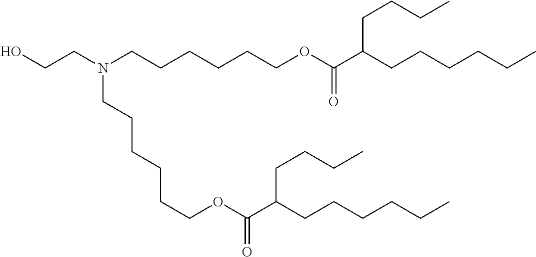 [SMILES: CCCCCCC(CCCC)C(=O)OCCCCCCN(CCO)CCCCCCOC(=O)C(CCCC)CCCCCC] | F |
| III-30 | 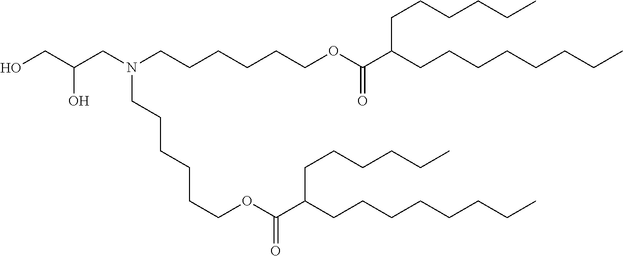 [SMILES: CCCCCCCCC(CCCCCC)C(=O)OCCCCCCN(CCCCCCOC(=O)C(CCCCCC)CCCCCCCC)CC(O)CO] | F |
| III-31 | 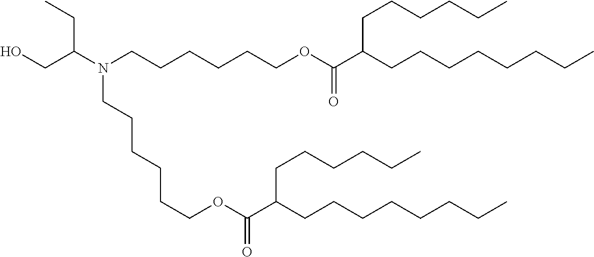 [SMILES: CCCCCCCCC(CCCCCC)C(=O)OCCCCCCN(CCCCCCOC(=O)C(CCCCCC)CCCCCCCC)C(CC)CO] | F |
| III-32 | 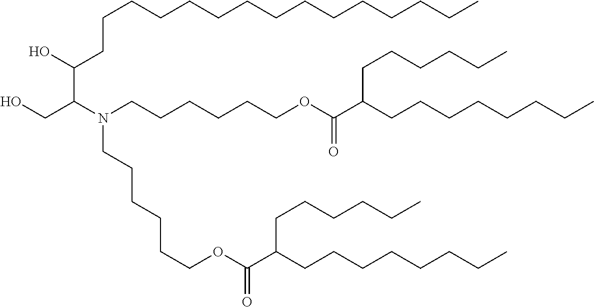 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] | F |
| III-33 | 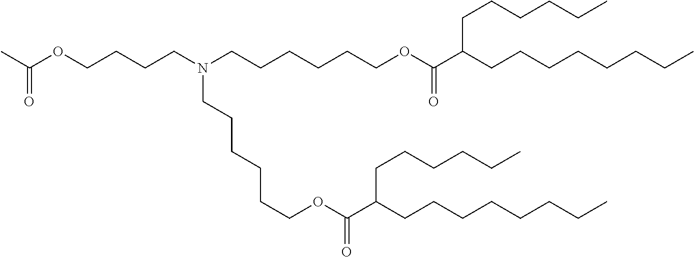 [SMILES: CCCCCCCCC(CCCCCC)C(=O)OCCCCCCN(CCCCCCOC(=O)C(CCCCCC)CCCCCCCC)CCCCOC(C)=O] | F |
| III-34 | 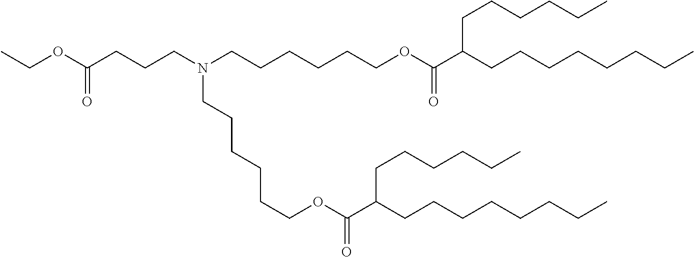 [SMILES: CCCCCCCCC(CCCCCC)C(=O)OCCCCCCN(CCCCCCOC(=O)C(CCCCCC)CCCCCCCC)CCCC(=O)OCC] | F |
| III-35 | 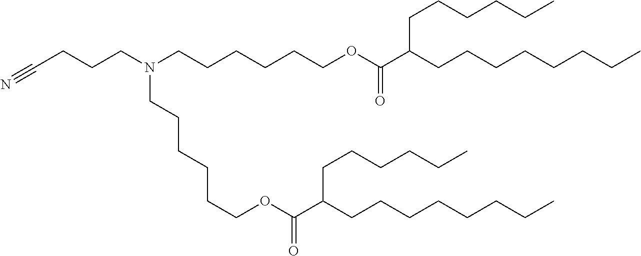 [SMILES: CCCCCCCCC(CCCCCC)C(=O)OCCCCCCN(CCCC#N)CCCCCCOC(=O)C(CCCCCC)CCCCCCCC] | F |
| III-36 | 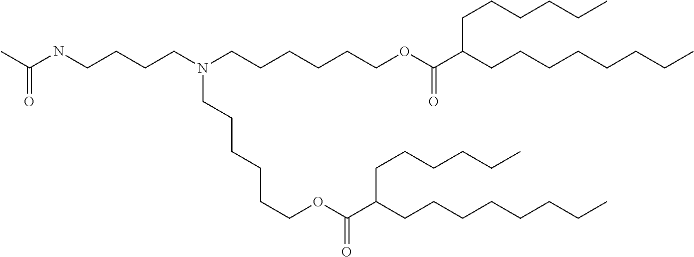 [SMILES: CCCCCCCCC(CCCCCC)C(=O)OCCCCCCN(CCCCCCOC(=O)C(CCCCCC)CCCCCCCC)CCCCNC(C)=O] | F |

In some embodiments, the cationic lipid is present in the LNP in an amount from about 30 to about 95 mole percent. In one embodiment, the cationic lipid is present in the LNP in an amount from about 30 to about 70 mole percent. In one embodiment, the cationic lipid is present in the LNP in an amount from about 40 to about 60 mole percent. In one embodiment, the cationic lipid is present in the LNP in an amount of about 50 mole percent. In one embodiment, the LNP comprises only cationic lipids.

In some embodiments, the LNP comprises one or more additional lipids which stabilize the formation of particles during their formation.

Suitable stabilizing lipids include neutral lipids and anionic lipids.

Exemplary anionic lipids include, but are not limited to, phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE). In one embodiment, the neutral lipid is 1,2-distearoyl-sn-glycero-3-phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid (e.g., lipid of Formula (I)) to the neutral lipid ranges from about 2:1 to about 8:1.

In various embodiments, the LNPs further comprise a steroid or steroid analogue. A “steroid” is a compound comprising the following carbon skeleton:

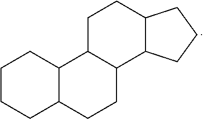 [SMILES: C1CCC2C(C1)CCC1C3CCCC3CCC21]

In some embodiments, the steroid or steroid analogue is cholesterol. In some of these embodiments, the molar ratio of the cationic lipid (e.g., lipid of Formula (I)) to cholesterol ranges from about 2:1 to 1:1.

In some embodiments, the LNP comprises glycolipids (e.g., monosialoganglioside GM₁). In some embodiments, the LNP comprises a sterol, such as cholesterol.

In some embodiments, the LNPs comprise a polymer conjugated lipid.

In some embodiments, the LNP comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)₂₀₀₀)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate. In various embodiments, the molar ratio of the cationic lipid to the pegylated lipid ranges from about 100:1 to about 25:1.

In some embodiments, the LNPs comprise a pegylated lipid having the following structure (IV):

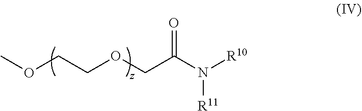

or a pharmaceutically acceptable salt, tautomer or stereoisomer thereof, wherein:
R¹⁰ and R¹¹ are each independently a straight or branched, saturated or unsaturated alkyl chain containing from 10 to 30 carbon atoms, wherein the alkyl chain is optionally interrupted by one or more ester bonds; and
z has mean value ranging from 30 to 60.

In some of the foregoing embodiments of the pegylated lipid (IV), R¹⁰ and R¹¹ are not both n-octadecyl when z is 42. In some other embodiments, R¹⁰ and R¹¹ are each independently a straight or branched, saturated or unsaturated alkyl chain containing from to 18 carbon atoms. In some embodiments, R¹⁰ and R¹¹ are each independently a straight or branched, saturated or unsaturated alkyl chain containing from 12 to 16 carbon atoms. In some embodiments, R¹⁰ and R¹¹ are each independently a straight or branched, saturated or unsaturated alkyl chain containing 12 carbon atoms. In some embodiments, R¹⁰ and R¹¹ are each independently a straight or branched, saturated or unsaturated alkyl chain containing 14 carbon atoms. In other embodiments, R¹⁰ and R¹¹ are each independently a straight or branched, saturated or unsaturated alkyl chain containing 16 carbon atoms. In still more embodiments, R¹⁰ and R¹¹ are each independently a straight or branched, saturated or unsaturated alkyl chain containing 18 carbon atoms. In still other embodiments, R¹⁰ is a straight or branched, saturated or unsaturated alkyl chain containing 12 carbon atoms and R¹¹ is a straight or branched, saturated or unsaturated alkyl chain containing 14 carbon atoms.

In various embodiments, z spans a range that is selected such that the PEG portion of (II) has an average molecular weight of about 400 to about 6000 g/mol. In some embodiments, the average z is about 45.

In other embodiments, the pegylated lipid has one of the following structures:

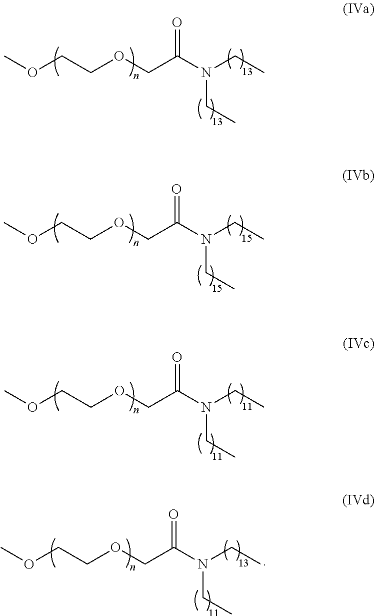

wherein n is an integer selected such that the average molecular weight of the pegylated lipid is about 2500 g/mol.

In some embodiments, the additional lipid is present in the LNP in an amount from about 1 to about 10 mole percent. In one embodiment, the additional lipid is present in the LNP in an amount from about 1 to about 5 mole percent. In one embodiment, the additional lipid is present in the LNP in about 1 mole percent or about 1.5 mole percent.

In some embodiments, the LNPs comprise a lipid of Formula (I), a nucleoside-modified RNA, a neutral lipid, a steroid and a pegylated lipid. In some embodiments the lipid of Formula (I) is compound I-6. In different embodiments, the neutral lipid is DSPC.

In other embodiments, the steroid is cholesterol. In still different embodiments, the pegylated lipid is compound IVa.

In some embodiments, the LNP comprises one or more targeting moieties, which are capable of targeting the LNP to a cell or cell population. For example, in one embodiment, the targeting moiety is a ligand, which directs the LNP to a receptor found on a cell surface.

In some embodiments, the LNP comprises one or more internalization domains. For example, in one embodiment, the LNP comprises one or more domains, which bind to a cell to induce the internalization of the LNP. For example, in one embodiment, the one or more internalization domains bind to a receptor found on a cell surface to induce receptor-mediated uptake of the LNP. In some embodiments, the LNP is capable of binding a biomolecule in vivo, where the LNP-bound biomolecule can then be recognized by a cell-surface receptor to induce internalization. For example, in one embodiment, the LNP binds systemic ApoE, which leads to the uptake of the LNP and associated cargo.

Other exemplary LNPs and their manufacture are described in the art, for example in U.S. Patent Application Publication No. US20120276209, Semple et al., 2010, Nat Biotechnol., 28(2):172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tam et al., 2013, Nanomedicine, 9(5): 665-74, each of which are incorporated by reference in their entirety.

The following Reaction Schemes illustrate methods to make lipids of Formula (I), (II) or (III).

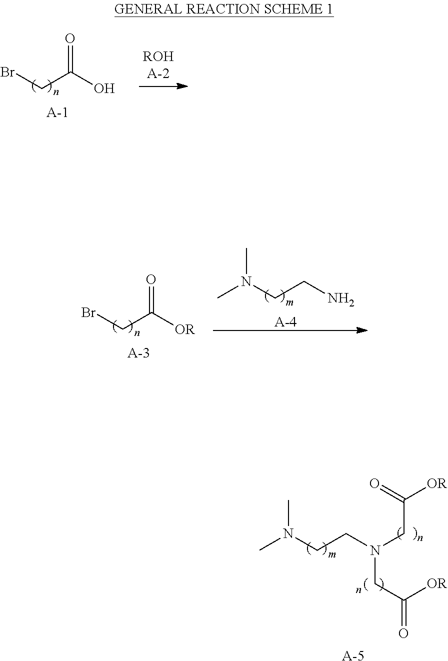

Embodiments of the lipid of Formula (I) (e.g., compound A-5) can be prepared according to General Reaction Scheme 1 (“Method A”), wherein R is a saturated or unsaturated C₁-C₂₄ alkyl or saturated or unsaturated cycloalkyl, m is 0 or 1 and n is an integer from 1 to 24. Referring to General Reaction Scheme 1, compounds of structure A-1 can be purchased from commercial sources or prepared according to methods familiar to one of ordinary skill in the art. A mixture of A-1, A-2 and DMAP is treated with DCC to give the bromide A-3. A mixture of the bromide A-3, a base (e.g., N,N-diisopropylethylamine) and the N,N-dimethyldiamine A-4 is heated at a temperature and time sufficient to produce A-5 after any necessarily workup and or purification step.

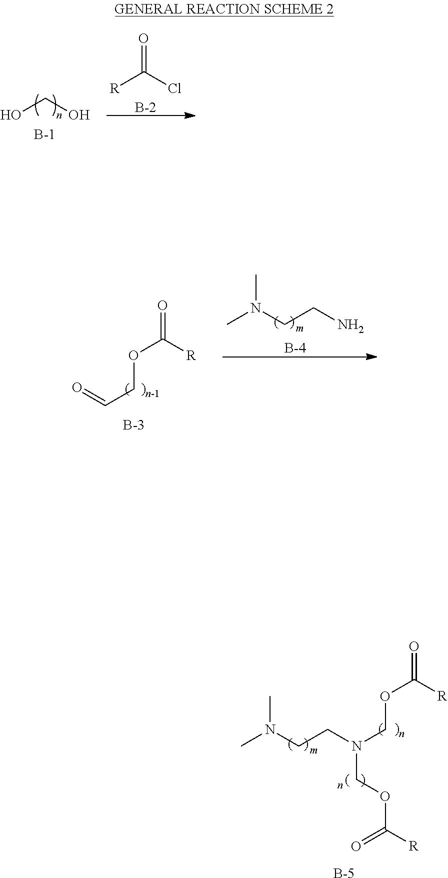

Other embodiments of the compound of Formula (I) (e.g., compound B-5) can be prepared according to General Reaction Scheme 2 (“Method B”), wherein R is a saturated or unsaturated C₁-C₂₄ alkyl or saturated or unsaturated cycloalkyl, m is 0 or 1 and n is an integer from 1 to 24. As shown in General Reaction Scheme 2, compounds of structure B-1 can be purchased from commercial sources or prepared according to methods familiar to one of ordinary skill in the art. A solution of B-1 (1 equivalent) is treated with acid chloride B-2 (1 equivalent) and a base (e.g., triethylamine). The crude product is treated with an oxidizing agent (e.g., pyridinum chlorochromate) and intermediate product B-3 is recovered. A solution of crude B-3, an acid (e.g., acetic acid), and N,N-dimethylaminoamine B-4 is then treated with a reducing agent (e.g., sodium triacetoxyborohydride) to obtain B-5 after any necessary work up and/or purification.

It should be noted that although starting materials A-1 and B-1 are depicted above as including only saturated methylene carbons, starting materials which include carbon-carbon double bonds may also be employed for preparation of compounds which include carbon-carbon double bonds.

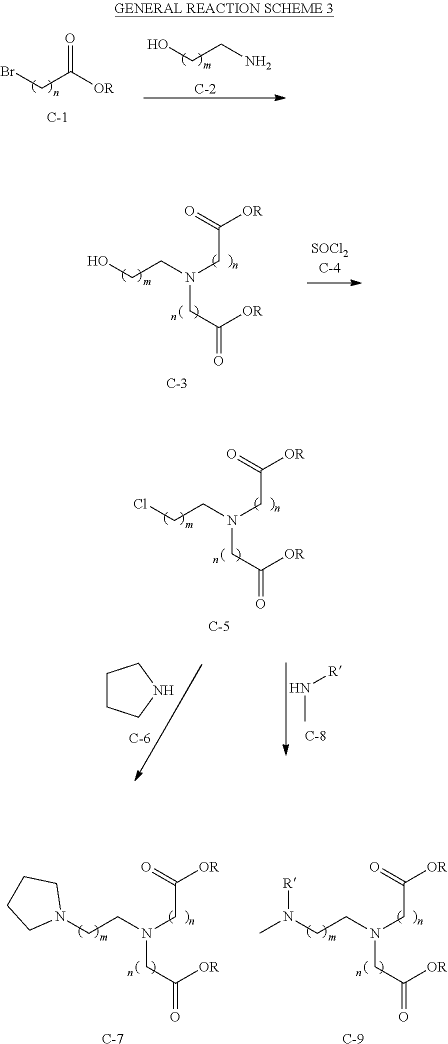

Different embodiments of the lipid of Formula (I) (e.g., compound C-7 or C9) can be prepared according to General Reaction Scheme 3 (“Method C”), wherein R is a saturated or unsaturated C₁-C₂₄ alkyl or saturated or unsaturated cycloalkyl, m is 0 or 1 and n is an integer from 1 to 24. Referring to General Reaction Scheme 3, compounds of structure C-1 can be purchased from commercial sources or prepared according to methods familiar to one of ordinary skill in the art.

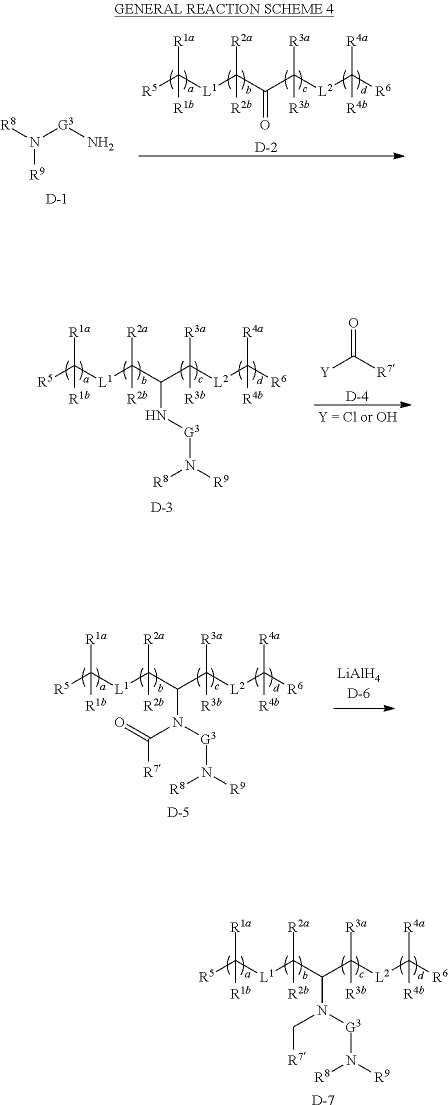

Embodiments of the compound of Formula (II) (e.g., compounds D-5 and D-7) can be prepared according to General Reaction Scheme 4 (“Method D”), wherein R^{1a}, R^{1b}, R^{2a}, R^{2b}, R^{3a}, R^{3b}, R^{4a}, R^{4b}, R⁵, R⁶, R⁸, R⁹, L¹, L², G¹, G², G³, a, b, c and d are as defined herein, and R^{7′} represents R⁷ or a C₃-C₁₉ alkyl. Referring to General Reaction Scheme 1, compounds of structure D-1 and D-2 can be purchased from commercial sources or prepared according to methods familiar to one of ordinary skill in the art. A solution of D-1 and D-2 is treated with a reducing agent (e.g., sodium triacetoxyborohydride) to obtain D-3 after any necessary work up. A solution of D-3 and a base (e.g. trimethylamine, DMAP) is treated with acyl chloride D-4 (or carboxylic acid and DCC) to obtain D-5 after any necessary work up and/or purification. D-5 can be reduced with LiAlH4 D-6 to give D-7 after any necessary work up and/or purification.

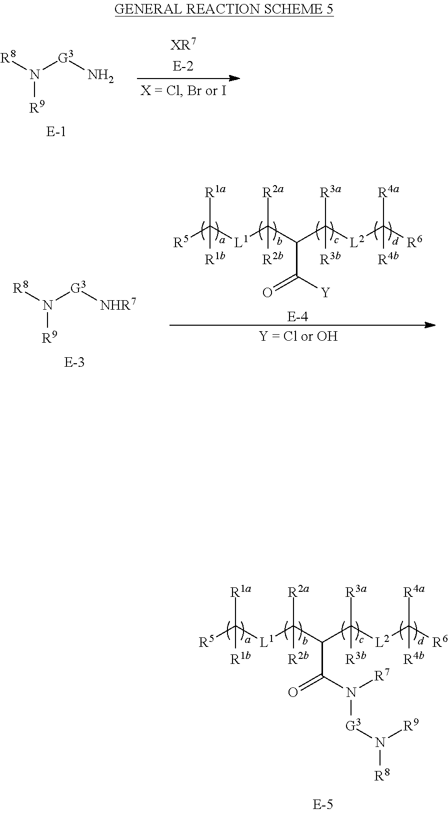

Embodiments of the lipid of Formula (II) (e.g., compound E-5) can be prepared according to General Reaction Scheme 5 (“Method E”), wherein R^{1a}, R^{1b}, R^{2a}, R^{2b}, R^{3a}, R^{3b}, R^{4a}, R^{4b}, R⁵, R⁶, R⁷, R⁸, R⁹, L¹, L², G³, a, b, c and d are as defined herein. Referring to General Reaction Scheme 2, compounds of structure E-1 and E-2 can be purchased from commercial sources or prepared according to methods familiar to one of ordinary skill in the art. A mixture of E-1 (in excess), E-2 and a base (e.g., potassium carbonate) is heated to obtain E-3 after any necessary work up. A solution of E-3 and a base (e.g. trimethylamine, DMAP) is treated with acyl chloride E-4 (or carboxylic acid and DCC) to obtain E-5 after any necessary work up and/or purification.

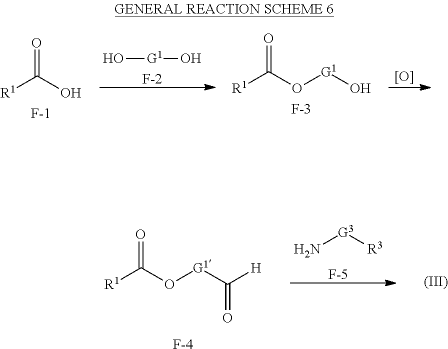

General Reaction Scheme 6 provides an exemplary method (Method F) for preparation of Lipids of Formula (III). G¹, G³, R¹ and R³ in General Reaction Scheme 6 are as defined herein for Formula (III), and G1′ refers to a one-carbon shorter homologue of G1. Compounds of structure F-1 are purchased or prepared according to methods known in the art. Reaction of F-1 with diol F-2 under appropriate condensation conditions (e.g., DCC) yields ester/alcohol F-3, which can then be oxidized (e.g., PCC) to aldehyde F-4. Reaction of F-4 with amine F-5 under reductive amination conditions yields a lipid of Formula (III).

It should be noted that various alternative strategies for preparation of lipids of Formula (III) are available to those of ordinary skill in the art. For example, other lipids of Formula (III) wherein L¹ and L² are other than ester can be prepared according to analogous methods using the appropriate starting material. Further, General Reaction Scheme 6 depicts preparation of a lipids of Formula (III), wherein G¹ and G² are the same; however, this is not a required aspect of the invention and modifications to the above reaction scheme are possible to yield compounds wherein G¹ and G² are different.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to subjects of all sorts. Modification of pharmaceutical compositions suitable for administration to humans in order to render the compositions suitable for administration to various subjects is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient, which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers. In some embodiments, the formulation may comprise dry particles which comprise the active ingredient and which have a diameter in the range from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some instances having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Methods of Treatment or Prevention

The present invention provides methods of inducing an adaptive immune response against one or more tick salivary antigen thus treating or preventing infection by one or more tick-borne pathogen or a disease or disorder associated with one or more tick-borne pathogen in a subject. In one embodiment, the method comprises administering an effective amount of a composition comprising one or more nucleoside-modified RNA encoding at least one tick salivary antigen or a fragment thereof.

In one embodiment, the method provides methods for treating or preventing in the subject one or more of a tick-borne pathogen infection, a tick-borne viral infection, tick-borne spirochete infection or to a disease or disorder associated with one or more tick-borne pathogens. The present invention thus provides a method of treating or preventing the infection, disease, or disorder associated with one or more tick-borne pathogen. In one embodiment, the present invention provides a method of treating or preventing Lyme disease.

In one embodiment, the composition is administered to a subject having an infection, disease, or disorder associated with one or more tick-borne pathogen. In one embodiment, the composition is administered to a subject at risk for developing a disease or disorder associated with one or more tick-borne pathogen. For example, the composition may be administered to a subject who is at risk for being in contact with ticks. In one embodiment, the composition is administered to a subject who lives in, traveled to, or is expected to travel to a geographic region in which ticks are prevalent. In one embodiment, the composition is administered to a subject who is in contact with or expected to be in contact with another person who lives in, traveled to, or is expected to travel to a geographic region in which ticks are prevalent. In one embodiment, the composition is administered to a subject who has knowingly been exposed to a tick through a bite, or other contact.

In one embodiment, the method comprises administering a composition comprising one or more nucleoside-modified nucleic acid molecules encoding one or more tick salivary antigen. In one embodiment, the method comprises administering a composition comprising a first nucleoside-modified nucleic acid molecule encoding one or more tick salivary antigen and at least one additional nucleoside-modified nucleic acid molecule encoding at least one additional tick salivary antigen.

In one embodiment, the method comprises administering one or more compositions, each composition comprising one or more nucleoside-modified nucleic acid molecules encoding one or more tick salivary antigen. In one embodiment, the method comprises administering a first composition comprising one or more nucleoside-modified nucleic acid molecules encoding one or more tick salivary antigen and administering a second composition comprising one or more nucleoside-modified nucleic acid molecules encoding one or more tick salivary antigen. In one embodiment, the method comprises administering a plurality of compositions, each composition comprising one or more nucleoside-modified nucleic acid molecules encoding one or more tick salivary antigen described herein. In some embodiments, the method comprises a staggered administration of the plurality of compositions.

In some embodiments, the method comprises administering to subject a plurality of nucleoside-modified nucleic acid molecules encoding a plurality of tick salivary antigens, adjuvants, or a combination thereof.

In some embodiments, the method of the invention allows for sustained expression of one or more tick salivary antigen or adjuvant, described herein, for at least several days following administration. In some embodiments, the method of the invention allows for sustained expression of the tick salivary antigen or adjuvant, described herein, for at least 2 weeks following administration. In some embodiments, the method of the invention allows for sustained expression of at least one tick salivary antigen or adjuvant, described herein, for at least 1 month following administration. However, the method, in some embodiments, also provides for transient expression, as in some embodiments, the nucleic acid is not integrated into the subject genome.

In some embodiments, the method comprises administering nucleoside-modified RNA, which provides stable expression of the tick salivary antigen or adjuvant described herein. In some embodiments, administration of nucleoside-modified RNA results in little to no innate immune response, while inducing an effective adaptive immune response.

In some embodiments, the method provides sustained protection against a disease or disorder associated with one or more tick-borne pathogen. In one embodiment, the disease or disorder associated with one or more tick-born pathogen is Lyme disease. For example, in some embodiments, the method provides sustained protection against Lyme disease for more than 2 weeks. In some embodiments, the method provides sustained protection against Lyme disease for 1 month or more. In some embodiments, the method provides sustained protection against Lyme disease for 2 months or more. In some embodiments, the method provides sustained protection against Lyme disease for 3 months or more. In some embodiments, the method provides sustained protection against Lyme disease for 4 months or more. In some embodiments, the method provides sustained protection against Lyme disease for 5 months or more. In some embodiments, the method provides sustained protection against Lyme disease for 6 months or more. In some embodiments, the method provides sustained protection against Lyme disease for 1 year or more.

In one embodiment, a single immunization of the composition induces a sustained protection against Lyme disease for 1 month or more, 2 months or more, 3 months or more, 4 months or more, 5 months or more, 6 months or more, or 1 year or more.

Administration of the compositions of the invention in a method of treatment can be achieved in a number of different ways, using methods known in the art. In one embodiment, the method of the invention comprises systemic administration of the subject, including for example enteral or parenteral administration. In some embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition. In one embodiment, the method comprises inhalation of the composition. In one embodiment, the method comprises intranasal delivery of the composition.

It will be appreciated that the composition of the invention may be administered to a subject either alone, or in conjunction with another agent.

The therapeutic and prophylactic methods of the invention thus encompass the use of pharmaceutical compositions encoding one or more tick salivary antigen, adjuvant, or a combination thereof, described herein to practice the methods of the invention. The pharmaceutical compositions useful for practicing the invention may be administered to deliver a dose of from 1 ng/kg/day and 100 mg/kg/day. In one embodiment, the invention envisions administration of a dose, which results in a concentration of the compound of the present invention from 10 nM and 10 μM in a mammal.

Typically, dosages which may be administered in a method of the invention to a mammal, such as a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. In some embodiments, the dosage of the compound will vary from about 0.1 μg to about 10 mg per kilogram of body weight of the mammal. In some embodiments, the dosage will vary from about 1 μg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months, several years, or even less frequently, such as every 10-20 years, 15-30 years, or even less frequently, such as every 50-100 years. The frequency of the dose will be readily apparent to the skilled artisan and will depend upon any number of factors, such as, but not limited to, the type and severity of the disease being treated, the type and age of the mammal, etc.

In some embodiments, administration of an immunogenic composition or vaccine of the present invention may be performed by single administration or boosted by multiple administrations.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1

Ixodes scapularis ticks transmit many pathogens that cause human disease, including Borrelia burgdorferi. The experiments presented herein were conducted to examine the ability of lipid nanoparticle-containing nucleoside-modified mRNAs encoding 19 I. scapularis salivary proteins (19ISP) to enhance recognition of a tick bite and diminish I. scapularis engorgement on a host—and thereby prevent B. burgdorferi infection. Guinea pigs were immunized with 19ISP and challenged with L scapularis. Animals administered 19ISP developed erythema at the bite site shortly after ticks began to attach, and these ticks fed poorly, marked by early detachment and decreased engorgement weights. 19ISP immunization also impeded B. burgdorferi transmission. The effective induction of local redness early after I. scapularis attachment and the inability of the ticks to take a normal blood meal, suggests that 19ISP may be used—either alone or in conjunction with traditional pathogen-based vaccines—for the prevention of Lyme disease, and potentially other tick-borne infections.

Tick saliva is a complex blend of several proteins that are expressed dynamically depending on tick feeding and resulting changes in host responses (Narasimhan, et al., 2007, PLoS One, 2:e451; Narasimhan, et al., 2020, Ticks Tick Borne Dis, 11:101369; Perner, et al., 2018, PLoS Negl Trop Dis, 12:e0006410; Kim et al., 2016, PLoS Negl Trop Dis, 10:e0004323; Francischetti, et al., 2009, Front Biosci (Landmark Ed), 14:2051-2088). Using this information and previous analysis of the tick sialome (Simo et al., 2017, Front Cell Infect Microbiol, 7:281; Narasimhan, et al., 2020, Ticks Tick Borne Dis, 11:101369; Schuijt, et al., 2013, Circulation, 128:254-266; Narasimhan, et al., 2007, Cell Host Microbe, 2:7-18; Dai, et al., 2009, Cell Host Microbe, 6:482-492; Hovius, et al., 2008, PLoS Med, 5:e43; Anguita, et al., 2002, Immunity, 16:849-859; Cao, et al., 2020, Infect Immun; Contreras, et al., 2019, Front Physiol, 10:977; Das, et al., 2001, J Infect Dis, 184:1056-1064; Liu, et al., 2011, EMBO Rep, 12:1196-1203; Murfin, et al., 2019, Ticks Tick Borne Dis, 10:1124-1134; Narasimhan, et al., 2004, Proc Natl Acad Sci USA, 101:1141-1146; Ramamoorthi, et al., 2005, Nature. 436:573-577; Schuijt, et al., 2011, Cell Host Microbe, 10:136-146; Schuijt, et al., 2011, PLoS One 6, e15926; Sukumaran, et al., 2006, J Exp Med, 203:1507-1517). 19 salivary proteins were selected to form a cocktail of antigens (Table 4). These antigens are carefully selected based on their high immunogenicity and in most cases, with known mode of action. Nucleoside-modified mRNAs encoding these 19 I. scapularis proteins were encapsulated in lipid nanoparticles (I9ISP), which protect mRNA from degradation and facilitate in vivo delivery (Pardi, et al., 2020, Curr Opin Immunol, 65:14-20; Pardi, et al., 2018, Nat Rev Drug Discov, 17:261-279). The nucleoside-modified mRNA-lipid nanoparticle vaccination platform has shown promising results for infectious diseases including the recent COVID-19 human trials (Pardi, et al., 2018, Nat Rev Drug Discov, 17:261-279; Maruggi, et al., 2019, Mol Ther, 27:757-772; Zhang, et al., 2019, Front Immunol, 10:594; Jackson, et al., 2020, N Engl J Med, 383:1920-1931; Keech, et al., 2020, N Engl J Med, 383:2320-2332). In the present study, it is demonstrated that 19ISP effectively induces tick immunity in guinea pigs.

The materials and methods used in these experiments are now described.

### Ticks and Animals

I. scapularis ticks were obtained and maintained in an incubator at 23° C. and 90% relative humidity under a 14 h light, 10 h dark photoperiod. 4-5-weeks old female Hartley guinea pigs (Charles River Laboratories, MA) were used to feed nymphal ticks, as described earlier (Narasimhan, et al., 2020, Ticks Tick Borne Dis, 11:101369). Six weeks old female C3H mice (Charles River Laboratories, MA) were used for tick infection.

Preparation of 19ISP mRNA-lipid nanoparticle (mRNA-LNP) mRNA-LNPs were generated as previously described (Freyn, et al., 2020, Mol Ther, 28:1569-1584). mRNA vaccines encoding individual salivary antigens with their own signal peptide or IL2-signal peptide, and IL21 or firefly luciferase (Luc) were codon-optimized, synthesized and cloned into the mRNA production plasmid as described (Freyn, et al., 2020, Mol Ther, 28:1569-1584). mRNA production and LNP encapsulation was performed as described (Freyn, et al., 2020, Mol Ther, 28:1569-1584). Briefly, the sequence of mRNAs was transcribed to contain 101 nucleotide-long poly(A) tails. N1-Methyl-Pseudouridine-5′-Triphosphate (TriLink) instead of UTP was used to generate modified nucleoside-containing mRNA. Capping of the in vitro transcribed mRNAs was performed co-transcriptionally using the trinucleotide cap1 analog, CleanCap (TriLink). mRNAs were purified by cellulose purification, as described (Baiersdorfer, et al., 2019, Mol Ther Nucleic Acids, 15:26-35). All mRNAs were analyzed by agarose gel electrophoresis and were stored frozen at −20° C. In the multivalent 19ISP formulation equal amounts (by weight) from each of the 19 mRNAs were combined prior to LNP formulation. mRNAs were encapsulated in LNPs using a self-assembly process in which an aqueous solution of mRNA at acidic pH 4.0 was rapidly mixed with a solution of lipids dissolved in ethanol (Maier, et al., 2013, Mol Ther, 21:1570-1578; Jayaraman, et al., 2012, Angew Chem Int Ed Engl, 51:8529-8533), which contain an ionizable cationic lipid/phosphatidylcholine/cholesterol/PEG-lipid (50:10:38.5:1.5 mol/mol) and were encapsulated at an RNA to total lipid ratio of ˜0.05 (wt/wt), were stored at −80° C. at a concentration of mRNA of ˜1 μg/μl. The LNPs had a diameter of ˜80 nm as measured by dynamic light scattering using a Zetasizer Nano ZS (Malvern Instruments Ltd, Malvern, UK) instrument, with a polydispersity index of 0.02-0.06 and an encapsulation efficiency of ˜95%. Two or three batches from each mRNA-LNP formulations were used in these studies and we did not observe variability in vaccine efficacy. LNPs used in this study was prepared by Acuitas Therapeutics; the lipid and LNP composition are described in US patent U.S. Pat. No. 10,221,127.

### Immunization of Guinea Pigs

Female Hartley guinea pigs (5-weeks old) were immunized intradermally with 50 μg of 19ISP mRNA-LNPs (˜2.63 μg per antigen) or murine IL-21/Firefly Luciferase (Luc) mRNA-LNP (control). Since, a combination of 19 antigens has never been used, the approximate dosage was estimated on the basis of previous studies performed in mice and guinea pigs (Pardi, et al., 2015, J Control Release, 217:345-351; Awasthi, et al., 2019, Sci Immunol, 4; Pardi, et al., 2017, Nature, 543:248-251). Intradermal immunization is more efficient when using lower doses of antigens, without compromising the efficacy (Pardi, et al., 2015, J Control Release, 217:345-351; Awasthi, et al., 2019, Sci Immunol, 4; Midoux, et al., 2015, Expert Rev Vaccines, 14:221-234). The required amount of frozen mRNA-LNPs were thawed at room temperature, diluted with sterile PBS and used within 2 hours for injection. The animals were boosted twice at 4-week intervals. The animals were bled retro-orbitally 2 weeks after the last immunization to obtain blood for RNAseq and the serum was separated for use in ELISA. A minimum of 3 animals were used in each group.

### Generation of Recombinant Salivary Proteins

RNA was isolated from salivary glands dissected from I. scapularis ticks fed to repletion and cDNA synthesized according to the manufacture's protocol (iScript cDNA synthesis kit, Bio-Rad). Gene-specific primers were used to amplify the mRNA region encoding the mature proteins listed in Table 4. Purified amplicons were then cloned into the pMT-Bip-V5-HisA cloning vector or the pET28A Escherichia coli expression vector and recombinant DNA sequenced to validate the clones. Recombinant proteins were generated using a Drosophila expression system as described earlier for different antigens (Narasimhan, et al., 2007, Cell Host Microbe, 2:7-18; Anguita, et al., 2002, Immunity, 16:849-859). For expression in E. coli, the clones were transformed in endotoxin-free ClearColi cells BL21(DE3) (Lucigen, WI) and expressed with an N-terminal His6-tag. The proteins were purified using Ni²⁺-NTA-Agarose resin according to the manufacturer's instructions (Qiagen, CA). Protein purity was assessed by SDS-PAGE using 4-20% gradient precast gels (Biorad, CA) and quantified using the BCA protein estimation kit (Thermo Scientific, MA). Recombinant IsPDIA3 was generated as a GST-fusion (glutathione-S-transferase) protein in E. coli using the pGEX-4T2 vector (Cao, et al., 2020, Infect Immun) and recombinant protein purified using GST-resin according to the manufacturer's protocol (GE Healthcare Life Sciences, PA).

### ELISA Assessment of Recombinant Salivary Proteins

To assess the 19ISP specific antibody response against individual proteins, 96-well Immunosorp ELISA plates were coated overnight with 250 ng of recombinant proteins, blocked with 3% BSA for 1 h at 37° C. and incubated with guinea pig anti-19ISP sera collected 2 weeks after the last immunization dose at 1:500, 1:5,000 or 1:50,000 dilutions for 2 h. Each step was separated by 3 washes with PBST (PBS with 0.025% Tween-20) Bound antibody was detected with HRP-conjugated goat anti-guinea pig IgG secondary antibody and TMB substrate solution (ThermoFisher Scientific, IL). The reaction was stopped by TMB stop solution and absorbance was read at 450 nm.

### RNAseg Analysis

Total RNA was extracted from whole blood obtained from guinea pigs 2 weeks after the final immunization. Trizol was added to the whole blood and RNA was isolated according to the manufacturer's instructions (Qiagen, CA). RNA was submitted for library preparation using TruSeq (Illumina, San Diego, CA, USA) and sequenced using Illumina HiSeq 2500 by paired-end sequencing at the Yale Center for Genome Analysis (YCGA). RNAseq analyses including alignment, quantitation, normalization, and differential gene expression analyses were performed using Partek Genomics Flow software (St. Louis, MO, USA). Specifically, RNAseq data were trimmed and aligned to the guinea pig genome (Cavea porcellus, Cavpor 3.0 from Ensembl), with associated annotation file using STAR (v 2.7.3a). The aligned reads were quantified to Ensembl transcripts using the Partek E/M algorithm and the subsequent steps were performed on gene-level annotation followed by total count normalization. The gene-level data were normalized by dividing the gene counts by the total number of reads followed by the addition of a small offset (0.0001). Principal components analysis (PCA) was performed using default parameters for the determination of the component number, with all components contributing equally in Partek Flow. Hierarchal clustering was performed on the genes that were differentially expressed across the conditions (P<0.05, fold change ≥2 for each comparison). Pathway enrichment was conducted by converting the guinea pigs Ensembl gene symbol to the Entrez gene ID for mice, since the guinea pig genome is not annotated in Partek Flow. The top 10 immune pathways were further plotted on a bubble diagram by ggplot2 in R studio.

Cytokine Expression Analysis from Peripheral Blood Mononuclear Cells (PBMCs)

PBMCs were isolated from guinea pig blood 2 weeks after third immunization and stimulated with 3 μl of tick saliva in a total volume of 100 μl (Patton, et al., 2012, J Vis Exp) for 24 h at 37° C. RNA was extracted from stimulated and unstimulated PBMCs using the Qiagen RNeasy kit and cDNA was prepared from the purified RNA using the iScript cDNA synthesis kit (BioRad). cDNA was analyzed by quantitative RT-PCR using the iTaq Sybr Green Supermix (Biorad, CA) for the expression of guinea pig-specific cytokines and chemokines, including interferon-γ (IFNγ), tumor necrosis factor-α (TNFα), CXCL10, Interleukin 2 (IL-2), IL-4 and IL-8. The relative expression was calculated using the deltaCq method and normalized to the expression of the guinea pig gapdh gene.

### Uninfected Tick Challenge of Guinea Pigs

Two weeks following the last immunization dose, guinea pigs were challenged with uninfected I. scapularis ticks, as described earlier (Narasimhan, et al., 2020, Ticks Tick Borne Dis, 11:101369). Briefly, after anesthetizing by intramuscular injection of a ketamine and xylazine mixture, the guinea pigs were challenged with 25 I. scapularis nymphs. Ticks were allowed to attach to shaved backs of guinea pigs. Guinea pigs were housed individually with 3 layers of tick containment (a pan of water below the wire-bottom of the cage, a hopper-inclusive lid, and grease around the outer edges of the cage). Daily monitoring was performed to assess the numbers of ticks attached, feeding patterns, and skin erythema, and to collect any detached ticks from the water pan. The numbers of ticks detached and recovered were used to calculate percent recovery and measure the engorgement weights. Erythema at the tick bite sites was assessed by two researchers blinded to the experimental groups and scored based on the percentage of erythematous bite sites on the total of attached ticks.

Infected Tick Challenge and B. burgdorferi Transmission of Guinea Pigs

To generate B. burgdorferi-infected mice and nymphs, B. burgdorferi N40 was inoculated in C3H mice as described previously (Narasimhan, et al., 2007, PLoS One, 2:e451). Approximately, 100 μl of 1×10⁵ N40 spirochetes/ml were injected subcutaneously. I. scapularis larvae were placed on B. burgdorferi-infected C3H mice and fed larvae molted to generate B. burgdorferi-infected nymphs. For Borrelia transmission to guinea pigs, 3 B. burgdorferi N40 infected nymphs were placed on each guinea pig (at least 5 animals in each group) and allowed to feed till the appearance of erythema (up to 120 h post-tick-challenge), after which ticks were pulled-off carefully using forceps. All control and experimental animals were examined for erythema in a double-blinded manner. After tick detachment, the transmission was assessed by culture and by quantitative PCR of skin punches at 3 weeks. In a previous study, skin, blood, spleen and bladder were examined after B. burgdorferi-infected ticks were allowed to engorge on guinea pigs and were not able to detect spirochetes tissues other than skin (Nazario, et al., 1998, Am J Trop Med Hyg, 58:780-785). In several of the animals in the current study, numerous internal sites were looked at, and consistent with the previous experiment, B. burgdorferi was only able to be detected in the skin of the guinea pigs. A additional experiment was performed in which one B. burgdorferi-infected tick was placed on each control and experimental animals, and the ticks were allowed to attempt to take a blood meal until they naturally detached from the animals.

### Quantitative PCR to Estimate Spirochete Burden

Guinea pig skin punch biopsies were obtained from sites near and distal to tick attachment sites at 3 weeks, after tick engorgement. The biopsies were suspended in DNAeasy suspension buffer (Qiagen, CA) containing proteinase K and processed for DNA isolation using the DNAeasy kit (Qiagen) according to the manufacturer's protocol. DNA was analyzed by quantitative PCR using the iTaq Sybr Green Supermix (Biorad, CA), for the presence of Borrelia using flaB primers (flaB F-5′ ttcaatcaggtaacggcaca 3′ (SEQ ID NO:56) and flaB R-5′ gacgcttgagaccctgaaag 3′ (SEQ ID NO:57) and results normalized using actin primers (actin F-5′agcgggaaatcgtgcgtg 3′ (SEQ ID NO:58) and actin R-5′cagggtacatggtggtgcc 3′ (SEQ ID NO:59)).

### Statistical Analysis

Data for ELISA, skin erythema, engorgement weights, and tick detachment/recovery were analyzed by Prism 9.0 software (GraphPad Software, CA). The significance of the difference between controls and experimental groups was analyzed by ordinary ANOVA, two-way ANOVA or Mann-Whitney/Welch's test (as mentioned in respective figure legends). P<0.05 was considered statistically significant.

The results of the experiments are now described.

Generation of the Lipid Nanoparticle-Containing Nucleoside-Modified mRNAs Encoding 19 I. Scapularis Salivary Proteins (19ISP)

19 genes known to be expressed in I. scapularis salivary glands were selected, many of which are secreted at the bite site (Table 4). Most of these genes have been characterized in individual studies and several have been tested for their efficacy against tick infestation. Salivary protein of 14 kDa (Salp14), TSLPI, Salp10, Salp15, Salp16A, Salp17, Salp25A, Salp25B, Salp25C, Salp25D, Salp26A, tick inhibitor of factor Xa (TIX5), and a 32 kDa salivary protein (P32) were initially identified by immunoscreening assays as secreted salivary proteins that reacted avidly with tick-resistant animal sera (Das, et al., 2001, J Infect Dis, 184:1056-1064; Schuijt, et al., 2011, PLoS One 6, e15926). Some of these salivary antigens regulate host immune responses, or influence pathogen infectivity (Schuijt, et al., 2013, Circulation, 128:254-266; Narasimhan, et al., 2007, Cell Host Microbe, 2:7-18; Dai, et al., 2009, Cell Host Microbe, 6:482-492; Anguita, et al., 2002, Immunity, 16:849-859; Narasimhan, et al., 2004, Proc Natl Acad Sci USA, 101:1141-1146; Ramamoorthi, et al., 2005, Nature. 436:573-577; Schuijt, et al., 2011, Cell Host Microbe, 10:136-146; Sukumaran, et al., 2006, J Exp Med, 203:1507-1517). P11 is a secreted salivary protein involved in A. phagocytophilum infection of salivary glands (Liu, et al., 2011, EMBO Rep, 12:1196-1203). Salp12, SG09, SG10, SG27, and I. scapularis protein disulfide isomerase (IsPDIA3) are secreted salivary proteins that may influence B. burgdorferi acquisition (Cao, et al., 2020, Infect Immun; Murfin, et al., 2019, Ticks Tick Borne Dis, 10:1124-1134). SG10 is a heme lipoprotein and SG09 is a hemelipoglyco-carrier protein, present in I. scapularis saliva with homologs identified in saliva, hemolymph, and tissues of a variety of other tick species, including Ixodes ricinus (Kim et al., 2016, PLoS Negl Trop Dis, 10:e0004323; Dupejova, et al., 2011, Parasit Vectors, 4:4; Kim, et al., 2020, PLoS Negl Trop Dis, 14:e0007758). Heme-binding class proteins are highly abundant in the saliva of I. scapularis, with known or putative functions in other tick species that include transport and storage of heme, detoxification, and involvement in innate immunity (Kim et al., 2016, PLoS Negl Trop Dis, 10:e0004323; Dupejova, et al., 2011, Parasit Vectors, 4:4; Kim, et al., 2020, PLoS Negl Trop Dis, 14:e0007758; Maya-Monteiro, et al., 2004, Insect Biochem Mol Biol, 34:81-88). A recent study included an I. ricinus heme lipoprotein homologous to SG10 among the many potential targets that may influence tick infestation in rabbits and dogs (Contreras, et al., 2019, Front Physiol, 10:977).

The individual nucleoside-modified mRNAs for 19 genes were synthesized in vitro and encapsulated in a lipid nanoparticle (LNP) in equal amounts to generate 19ISP, as outlined in the methods section. The 19ISP mRNA-LNP vaccine was used to immunize guinea pigs to test for the generation of immunity against tick bites.

TABLE 4
Genes incorporated into the 19ISP mRNA vaccine

| | Gene/protein | |
|---|---|---|
| Gene | accession | Characteristics^{a} |
| Salp10 | AAK97828/ | Elicits antibodies in host, homology |
| | AF278575 | with Kunitz-type protease inhibitors, |
| | | altered expression in response to A. |
| | | phagocytophilum |
| Salp15 | AAK97817/ | Alters CD4⁺ T cell function, influences |
| | AF209914 | B. burgdorferi infection |
| Salp25A | AAK97825/ | Generates antibodies in host, putative |
| | AF209922 | histamine binding protein |
| Salp25B | AAK97821/ | Produces antibodies in host, probable |
| | AF209918 | histamine binding protein |
| Salp25C | AAK97816/ | Potential histamine binding protein, |
| | AF209913 | altered expression in response to |
| | | A. phagocytophilum |
| Salp25D | AAK97814/ | Peroxiredoxin, role in acquisition of |
| | AF209911 | B. burgdorferi by I. scapularis in |
| | | the host |
| Salp14 | AAK97824/ | Anticoagulant that inhibits factor Xa |
| | AF209921 | |
| TSLPI | HQ605983/ | Lectin complement pathway inhibition |
| | AEE89466 | |
| Salp26A | AAK97822/ | Generates antibodies in host |
| | AF209919 | |
| P11 | DQ066011/ | Facilitates A. phagocytophilum |
| | AAY66648 | migration to tick salivary glands |
| Salp16A | AAK97818/ | Elicits antibodies in host, homology |
| | AF209915 | with Salp15 |
| Salp17 | AAK97819/ | Expression decreased by |
| | AF209916 | A. phagocytophilum |
| TIX5 | AEE89467/ | Factor Xa Inhibitor |
| | HQ605984 | |
| P32 | ADO95260/ | Reacts with tick immune serum |
| | HM802761 | |
| Salp12 | XP_002433874/ | Chemoattractant for B. burgdorferi |
| | XM_002433829 | and influences acquisition |
| SG27 | XP_002405832/ | Putative B. burgdorferi attractant, |
| | XM_002405788 | protein disulfide-isomerase |
| IsPDIA3 | XP_002406442/ | Protein disulfide-isomerase, |
| | XM_002406398 | Contributes to B. burgdorferi |
| | | colonization of ticks |
| SG10 | XP_002411436/ | Heme lipoprotein |
| | XM_002411391 | |
| SG09 | XP_002411435/ | Hemelipoglyco-carrier protein |
| | XM_002411390 | |

^{a}Data from references, Pfam and Simple modular architecture research mtool (SMART) domain analysis

Immunization with 19ISP Generates Antibody Responses to Specific Antigens in Guinea Pigs

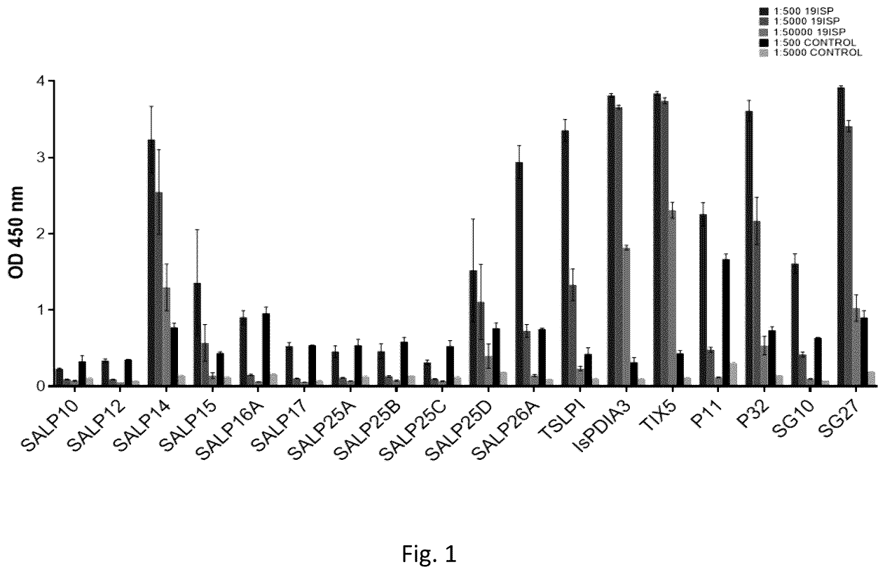

Guinea pigs were immunized intradermally three times at 4-week intervals with 50 μg 19ISP mRNA-LNP and IL-21 mRNA-LNP as a control. Two weeks after the last dose and prior to tick challenge, blood was collected from the immunized guinea pigs and sera were isolated. Sera IgG titers were evaluated by ELISA using recombinant salivary protein antigens. Eighteen antigens were tested for the presence of specific antibodies. The primary sequences of SG09 and SG10 share 75% identity which precludes conclusive determination of antibodies specific to these two proteins. Therefore, recombinant SG09 was not generated for ELISA assays. Interestingly, antibodies were detected against 10 of the selected proteins—Salp14, Salp15, Salp25D, Salp26A, TSLPI, IsPDIA3, TIX5, P32, SG10, and SG27 (FIG. 1). Antibodies for these proteins were not detected with the sera obtained from the control animals. These results suggest that 19ISP vaccination elicits antigen-specific humoral responses in guinea pigs.

### 19ISP Immunization Elicits Protective Responses Against Tick Challenge in Guinea Pigs

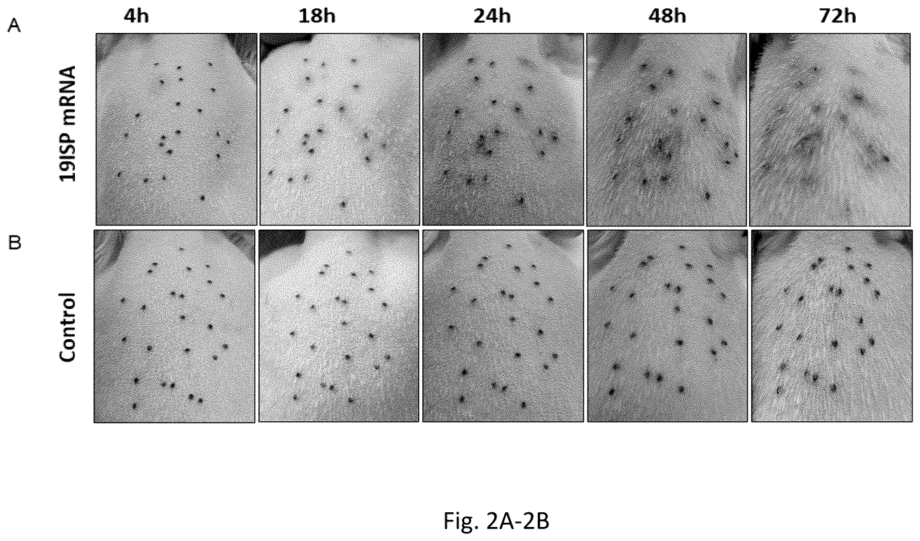

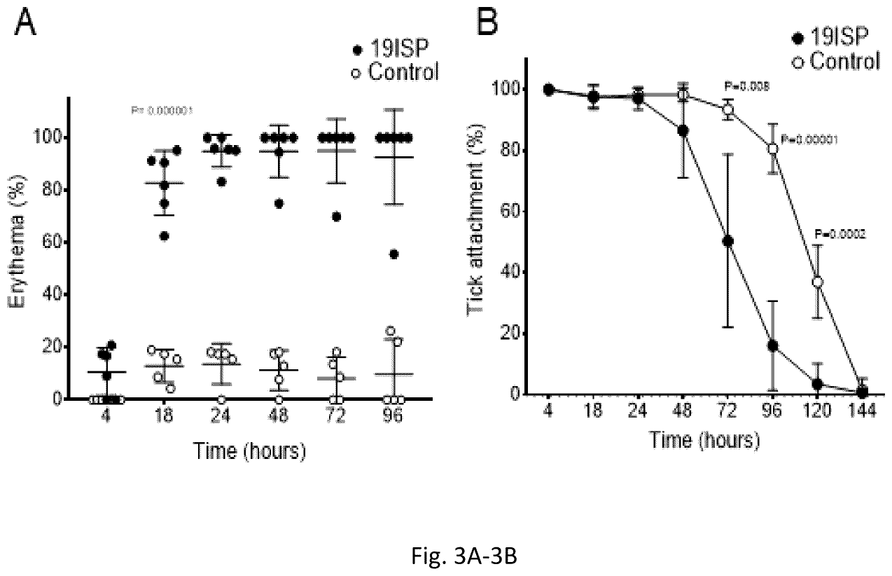

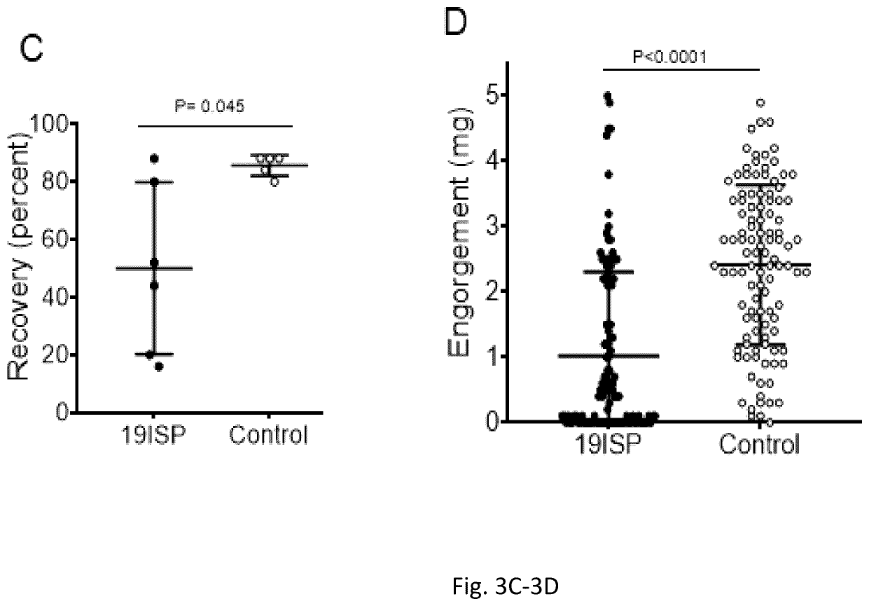

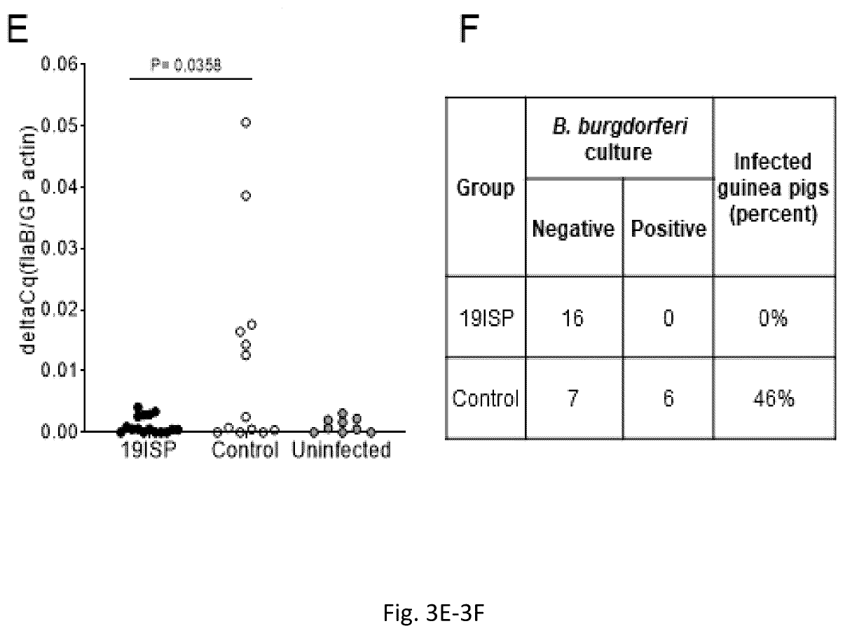

25 uninfected I. scapularis nymphs were placed on 19ISP-immunized or control (IL21-immunized) guinea pigs and allowed to naturally attach. The guinea pigs were monitored for the development of erythema at the bite site, which is the earliest hallmark associated with acquired tick resistance. As shown in FIG. 2, significant erythema was observed in 19ISP-immunized animals as early as 18 hours post tick challenge. The erythema peaked at 24 hours at all the bite sites and persisted throughout the tick challenge. Tick bite in the control animals did not show substantial redness (FIG. 2 and FIG. 3A).

The immunized guinea pigs were also monitored for other hallmarks of tick immunity that occur after the appearance of erythema, including tick rejection, feeding, and engorgement weights. For 19ISP-immunized animals, the ticks fed poorly and started to detach by 48 hours post tick challenge (FIG. 3B). Being small in size and poorly fed, the recovery of I. scapularis was also reduced with many of the dead tick shells merely attached to the guinea pigs (FIG. 2, 72h and FIG. 3C). By 96 hours post tick challenge, 80% of the ticks detached from the 19ISP-immunized guinea pigs, as compared to 20% detachment in control animals. Due to poor feeding and early detachment, the engorgement weights of the ticks fed upon 19ISP-immunized animals were significantly lower (mean weight=1.02 mg, n=109) as compared to the ticks which fed upon control animals (mean weight=2.42 mg, n=108) (FIG. 3D). These data indicate that 19ISP vaccination induces acquired resistance to tick bites in guinea pigs.

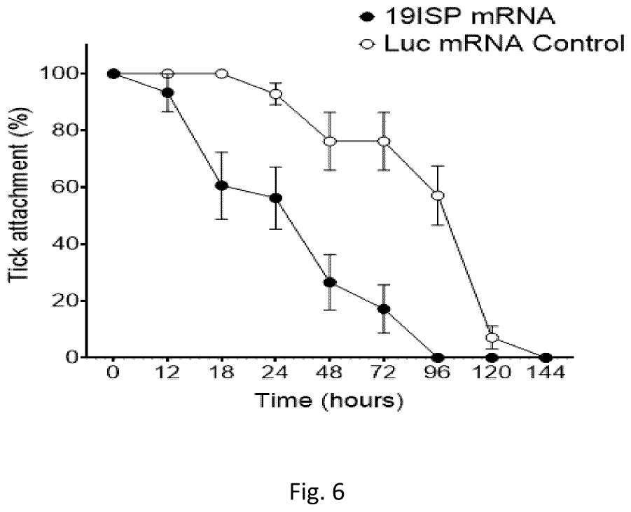

A subsequent study was performed to determine whether 19ISP immunization could influence the transmission of B. burgdorferi to guinea pigs. Although a single B. burgdorferi-infected tick is sufficient to transmit full infection (64), three B. burgdorferi-infected I. scapularis nymphs were placed on each guinea pig immunized either with 19ISP or Luc-mRNA control. As most humans do not commonly get 3 tick bites at the same time, and because many I. scapularis in nature are not infected with B. burgdorferi, it was assumed that 3 ticks on guinea pigs represent a substantial degree of exposure to the Lyme disease agent. In addition, as humans are likely to remove a tick that causes a bite with concomitant erythema or pruritis, the ticks were removed from the experimental and control guinea pigs, in a double-blind manner, when redness became evident (FIG. 6). At 3 weeks after exposure to ticks, the guinea pigs were euthanized, and biopsies were taken adjacent to bite site to test the infection levels. The experiment was repeated three times. In total, almost half (46%) of the control guinea pigs were PCR-positive (6/13) for B. burgdorferi while none (0%) of the 19ISP-immunized guinea pigs were PCR-positive (0/16) for B. burgdorferi (FIG. 3E). To further confirm these results, B. burgdorferi was cultured from skin biopsy specimens from all PCR-positive guinea pigs. All cultures were negative from PCR-negative animals (FIG. 3F). In a subsequent experiment we allowed a single B. burgdorferi-infected tick to feed without removal on 19ISP-immunized or control guinea pigs, to mimic an un-noticed human infection from single tick bite. It was found that 60% (3/5) of control guinea pigs were infected with B. burgdorferi, whereas none (0/5) of the 19ISP-immunized animals were infected, as tested by PCR and culture. Collectively, these data show that 19ISP-immunization can protect against tick-borne B. burgdorferi infection.

Differential Host Responses Associated with 19ISP-mRNA Vaccination

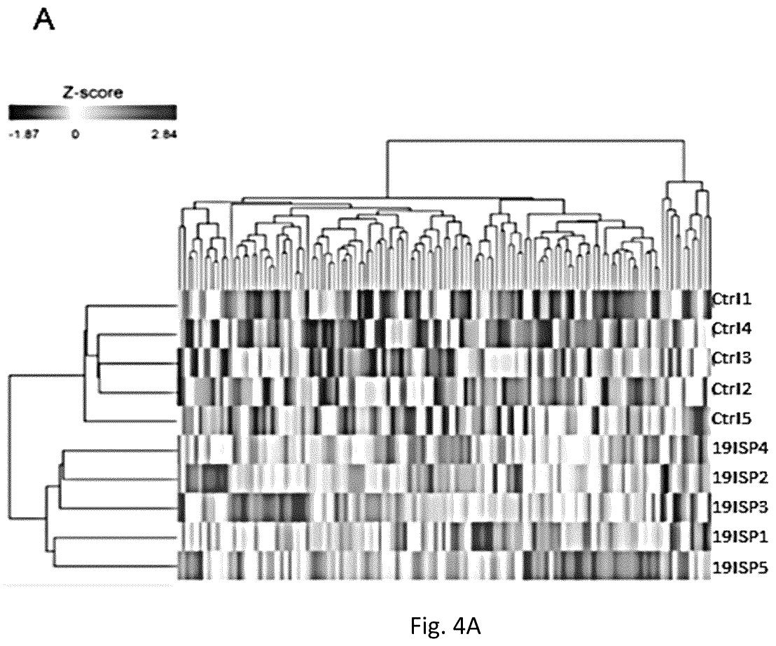

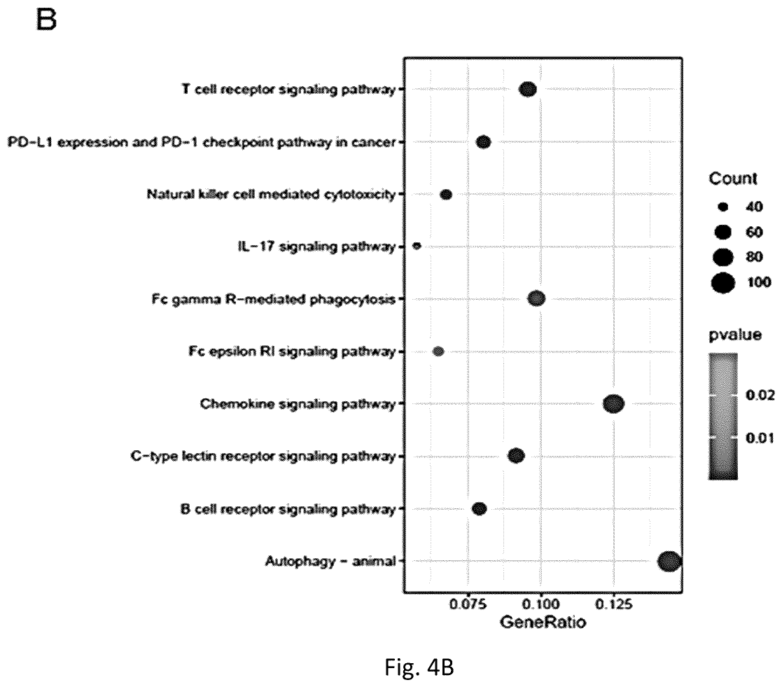

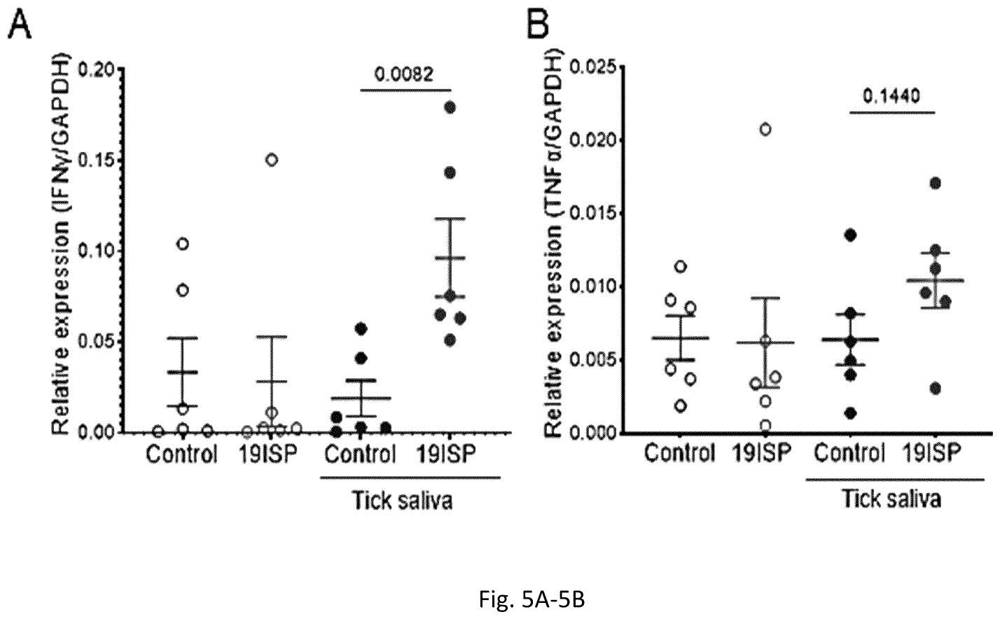

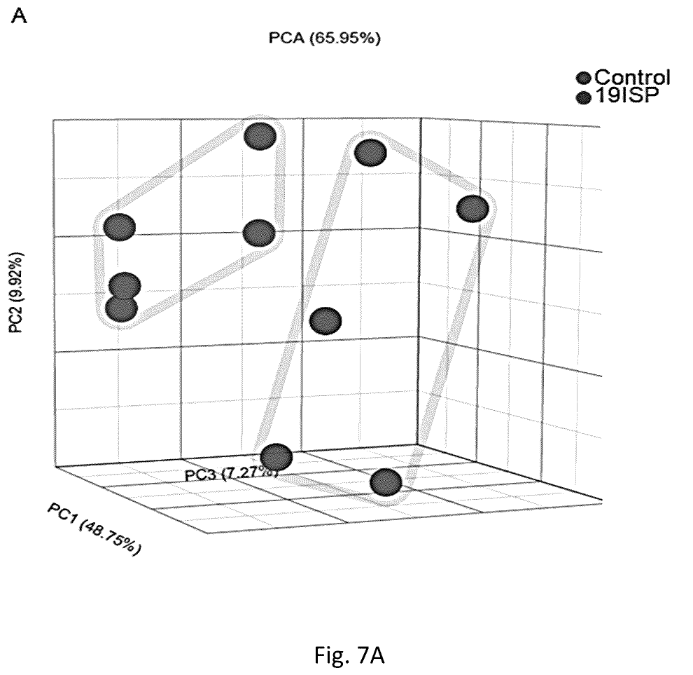

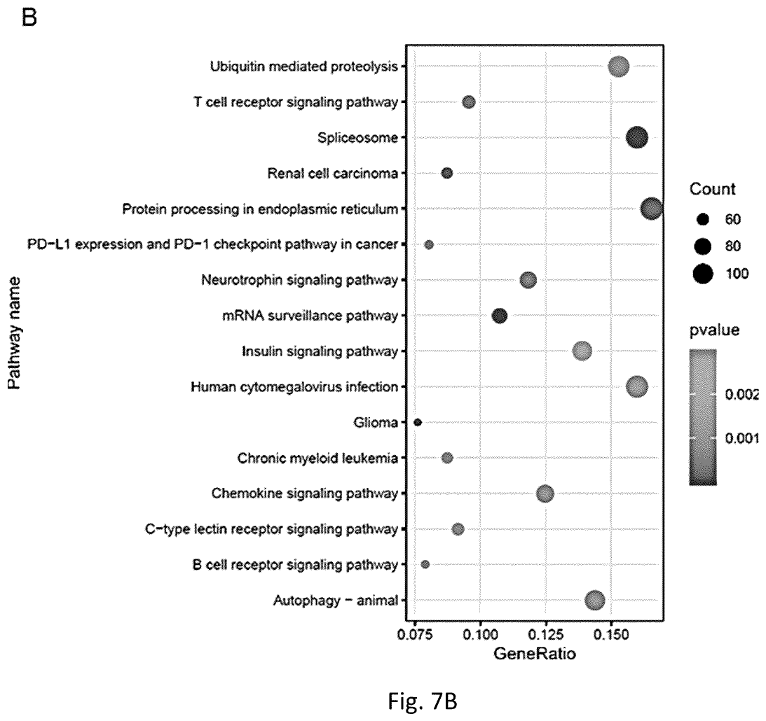

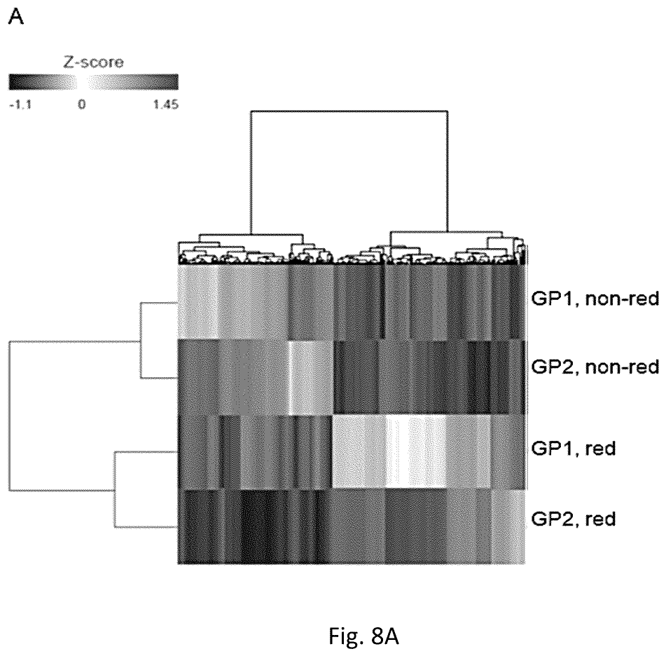

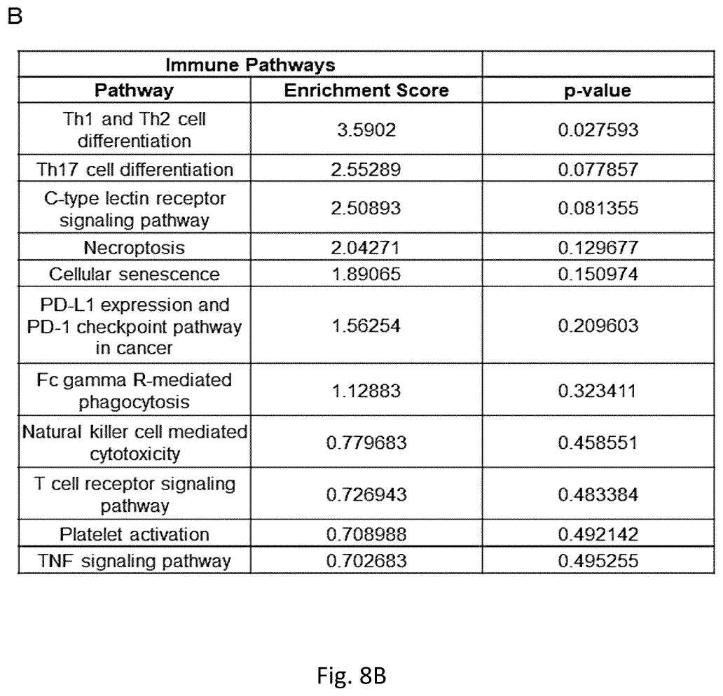

To further understand the immune responses associated with I9ISP vaccination and host protection, RNA was isolated from immunized guinea pigs for gene expression analysis two weeks after the final immunization. Significant differences in whole blood gene expression in 19ISP-immunized animals was found as compared to controls. Principal component analysis (PCA) and cluster dendrogram revealed that the 19ISP-vaccinated animal group formed a separate cluster from the control animal groups (FIG. 7A and FIG. 4A). A total of 125 differentially expressed genes were identified with a p-value less than 0.05 and a fold change greater than or equal to 2.0. 113 genes were up-regulated, and 12 genes were down-regulated in the 19ISP-immunized group as compared to the control group (FIG. 4A). To identify signaling pathways involved in response to 19ISP-mRNA vaccination, KEGG pathway enrichment analysis was utilized. The top 20 enriched pathways include many immune pathways (FIG. 7B). The top immune pathways enriched following vaccination are T cell receptor, B-cell receptor signaling pathways, chemokine signaling pathways, IL-17 signaling, Natural killer cell-mediated cytotoxicity, FcεRI-mediated signaling, and C type lectin receptor signaling pathways (FIG. 4B). In addition, gene expression at the erythematic bite site was also compared with a non-erythematic site in the same guinea pigs immunized with 19ISP. The data shows enrichment of T-cell-related pathways, indicating elicited T-cell response (FIG. 8).

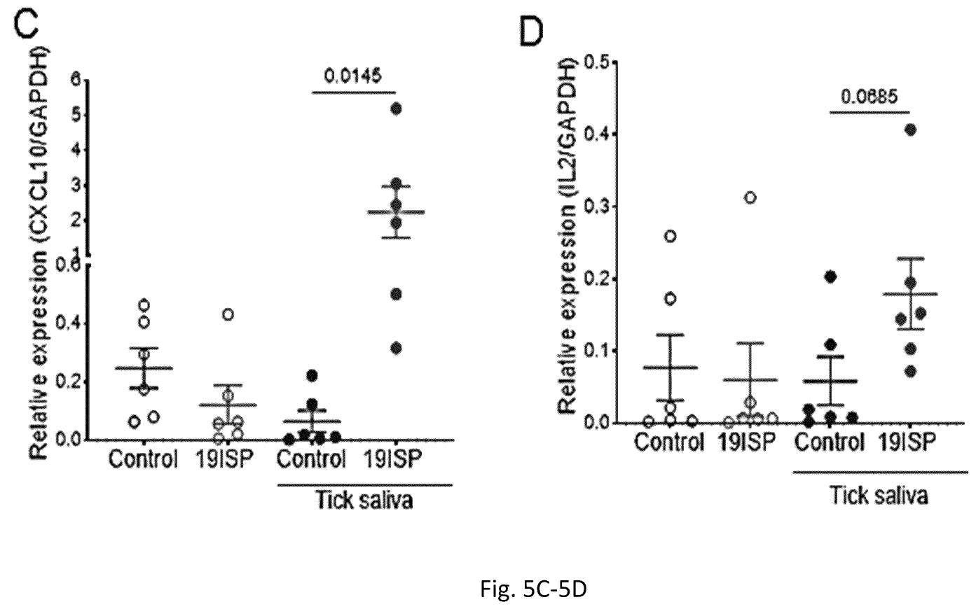

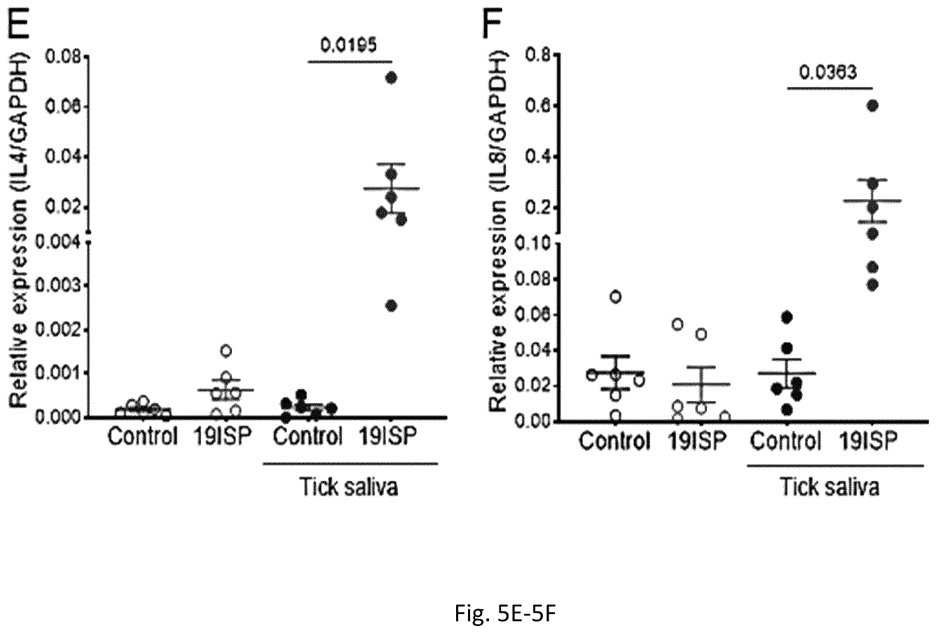

To further understand some of the cell-mediated immune responses due to the 19ISP vaccination, PBMCs were isolated from 19ISP- and control (Luc)-mRNA immunized guinea pigs, 2-weeks after the second boost. PBMCs were stimulated with I. scapularis saliva, total RNA was extracted, and the expression of selected cytokines/chemokines commonly induced by activated T- and B-cells following vaccinations, including IFNγ, TNFα, CXCL10, IL2, IL4, and IL8, were examined. The expression of these cytokines was increased in 19ISP-immunized animals (FIG. 5) as compared to controls.

### Discussion

In the present study, attempts were made to generate acquired tick resistance, or “tick immunity” to I. scapularis, using 19 salivary proteins that have a spectrum of functions in tick feeding, interaction with the pathogen, or host responses, reflecting a portion of the tick sialome (Table 4). To optimize the immune response, a nucleoside-modified mRNA-LNP platform was chosen that allows for more continuous delivery of the antigen (Pardi, et al., 2015, J Control Release, 217:345-351; Pardi, et al., 2018, J Exp Med, 215:1571-1588), and therefore perhaps more closely resembling part of a tick bite. Safe and effective nucleoside-modified mRNA-LNP vaccines are currently being used in humans against SARS-CoV-2, therefore it is an ideal vaccine platform to deliver tick antigens to a host.

Guinea pigs were used as the primary animal model because they are not part of the natural life cycle of I. scapularis and readily develop tick immunity following repeated exposure to I. scapularis (Narasimhan, et al., 2019, Ticks Tick Borne Dis, 10:386-397; Wikel, 1996, Annu Rev Entomol, 41:1-22; Willadsen, in Advances in Parasitology, W. H. R. Lumsden, R. Muller, J. R. Baker, Eds. (Academic Press, 1980), vol. 18, pp. 293-313; Allen, 1989, Exp Appl Acarol, 7:5-13). In addition, guinea pigs can be infected with tick-borne B. burgdorferi (Nazario, et al., 1998, Am J Trop Med Hyg, 58:780-785), and can therefore be used to determine whether acquired tick resistance can influence the transmission of the Lyme disease agent. It is shown that immunization with 19ISP provides robust tick immunity in guinea pigs, including significant early erythema after tick placement on the animals and rapid tick detachment, along with severely impaired tick feeding and low engorgement weights (Pardi, et al., 2018, J Exp Med, 215:1571-1588; Awasthi, et al., 2019, Sci Immunol, 4; Lindgren, et al., 2017, Front Immunol, 8: 1539).

Host responses in the 191SP-immunized guinea pigs were further assessed, using the relatively limited reagents for this species. Robust antibody responses to a subset of the 19 antigens—Salp14, Salp15, Salp25D, Salp26A, TSLPI, IsPDIA3, TIX5, P32, SG10, and SG27—were detected in 19ISP-immunized guinea pigs, suggesting that these may be the targets most closely associated with tick immunity (FIG. 1). Nine of the proteins did not elicit detectable responses, suggesting that they are not highly antigenic and may not be directly linked with tick immunity (FIG. 1). PBMCs from 19ISP-immunized guinea pigs stimulated with I. scapularis saliva elicited the production of several T-cell-related cytokines (FIG. 5), suggesting that a combination of humoral and cell-mediated immunity accounts for the magnitude of response by 19ISP mRNA vaccination. Finally, RNA-seq elucidated the specific genetic signatures associated with 19ISP-immunization in guinea pigs. The activated pathways included T cell receptor, B-cell receptor signaling pathways, chemokine signaling pathways, IL-17 signaling, natural killer cell-mediated cytotoxicity, FcεRI mediated signaling, and C-type lectin receptor signaling pathways. FcεRI signaling and B-cell receptor signaling pathways were also activated in our previous study to delineate pathways that are differentially activated at the bite site of guinea pigs with naturally acquired tick resistance following exposure to multiple tick-bites (Kurokawa, et al., 2020, Ticks Tick Borne Dis, 11:101529). The activation of common pathways in naturally acquired tick resistance and after 19ISP-immunization can help to guide future vaccine development and studies with mRNA LNPs containing selected subsets of the genes will help address the contribution of each of the 19 genes in the genesis of acquired tick resistance.

The I9ISP-immunized guinea pigs were protected from tick-borne B. burgdorferi infection when the ticks were removed when erythema became pronounced. This time point was chosen because when humans notice redness or irritation due to a tick bite, the immediate response is to remove the tick. Such erythema-associated itch is apparent in tick immune guinea pigs and likely to occur in humans (Anderson, et al., 2017, Front Immunol, 8:1784). Additionally, when challenged with a B. burgdorferi-infected tick that was allowed to continue to try to feed until it fell off, none of 19ISP immunized animals were infected, while 60% of the control guinea pigs were infected. Humans, like guinea pigs, are not important in the natural life cycle of I. scapularis, and it is, therefore possible that vaccination of humans with I9ISP can help to prevent Lyme disease, either because the ticks naturally detach early or because humans more readily remove ticks when erythema is present. Ticks that attach and feed to repletion on humans are much more likely to transmit B. burgdorferi than ticks that are removed early in the feeding process (Sood, et al., 1997, J Infect Dis, 175:996-999; Piesman, et al., 1987, J Clin Microbiol, 25:557-558; Kahl, et al., 1998, Zentralbl Bakteriol, 287:41-52; des Vignes, et al., 2001, J Infect Dis, 183:773-778).

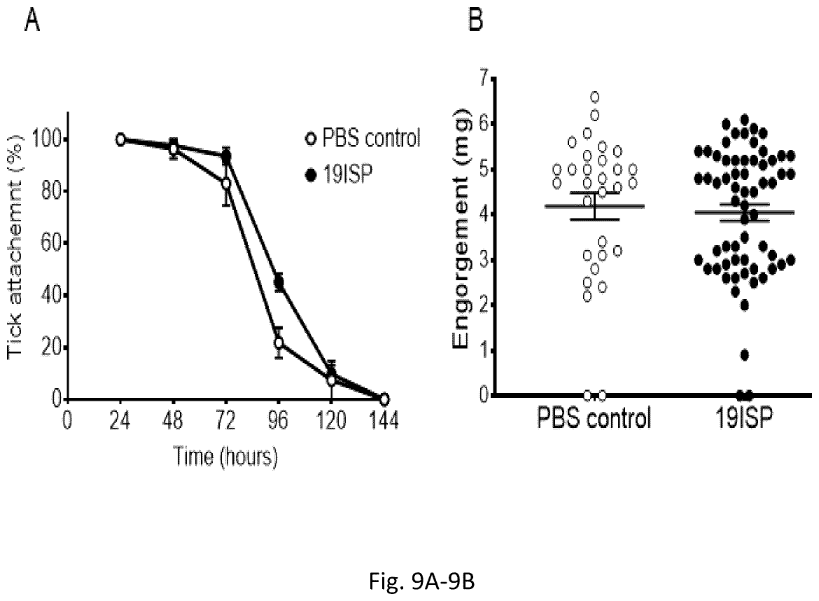

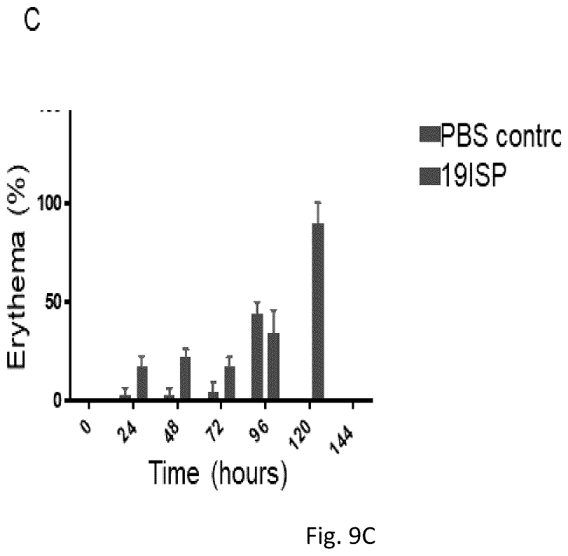

Mice, in contrast to guinea pigs, are important in the natural life cycle of L scapularis and do not develop significant acquired tick resistance following repeated exposure to I. scapularis (Tabakawa, et al., 2018, Front Immunol, 9:1540; Narasimhan, et al., 2019, Ticks Tick Borne Dis, 10:386-397; Wikel, 1996, Annu Rev Entomol, 41:1-22; Anderson, et al., 2017, Front Immunol, 8:1784). It is possible that ticks have co-evolved with animals important in their natural life cycle, in part, to optimize their capacity to feed successfully. Consistent with this, mice immunized with 19ISP did not demonstrate significant erythema at the tick bite site, and there was no impact on tick feeding (FIG. 9). As humans are not important in the natural life cycle of I. scapularis, they are therefore more likely to respond in a manner similar to guinea pigs.

In conclusion, 19ISP immunization can elicit acquired resistance against L scapularis and prevent tick-borne B. burgdorferi infection in guinea pigs. As some of these infectious agents are transmitted rapidly by ticks, 19ISP vaccination in humans would need to elicit almost immediate recognition of a tick-bite to prevent infection. To date, all human vaccines against infectious disease directly target pathogens or microbial targets. There have been promising studies with anti-sandfly proteins that protect the host against Leishmaniasis (Oliveira, et al., 2015, Sci Transl Med, 7:290ra290; Cecilio, et al., 2020, Sci Rep, 10:18653). In addition, commercial anti-tick vaccines in animals reduce the tick burden but have not been generally shown to prevent the transmission of an infectious disease (Valle, et al., 2018, Front Biosci (Elite Ed), 10:122-136; de la Fuente, et al., 2007, Vet Res Commun, 31:Suppl 1, 85-90; de la Fuente, et al., 2007, Anim Health Res Rev, 8:23-28; Almazan, et al., 2018, Front Biosci (Landmark Ed), 23:1535-1551). A study in mice however, demonstrated that vaccination with Rhipicephalus appendiculatus cement protein 64TRP protects against Ixodes ricinus transmitted tick-borne encephalitis virus (Labuda, et al., 2006, PLoS Pathog, 2:e27). I9ISP may change that paradigm by demonstrating that a human anti-tick vaccine may prevent infectious diseases, and also suggest that a vaccine that elicits more rapid tick recognition may be sufficient to prevent infection. These data demonstrate that a multivalent mRNA vaccine that targets I. scapularis antigens has the potential to prevent a common tick-borne infectious disease.

Sequences

Salp10 AAK97828
SEQ ID NO: 1
MKAAIAVLCFLVAVAYAIVVEARMANQPIDDGPLNPKCVKPKGPGDFKTVSYYDPNKGCQLIKLG
ENCTDNGNYPTLEDCNRHCLPPPGKQTRLS
SEQ ID NO: 2
>AF278575.1 Ixodes scapularis Salp10 mRNA, complete cds
ATGAAGGCAGCCATTGCAGTTCTTTGCTTTCTCGTTGCAGTAGCGTACGCCATTGTGGTGGAGGC
GAGGATGGCGAACCAACCGATTGACGACGGCCCCCTCAATCCAAAATGCGTAAAACCGAAAGGTT
GCCCCGGAGATTTCAAGACGGTTTCCTATTACGACCCGAATAAAGGATGTCAACTTATTAAACTG
GGAGAAAACTGTACCGACAATGGCAATTACCCCACACTTGAGGATTGCAATCGGCATTGTCTACC
TCCTCCAGGGAAGCAAACGAGACTCAGCTGA
Salp 15 AAK97817.1
SEQ ID NO: 3
MESFVAMKVVCILFLVGVVAANESGPTKADASTADKDTKKNNVQLRFPNYISNHQKLALKLLKIC
KDSKSSHNSLSSRSSDVINDKYVDFKNCTFLCKHGNDVNVTLNLPEDTPCGPNGQTCAEKNKCVG
HIPGC
SEQ ID NO: 4
>AF209914.1 Ixodes scapularis 15 kDa salivary gland protein
(Salp15) mRNA, complete cds
ATGGAATCTTTCGTCGCAATGAAGGTGGTGTGCATACTATTTTTGGTTGGTGTTGTCGCTGCGAA
TGAAAGCGGCCCAACTAAAGCAGACGCATCAACCGCTGACAAAGATACGAAGAAAAACAATGTGC
AACTTCGATTCCCTAATTATATTTCTAACCACCAAAAGCTTGCCTTGAAACTTCTGAAAATTTGC
AAGGATAGCAAATCTTCTCACAATTCCCTTAGTTCCCGTTCGTCCGATGTGATAAACGACAAATA
CGTGGACTTCAAGAACTGTACATTTCTTTGCAAACATGGAAATGATGTTAACGTGACATTGAATT
TGCCAGAAGACACGCCTTGTGGACCGAATGGACAGACATGCGCTGAAAAGAATAAATGCGTTGGC
CACATTCCCGGATGTTAG
Salp25AAAK97825
SEQ ID NO: 5
MKLVLSLAVLLCGVLVRTYGKAPQGGSYGKTSSTTPRGILYGSTTTVPPEEDPSKLREQNATRVV
QMNATQWVKWRTYNVTDPLRGNYPLQCENFKVMEKRTPFNYSLQYKYRSGYSWVTRTETLVLGIL
GLQPLPPNDMFFQRTPIGIPTNNYVLYSNYVNCTILRIPMPGQGQNHCDLWMANMTVSQEPPKIC
SDKFYEYCNTTQKFTVYFPNCTEISSF
SEQ ID NO: 6
>AF209922.1 Ixodes scapularis 25 kDa salivary gland protein A
(Salp25A) mRNA, complete cds
ATGAAGCTTGTTTTAAGCTTGGCCGTTCTTTTGTGTGGAGTTCTTGTGAGAACCTATGGTAAAGC
ACCTCAAGGGGGAAGCTACGGCAAAACAAGTAGTACTACACCTCGAGGGATATTATATGGCAGTA
CGACTACAGTTCCCCCTGAAGAAGACCCGTCAAAACTACGAGAACAAAATGCAACCAGAGTGGTC
CAAATGAACGCTACACAATGGGTCAAGTGGCGAACGTATAATGTGACAGATCCTTTACGTGGTAA
CTACCCGCTGCAGTGTGAAAATTTCAAGGTTATGGAAAAGAGAACACCGTTCAACTACTCCTTAC
AGTACAAATATAGGAGCGGATATAGTTGGGTGACACGTACCGAAACCCTAGTTTTGGGGATTCTC
GGATTACAGCCTCTCCCACCAAATGACATGTTCTTTCAAAGGACTCCTATTGGCATACCAACAAA
CAATTACGTGTTGTATTCCAACTACGTGAACTGCACCATTCTTCGAATCCCAATGCCAGGACAAG
GACAGAACCATTGTGACCTGTGGATGGCGAATATGACTGTTTCTCAAGAACCACCCAAAATCTGC
AGCGACAAATTTTATGAATATTGCAACACTACACAAAAATTCACCGTTTACTTTCCAAATTGTAC
TGAAATAAGTAGTTTTTAG
Salp25B AAK97821
SEQ ID NO: 7
MKLVLSLAVLVCGVLVRTYGKTHQVGSYGKPSSTTPRGILYGSTTTVPPEEDPAKLREQNATRVV
QMNATQWVKWRTFNVTDPFRGNYPLQCENFKVMEKRTPFNYSLQYKYRSGYSWVTLTETLVLGIL
GEQRLPPNDMFFQRTPIGIPTNNYVLYSNYVNCTILRIPMPGQGRNHCDLWMANMTVSQEPPKIC
SDKFYEYCNTTQKFTVYFPNCTEISGF
SEQ ID NO: 8
>AF209918.1 Ixodes scapularis 25 kDa salivary gland protein B
(Salp25B) mRNA, complete cds
ATGAAGCTTGTTTTAAGCTTGGCCGTTCTTGTGTGTGGAGTTCTTGTGAGAACCTATGGCAAAAC
ACATCAAGTGGGAAGCTACGGCAAACCAAGTAGTACTACACCTCGAGGGATATTATATGGCAGTA
CGACTACAGTTCCCCCTGAAGAAGACCCGGCAAAACTACGAGAACAAAATGCAACCAGAGTGGTC
CAAATGAACGCTACACAATGGGTCAAGTGGCGCACGTTTAATGTGACAGATCCTTTCCGTGGTAA
CTACCCGCTGCAGTGTGAAAACTTCAAGGTTATGGAAAAGAGAACACCGTTCAACTACTCCTTAC
AGTACAAATATAGGAGCGGATATAGTTGGGTGACACTTACCGAAACCCTAGTTTTGGGGATTCTC
GGAGAACAGCGTCTCCCACCAAATGACATGTTCTTTCAGAGGACTCCTATTGGCATACCAACAAA
CAATTACGTGTTGTATTCCAACTACGTGAACTGCACCATTCTTCGAATCCCAATGCCAGGACAAG
GACGGAACCATTGTGACCTGTGGATGGCGAATATGACTGTTTCTCAAGAACCACCCAAAATCTGC
AGCGACAAATTTTATGAATATTGCAACACTACACAAAAATTCACCGTTTACTTTCCAAATTGTAC
TGAAATAAGTGGCTTTTAG
Salp25C AAK97816
SEQ ID NO: 9
MKLVLSLAVFVCGVFWGTSGSTSTTTRRVGTYGSTGTTTRPGTRGARMIVTTAPPEEDPSKYKEQ
NATRVVEMNATQWVKWRTYDVTDFSGNPVQCENFRVMEKRTPTNYSFQYRYKSKNSWETIDETLI
LKDIGEHGFPNVMNFQRTPIGIATDNLVLYSNYVNCTVLRIPFTNQGERHCDLWMANLTLSQETP
DDCLNKFFEYCNTTQIYRVYYPSCTN
SEQ ID NO: 10
>AF209913.1 Ixodes scapularis histamine binding protein (HBP)
mRNA, complete cds
ATGAAGCTTGTTTTAAGCTTGGCCGTTTTTGTGTGTGGAGTGTTTTGGGGAACCAGTGGCTCCAC
AAGTACTACTACACGTCGAGTGGGAACGTATGGCTCTACAGGAACGACTACTCGTCCAGGGACAC
GCGGTGCCAGAATGATTGTGACTACAGCTCCCCCTGAAGAAGACCCTTCGAAATACAAAGAACAA
AATGCCACCAGAGTGGTCGAAATGAACGCTACGCAATGGGTCAAGTGGCGCACGTATGATGTGAC
AGATTTCAGCGGTAACCCAGTGCAGTGTGAAAATTTTAGGGTTATGGAAAAGAGAACTCCAACAA
ACTACTCATTCCAGTACAGATATAAAAGTAAAAATAGTTGGGAGACAATCGACGAAACCCTAATC
TTGAAGGATATAGGTGAGCACGGTTTCCCGAACGTCATGAACTTTCAGAGGACTCCTATTGGCAT
AGCAACGGACAATCTCGTGTTGTATTCCAACTACGTGAACTGCACCGTTCTTCGAATCCCATTCA
CAAATCAAGGAGAGAGGCATTGCGACCTGTGGATGGCCAATCTGACTCTTTCCCAAGAAACCCCC
GACGACTGCCTGAATAAGTTTTTTGAATATTGCAACACGACGCAAATTTACCGCGTGTACTACCC
TAGTTGTACAAACTAG
Salp25D AAK97814
SEQ ID NO: 11
MGPLNLGDPFPNFTCDTTEGKIDFHEWLGNSWGILFSHPADYTPVCTSELARAAQLHHVFQKKGV
KLIALSCDSVESHRGWIKDINAFGELPDGPFPYPIIADEKRDIAVKLGMLDPVEKDKEGLPLTCR
AVFIIGPDKKMKLSMLYPATTGRNFDEVLRATDSLLVTETRKVATPAGWQKGTPCMVLPSVTEEE
ILKLFPTGIKQYEVPSGKNYLRTTMD
SEQ ID NO: 12
>AF209911.1 Ixodes scapularis glutathione peroxidase (gph) mRNA,
complete cds
ATGGGTCCCCTGAACCTCGGCGATCCTTTCCCCAACTTCACCTGCGACACGACCGAGGGCAAGAT
CGACTTCCACGAATGGCTCGGCAACTCGTGGGGCATCCTGTTCTCGCACCCCGCCGACTACACCC
CGGTGTGCACAAGTGAGCTCGCCAGGGCAGCACAGCTGCACCACGTCTTTCAGAAGAAGGGTGTC
AAGCTCATCGCTCTCTCCTGTGACAGTGTGGAGAGCCACCGTGGTTGGATCAAGGACATCAACGC
CTTTGGGGAGCTGCCGGACGGGCCCTTCCCGTACCCCATCATCGCCGATGAGAAGCGCGACATTG
CCGTCAAGCTGGGAATGTTGGACCCCGTGGAGAAGGACAAGGAAGGGCTGCCTCTCACCTGCAGG
GCGGTGTTCATCATTGGTCCCGACAAGAAAATGAAGCTCTCCATGCTGTATCCCGCCACGACTGG
AAGGAACTTTGACGAGGTCCTGCGTGCCACCGATTCCCTGCTGGTGACGGAGACCAGGAAGGTGG
CGACGCCTGCTGGTTGGCAGAAGGGCACCCCGTGCATGGTCCTGCCTTCGGTGACCGAGGAAGAG
ATTCTCAAGCTGTTCCCGACAGGCATCAAGCAGTACGAAGTTCCGTCTGGCAAGAACTACCTCCG
AACAACCATGGACTGA
Salp14 AAK97824
SEQ ID NO: 13
MGLTGTMLVLVSLAFFGSAAAHNCQNGTRPASEQDREGCDYYCWNAETKSWDQFFFGNGEKCFYN
SGDHGTCQNGECHLTNNSGGPNETDDYTPAPTEKPKQKKKKTKKTKKPKRKSKKDQEKNL
SEQ ID NO: 14
>AF209921.1 Ixodes scapularis 14 kDa salivary gland protein
(Salp14) mRNA, complete cds
ATGGGGTTGACCGGAACCATGCTGGTGTTGGTATCTCTGGCCTTCTTCGGGAGCGCTGCAGCCCA
CAATTGCCAGAACGGAACGAGACCCGCATCGGAGCAAGATAGAGAAGGCTGCGACTATTACTGCT
GGAACGCTGAGACCAAATCATGGGACCAATTTTTCTTTGGAAACGGAGAAAAATGCTTTTACAAC
AGTGGTGATCACGGAACATGTCAAAACGGAGAATGTCATTTGACAAATAATTCAGGTGGGCCCAA
CGAAACTGATGACTATACTCCTGCGCCCACTGAGAAGCCGAAGCAAAAAAAGAAGAAAACTAAGA
AGACTAAGAAACCTAAGCGCAAGTCAAAGAAAGATCAGGAGAAAAACTTATGA
TSLPI AEE89466
SEQ ID NO: 15
MGLTETTLVLVSLAFFASAVAHNCQNGTRPASEEKREGCDYYCWNAETNSWDKFFFGNGERCFYN
DGGEGLCQNGECHLTTDSSVPNDSDV
SEQ ID NO: 16
ATGGGGTTGACTGAGACCACGCTGGTGTTGGTGTCTCTGGCCTTCTTCGCAAGCGCTGTGGCCCA
CAATTGCCAAAACGGAACAAGACCCGCCTCGGAGGAAAAAAGAGAAGGCTGCGACTATTACTGCT
GGAACGCTGAGACCAATTCATGGGACAAATTTTTCTTCGGGAACGGAGAACGATGCTTTTACAAC
GATGGTGGTGAGGGACTATGTCAAAATGGAGAATGCCATTTGACAACCGATTCAAGTGTGCCCAA
TGACAGTGATGTCTAA
SEQ ID NO: 17
>HQ605983.1 Ixodes scapularis salivary protein antigen P8 mRNA,
complete cds
ATGGGGTTGACTGAGACCACGCTGGTGTTGGTGTCTCTGGCCTTCTTCGCAAGCGCTGTGGCCCA
CAATTGCCAAAACGGAACAAGACCCGCCTCGGAGGAAAAAAGAGAAGGCTGCGACTATTACTGCT
GGAACGCTGAGACCAATTCATGGGACAAATTTTTCTTCGGGAACGGAGAACGATGCTTTTACAAC
GATGGTGGTGAGGGACTATGTCAAAATGGAGAATGCCATTTGACAACCGATTCAAGTGTGCCCAA
TGACAGTGATGTCTAAACTCCTGCTCCCACTGAAAAGCCGAAGCAAAAAATGGAGAAACCGAAGA
CGAGTAAGAAGTCTAAGGGCAACTCAACGAAAGATTAGGAAGGAAACTTTTCATTAAAAGTCTCG
AAACTAAACCAGGCTAAATAAAGCTAAGGCACTGACTCTTTAAAAAAAAAAAAAAAAAAAAAAAA
AAAAAAA
Salp26A AAK97822
SEQ ID NO: 18
MRAVIGALMYILFAFCFASNEVFEKYPKNIGSDEKLITVAFVLAGFDNADVNMKSDVGVWLQDSY
DEAADKLSKELQVQLKFEITDIQHAPPALTDEIAYRTVGGQMHGPTILDAVKNTYKKHLNPDIIS
VITKDKFYDDKFSNKIGFSGFTTLCEDMVPILLTENSDIEDDVDATATLLSKLIESSLDPVKWKE
FGPRKDYFDGCNIRPKPKGDTYNEYYVFPIDKAPFYDF
SEQ ID NO: 19
>AF209919.1 Ixodes scapularis 26 kDa salivary gland protein A
(Salp26A) mRNA, complete cds
ATGAGGGCTGTCATTGGAGCGCTTATGTATATATTATTTGCCTTCTGCTTTGCAAGCAATGAAGT
GTTTGAGAAATATCCGAAAAACATAGGTTCTGATGAGAAACTTATAACGGTTGCCTTTGTTCTGG
CCGGATTTGACAATGCGGATGTGAATATGAAATCTGATGTTGGTGTTTGGCTTCAGGATTCTTAT
GATGAGGCTGCAGATAAGTTAAGCAAAGAATTACAAGTTCAACTTAAATTTGAGATTACCGATAT
CCAACACGCGCCTCCTGCCCTTACGGACGAAATTGCGTACCGGACGGTTGGTGGGCAAATGCACG
GCCCTACAATTCTGGATGCCGTGAAAAATACTTATAAAAAACACCTCAACCCCGATATCATATCT
GTTATTACGAAAGACAAATTCTACGATGACAAGTTCAGCAATAAAATAGGTTTTTCAGGGTTCAC
CACACTCTGCGAAGATATGGTGCCTATTCTTTTGACGTTCAATTCGGATATTGAGGACGACGTTG
ATGCGACCGCTACCCTGTTGTCCAAGCTAATTGAGAGCAGTTTGGACCCAGTTAAATGGAAAGAA
TTTGGACCGAGAAAAGACTACTTCGATGGATGTAACATAAGACCCAAACCTAAAGGAGATACTTA
TAATGAATATTACGTTTTTCCTATTGACAAAGCTCCTTTTTACGATTTTTAA
P11 AAY66648
SEQ ID NO: 20
MKKTLCAAFFFTVVVAATLAYSDTFEGQVPVFPPGVFTKPPGKVGEPCMTGADCRNGTCCRKEKG
GSKTCRRLRKTGERCSDAPIKGEIYEGHCPCKLGLQCFGERVHRCYAVPQASVDPTYTDGREHVL
SEQ ID NO: 21
>DQ066011.1 Ixodes scapularis isolate is-all-704 putative
secreted protein mRNA, complete cds
ATGAAAAAGACACTGTGTGCAGCGTTTTTTTTCACCGTTGTCGTGGCAGCAACGCTGGCATACAG
CGATACATTTGAGGGACAGGTTCCAGTATTTCCCCCTGGCGTTTTTACTAAACCTCCTGGAAAAG
TGGGCGAGCCTTGCATGACCGGCGCCGATTGCCGGAATGGCACTTGCTGTAGAAAAGAAAAAGGC
GGTTCAAAGACATGCCGGCGTCTACGAAAGACTGGCGAACGCTGTTCGGACGCTCCAATTAAGGG
TGAAATCTACGAAGGTCACTGTCCTTGCAAGCTAGGTCTGCAATGTTTCGGGGAACGGGTCCACA
GATGTTACGCAGTTCCTCAAGCTAGTGTGGATCCTACATACACTGACGGCCGCGAGCACGTGTTG
TAA
Salp16A AAK97818
SEQ ID NO: 22
MVEDGMKNTGILSFIMRGTRLIMATFVLFAICKPIVAGVVIRGTENLAPKCEQKIKHLCENPTHG
ELTEVTVTARQCQATCTYRPDPTRDTVEVDGIPIKNRHYETVTLPDKMPCGFGARCNKKGECSCN
SCNENINHNEPRQT
SEQ ID NO: 23
>AF209915.1 Ixodes scapularis 16 kDa salivary gland protein A
(Salp16A) mRNA, complete cds
ATGGTGGAGGACGGTATGAAGAACACCGGTATTTTATCCTTTATTATGAGAGGAACAAGACTGAT
CATGGCAACGTTTGTGCTGTTTGCAATTTGTAAACCAATTGTCGCCGGCGTTGTAATCAGAGGTA
CGGAAAACCTTGCGCCGAAATGTGAACAAAAGATTAAGCATCTTTGTGAGAACCCTACACATGGT
GAGCTAACGGAGGTCACTGTGACGGCTCGCCAGTGCCAAGCCACATGCACTTATCGACCCGACCC
TACTAGAGATACGGTGGAAGTTGATGGTATACCTATTAAGAACCGTCATTATGAAACAGTCACCC
TGCCAGATAAAATGCCGTGCGGTTTTGGGGCGAGATGCAACAAGAAAGGGGAGTGCAGTTGCAAC
TCTTGCAATGAAAATATAAACCACAACGAGCCAAGGCAAACTTAA
Salp17 AAK97819
SEQ ID NO: 24
MKLAISLAVFVCGVLVGAYGKMTTTTPRGRLRGSMTTVPPEYDLSKQEEQNATRVVQMNATQWVK
WRTYNVTDLVSGYPLQCENFKVMEKRTPTNYSLQYNYRSGYSWETINEDLILGFLNLRPLPPNDM
FFARTPIGDQPTIWCCIPTT
SEQ ID NO: 25
>AF209916.1 Ixodes scapularis 17 kDa salivary gland protein
(Salp17) mRNA, complete cds
ATGAAGCTTGCTATAAGCTTGGCCGTTTTTGTGTGTGGAGTTCTAGTGGGAGCCTATGGCAAAAT
GACTACTACCACACCACGAGGGAGATTGCGTGGCAGTATGACTACAGTTCCCCCTGAATACGACC
TATCAAAACAAGAAGAACAAAATGCAACCAGAGTGGTCCAAATGAACGCTACACAATGGGTCAAG
TGGCGCACGTATAATGTGACAGATCTTGTCAGCGGTTACCCTCTGCAGTGCGAAAATTTCAAGGT
TATGGAAAAGAGAACACCGACAAACTACTCTTTACAATACAATTATAGGAGCGGATATAGTTGGG
AGACAATCAACGAAGACCTAATTTTGGGATTTCTCAATCTACGGCCCCTTCCCCCAAATGACATG
TTTTTTGCGAGGACTCCTATAGGGGACCAACCCACAATTTGGTGTTGTATTCCGACTACATGA
TIX5 AEE89467
SEQ ID NO: 26
MLTVSLLTLSLAAYASAVATVTDANNFMDAVLHTRIPALITSEPILFPFATIPPFNFTVAGTNIL
TNRELQVNVSRGEIRGFSTEVKRVGDCMPPVLREGKTSIRCTLNFTGINATFDTHTRGDNIVASD
KNIWVRASVIDTTGQFEAVAERGKQGNVHTFLVDKIHVKVKNDKALSLNDKRKKKFRQHFEDKVL
TVLPQIFYGAYLHLLGAAVSSVPFPHV
SEQ ID NO: 27
ATGCTGACTGTAAGCCTTCTCACGCTGTCTCTAGCGGCCTATGCCTCCGCTGTGGCCACGGTCAC
CGACGCCAACAACTTCATGGACGCGGTGCTCCACACCAGAATCCCGGCACTGATCACGTCGGAGC
CCATTCTTTTCCCATTCGCGACCATTCCTCCTTTCAACTTCACGGTGGCCGGAACCAACATCCTG
ACGAACCGGGAACTACAGGTGAACGTGTCCCGGGGTGAAATCCGGGGCTTCAGCACTGAGGTGAA
GCGCGTGGGAGACTGCATGCCGCCTGTGCTCAGGGAAGGCAAGACGTCCATCAGGTGTACCCTCA
ACTTCACCGGGATCAACGCTACTTTCGACACGCACACGCGGGGTGACAACATTGTGGCCTCGGAT
AAAAACATCTGGGTCCGCGCTAGCGTCATCGACACTACGGGACAGTTCGAAGCCGTCGCCGAGCG
CGGCAAGCAGGGCAATGTTCACACCTTCCTGGTAGACAAGATTCACGTGAAGGTCAAGAACGACA
AGGCACTCAGCCTGAACGACAAACGCAAGAAGAAGTTCAGGCAGCATTTTGAGGACAAGGTTCTG
ACCGTGTTGCCGCAGATATTCTACGGCGCCTATCTCCATCTGCTCGGTGCAGCTGTGAGCTCTGT
TCCTTTCCCTCATGTTTGA
SEQ ID NO: 28
>HQ605984.1 Ixodes scapularis salivary protein antigen P23 mRNA,
complete cds
ATGCTGACTGTAAGCCTTCTCACGCTGTCTCTAGCGGCCTATGCCTCCGCTGTGGCCACGGTCAC
CGACGCCAACAACTTCATGGACGCGGTGCTCCACACCAGAATCCCGGCACTGATCACGTCGGAGC
CCATTCTTTTCCCATTCGCGACCATTCCTCCTTTCAACTTCACGGTGGCCGGAACCAACATCCTG
ACGAACCGGGAACTACAGGTGAACGTGTCCCGGGGTGAAATCCGGGGCTTCAGCACTGAGGTGAA
GCGCGTGGGAGACTGCATGCCGCCTGTGCTCAGGGAAGGCAAGACGTCCATCAGGTGTACCCTCA
ACTTCACCGGGATCAACGCTACTTTCGACACGCACACGCGGGGTGACAACATTGTGGCCTCGGAT
AAAAACATCTGGGTCCGCGCTAGCGTCATCGACACTACGGGACAGTTCGAAGCCGTCGCCGAGCG
CGGCAAGCAGGGCAATGTTCACACCTTCCTGGTAGACAAGATTCACGTGAAGGTCAAGAACGACA
AGGCACTCAGCCTGAACGACAAACGCAAGAAGAAGTTCAGGCAGCATTTTGAGGACAAGGTTCTG
ACCGTGTTGCCGCAGATATTCTACGGCGCCTATCTCCATCTGCTCGGTGCAGCTGTGAGCTCTGT
TCCTTTCCCTCATGTTTGATCAGAAAACCACACATTTATTTCTGTTGGAGCTTGAGGTCACGCTG
CTATGATTAAATTTGGCCTAAAATCAAATAGGGTTTCACTTTGAATCTCGACGTCATTCACAATC
GGGGAAAGTGATGGTCCCGAACTTTAACTTAGGTTTTAAGACTAGCTTCATTCAATGTATTTGCC
TCCTGAAATATGTTATGTGCCACTTGAAGTGTATGCGTGCAAAGATCGCTAGATATTATAGTACT
TTGTTTTGCAGTAGGATTCTGTAAAAATCAATATACAGTGAAAACAAGCGCTTTAAGTTCTTTAC
GTTGTATGCGATGCAAATATTTCCTGATATGAAATAAAACTGTCAAAATACATGAAAAAAAAAAA
AAAAAAAAAAAAAA
P32 ADO95260
SEQ ID NO: 29
MDAHRLMLCVWLWLVSSYVECLPRVRLSSRQQIEGETRITIDFYIDDSVSATEQQVKDYLHKVIF
TTTADLQSYFVVDDIHLPYWIKYIGNEPALKSALQSYKNPEFIHLDGAIDALTAYFINQNPPDII
CLVTNYTIHNGDNIRKAYGYSKDHTLCKSVVSMLLAYAPYTPGHAGRMLSEMIRASVNPQEMPNL
YHRASRQGSFETHMKEYLSTCNKGFKRPGPPEDDMPPRPPPEIPPGPPNVPPPPPPPDTTAEPPQ
VKPQPPLPPVPPAPPVPPPSQPDKPPNPVPEATTPAEPQPDYC
SEQ ID NO: 30
ATGGACGCACATCGCTTAATGTTATGCGTATGGCTATGGTTGGTATCTTCTTATGTAGAATGTTT
GCCCAGAGTTCGATTAAGTTCAAGACAGCAAATTGAAGGAGAGACACGTATTACAATAGATTTTT
ATATAGATGATTCCGTCAGTGCGACTGAACAACAGGTGAAGGATTATTTGCACAAAGTCATTTTT
ACGACAACGGCCGATCTCCAAAGTTATTTTGTCGTCGACGATATACATCTTCCGTACTGGATAAA
ATACATTGGGAACGAGCCTGCACTGAAATCTGCGCTGCAGAGTTATAAAAACCCGGAATTTATTC
ACCTGGATGGAGCAATTGATGCATTAACAGCCTATTTTATAAACCAGAACCCTCCTGACATCATT
TGCCTGGTAACAAACTATACAATACACAATGGAGATAATATACGAAAGGCTTATGGATATTCTAA
GGATCACACTCTGTGCAAGAGTGTTGTATCTATGTTGTTAGCATACGCCCCCTACACACCGGGAC
ACGCCGGCAGAATGTTGTCCGAAATGATCAGAGCCAGCGTAAATCCACAGGAAATGCCAAATCTG
TATCATCGAGCATCACGACAAGGCAGTTTCGAAACCCATATGAAGGAATACTTAAGCACATGCAA
CAAAGGGTTCAAACGTCCCGGCCCTCCCGAAGATGACATGCCTCCACGACCCCCACCAGAAATTC
CTCCCGGACCCCCCAATGTCCCTCCACCACCACCACCGCCGGACACAACTGCCGAACCGCCACAA
GTTAAACCACAGCCACCATTACCGCCTGTGCCACCAGCGCCACCAGTGCCACCACCATCCCAACC
AGACAAGCCTCCTAATCCAGTACCAGAGGCAACAACACCAGCGGAGCCTCAACCTGATTACTGCT
AA
SEQ ID NO: 31
>HM802761.1 Ixodes scapularis salivary protein antigen P32 mRNA,
complete cds
ATGGACGCACATCGCTTAATGTTATGCGTATGGCTATGGTTGGTATCTTCTTATGTAGAATGTTT
GCCCAGAGTTCGATTAAGTTCAAGACAGCAAATTGAAGGAGAGACACGTATTACAATAGATTTTT
ATATAGATGATTCCGTCAGTGCGACTGAACAACAGGTGAAGGATTATTTGCACAAAGTCATTTTT
ACGACAACGGCCGATCTCCAAAGTTATTTTGTCGTCGACGATATACATCTTCCGTACTGGATAAA
ATACATTGGGAACGAGCCTGCACTGAAATCTGCGCTGCAGAGTTATAAAAACCCGGAATTTATTC
ACCTGGATGGAGCAATTGATGCATTAACAGCCTATTTTATAAACCAGAACCCTCCTGACATCATT
TGCCTGGTAACAAACTATACAATACACAATGGAGATAATATACGAAAGGCTTATGGATATTCTAA
GGATCACACTCTGTGCAAGAGTGTTGTATCTATGTTGTTAGCATACGCCCCCTACACACCGGGAC
ACGCCGGCAGAATGTTGTCCGAAATGATCAGAGCCAGCGTAAATCCACAGGAAATGCCAAATCTG
TATCATCGAGCATCACGACAAGGCAGTTTCGAAACCCATATGAAGGAATACTTAAGCACATGCAA
CAAAGGGTTCAAACGTCCCGGCCCTCCCGAAGATGACATGCCTCCACGACCCCCACCAGAAATTC
CTCCCGGACCCCCCAATGTCCCTCCACCACCACCACCGCCGGACACAACTGCCGAACCGCCACAA
GTTAAACCACAGCCACCATTACCGCCTGTGCCACCAGCGCCACCAGTGCCACCACCATCCCAACC
AGACAAGCCTCCTAATCCAGTACCAGAGGCAACAACACCAGCGGAGCCTCAACCTGATTACTGCT
AAAGTGGATCGCACACTAGAAGGGGTCATCAGTGGCACGAGTATTTGGTAGCACGTGGATTGCAT
GTCCGGGATTGAGTTTTGTTTTTAATAAATATATTCTGGCTTAACAAAAAAAAAAAAAAAAAAAA
AAAAAAAAAAA
Salp12 XP_002433874
SEQ ID NO: 32
MNKVLVVLYFVLLVVAAWPDDDNIQQVATGPRGQDLADGTICSTGDECASKCCLKHFTVTGSDGP
AQCHVKSDLGESCSDDQVKGGASVNHCPCSRGSCENNICTLENTDEDKDD
SEQ ID NO: 33
>XM 002433829.1 Ixodes scapularis Is6, putative, mRNA
ATGAATAAGGTTCTCGTAGTCCTCTACTTTGTTCTCCTGGTGGTGGCGGCGTGGCCGGATGACGA
TAACATACAGCAAGTGGCCACAGGACCACGAGGACAGGACCTGGCAGATGGTACGATCTGCTCCA
CGGGGGATGAGTGCGCCAGCAAGTGCTGCCTGAAGCACTTTACCGTTACGGGGAGCGATGGTCCT
GCCCAGTGTCACGTTAAGAGTGATCTCGGGGAGAGCTGCTCTGACGACCAGGTGAAAGGTGGTGC
ATCCGTGAACCACTGTCCTTGCTCCAGAGGGTCTTGCGAAAACAACATCTGCACTCTCGAAAACA
CCGACGAAGATAAGGACGACTGA
SG27 XP_002405832
SEQ ID NO: 34
MLSLKFVTLLCALVAVHCEDYEKDEHVLVLKQTNFDKAVTEHKHVLVKFYAPWCGHCKAMAPEYV
KAAKQLVDESSDIKLAKVDATIETQLAETYEVRGYPTLKFFRDGKPYDYKGGRTADEMVRWLKKR
TGPAAEDLKSADAARTFVDASKVSVVGFFKDQASSEAMQFLEAAEAIDAHPFAITSDDAVYKELG
ASKDGVILFKKFDEGRSLMEGAVTSESVQSFVKTNSLPLVVEFTHESAQTVFGGQIKLHNLLFVS
KKSPGFEDILKDYREAAKDFRHKVLFVTIDVDDEDHERILEFFGLKKDQVPVMRFVKLEGEMTKY
KPEKDDLTPENVRSFVQDVLDGKLKQSLLSQDLPEDWDRHAVKVLVNKNFDEVVFDKEKDVLVEF
YAPWCGHCKQLAPIYDELAEKYKEKRPDLVIAKFDGTANELEHTKMQGFPTIRLYKKGTNEAVEY
NGERTLEGLSKFIDTDGEYGKAAPEEVTEEEEEEEDKEDKKRDEL
SEQ ID NO: 35
ATGTTGTCGCTAAAGTTCGTCACGTTACTCTGCGCTCTCGTCGCCGTCCACTGTGAAGACTACGA
GAAGGACGAGCACGTCCTGGTCCTGAAACAGACCAACTTCGACAAAGCAGTTACCGAACACAAGC
ATGTGCTGGTCAAGTTCTATGCCCCCTGGTGCGGCCACTGCAAGGCGATGGCGCCAGAGTATGTC
AAGGCCGCCAAGCAGCTAGTGGACGAAAGCTCGGACATCAAGCTTGCCAAGGTCGACGCCACAAT
AGAGACTCAGCTCGCCGAGACCTACGAGGTGCGAGGCTACCCGACCCTCAAGTTCTTCCGCGATG
GCAAGCCCTACGACTACAAGGGTGGCCGTACTGCCGACGAGATGGTGCGTTGGCTCAAGAAGAGG
ACTGGACCCGCAGCTGAGGACCTCAAGTCCGCCGATGCTGCACGTACCTTCGTGGATGCCTCCAA
AGTGTCTGTGGTTGGCTTTTTCAAGGACCAGGCAAGCAGCGAAGCCATGCAGTTCCTAGAGGCTG
CAGAGGCCATCGACGCCCACCCCTTTGCCATCACGTCCGACGACGCGGTCTACAAGGAGCTGGGA
GCCTCCAAGGATGGAGTCATACTCTTCAAGAAGTTTGACGAGGGCCGCAGCCTGATGGAGGGTGC
TGTGACGTCCGAGAGTGTCCAGTCCTTCGTCAAGACCAACAGCCTGCCCCTGGTGGTAGAGTTCA
CCCACGAGAGTGCCCAGACGGTCTTCGGAGGACAGATCAAACTGCACAACCTGCTCTTTGTGAGC
AAGAAGAGCCCCGGCTTCGAGGACATTCTCAAGGACTACCGGGAGGCGGCCAAGGACTTCCGCCA
CAAGGTGCTCTTCGTCACCATCGACGTGGACGACGAGGACCACGAGCGCATCCTCGAGTTCTTTG
GCCTCAAGAAAGACCAGGTGCCCGTAATGCGCTTCGTCAAGCTGGAGGGCGAGATGACCAAGTAC
AAGCCGGAGAAGGACGACCTCACGCCCGAGAATGTGCGCTCATTTGTGCAAGACGTGCTGGACGG
CAAGCTGAAGCAGAGCCTTTTGTCTCAAGACCTGCCGGAGGACTGGGACAGGCACGCCGTCAAGG
TCCTCGTCAACAAGAACTTCGACGAGGTGGTCTTTGACAAGGAGAAGGACGTCCTGGTCGAGTTC
TACGCTCCCTGGTGCGGACACTGCAAGCAGCTGGCCCCGATCTACGACGAGCTGGCTGAGAAGTA
CAAGGAGAAGCGGCCGGACCTGGTGATTGCCAAGTTCGACGGCACGGCCAACGAGCTGGAGCACA
CCAAGATGCAGGGCTTCCCCACCATCCGGTTGTACAAGAAGGGCACCAACGAGGCCGTGGAGTAC
AACGGGGAGCGCACCCTCGAGGGGCTGTCCAAGTTCATCGACACGGACGGGGAGTACGGCAAGGC
GGCCCCCGAGGAGGTCACGGAAGAGGAGGAGGAAGAGGAAGACAAGGAGGACAAGAAGCGTGACG
AGCTCTAA
SEQ ID NO: 36
>XM_002405788.4 PREDICTED: Ixodes scapularis protein disulfide-
isomerase (LOC8037869), mRNA
GAGTATGGCGACCCCTCATAAATTGAGCATGCGCAAACGCGCCGCGCTAGCCTCTGCCACGTTTG
TAACACTCGAAGGCTGGTCACCGGTGCCGTAGGAGGGGAGCAAGTTAAAACAAACTTTCTTTTAT
ACACGCGACATAGGAACCATGTTGTCGCTAAAGTTCGTCACGTTACTCTGCGCTCTCGTCGCCGT
CCACTGTGAAGACTACGAGAAGGACGAGCACGTCCTGGTCCTGAAACAGACCAACTTCGACAAAG
CAGTTACCGAACACAAGCATGTGCTGGTCAAGTTCTATGCCCCCTGGTGCGGCCACTGCAAGGCG
ATGGCGCCAGAGTATGTCAAGGCCGCCAAGCAGCTAGTGGACGAAAGCTCGGACATCAAGCTTGC
CAAGGTCGACGCCACAATAGAGACTCAGCTCGCCGAGACCTACGAGGTGCGAGGCTACCCGACCC
TCAAGTTCTTCCGCGATGGCAAGCCCTACGACTACAAGGGTGGCCGTACTGCCGACGAGATGGTG
CGTTGGCTCAAGAAGAGGACTGGACCCGCAGCTGAGGACCTCAAGTCCGCCGATGCTGCACGTAC
CTTCGTGGATGCCTCCAAAGTGTCTGTGGTTGGCTTTTTCAAGGACCAGGCAAGCAGCGAAGCCA
TGCAGTTCCTAGAGGCTGCAGAGGCCATCGACGCCCACCCCTTTGCCATCACGTCCGACGACGCG
GTCTACAAGGAGCTGGGAGCCTCCAAGGATGGAGTCATACTCTTCAAGAAGTTTGACGAGGGCCG
CAGCCTGATGGAGGGTGCTGTGACGTCCGAGAGTGTCCAGTCCTTCGTCAAGACCAACAGCCTGC
CCCTGGTGGTAGAGTTCACCCACGAGAGTGCCCAGACGGTCTTCGGAGGACAGATCAAACTGCAC
AACCTGCTCTTTGTGAGCAAGAAGAGCCCCGGCTTCGAGGACATTCTCAAGGACTACCGGGAGGC
GGCCAAGGACTTCCGCCACAAGGTGCTCTTCGTCACCATCGACGTGGACGACGAGGACCACGAGC
GCATCCTCGAGTTCTTTGGCCTCAAGAAAGACCAGGTGCCCGTAATGCGCTTCGTCAAGCTGGAG
GGCGAGATGACCAAGTACAAGCCGGAGAAGGACGACCTCACGCCCGAGAATGTGCGCTCATTTGT
GCAAGACGTGCTGGACGGCAAGCTGAAGCAGAGCCTTTTGTCTCAAGACCTGCCGGAGGACTGGG
ACAGGCACGCCGTCAAGGTCCTCGTCAACAAGAACTTCGACGAGGTGGTCTTTGACAAGGAGAAG
GACGTCCTGGTCGAGTTCTACGCTCCCTGGTGCGGACACTGCAAGCAGCTGGCCCCGATCTACGA
CGAGCTGGCTGAGAAGTACAAGGAGAAGCGGCCGGACCTGGTGATTGCCAAGTTCGACGGCACGG
CCAACGAGCTGGAGCACACCAAGATGCAGGGCTTCCCCACCATCCGGTTGTACAAGAAGGGCACC
AACGAGGCCGTGGAGTACAACGGGGAGCGCACCCTCGAGGGGCTGTCCAAGTTCATCGACACGGA
CGGGGAGTACGGCAAGGCGGCCCCCGAGGAGGTCACGGAAGAGGAGGAGGAAGAGGAAGACAAGG
AGGACAAGAAGCGTGACGAGCTCTAAGCAACTCCCCTGTGACCCATTTCTCCCACCGCTCATTTT
CCCCCCTCCCCCCGCTGCCCTCTCATTGGTCACTCACCCCCGTCCCTGTTGTTGTTCGCATGTGG
TCCCCCTCTTGCGTTTGATCCCCTTTTCCCGATCTCTTCCAAGGCCTCACATGAGGGTGCAAAGA
AGCCCCCGTTTAAAGGAGGGTGACCGGAAGTACCACGCACACATTTGGGGGGTGCGGTTTCGGTC
GTAGTCTGCCGCCACCTCCCTGTGGTCCCCCCCGGCCCTTGTGAGGGGGGGGGAGGGCCCTTCTG
GACTTCCGTGCGCCGAACCCCCTTTGCTTGTGGGGTGGACGGTGCTGCATTTGTGTGCGCGAGTG
TGTGCTGGGGGGTGCCGTCTGGGCACCTTTCGATTAACAAAGAAAAGGGCTCTGCGGAGCTTCAT
TAAACTGTACAAGCTGTCACGTGGAA
IsPDIA3 002406442
SEQ ID NO: 37
MLTSAGNQGQRTLSVTSQLTKAPQTNQRPTAQLARRRSAFSHVVFPTLKKPALGTAFFFRREVRV
AVRSAAMRWFPLLAVLIPAALASDVLDYSADFDTKIQDHDAALVEFFAPWCGHCKRLAPEYEKAA
TELKTNDPPVPLIKVDCTSDGGKDTCSKHGVSGYPTLKIFRGGEFSADYNGPREAGGIVKYMKAQ
VGPSSKELLSVAEVEKYLSKDDVVIFGFFESKDASLHENFLKVADKQREAWTFGHSFDKDVLKKY
GYKNQVVLFRPKILKNKFEESFAVYSGSDDKAELETFIKENYHGLVGHRTQDNYNMFQAPLLVAY
YDVDYTKNAKGTNYWRNRILKVAQNYKGKLNFAVSNKDSFAAEMDDYGVTVKANKPAIAVRNSEN
EKFRMTNDFSVENLEKFLEEYLAGKVKAHLKSEPVPETNDGPVKVAVAENFKSLVTESTKDVLIE
FYAPWCGHCKKLAPTYEEVGKTLADEDVLVVKMDATANDVPSAFEVSGFPTLYWLPKNDKQNPRR
YEGGREHDDFIKWIAKHATDELKAYDRSGAKRAKEEL
SEQ ID NO: 38
ATGCTAACCTCTGCTGGCAACCAAGGACAGCGGACGCTGTCAGTGACGTCACAACTTACAAAGGC
GCCCCAAACCAATCAGCGCCCGACAGCACAACTCGCCCGGCGACGCTCTGCGTTTAGCCACGTTG
TTTTCCCAACTTTAAAGAAGCCGGCACTTGGCACTGCCTTTTTTTTCCGTCGTGAGGTTAGAGTC
GCGGTTCGGTCAGCAGCCATGAGGTGGTTTCCCTTGCTAGCGGTTTTAATCCCCGCTGCGCTTGC
CAGCGATGTCCTCGACTACTCGGCGGACTTCGACACGAAGATTCAGGACCACGACGCAGCACTAG
TCGAATTCTTCGCTCCATGGTGCGGACACTGCAAGAGGTTAGCGCCCGAGTACGAAAAAGCAGCT
ACCGAACTTAAAACCAACGACCCTCCAGTTCCATTGATAAAGGTGGACTGTACCTCCGACGGTGG
AAAAGATACGTGTTCAAAGCATGGAGTCAGCGGCTACCCCACCCTCAAGATCTTCAGAGGTGGAG
AGTTTTCGGCGGATTACAATGGCCCAAGGGAAGCCGGTGGCATCGTTAAATACATGAAGGCCCAG
GTGGGACCAAGCTCGAAGGAACTTCTGTCTGTTGCTGAAGTCGAGAAGTATCTGTCGAAGGACGA
CGTTGTCATTTTTGGTTTCTTCGAGAGCAAGGATGCCAGCCTGCACGAAAACTTCCTCAAGGTTG
CAGACAAGCAGCGGGAGGCCTGGACTTTCGGGCACTCGTTTGACAAGGATGTGCTCAAGAAGTAT
GGCTACAAAAACCAAGTTGTGCTGTTCCGACCCAAGATTCTCAAGAACAAGTTTGAGGAGAGCTT
TGCAGTTTACTCTGGCTCTGATGACAAGGCGGAACTGGAGACATTCATCAAGGAGAACTACCATG
GCCTGGTGGGCCACAGGACTCAGGACAACTATAACATGTTCCAGGCTCCGCTCCTGGTTGCCTAC
TACGACGTAGACTACACCAAGAACGCCAAGGGCACCAACTACTGGCGCAACCGCATCCTCAAGGT
GGCCCAGAACTACAAGGGCAAGCTCAATTTTGCTGTGAGCAACAAGGACAGCTTTGCTGCCGAGA
TGGACGACTACGGCGTGACGGTCAAGGCCAACAAGCCTGCCATCGCCGTGCGAAACAGCGAGAAC
GAGAAGTTCCGCATGACCAACGACTTCAGCGTCGAGAACCTGGAGAAGTTCCTGGAGGAGTACCT
TGCTGGCAAAGTGAAGGCCCACCTAAAGTCCGAGCCCGTGCCGGAGACCAACGACGGACCGGTCA
AGGTCGCGGTGGCAGAGAACTTCAAGTCCCTGGTCACGGAGAGCACCAAGGACGTCCTCATCGAG
TTCTATGCCCCCTGGTGTGGACACTGCAAGAAGTTGGCCCCTACCTACGAGGAAGTGGGCAAGAC
GCTTGCTGACGAGGATGTCCTAGTTGTGAAGATGGATGCCACAGCCAATGATGTGCCGTCTGCCT
TTGAGGTGTCTGGTTTCCCCACCCTGTACTGGCTGCCCAAGAACGACAAGCAGAACCCGCGGCGG
TACGAAGGAGGCCGGGAGCACGACGACTTCATCAAATGGATTGCCAAGCACGCCACGGACGAGCT
CAAGGCCTACGACCGCTCCGGAGCCAAGAGGGCCAAGGAGGAGTTATGA
SEQ ID NO: 39
>XM_002406398.5 PREDICTED: Ixodes scapularis protein disulfide-
isomerase A3 (LOC8024633), mRNA
AGGAAAAACTCGCGAGCGCCGAGCACTGCACGCGATGCTAACCTCTGCTGGCAACCAAGGACAGC
GGACGCTGTCAGTGACGTCACAACTTACAAAGGCGCCCCAAACCAATCAGCGCCCGACAGCACAA
CTCGCCCGGCGACGCTCTGCGTTTAGCCACGTTGTTTTCCCAACTTTAAAGAAGCCGGCACTTGG
CACTGCCTTTTTTTTCCGTCGTGAGGTTAGAGTCGCGGTTCGGTCAGCAGCCATGAGGTGGTTTC
CCTTGCTAGCGGTTTTAATCCCCGCTGCGCTTGCCAGCGATGTCCTCGACTACTCGGCGGACTTC
GACACGAAGATTCAGGACCACGACGCAGCACTAGTCGAATTCTTCGCTCCATGGTGCGGACACTG
CAAGAGGTTAGCGCCCGAGTACGAAAAAGCAGCTACCGAACTTAAAACCAACGACCCTCCAGTTC
CATTGATAAAGGTGGACTGTACCTCCGACGGTGGAAAAGATACGTGTTCAAAGCATGGAGTCAGC
GGCTACCCCACCCTCAAGATCTTCAGAGGTGGAGAGTTTTCGGCGGATTACAATGGCCCAAGGGA
AGCCGGTGGCATCGTTAAATACATGAAGGCCCAGGTGGGACCAAGCTCGAAGGAACTTCTGTCTG
TTGCTGAAGTCGAGAAGTATCTGTCGAAGGACGACGTTGTCATTTTTGGTTTCTTCGAGAGCAAG
GATGCCAGCCTGCACGAAAACTTCCTCAAGGTTGCAGACAAGCAGCGGGAGGCCTGGACTTTCGG
GCACTCGTTTGACAAGGATGTGCTCAAGAAGTATGGCTACAAAAACCAAGTTGTGCTGTTCCGAC
CCAAGATTCTCAAGAACAAGTTTGAGGAGAGCTTTGCAGTTTACTCTGGCTCTGATGACAAGGCG
GAACTGGAGACATTCATCAAGGAGAACTACCATGGCCTGGTGGGCCACAGGACTCAGGACAACTA
TAACATGTTCCAGGCTCCGCTCCTGGTTGCCTACTACGACGTAGACTACACCAAGAACGCCAAGG
GCACCAACTACTGGCGCAACCGCATCCTCAAGGTGGCCCAGAACTACAAGGGCAAGCTCAATTTT
GCTGTGAGCAACAAGGACAGCTTTGCTGCCGAGATGGACGACTACGGCGTGACGGTCAAGGCCAA
CAAGCCTGCCATCGCCGTGCGAAACAGCGAGAACGAGAAGTTCCGCATGACCAACGACTTCAGCG
TCGAGAACCTGGAGAAGTTCCTGGAGGAGTACCTTGCTGGCAAAGTGAAGGCCCACCTAAAGTCC
GAGCCCGTGCCGGAGACCAACGACGGACCGGTCAAGGTCGCGGTGGCAGAGAACTTCAAGTCCCT
GGTCACGGAGAGCACCAAGGACGTCCTCATCGAGTTCTATGCCCCCTGGTGTGGACACTGCAAGA
AGTTGGCCCCTACCTACGAGGAAGTGGGCAAGACGCTTGCTGACGAGGATGTCCTAGTTGTGAAG
ATGGATGCCACAGCCAATGATGTGCCGTCTGCCTTTGAGGTGTCTGGTTTCCCCACCCTGTACTG
GCTGCCCAAGAACGACAAGCAGAACCCGCGGCGGTACGAAGGAGGCCGGGAGCACGACGACTTCA
TCAAATGGATTGCCAAGCACGCCACGGACGAGCTCAAGGCCTACGACCGCTCCGGAGCCAAGAGG
GCCAAGGAGGAGTTATGAGTGGTCTCTGCTTCACCTGTGGGGGACCAGTTGGGACAAGGACATTG
TTCAATCACTTTCACCGGGGGTTCTGTTTTTCGCCATCTCTGTACAATAAACCAAGGGTGTTTTT
GTAATCGCGA
SG10 XP_002411436
SEQ ID NO: 40
MRVPYVLPVLFLAAASAFEVGKDYVYHYNGKMQVYNPEQPLQSSGFAFRSKVVAQPRPDHTHFKI
IDFEVDSFNGDHVHVGEHEFNYHSTEALKQFIERPFAGKFSEGKLEEAELSKSEPKWARNLKKGV
LSIFQLDLVKGRHDHPHAKQFHVREEGLHGNCDTLYVVAEEEGHLKVTKIKNLEKCDKEHYAVYG
RIKGHECVDCEAQETHPFVATSQVKYRLDGTPEHYVINHACATSENVFRPFGQGKTFVAQLNRTL
DLEEVHDANTDTQLPEDLEKVHHIAQTFPESDEVESLEELKHVNRYVTTFDLSTDKDKFISGLNH
LAALEYEDSDIKDVHSKESGGLNFLILFGSLASMPFEDIAHVYEQAVANAPEASKSQVRKVELDL
LSAVGNNPHAAFGLQLVKEDKLTDEEAEHFLAKLALNLKENSPALLTELAEVCEHVKPKRPVWVN
CQLALSTLAGQEGCVRAKTDKEQDEGFCKPSIVSHFFNYEIKPEDKKDQPEYKRTVYMKAAGNLA
TRGAVHYLERYVSDTNQPEYRRSAALWAMVRAAPHHHELVRDVALPLYKNKSETAYLRIGAFVNV
LMTKPDLYLLKYIGHNIIDDPSDQLASYVTSAFRSLVKSKYPCHQELAQHLRYVVPMWNDVSRFA
KPLDHTKSQVILSSGYDPKYDYGGSSVFAQVRADDSYLPREVYFSAKDYFHGRSFESVSLKFESW
GLDKLLNHVVGPQPGSTKNLWNVFGRRRFTRDASAKDLQEVEAALPITDRDYDHVYGRLSLDLYG
HAFDTWEFDESILQELSKGEGEEAPGDKLKNLLGESKRKKSFILSKDICVMVPSELGVPVFYHIL
QVEYMYINPKKFAFDQTQDAKFAVDLERHYVHESRGYSMFGVALTFNKTSMGTGSDSQTIMNLPI
DLKITLDPIHKKLSMKRPLSLPWNVLNHHFRPFTFVMSYDMHKDATTTETMMTQAMHPLYNKEEL
TEFDRNYFGEVLGYGLNVKGSLLSKGLKKGLHDYWYESDDRQKFYYATLNPEWHPRELQFTVVPA
AQDATTEVEVDLGYKFLEPDDPRESHFEAHDNVGDDAEVPSTHVLNLDYTLKGTKERKVSAELRY
SFTKDLLKHKVQLFYDRTPFHPKEQDHTKVEVDLGYKFLEPDDPRESHFEAHDNVGDDAEVPSTH
VLNLDYALKGTKERKVSAELRYSFTKDLLKHKVQLFYDRTPFHPKEQDHTKITFKGKYTHTDDEE
KQIRENAEGKPLGINRMKKYTLRRPYKKCTEHQKRGVPLNFECMKYLYYTSRLGKLTTDVEYKNL
KPVSVEGLHPVNQGRAPASFRRFRTGAPR
SEQ ID NO: 41
>XM_002411391.1 Ixodes scapularis heme lipoprotein precursor,
putative, mRNA
ATGAGAGTCCCCTACGTTCTGCCCGTCCTGTTCCTTGCCGCCGCTTCGGCCTTCGAAGTGGGCAA
GGACTATGTGTACCACTACAACGGAAAGATGCAAGTGTACAACCCGGAGCAGCCCCTGCAGTCCT
CGGGTTTCGCCTTCCGAAGCAAGGTCGTCGCCCAGCCCAGGCCCGACCATACGCACTTCAAGATA
ATCGACTTTGAGGTGGACAGCTTCAACGGCGATCATGTTCATGTCGGTGAACACGAGTTCAACTA
CCACTCCACCGAGGCGCTGAAGCAATTCATCGAGAGGCCCTTCGCCGGCAAGTTCTCCGAAGGAA
AGCTCGAAGAAGCTGAGCTGAGCAAGAGCGAACCTAAGTGGGCCAGGAACCTCAAGAAGGGAGTC
CTCAGCATTTTCCAGCTGGACTTGGTCAAGGGTCGCCACGACCACCCCCACGCCAAGCAGTTCCA
CGTGAGGGAGGAAGGTCTCCACGGGAACTGCGACACACTCTACGTTGTCGCCGAGGAGGAAGGTC
ACCTCAAGGTCACCAAGATCAAGAATCTCGAGAAATGCGACAAGGAACACTACGCCGTTTATGGA
AGGATCAAGGGACATGAGTGCGTCGACTGCGAAGCCCAGGAGACTCACCCTTTCGTAGCCACCTC
TCAGGTCAAGTACAGACTCGACGGAACTCCAGAACACTACGTCATCAACCACGCCTGCGCCACCT
CAGAGAATGTCTTCAGGCCCTTCGGTCAAGGGAAAACCTTCGTTGCCCAGCTCAACCGTACCTTG
GATTTGGAGGAGGTGCACGACGCCAACACCGACACTCAGCTTCCGGAGGACCTGGAGAAGGTGCA
CCACATCGCGCAGACATTCCCCGAGTCTGATGAGGTGGAAAGCCTGGAGGAACTGAAGCACGTCA
ACCGCTACGTGACCACCTTTGATCTCTCGACCGACAAAGACAAGTTCATCTCTGGTCTCAACCAC
CTTGCTGCATTGGAGTACGAAGATTCCGACATCAAGGACGTTCACAGCAAGGAAAGCGGTGGACT
AAACTTCCTGATTTTGTTCGGCTCGCTTGCCTCCATGCCCTTTGAAGATATCGCCCACGTGTACG
AGCAAGCCGTTGCCAATGCACCGGAAGCAAGCAAGAGTCAAGTCAGAAAGGTGTTCCTGGACCTG
CTCTCAGCTGTCGGAAACAACCCTCACGCAGCGTTCGGTCTGCAACTGGTCAAGGAAGACAAACT
CACTGACGAAGAGGCTGAGCACTTCCTCGCCAAGCTGGCGCTGAACCTTAAGGAGAACAGCCCCG
CTCTTCTGACCGAACTTGCCGAGGTGTGTGAGCACGTGAAGCCTAAGCGACCAGTATGGGTCAAC
TGCCAACTCGCTCTGAGTACACTCGCAGGCCAAGAGGGCTGCGTCCGTGCCAAGACTGACAAGGA
ACAGGACGAGGGATTCTGCAAGCCCTCTATTGTTTCGCACTTCTTTAACTATGAGATTAAGCCAG
AAGACAAGAAGGACCAGCCAGAATACAAGAGGACGGTGTACATGAAGGCTGCGGGCAACTTGGCC
ACCAGGGGCGCCGTGCACTACCTGGAGCGCTACGTTTCGGACACAAACCAGCCGGAGTACCGGAG
GTCAGCCGCCCTCTGGGCAATGGTTCGGGCTGCTCCGCACCACCACGAACTGGTACGCGACGTCG
CACTCCCACTGTACAAAAACAAGAGCGAGACGGCCTACTTGCGCATCGGGGCATTTGTGAACGTC
CTAATGACCAAACCCGACCTCTACCTGCTCAAGTACATCGGACACAACATCATCGACGACCCCAG
CGACCAGCTGGCGTCCTACGTGACCAGCGCCTTCCGCAGCCTTGTGAAATCCAAGTACCCGTGCC
ACCAAGAACTGGCTCAGCACCTTCGCTACGTCGTGCCCATGTGGAACGACGTAAGCCGGTTTGCC
AAGCCGCTGGACCATACCAAATCGCAGGTCATCCTCTCTTCCGGCTACGACCCCAAGTACGACTA
CGGTGGCTCCAGTGTCTTCGCCCAAGTACGGGCCGACGACAGCTACCTACCGAGAGAGGTGTATT
TCAGCGCAAAGGACTATTTCCACGGCCGCTCCTTCGAGTCAGTTTCACTAAAGTTTGAGAGCTGG
GGTCTGGACAAGCTTCTGAACCACGTCGTGGGACCTCAGCCCGGATCCACCAAGAACCTGTGGAA
CGTCTTTGGGCGACGTCGCTTCACCAGGGACGCCTCGGCAAAGGATCTCCAGGAAGTTGAGGCTG
CTCTTCCCATCACTGACCGGGATTATGACCATGTCTACGGCCGCCTGAGTCTCGATCTCTACGGT
CACGCCTTCGACACCTGGGAATTCGACGAGAGCATTCTGCAGGAACTGAGCAAGGGAGAGGGAGA
AGAGGCACCAGGCGACAAGCTGAAGAACCTGCTAGGCGAATCAAAGCGTAAGAAGAGCTTCATCC
TCAGCAAGGACATCTGCGTTATGGTACCCAGCGAACTTGGTGTTCCCGTATTTTACCACATCCTG
CAAGTCGAATACATGTACATCAACCCCAAGAAGTTTGCCTTCGATCAAACGCAAGATGCCAAATT
TGCCGTCGACCTAGAAAGGCACTACGTACACGAAAGCCGGGGCTACAGCATGTTCGGTGTCGCTC
TCACCTTCAACAAGACGTCCATGGGAACGGGCTCCGATAGTCAGACCATCATGAATCTGCCGATC
GACCTGAAGATTACCCTGGATCCTATTCACAAGAAGCTGAGTATGAAGCGCCCGCTGAGCCTGCC
CTGGAACGTCCTGAACCACCACTTCCGCCCGTTTACCTTCGTCATGTCCTACGACATGCACAAAG
ATGCTACCACCACCGAAACCATGATGACCCAGGCTATGCACCCACTCTACAACAAGGAAGAACTT
ACCGAGTTTGATCGTAACTACTTCGGCGAGGTGTTGGGCTATGGCCTGAACGTGAAGGGAAGCCT
CCTCAGCAAGGGACTCAAGAAGGGACTGCACGACTACTGGTACGAGAGCGACGACCGCCAGAAGT
TCTACTATGCCACTCTGAACCCTGAGTGGCACCCACGTGAACTGCAGTTCACCGTCGTTCCAGCT
GCACAAGATGCCACAACCGAGGTAGAGGTCGATCTCGGCTACAAGTTCCTCGAGCCCGATGACCC
ACGGGAGAGTCACTTCGAAGCTCACGACAACGTCGGCGACGATGCTGAGGTTCCGTCCACTCACG
TGCTGAACCTCGACTACACTCTGAAGGGCACCAAGGAACGCAAGGTGTCCGCGGAACTGAGATAC
TCCTTCACCAAGGACCTCCTCAAGCACAAAGTACAGCTCTTCTATGACCGAACACCATTCCACCC
GAAGGAGCAAGACCACACGAAGGTAGAGGTCGATCTCGGCTACAAGTTCCTCGAGCCCGATGACC
CACGGGAGAGTCACTTCGAAGCTCACGACAACGTCGGCGACGATGCTGAGGTTCCGTCCACTCAC
GTGCTGAACCTCGACTACGCTCTGAAGGGCACCAAGGAACGCAAGGTGTCCGCGGAACTGAGATA
CTCCTTCACCAAGGACCTCCTCAAGCACAAAGTACAGCTCTTCTACGACCGAACACCATTCCACC
CGAAGGAGCAAGACCACACCAAGATTACCTTTAAGGGAAAGTATACGCACACGGACGACGAAGAG
AAGCAGATTCGAGAGAACGCAGAAGGCAAGCCGCTGGGAATCAACCGAATGAAGAAGTACACCTT
GCGTCGGCCGTACAAGAAGTGCACCGAGCACCAGAAGCGGGGCGTTCCCCTAAACTTTGAGTGCA
TGAAGTACCTGTACTACACCAGTCGCCTGGGTAAATTGACAACGGACGTCGAGTACAAGAACCTC
AAGCCGGTGAGCGTTGAAGGCCTTCATCCAGTCAACCAAGGTCGAGCTCCTGCCAGTTTCCGCCG
ATTCCGGACTGGTGCTCCGCGTTGA
SG09_XP002411435
SEQ ID NO: 42
MRVPYVLPVLFLAAASAFEVGKDYVYHYNGKLQVYNPEQPLQSSGFAFQSKVVAQPRPDHTHFKI
IDFEVDTFNGEHVHLSDHQFHYHSTDALKQFIERPFAGKFSEGKLEAAELGKSEPMWSQNIKKGV
LSVFQLDLVKGRHDDPHAKQFYVKEDGLHGNCDTLYVVAEKEGHLEVTKIKNLEKCDKDHFAIYG
RIKAHECVDCEAQESHPLVATAQVKYRLDGTPEHYVIDHACATSETVLRPYGKGKTFVVQLNRTL
DLEEVHDANTDTQLPEDLERVDHLSQTLPVGDHVETLQDLKKVNHFVDYFQLTNNKEKFVAGLNH
LAALEYEDDDVKDVHGKESGGLQFLVLFNALSTLHFEDVVQVYEQAVASAPEASQSHVKRLFLDL
LSAAGTNPQVAFGLQLVKEDKLSDDEAEHFFTKLALNLKENSPALLTELAEVCEHVKPKRQVWVN
CQLALSILAGQEGCVRAKTDKEHDEGFCKPSLVSHFFNYSVKPEDKKDQPEYKRTAYIKAAGNLA
TRGAVHYLERFISDTNQPEYTRAAAFWALRRAGSKHPELVRDIALPVYKNKSESSYLRIAAFTAV
MKTHPDLYLLKYIGHNIIDDPSDQVASYVTSLFRSFEKSKYPCHQSLAQHLRYVVPMWNDVYRFS
KPPDYTKSHVHLSSGYDPKYDYGGATSFGMVRADDSYLPRDVYVSVKDYMSGHSLSTATLWFENW
GMDKLLNHVVGPQPGSSKNLWNVFGRRRFTRDASAKELKEVEDALPVTDREYDHVYGRLNLDLFG
HAIDSWEFDEDLLKEVGKEDGEGKCLESLKLLILTDRLRKHKPGTKLKNLLGQVQRKKNFYLSTD
IIFVAPTEVGVPIFFDFKQAEFTYINRKKFSVDQTEDAKFTFDVQRHYVHESRGYTMVGVGLTFN
KTSLGTGSDVQTIFNLPIDLHLTVDPIHKTLSLKRPLSLPWNVLNHHFRPFTFTMPYDIATDVSH
AVTTLEQPQYPLYNKEEVTEIDHTYLTDVFGYGLNVKGSLLSKGLKKGLHDFWYESDCRQRYYYA
LMNPEWHPRELQFTVVPAEHDATTEVEVDLGYKFLEPDDARESHFPVQDNVGGDAEVPSTHVLSL
DYTLKGSKERKASAELRYSFTKDNLKHKVQFFYDRTPFHPKEQVHTKVCMDVTSKFPKPEWSKLK
NLAVFHEGKDLESELNLHYGSNCVDQSSITFKGKYTHTDDEERQIRENAEGKPRGINRMDKYNLR
DPFNKCTEHQKRGVPLNLYCMKYLYYTSRLGKFTTDVEYKNLKPLLPMLTKYYKKVHPEGGFLST
VVSHFHGPNGKLHVVSQVPPVQDYADIVVTTEDGHSHNHSHVPLYSHLLEPRIMFSTLGYSNMAH
YISYYKQKYCDLQGQSVRTFDNVVVQLPETDCYKVVAKDCSPNKRFTVLARATGNAELPKALKAF
IQTTQVELLPVAEDSGLVLRVDGNRVPLTPGVPYSHTAHDAELFTVTLENKYFEVTSKPYGLNLG
FNGNMLFVQTANFYRGKLCGLCGDYNYDRQHELVGPNLHHYNDTLEFAKSYVVPSSDCTPP
SEQ ID NO: 43
ATGAGAGTCCCCTACGTTCTGCCTGTCCTGTTCCTTGCTGCAGCTTCGGCCTTCGAAGTGGGCAA
GGACTACGTGTACCACTACAACGGAAAGCTACAAGTCTATAACCCGGAGCAGCCCCTACAGTCTT
CGGGATTCGCGTTCCAGAGCAAGGTCGTCGCCCAACCCAGGCCCGACCACACGCACTTCAAGATA
ATCGATTTCGAGGTGGACACCTTCAACGGCGAACATGTCCATCTCAGTGACCACCAGTTCCACTA
CCACTCCACCGACGCACTGAAGCAATTTATTGAGAGACCATTTGCCGGCAAGTTCTCCGAAGGAA
AGCTCGAGGCCGCGGAGCTGGGAAAGAGCGAGCCCATGTGGTCTCAGAACATCAAGAAGGGTGTC
CTCAGCGTGTTCCAACTGGACCTCGTCAAGGGACGTCATGACGATCCCCACGCCAAGCAGTTCTA
CGTGAAGGAGGACGGCCTTCACGGAAACTGCGACACCCTCTACGTTGTCGCCGAGAAAGAGGGTC
ACCTCGAGGTTACCAAGATCAAGAATCTTGAGAAATGCGACAAAGACCACTTCGCCATCTACGGG
AGGATTAAGGCGCACGAGTGCGTCGATTGTGAAGCCCAGGAGTCGCACCCCCTCGTGGCCACCGC
CCAGGTCAAGTACAGGCTGGATGGAACTCCAGAGCACTACGTCATTGACCACGCCTGCGCCACCT
CGGAGACTGTCTTGAGGCCTTACGGCAAAGGAAAAACCTTCGTTGTCCAGCTCAACCGCACCTTG
GATTTGGAGGAGGTGCACGACGCCAACACCGACACCCAGCTCCCAGAGGACCTGGAAAGGGTGGA
CCACCTGAGTCAGACGCTTCCAGTAGGTGATCATGTCGAAACACTCCAAGATCTGAAGAAGGTCA
ACCACTTCGTCGACTACTTTCAACTCACAAACAACAAAGAAAAGTTCGTCGCGGGTCTGAACCAC
CTCGCTGCCCTTGAGTACGAAGATGACGACGTCAAGGATGTTCACGGCAAGGAAAGCGGAGGCCT
CCAGTTCCTGGTCTTGTTCAACGCTCTTTCCACTCTGCATTTCGAAGATGTCGTCCAGGTGTACG
AGCAAGCCGTCGCCAGCGCACCGGAAGCAAGCCAATCTCACGTTAAGAGACTGTTCCTGGACCTG
CTGTCAGCTGCTGGAACGAACCCTCAAGTTGCGTTCGGCCTGCAACTGGTCAAGGAAGATAAGCT
CTCGGACGATGAGGCTGAGCACTTCTTCACCAAGTTGGCGCTCAACCTTAAGGAGAACAGCCCTG
CTCTTCTGACTGAACTTGCCGAGGTGTGTGAGCACGTGAAGCCGAAACGACAAGTGTGGGTCAAC
TGTCAGCTCGCCCTAAGTATCCTCGCAGGCCAAGAGGGTTGCGTTCGCGCAAAGACCGACAAGGA
ACACGACGAGGGATTCTGCAAGCCCTCTCTGGTGTCGCACTTCTTTAACTATTCGGTCAAGCCTG
AAGACAAGAAGGACCAGCCAGAATATAAGAGGACCGCGTACATCAAGGCTGCTGGCAACCTAGCC
ACCAGGGGCGCCGTGCATTACCTGGAACGCTTCATCTCCGATACCAACCAGCCAGAGTACACGAG
GGCCGCCGCCTTCTGGGCACTGAGGCGAGCTGGATCGAAGCACCCCGAACTGGTGCGCGATATCG
CACTTCCCGTGTACAAGAACAAGAGCGAGTCATCGTATCTGCGTATCGCGGCGTTCACAGCCGTC
ATGAAGACACATCCCGACCTCTACCTGCTCAAGTACATCGGCCACAACATCATCGACGACCCTAG
CGACCAAGTGGCATCTTACGTGACCAGCCTGTTCCGTAGTTTCGAGAAATCCAAGTATCCCTGCC
ACCAATCACTAGCTCAGCACCTTCGCTACGTCGTGCCAATGTGGAACGACGTGTACCGGTTCTCC
AAGCCCCCGGACTACACCAAATCGCATGTGCATCTTTCCTCTGGTTACGACCCCAAGTACGACTA
CGGTGGCGCCACGTCCTTCGGTATGGTCCGGGCTGACGACAGCTACCTCCCACGAGACGTCTACG
TTAGCGTCAAGGACTACATGAGCGGTCACTCCCTCAGCACGGCTACCTTGTGGTTTGAGAATTGG
GGCATGGACAAGCTGTTGAACCACGTCGTGGGACCTCAGCCCGGATCCTCAAAGAACCTGTGGAA
CGTCTTTGGGCGACGTCGGTTTACCAGGGATGCGTCGGCGAAGGAGCTCAAGGAAGTCGAGGATG
CGCTTCCCGTCACCGATCGGGAGTACGACCACGTCTACGGCAGACTGAACCTCGACCTTTTCGGT
CACGCCATCGACAGCTGGGAGTTCGACGAGGATCTTCTCAAAGAAGTCGGCAAGGAAGACGGAGA
AGGCAAGTGCTTAGAATCTTTGAAGCTTTTAATATTAACTGACAGGCTAAGGAAACACAAACCAG
GAACCAAGCTGAAGAACCTCTTGGGCCAAGTGCAGCGCAAGAAAAACTTCTACCTGAGCACTGAC
ATAATCTTTGTTGCGCCCACCGAGGTTGGTGTTCCAATCTTCTTCGACTTCAAGCAAGCCGAATT
TACGTACATCAACCGCAAGAAGTTCAGCGTGGACCAGACTGAAGACGCCAAGTTCACTTTTGATG
TTCAGAGGCACTACGTGCACGAGAGCCGTGGCTACACCATGGTCGGCGTGGGCCTGACCTTTAAC
AAGACGTCCTTGGGAACGGGGTCCGACGTCCAGACCATCTTCAACCTGCCGATCGACCTACACCT
CACTGTGGATCCCATTCACAAGACGCTGAGCCTGAAGCGCCCGCTGAGCCTGCCCTGGAACGTCC
TGAACCACCACTTCCGTCCGTTCACCTTTACCATGCCCTATGACATCGCCACAGATGTGTCCCAC
GCTGTCACCACACTGGAGCAGCCTCAGTACCCCCTCTACAACAAGGAAGAAGTAACTGAGATTGA
TCATACCTACCTCACCGACGTCTTCGGCTATGGCTTGAACGTGAAGGGTAGCCTCCTCAGCAAAG
GACTCAAGAAAGGACTGCACGACTTCTGGTACGAGAGCGATTGCCGACAGAGGTACTACTATGCC
CTCATGAATCCAGAGTGGCACCCCCGTGAACTGCAGTTCACCGTCGTTCCAGCTGAGCATGACGC
CACGACTGAGGTAGAGGTCGATCTCGGCTACAAGTTCCTCGAACCCGATGACGCAAGAGAGAGCC
ACTTCCCCGTTCAGGACAACGTCGGCGGAGATGCTGAGGTTCCTTCCACCCATGTGCTCAGCCTC
GACTACACTCTGAAGGGCTCCAAGGAGCGCAAGGCGTCCGCAGAACTGAGGTACTCGTTTACCAA
GGACAACCTCAAGCACAAAGTGCAGTTCTTCTATGACCGGACGCCCTTCCACCCAAAGGAGCAAG
TCCACACTAAGGTCTGCATGGATGTCACGTCTAAATTCCCAAAGCCCGAGTGGTCCAAGTTGAAG
AACCTCGCCGTTTTCCACGAGGGCAAGGATCTGGAATCTGAGCTGAACCTTCATTACGGAAGCAA
CTGCGTAGACCAGTCTAGCATCACATTCAAGGGAAAATATACTCACACGGATGACGAAGAAAGGC
AGATTCGAGAGAACGCTGAAGGCAAGCCGCGGGGAATCAACCGAATGGACAAGTATAACCTCCGA
GATCCGTTCAACAAATGCACCGAGCATCAGAAGCGTGGCGTTCCACTAAACCTTTACTGCATGAA
GTACCTGTACTACACCAGCCGCCTGGGTAAATTCACAACAGACGTCGAGTACAAGAACCTGAAGC
CGTTGCTCCCCATGCTAACGAAGTACTACAAGAAGGTCCACCCCGAGGGAGGTTTCCTGAGCACC
GTAGTGTCCCACTTCCATGGACCCAACGGAAAGCTCCACGTCGTCTCTCAAGTGCCTCCTGTTCA
GGACTACGCGGACATCGTTGTAACCACGGAGGACGGTCACTCCCACAACCACTCCCACGTGCCGC
TCTACAGCCACTTGCTCGAGCCCAGGATCATGTTCTCCACGCTAGGGTACTCGAACATGGCTCAC
TACATCTCCTACTACAAACAGAAATACTGCGATCTGCAAGGACAGAGCGTCAGGACTTTCGACAA
CGTCGTCGTGCAGCTGCCGGAGACCGACTGCTACAAGGTGGTCGCCAAGGACTGCTCGCCCAACA
AGAGGTTCACGGTCCTGGCACGGGCTACCGGAAACGCCGAACTCCCCAAGGCGCTGAAGGCATTC
ATCCAGACCACCCAGGTCGAGCTCCTGCCAGTTGCCGAAGACTCCGGTCTTGTGCTCCGCGTTGA
CGGAAACAGAGTTCCACTGACCCCGGGAGTACCTTACAGTCACACCGCGCACGACGCCGAGCTCT
TCACCGTCACGCTGGAAAACAAATACTTCGAAGTAACGTCGAAGCCATACGGTCTCAATCTAGGC
TTCAACGGAAACATGCTCTTCGTACAGACCGCCAACTTCTACCGCGGCAAGCTGTGCGGCCTTTG
CGGCGACTACAACTACGACCGGCAGCACGAGCTCGTGGGCCCCAACCTCCACCACTACAACGACA
CCCTGGAGTTCGCCAAGAGCTACGTCGTTCCCTCTTCCGACTGCACCCCGCCCTGA
SEQ ID NO: 44
>XM_002411390.1 Ixodes scapularis hemelipoglycoprotein precursor,
putative, mRNA
GTAAAGAACTGCATCGTCATGAGAGTCCCCTACGTTCTGCCTGTCCTGTTCCTTGCTGCAGCTTC
GGCCTTCGAAGTGGGCAAGGACTACGTGTACCACTACAACGGAAAGCTACAAGTCTATAACCCGG
AGCAGCCCCTACAGTCTTCGGGATTCGCGTTCCAGAGCAAGGTCGTCGCCCAACCCAGGCCCGAC
CACACGCACTTCAAGATAATCGATTTCGAGGTGGACACCTTCAACGGCGAACATGTCCATCTCAG
TGACCACCAGTTCCACTACCACTCCACCGACGCACTGAAGCAATTTATTGAGAGACCATTTGCCG
GCAAGTTCTCCGAAGGAAAGCTCGAGGCCGCGGAGCTGGGAAAGAGCGAGCCCATGTGGTCTCAG
AACATCAAGAAGGGTGTCCTCAGCGTGTTCCAACTGGACCTCGTCAAGGGACGTCATGACGATCC
CCACGCCAAGCAGTTCTACGTGAAGGAGGACGGCCTTCACGGAAACTGCGACACCCTCTACGTTG
TCGCCGAGAAAGAGGGTCACCTCGAGGTTACCAAGATCAAGAATCTTGAGAAATGCGACAAAGAC
CACTTCGCCATCTACGGGAGGATTAAGGCGCACGAGTGCGTCGATTGTGAAGCCCAGGAGTCGCA
CCCCCTCGTGGCCACCGCCCAGGTCAAGTACAGGCTGGATGGAACTCCAGAGCACTACGTCATTG
ACCACGCCTGCGCCACCTCGGAGACTGTCTTGAGGCCTTACGGCAAAGGAAAAACCTTCGTTGTC
CAGCTCAACCGCACCTTGGATTTGGAGGAGGTGCACGACGCCAACACCGACACCCAGCTCCCAGA
GGACCTGGAAAGGGTGGACCACCTGAGTCAGACGCTTCCAGTAGGTGATCATGTCGAAACACTCC
AAGATCTGAAGAAGGTCAACCACTTCGTCGACTACTTTCAACTCACAAACAACAAAGAAAAGTTC
GTCGCGGGTCTGAACCACCTCGCTGCCCTTGAGTACGAAGATGACGACGTCAAGGATGTTCACGG
CAAGGAAAGCGGAGGCCTCCAGTTCCTGGTCTTGTTCAACGCTCTTTCCACTCTGCATTTCGAAG
ATGTCGTCCAGGTGTACGAGCAAGCCGTCGCCAGCGCACCGGAAGCAAGCCAATCTCACGTTAAG
AGACTGTTCCTGGACCTGCTGTCAGCTGCTGGAACGAACCCTCAAGTTGCGTTCGGCCTGCAACT
GGTCAAGGAAGATAAGCTCTCGGACGATGAGGCTGAGCACTTCTTCACCAAGTTGGCGCTCAACC
TTAAGGAGAACAGCCCTGCTCTTCTGACTGAACTTGCCGAGGTGTGTGAGCACGTGAAGCCGAAA
CGACAAGTGTGGGTCAACTGTCAGCTCGCCCTAAGTATCCTCGCAGGCCAAGAGGGTTGCGTTCG
CGCAAAGACCGACAAGGAACACGACGAGGGATTCTGCAAGCCCTCTCTGGTGTCGCACTTCTTTA
ACTATTCGGTCAAGCCTGAAGACAAGAAGGACCAGCCAGAATATAAGAGGACCGCGTACATCAAG
GCTGCTGGCAACCTAGCCACCAGGGGCGCCGTGCATTACCTGGAACGCTTCATCTCCGATACCAA
CCAGCCAGAGTACACGAGGGCCGCCGCCTTCTGGGCACTGAGGCGAGCTGGATCGAAGCACCCCG
AACTGGTGCGCGATATCGCACTTCCCGTGTACAAGAACAAGAGCGAGTCATCGTATCTGCGTATC
GCGGCGTTCACAGCCGTCATGAAGACACATCCCGACCTCTACCTGCTCAAGTACATCGGCCACAA
CATCATCGACGACCCTAGCGACCAAGTGGCATCTTACGTGACCAGCCTGTTCCGTAGTTTCGAGA
AATCCAAGTATCCCTGCCACCAATCACTAGCTCAGCACCTTCGCTACGTCGTGCCAATGTGGAAC
GACGTGTACCGGTTCTCCAAGCCCCCGGACTACACCAAATCGCATGTGCATCTTTCCTCTGGTTA
CGACCCCAAGTACGACTACGGTGGCGCCACGTCCTTCGGTATGGTCCGGGCTGACGACAGCTACC
TCCCACGAGACGTCTACGTTAGCGTCAAGGACTACATGAGCGGTCACTCCCTCAGCACGGCTACC
TTGTGGTTTGAGAATTGGGGCATGGACAAGCTGTTGAACCACGTCGTGGGACCTCAGCCCGGATC
CTCAAAGAACCTGTGGAACGTCTTTGGGCGACGTCGGTTTACCAGGGATGCGTCGGCGAAGGAGC
TCAAGGAAGTCGAGGATGCGCTTCCCGTCACCGATCGGGAGTACGACCACGTCTACGGCAGACTG
AACCTCGACCTTTTCGGTCACGCCATCGACAGCTGGGAGTTCGACGAGGATCTTCTCAAAGAAGT
CGGCAAGGAAGACGGAGAAGGCAAGTGCTTAGAATCTTTGAAGCTTTTAATATTAACTGACAGGC
TAAGGAAACACAAACCAGGAACCAAGCTGAAGAACCTCTTGGGCCAAGTGCAGCGCAAGAAAAAC
TTCTACCTGAGCACTGACATAATCTTTGTTGCGCCCACCGAGGTTGGTGTTCCAATCTTCTTCGA
CTTCAAGCAAGCCGAATTTACGTACATCAACCGCAAGAAGTTCAGCGTGGACCAGACTGAAGACG
CCAAGTTCACTTTTGATGTTCAGAGGCACTACGTGCACGAGAGCCGTGGCTACACCATGGTCGGC
GTGGGCCTGACCTTTAACAAGACGTCCTTGGGAACGGGGTCCGACGTCCAGACCATCTTCAACCT
GCCGATCGACCTACACCTCACTGTGGATCCCATTCACAAGACGCTGAGCCTGAAGCGCCCGCTGA
GCCTGCCCTGGAACGTCCTGAACCACCACTTCCGTCCGTTCACCTTTACCATGCCCTATGACATC
GCCACAGATGTGTCCCACGCTGTCACCACACTGGAGCAGCCTCAGTACCCCCTCTACAACAAGGA
AGAAGTAACTGAGATTGATCATACCTACCTCACCGACGTCTTCGGCTATGGCTTGAACGTGAAGG
GTAGCCTCCTCAGCAAAGGACTCAAGAAAGGACTGCACGACTTCTGGTACGAGAGCGATTGCCGA
CAGAGGTACTACTATGCCCTCATGAATCCAGAGTGGCACCCCCGTGAACTGCAGTTCACCGTCGT
TCCAGCTGAGCATGACGCCACGACTGAGGTAGAGGTCGATCTCGGCTACAAGTTCCTCGAACCCG
ATGACGCAAGAGAGAGCCACTTCCCCGTTCAGGACAACGTCGGCGGAGATGCTGAGGTTCCTTCC
ACCCATGTGCTCAGCCTCGACTACACTCTGAAGGGCTCCAAGGAGCGCAAGGCGTCCGCAGAACT
GAGGTACTCGTTTACCAAGGACAACCTCAAGCACAAAGTGCAGTTCTTCTATGACCGGACGCCCT
TCCACCCAAAGGAGCAAGTCCACACTAAGGTCTGCATGGATGTCACGTCTAAATTCCCAAAGCCC
GAGTGGTCCAAGTTGAAGAACCTCGCCGTTTTCCACGAGGGCAAGGATCTGGAATCTGAGCTGAA
CCTTCATTACGGAAGCAACTGCGTAGACCAGTCTAGCATCACATTCAAGGGAAAATATACTCACA
CGGATGACGAAGAAAGGCAGATTCGAGAGAACGCTGAAGGCAAGCCGCGGGGAATCAACCGAATG
GACAAGTATAACCTCCGAGATCCGTTCAACAAATGCACCGAGCATCAGAAGCGTGGCGTTCCACT
AAACCTTTACTGCATGAAGTACCTGTACTACACCAGCCGCCTGGGTAAATTCACAACAGACGTCG
AGTACAAGAACCTGAAGCCGTTGCTCCCCATGCTAACGAAGTACTACAAGAAGGTCCACCCCGAG
GGAGGTTTCCTGAGCACCGTAGTGTCCCACTTCCATGGACCCAACGGAAAGCTCCACGTCGTCTC
TCAAGTGCCTCCTGTTCAGGACTACGCGGACATCGTTGTAACCACGGAGGACGGTCACTCCCACA
ACCACTCCCACGTGCCGCTCTACAGCCACTTGCTCGAGCCCAGGATCATGTTCTCCACGCTAGGG
TACTCGAACATGGCTCACTACATCTCCTACTACAAACAGAAATACTGCGATCTGCAAGGACAGAG
CGTCAGGACTTTCGACAACGTCGTCGTGCAGCTGCCGGAGACCGACTGCTACAAGGTGGTCGCCA
AGGACTGCTCGCCCAACAAGAGGTTCACGGTCCTGGCACGGGCTACCGGAAACGCCGAACTCCCC
AAGGCGCTGAAGGCATTCATCCAGACCACCCAGGTCGAGCTCCTGCCAGTTGCCGAAGACTCCGG
TCTTGTGCTCCGCGTTGACGGAAACAGAGTTCCACTGACCCCGGGAGTACCTTACAGTCACACCG
CGCACGACGCCGAGCTCTTCACCGTCACGCTGGAAAACAAATACTTCGAAGTAACGTCGAAGCCA
TACGGTCTCAATCTAGGCTTCAACGGAAACATGCTCTTCGTACAGACCGCCAACTTCTACCGCGG
CAAGCTGTGCGGCCTTTGCGGCGACTACAACTACGACCGGCAGCACGAGCTCGTGGGCCCCAACC
TCCACCACTACAACGACACCCTGGAGTTCGCCAAGAGCTACGTCGTTCCCTCTTCCGACTGCACC
CCGCCCTGAGCATCATTTTCCGTCGACATCATCGTTCAACCCTATTTCGGCCGCTCTTCGTGAGC
AGGCATTTTCTTCGATAAGTACTGACACGAAGGGACAGTGCGAGAGTTGAGCAGCAACTGTAACC
TCTAGAACGAACGTAACGCCGCGGAAGCCTCAAGACAACGTGATAAAGGCTTTATTGGACGTGTT
CACTGCCATGGACGTTTTAGCGAAGTAAAAAAAGCTAATAAATGCATTGAAAAAATTGT
vitellogenin-6
SEQ ID NO: 45
[Ixodes
>XP_029826419.1 vitellogenin-6 [Ixodes scapularis]
MRVPYVLPVLFLAAASAFEVGKDYVYHYNGKMQVYNPEQPLQSSGFAFRSKVVAQPRPDHTHFKI
IDFEVDSFNGDHVHVGEHEFNYHSTEALKQFIERPFAGKFSEGKLEEAELSKSEPKWARNLKKGV
LSIFQLDLVKGRHDNPHAKQFHVREEGLHGNCDTLYVVAEEEGHLKVTKIKNLEKCDKEHYAVYG
RIKGHECVDCEAQETHPFVATSQVKYRLDGTPEHYVINHACATSENVFRPFGQGKTFVAQLNRTL
DLEEVHDANTDTQLPEDLEKVHHIAQAFPESDEVESLEELKHVNRYVTTFELSTDKDKFISGLNH
LAALEYEDSDIKDVHSKESGGLNFLILFGSLASMPFEDIAHVYEQAVANAPEASKSQVRKVELDL
LSAVGNNPHAAFGLQLVKEDKLTDEEAEHFLAKLALNLKENSPALLTELAEVCEHVKPKRPVWVN
CQLALSSLAGQEGCVRAKTDKEHDEGFCKPSIVSHFFNYEIKPEDKKDQPEYKRTVYMKAAGNLA
TRGAVHYLERYVSDTNQPEYRRSAALWAMVRAAPHHHELVRDVALPVYKNKSETAYLRIGAFVNV
LMTKPDLYLLKYIGHNIIDDPSDQLASYVTSAFRSLVKSKYPCHQELAQHLRYVVPMWNDVSRFA
KPLDHTKSQVILSSGYDPKFDYGGSSVFAQVRADDSYLPREVYFSAKDYFHGRSFESVSLKFESW
GLDKLLNHVVGPQPGSTKNLWNVFGRRRFTRDASAKDLQEVEAALPITDRDYDHVYGRLSLDLYG
HAFDTWEFDESILQELSKGEGEEAPGDKLKNLLGESKRKKAFILSKDICIIVPSELGVPVFYDIK
QVEYMYINPKKFAFDQTQDAKFAMDVERHYVHESRGFSMFGVALTFNKTSLGTGSDSQTIINLPI
DLKITLDPIHKKLSMKRPLSLPWNVLNHHFRPFTFVMSYDINEDVTTAETKMTRAMYPLYNKEEL
TEFDRNYFGEVLGYGLNVKGSLLSKGLKKGLHDYWYESDDRQKFYYATLNPEWHPRELQFTVVPA
AQDATTEVEVDLGYKFLEPDDPRESHFEAHDNVGDDAEVPSTHVLNLDYTLKGTKERKVSAELRY
SFTKDLLKHKVQLFYDRTPFHPKEQDHTKVCLDVISKFPEPDWERLNNLAVFHEGKDIESQLNLH
YGSNCVDQSSITFKGKYTHTDDEEKQIRENAEGKPLGINRMKKYTLRRPYKKCTEHQKRGVPLNF
ECMKYLYYTSRLGKLTTDVEYKNLKPLFPMLLKYYKKVHKEGGFLSTLASHVHGPTGKLHVVSQV
PPVEKYSDIVVTTEDGHSFHHDHVPIYSHLLEPRIFSVLGYSNMVNYISYYKQKHCDLQGKSLRT
FDNVVVNLPETDCFKVVAKDCSPNKRFTVLARATGNAALPKALKAFIQSTKVELLPVSADSGLVL
RVDGNKVDLTAGVPYSHTAHDVELFTVMQHNKYFEVMSQPYGVFIGFDGNVLFVQTANFYRGKLC
GLCGDYNYDRQHELVGPNLHHYNDTLEFAKSYVVPASDCTAP
SEQ ID NO: 46
ATGAGAGTCCCCTACGTTCTGCCCGTCCTGTTCCTTGCCGCCGCTTCGGCCTTCGAAGTGGGCAA
GGACTATGTGTACCACTACAACGGAAAGATGCAAGTGTACAACCCCGAGCAGCCCCTGCAGTCCT
CGGGTTTCGCCTTCCGAAGCAAGGTCGTCGCCCAGCCCAGGCCCGACCATACGCACTTCAAGATA
ATCGACTTTGAGGTGGACAGCTTCAACGGTGATCATGTTCATGTCGGTGAACACGAGTTCAACTA
CCACTCCACAGAGGCGCTGAAGCAATTCATCGAGAGGCCCTTCGCCGGCAAGTTCTCCGAAGGAA
AGCTCGAAGAAGCTGAGCTGAGCAAGAGCGAACCTAAGTGGGCCAGGAACCTCAAGAAGGGAGTC
CTCAGCATTTTCCAGCTGGACTTGGTCAAGGGTCGCCATGACAACCCCCACGCCAAGCAGTTCCA
CGTGAGAGAGGAGGGTCTCCACGGGAACTGCGACACACTCTACGTTGTCGCCGAGGAGGAAGGTC
ACCTCAAGGTCACCAAGATCAAGAATCTCGAGAAATGCGACAAGGAACACTACGCCGTTTATGGA
AGGATCAAGGGACATGAGTGCGTCGACTGCGAAGCCCAGGAGACTCACCCTTTCGTAGCTACCTC
CCAGGTCAAGTACAGACTCGACGGAACGCCAGAACACTACGTCATCAACCACGCCTGCGCCACCT
CAGAGAATGTCTTCAGGCCCTTCGGTCAAGGGAAAACCTTCGTTGCCCAGCTCAACCGCACCTTG
GATTTGGAGGAGGTGCACGACGCCAACACCGACACTCAGCTTCCAGAGGACCTGGAGAAGGTGCA
CCACATCGCGCAGGCATTCCCCGAGTCGGATGAGGTGGAAAGCCTGGAGGAACTGAAGCACGTCA
ACCGCTACGTGACCACCTTTGAGCTCTCGACCGACAAAGACAAGTTCATCTCTGGTCTCAACCAC
CTTGCTGCATTGGAGTACGAAGATTCCGACATCAAGGACGTTCACAGCAAGGAAAGCGGTGGACT
CAACTTCCTGATTTTGTTCGGATCGCTTGCCTCCATGCCCTTTGAAGATATCGCCCACGTGTACG
AGCAAGCCGTTGCCAATGCACCGGAAGCAAGCAAGAGTCAAGTCAGAAAGGTGTTCCTGGACCTG
CTCTCAGCTGTCGGAAACAACCCTCACGCAGCGTTCGGTCTGCAATTGGTCAAGGAAGACAAACT
CACTGACGAGGAGGCCGAGCACTTCCTCGCCAAGCTGGCGCTGAACCTAAAGGAGAACAGCCCCG
CTCTTCTGACCGAACTTGCCGAGGTGTGTGAGCACGTGAAGCCTAAACGACCAGTGTGGGTCAAC
TGCCAACTCGCTCTGAGTAGCCTCGCTGGCCAAGAGGGCTGCGTCCGTGCCAAGACTGACAAGGA
ACACGACGAGGGATTCTGCAAGCCCTCTATTGTTTCGCACTTCTTTAACTATGAGATTAAGCCAG
AAGACAAGAAGGACCAGCCAGAATACAAGAGGACGGTGTACATGAAGGCTGCGGGCAACTTGGCC
ACCAGGGGCGCCGTGCACTACCTGGAGCGCTACGTCTCGGACACAAACCAGCCGGAGTACCGGAG
GTCAGCCGCCCTCTGGGCAATGGTTCGAGCTGCTCCGCACCACCACGAACTGGTACGCGACGTCG
CACTCCCAGTGTACAAAAACAAGAGCGAGACGGCCTACCTGCGTATCGGGGCATTTGTGAACGTC
CTAATGACCAAACCCGACCTCTACCTGCTCAAGTACATCGGACACAACATCATCGACGACCCCAG
CGACCAGCTGGCGTCCTACGTGACCAGCGCCTTCCGCAGCCTTGTGAAATCCAAGTACCCGTGCC
ACCAAGAACTGGCTCAGCACCTTCGCTACGTCGTGCCCATGTGGAATGACGTAAGCCGGTTTGCC
AAGCCGCTGGACCATACCAAATCGCAAGTCATCCTCTCTTCCGGCTACGATCCCAAGTTCGACTA
CGGCGGCTCCAGTGTCTTCGCCCAAGTACGGGCCGACGACAGCTACCTACCGAGAGAAGTGTATT
TCAGCGCAAAGGACTATTTCCACGGCCGCTCCTTCGAGTCAGTTTCACTAAAGTTTGAGAGCTGG
GGTCTGGACAAGCTTCTGAACCACGTCGTGGGACCTCAGCCCGGATCCACCAAGAACCTGTGGAA
CGTCTTTGGGCGACGTCGCTTTACCAGGGACGCCTCGGCAAAGGATCTCCAGGAAGTTGAGGCTG
CTCTTCCCATCACCGACCGGGATTATGACCATGTCTACGGCCGCCTGAGTCTCGATCTCTACGGT
CACGCCTTCGACACCTGGGAATTCGACGAGAGCATTCTGCAGGAACTGAGCAAGGGAGAGGGAGA
AGAGGCACCAGGCGACAAGCTGAAGAACCTGCTAGGCGAATCAAAGCGTAAGAAGGCCTTCATCC
TCAGCAAGGACATCTGCATTATTGTACCCAGCGAACTTGGTGTTCCCGTATTCTACGACATCAAG
CAAGTCGAATACATGTACATCAACCCCAAGAAGTTTGCCTTCGATCAAACGCAAGATGCCAAATT
TGCCATGGACGTAGAAAGGCACTACGTACACGAAAGCCGTGGCTTCAGCATGTTCGGTGTTGCTC
TCACCTTCAACAAGACGTCCTTGGGAACGGGCTCCGATAGTCAGACCATCATAAATCTGCCGATC
GACCTGAAGATTACCCTGGATCCTATTCACAAGAAGCTGAGTATGAAGCGCCCGCTGAGCCTGCC
CTGGAACGTCCTGAACCACCACTTCCGCCCGTTTACCTTTGTCATGTCCTACGACATAAACGAAG
ATGTTACCACAGCCGAAACCAAGATGACCCGGGCTATGTACCCTCTCTACAACAAGGAAGAACTT
ACCGAGTTTGATCGTAACTACTTCGGCGAGGTCTTGGGCTATGGCCTGAACGTGAAGGGAAGCCT
CCTCAGCAAGGGGCTCAAGAAGGGACTGCACGACTACTGGTACGAGAGCGATGATCGCCAGAAGT
TCTACTATGCCACTCTAAACCCTGAGTGGCACCCACGTGAACTGCAGTTCACCGTCGTTCCAGCT
GCACAAGATGCCACAACTGAGGTAGAGGTCGATCTCGGCTACAAGTTCCTCGAGCCCGATGACCC
ACGGGAGAGTCACTTCGAAGCTCACGACAACGTCGGCGACGATGCTGAGGTTCCGTCCACTCACG
TGCTGAACCTCGACTACACTCTGAAGGGCACCAAGGAACGCAAGGTGTCCGCGGAACTGAGATAC
TCCTTCACCAAGGACCTCCTCAAGCACAAAGTACAGCTCTTCTATGACCGAACACCATTCCACCC
GAAGGAGCAAGACCACACGAAGGTCTGCCTGGATGTCATATCGAAGTTCCCAGAGCCCGACTGGG
AAAGGCTGAATAACCTCGCCGTTTTCCATGAGGGCAAGGACATTGAATCTCAGCTGAACCTCCAC
TACGGAAGCAACTGTGTTGACCAGTCTAGCATTACTTTTAAGGGAAAGTATACGCACACGGACGA
CGAGGAGAAGCAGATTCGAGAGAACGCAGAAGGCAAGCCGCTGGGAATCAACCGAATGAAGAAGT
ACACCCTGCGTCGGCCGTACAAAAAGTGCACCGAGCACCAGAAGCGGGGCGTTCCCCTAAACTTT
GAGTGCATGAAGTACCTGTACTACACCAGTCGCCTGGGTAAATTGACAACGGACGTCGAGTACAA
GAACCTCAAGCCGCTGTTCCCCATGCTGCTGAAGTACTACAAGAAGGTCCACAAGGAGGGCGGTT
TCCTAAGCACCCTCGCGTCCCACGTCCACGGACCCACCGGAAAGCTCCACGTCGTCTCTCAAGTG
CCTCCGGTTGAGAAGTACTCGGACATTGTTGTGACCACCGAGGACGGTCACTCCTTCCACCATGA
CCACGTTCCCATCTACAGCCACTTGCTCGAGCCTAGGATTTTCTCTGTGCTGGGATACTCGAACA
TGGTTAACTACATCTCCTACTACAAACAGAAACATTGCGATCTTCAAGGCAAGAGCCTCCGAACC
TTCGACAACGTCGTCGTGAACCTTCCCGAGACTGACTGCTTCAAGGTGGTTGCCAAGGACTGCTC
TCCCAACAAGAGGTTCACGGTTCTGGCACGAGCTACCGGAAACGCCGCACTCCCCAAGGCGTTGA
AGGCCTTCATCCAGTCAACCAAGGTCGAGCTCCTGCCAGTTTCCGCCGATTCTGGACTGGTGCTC
CGCGTCGACGGCAACAAGGTGGATCTGACGGCGGGAGTTCCTTACAGCCACACCGCGCACGATGT
CGAGCTCTTCACGGTCATGCAGCACAACAAGTACTTTGAAGTGATGTCGCAGCCTTACGGGGTCT
TCATTGGCTTCGACGGGAACGTGCTCTTTGTGCAGACCGCCAACTTCTACCGCGGCAAGCTGTGC
GGTCTTTGCGGCGACTACAACTACGACCGGCAGCACGAGCTCGTGGGCCCCAACCTCCACCACTA
CAACGACACCCTGGAGTTCGCCAAGAGCTACGTCGTTCCCGCGTCCGACTGCACCGCGCCTTGA
SEQ ID NO: 47
>XM_029970559.1 (26419)-Vg6
ATGCCTTCAGTGCTCACTCCTCGGGGAATGGGAGCGAGTTGCTAAGGAACTGCATCGTCATGAGA
GTCCCCTACGTTCTGCCCGTCCTGTTCCTTGCCGCCGCTTCGGCCTTCGAAGTGGGCAAGGACTA
TGTGTACCACTACAACGGAAAGATGCAAGTGTACAACCCCGAGCAGCCCCTGCAGTCCTCGGGTT
TCGCCTTCCGAAGCAAGGTCGTCGCCCAGCCCAGGCCCGACCATACGCACTTCAAGATAATCGAC
TTTGAGGTGGACAGCTTCAACGGTGATCATGTTCATGTCGGTGAACACGAGTTCAACTACCACTC
CACAGAGGCGCTGAAGCAATTCATCGAGAGGCCCTTCGCCGGCAAGTTCTCCGAAGGAAAGCTCG
AAGAAGCTGAGCTGAGCAAGAGCGAACCTAAGTGGGCCAGGAACCTCAAGAAGGGAGTCCTCAGC
ATTTTCCAGCTGGACTTGGTCAAGGGTCGCCATGACAACCCCCACGCCAAGCAGTTCCACGTGAG
AGAGGAGGGTCTCCACGGGAACTGCGACACACTCTACGTTGTCGCCGAGGAGGAAGGTCACCTCA
AGGTCACCAAGATCAAGAATCTCGAGAAATGCGACAAGGAACACTACGCCGTTTATGGAAGGATC
AAGGGACATGAGTGCGTCGACTGCGAAGCCCAGGAGACTCACCCTTTCGTAGCTACCTCCCAGGT
CAAGTACAGACTCGACGGAACGCCAGAACACTACGTCATCAACCACGCCTGCGCCACCTCAGAGA
ATGTCTTCAGGCCCTTCGGTCAAGGGAAAACCTTCGTTGCCCAGCTCAACCGCACCTTGGATTTG
GAGGAGGTGCACGACGCCAACACCGACACTCAGCTTCCAGAGGACCTGGAGAAGGTGCACCACAT
CGCGCAGGCATTCCCCGAGTCGGATGAGGTGGAAAGCCTGGAGGAACTGAAGCACGTCAACCGCT
ACGTGACCACCTTTGAGCTCTCGACCGACAAAGACAAGTTCATCTCTGGTCTCAACCACCTTGCT
GCATTGGAGTACGAAGATTCCGACATCAAGGACGTTCACAGCAAGGAAAGCGGTGGACTCAACTT
CCTGATTTTGTTCGGATCGCTTGCCTCCATGCCCTTTGAAGATATCGCCCACGTGTACGAGCAAG
CCGTTGCCAATGCACCGGAAGCAAGCAAGAGTCAAGTCAGAAAGGTGTTCCTGGACCTGCTCTCA
GCTGTCGGAAACAACCCTCACGCAGCGTTCGGTCTGCAATTGGTCAAGGAAGACAAACTCACTGA
CGAGGAGGCCGAGCACTTCCTCGCCAAGCTGGCGCTGAACCTAAAGGAGAACAGCCCCGCTCTTC
TGACCGAACTTGCCGAGGTGTGTGAGCACGTGAAGCCTAAACGACCAGTGTGGGTCAACTGCCAA
CTCGCTCTGAGTAGCCTCGCTGGCCAAGAGGGCTGCGTCCGTGCCAAGACTGACAAGGAACACGA
CGAGGGATTCTGCAAGCCCTCTATTGTTTCGCACTTCTTTAACTATGAGATTAAGCCAGAAGACA
AGAAGGACCAGCCAGAATACAAGAGGACGGTGTACATGAAGGCTGCGGGCAACTTGGCCACCAGG
GGCGCCGTGCACTACCTGGAGCGCTACGTCTCGGACACAAACCAGCCGGAGTACCGGAGGTCAGC
CGCCCTCTGGGCAATGGTTCGAGCTGCTCCGCACCACCACGAACTGGTACGCGACGTCGCACTCC
CAGTGTACAAAAACAAGAGCGAGACGGCCTACCTGCGTATCGGGGCATTTGTGAACGTCCTAATG
ACCAAACCCGACCTCTACCTGCTCAAGTACATCGGACACAACATCATCGACGACCCCAGCGACCA
GCTGGCGTCCTACGTGACCAGCGCCTTCCGCAGCCTTGTGAAATCCAAGTACCCGTGCCACCAAG
AACTGGCTCAGCACCTTCGCTACGTCGTGCCCATGTGGAATGACGTAAGCCGGTTTGCCAAGCCG
CTGGACCATACCAAATCGCAAGTCATCCTCTCTTCCGGCTACGATCCCAAGTTCGACTACGGCGG
CTCCAGTGTCTTCGCCCAAGTACGGGCCGACGACAGCTACCTACCGAGAGAAGTGTATTTCAGCG
CAAAGGACTATTTCCACGGCCGCTCCTTCGAGTCAGTTTCACTAAAGTTTGAGAGCTGGGGTCTG
GACAAGCTTCTGAACCACGTCGTGGGACCTCAGCCCGGATCCACCAAGAACCTGTGGAACGTCTT
TGGGCGACGTCGCTTTACCAGGGACGCCTCGGCAAAGGATCTCCAGGAAGTTGAGGCTGCTCTTC
CCATCACCGACCGGGATTATGACCATGTCTACGGCCGCCTGAGTCTCGATCTCTACGGTCACGCC
TTCGACACCTGGGAATTCGACGAGAGCATTCTGCAGGAACTGAGCAAGGGAGAGGGAGAAGAGGC
ACCAGGCGACAAGCTGAAGAACCTGCTAGGCGAATCAAAGCGTAAGAAGGCCTTCATCCTCAGCA
AGGACATCTGCATTATTGTACCCAGCGAACTTGGTGTTCCCGTATTCTACGACATCAAGCAAGTC
GAATACATGTACATCAACCCCAAGAAGTTTGCCTTCGATCAAACGCAAGATGCCAAATTTGCCAT
GGACGTAGAAAGGCACTACGTACACGAAAGCCGTGGCTTCAGCATGTTCGGTGTTGCTCTCACCT
TCAACAAGACGTCCTTGGGAACGGGCTCCGATAGTCAGACCATCATAAATCTGCCGATCGACCTG
AAGATTACCCTGGATCCTATTCACAAGAAGCTGAGTATGAAGCGCCCGCTGAGCCTGCCCTGGAA
CGTCCTGAACCACCACTTCCGCCCGTTTACCTTTGTCATGTCCTACGACATAAACGAAGATGTTA
CCACAGCCGAAACCAAGATGACCCGGGCTATGTACCCTCTCTACAACAAGGAAGAACTTACCGAG
TTTGATCGTAACTACTTCGGCGAGGTCTTGGGCTATGGCCTGAACGTGAAGGGAAGCCTCCTCAG
CAAGGGGCTCAAGAAGGGACTGCACGACTACTGGTACGAGAGCGATGATCGCCAGAAGTTCTACT
ATGCCACTCTAAACCCTGAGTGGCACCCACGTGAACTGCAGTTCACCGTCGTTCCAGCTGCACAA
GATGCCACAACTGAGGTAGAGGTCGATCTCGGCTACAAGTTCCTCGAGCCCGATGACCCACGGGA
GAGTCACTTCGAAGCTCACGACAACGTCGGCGACGATGCTGAGGTTCCGTCCACTCACGTGCTGA
ACCTCGACTACACTCTGAAGGGCACCAAGGAACGCAAGGTGTCCGCGGAACTGAGATACTCCTTC
ACCAAGGACCTCCTCAAGCACAAAGTACAGCTCTTCTATGACCGAACACCATTCCACCCGAAGGA
GCAAGACCACACGAAGGTCTGCCTGGATGTCATATCGAAGTTCCCAGAGCCCGACTGGGAAAGGC
TGAATAACCTCGCCGTTTTCCATGAGGGCAAGGACATTGAATCTCAGCTGAACCTCCACTACGGA
AGCAACTGTGTTGACCAGTCTAGCATTACTTTTAAGGGAAAGTATACGCACACGGACGACGAGGA
GAAGCAGATTCGAGAGAACGCAGAAGGCAAGCCGCTGGGAATCAACCGAATGAAGAAGTACACCC
TGCGTCGGCCGTACAAAAAGTGCACCGAGCACCAGAAGCGGGGCGTTCCCCTAAACTTTGAGTGC
ATGAAGTACCTGTACTACACCAGTCGCCTGGGTAAATTGACAACGGACGTCGAGTACAAGAACCT
CAAGCCGCTGTTCCCCATGCTGCTGAAGTACTACAAGAAGGTCCACAAGGAGGGCGGTTTCCTAA
GCACCCTCGCGTCCCACGTCCACGGACCCACCGGAAAGCTCCACGTCGTCTCTCAAGTGCCTCCG
GTTGAGAAGTACTCGGACATTGTTGTGACCACCGAGGACGGTCACTCCTTCCACCATGACCACGT
TCCCATCTACAGCCACTTGCTCGAGCCTAGGATTTTCTCTGTGCTGGGATACTCGAACATGGTTA
ACTACATCTCCTACTACAAACAGAAACATTGCGATCTTCAAGGCAAGAGCCTCCGAACCTTCGAC
AACGTCGTCGTGAACCTTCCCGAGACTGACTGCTTCAAGGTGGTTGCCAAGGACTGCTCTCCCAA
CAAGAGGTTCACGGTTCTGGCACGAGCTACCGGAAACGCCGCACTCCCCAAGGCGTTGAAGGCCT
TCATCCAGTCAACCAAGGTCGAGCTCCTGCCAGTTTCCGCCGATTCTGGACTGGTGCTCCGCGTC
GACGGCAACAAGGTGGATCTGACGGCGGGAGTTCCTTACAGCCACACCGCGCACGATGTCGAGCT
CTTCACGGTCATGCAGCACAACAAGTACTTTGAAGTGATGTCGCAGCCTTACGGGGTCTTCATTG
GCTTCGACGGGAACGTGCTCTTTGTGCAGACCGCCAACTTCTACCGCGGCAAGCTGTGCGGTCTT
TGCGGCGACTACAACTACGACCGGCAGCACGAGCTCGTGGGCCCCAACCTCCACCACTACAACGA
CACCCTGGAGTTCGCCAAGAGCTACGTCGTTCCCGCGTCCGACTGCACCGCGCCTTGAGCACCGT
CTTGCGTCAACATCGTCGTTCAACCTTATTTCTGCCGCTCCTTGCGAGCCGGTATTTTCATTCAT
TAGTAACGAGATGATGTGAAAGTGCAAGTGTCGTGTCAAGACTGTAGTTGTTAGAATAATGGCAA
AGCCCCGAGAGCCTCGAGACAACGGGATGACGGTTTGTGTGAACGTTTGCAATGCCATGGGCGCT
TGTTCGACATGAGAAAAACAAATAAATACATGAAGCAGAA
>EEC10299.1 peroxinectin, putative [Ixodes scapularis]
SEQ ID NO: 48
MRLSYWNVAAALACFLLLVSIGQAQRRGNGGRGRGQGRSRGQSQRQQAAPAAAPAANKPASDCTL
STGEPGICQPLSHCVFQFNAIDDLALSKCGATTKVCCPLNTPAATGTLEFSRPVTVTARRVSIPK
ISKDDLDIAGRASLSIVRGIEDAERTLLNDGKFAKKRTTASLHAAFFGSKPIVQSLGRDGLIGLE
ATRELARKFNLDSTQGRDGLQRFNIQNTVISDACPPVPRCPSTKYRSPDGRCNNLQNREWGKSLT
AFERFMPPNYADGLTLPRVSLDGNPLPNPRTISVAADPDGDFPNERNTLMLMQFAQFVDHDLTLT
GVTRFRNGSAITCCDEEFLTNPTKRHFACMPIDLDANDHFYSEFNLRCIEFVRSVPAPRPQCTFG
PREQLNQLTAYMDSSNIYGSTEEEAKSLRSFRDGRLASTFFSRDELLPRQTDGTQECNEQGTDFV
CFRAGDERVNEQVSLTAMHTLWLREHNRVAGELHRLNPGWKDEILYQEARRIVAAEFQHICFNEF
LPLLLGRKVMEQFDLLLTPYGYSHSYDPNLNAGIGNVFATAAYRYGHTLVQGNIELINEDGSVHK
RIPLAAQFFNPNEIYHPGNLDRFFRGLITQPAQTYDRFVTQQLTNHLYEPIGQGFGMDLVALNIQ
RGRDHGIPSYNDWREHCGMSRITDFAQLADIMTPESAKAFAQVYKYPDDIDLFPAGVNEKSVPGG
TLGPTFACLVAEQFRRMKNGDRFWYENGGLESSFNEVQIEEIRKASLARVICDNSNLNYVQPLVM
IREAQWNPKVDCNGEDIPRVSLDNWQNEPVWT
SEQ ID NO: 49
ATGCGGCTCTCCTACTGGAACGTGGCCGCTGCTCTGGCCTGCTTCCTGCTCCTGGTCAGC
ATCGGTCAGGCACAGCGAAGAGGAAATGGCGGCCGAGGTCGCGGCCAGGGCCGTAGTCGG
GGCCAGAGCCAGCGCCAGCAGGCTGCGCCGGCGGCAGCCCCCGCGGCCAACAAGCCGGCG
TCCGACTGCACGCTGTCCACGGGCGAGCCCGGGATCTGCCAGCCCCTGAGCCACTGCGTA
TTCCAGTTCAACGCCATCGATGACCTGGCGCTGTCCAAGTGCGGCGCCACGACCAAAGTT
TGCTGCCCGCTGAACACGCCAGCTGCCACCGGAACACTGGAGTTCAGTCGCCCGGTGACG
GTGACGGCGAGGAGGGTGTCCATCCCCAAGATCAGCAAGGACGACCTGGACATCGCCGGC
AGAGCCAGTCTCAGCATCGTCCGCGGAATTGAAGACGCCGAGAGGACCCTTCTCAATGAT
GGCAAGTTCGCGAAAAAACGAACAACAGCGTCGCTGCACGCCGCTTTCTTCGGGTCCAAG
CCCATCGTGCAGTCTCTTGGAAGGGACGGCCTCATCGGACTGGAAGCCACCAGGGAACTG
GCAAGAAAGTTCAACCTGGACTCGACGCAGGGCCGCGATGGCCTGCAGCGCTTCAACATC
CAGAACACGGTGATCTCGGACGCCTGTCCTCCCGTGCCGCGCTGCCCCTCGACCAAGTAC
CGGTCCCCCGACGGACGCTGCAACAACCTCCAGAACAGGGAGTGGGGCAAGTCCCTCACC
GCCTTCGAGAGGTTCATGCCGCCCAACTACGCCGACGGCCTGACCCTGCCCCGAGTGAGC
TTGGACGGCAACCCGCTGCCCAACCCCCGCACCATCTCGGTGGCCGCCGATCCCGACGGA
GACTTCCCCAACGAGCGGAACACGCTGATGCTCATGCAGTTCGCTCAGTTCGTCGACCAC
GACCTCACGCTCACCGGAGTCACCAGATTCCGCAACGGCTCGGCCATCACCTGCTGCGAC
GAGGAGTTCCTTACGAACCCGACCAAAAGGCACTTCGCCTGCATGCCCATCGACCTGGAT
GCCAACGATCACTTCTACTCAGAGTTCAACCTCAGGTGCATCGAGTTCGTACGCTCCGTG
CCGGCGCCCCGACCACAGTGCACGTTCGGACCTCGTGAACAGTTGAACCAGCTGACTGCT
TACATGGACTCCTCCAACATCTACGGCTCGACGGAAGAAGAAGCCAAGTCCCTGCGGAGC
TTCAGGGACGGTCGGCTGGCGTCCACGTTCTTCAGCAGGGACGAGCTGCTGCCTCGTCAA
ACAGATGGCACGCAGGAGTGCAACGAACAAGGGACCGACTTCGTTTGCTTCAGGGCAGGA
GACGAGCGCGTGAACGAGCAGGTGAGCCTGACGGCGATGCACACGCTGTGGCTACGAGAG
CACAACCGGGTCGCCGGAGAGCTGCACCGCCTCAACCCGGGCTGGAAGGACGAGATCCTG
TACCAGGAGGCCAGGAGGATCGTTGCCGCAGAGTTCCAGCACATCTGCTTCAACGAGTTT
CTGCCACTGCTCCTGGGTCGCAAAGTGATGGAACAGTTCGACCTGCTGCTTACGCCGTAC
GGATACAGCCATAGCTACGATCCCAATCTGAACGCCGGCATTGGCAACGTGTTCGCCACC
GCTGCATACAGATACGGGCACACCCTCGTGCAGGGCAACATCGAGCTGATCAACGAGGAC
GGATCCGTGCACAAGCGGATACCGCTGGCGGCCCAGTTCTTCAATCCGAACGAGATCTAT
CACCCGGGCAACCTGGATCGCTTCTTCAGGGGCCTGATCACGCAGCCTGCACAGACCTAC
GATCGCTTCGTCACGCAACAACTGACGAACCACCTGTACGAGCCCATCGGCCAGGGCTTC
GGTATGGACCTGGTGGCGTTGAACATCCAGCGGGGCCGAGACCACGGCATCCCCAGCTAC
AACGACTGGCGGGAACACTGCGGCATGTCCAGGATTACGGACTTCGCCCAGCTGGCCGAC
ATCATGACGCCGGAGTCCGCCAAGGCCTTCGCACAAGTCTACAAGTACCCCGACGACATC
GACTTGTTCCCGGCTGGCGTGAACGAGAAATCTGTGCCCGGCGGCACGCTAGGACCCACC
TTCGCCTGTCTCGTGGCCGAGCAGTTCCGACGGATGAAGAACGGCGACCGCTTCTGGTAC
GAGAACGGAGGGCTCGAGTCGTCCTTCAACGAAGTTCAAATCGAGGAAATTCGAAAGGCC
AGCCTGGCGCGGGTCATCTGCGACAACTCCAACCTGAACTACGTTCAGCCTCTGGTCATG
ATCAGAGAAGCGCAGTGGAACCCGAAGGTGGATTGCAATGGCGAAGACATTCCGAGAGTG
AGCTTGGACAACTGGCAGAACGAGCCGGTCTGGACGTAG
SEQ ID NO: 50
>ISCW007552 Peroxinectin, putative
ATGCGGCTCTCCTACTGGAACGTGGCCGCTGCTCTGGCCTGCTTCCTGCTCCTGGTCAGC
ATCGGTCAGGCACAGCGAAGAGGAAATGGCGGCCGAGGTCGCGGCCAGGGCCGTAGTCGG
GGCCAGAGCCAGCGCCAGCAGGCTGCGCCGGCGGCAGCCCCCGCGGCCAACAAGCCGGCG
TCCGACTGCACGCTGTCCACGGGCGAGCCCGGGATCTGCCAGCCCCTGAGCCACTGCGTA
TTCCAGTTCAACGCCATCGATGACCTGGCGCTGTCCAAGTGCGGCGCCACGACCAAAGTT
TGCTGCCCGCTGAACACGCCAGCTGCCACCGGAACACTGGAGTTCAGTCGCCCGGTGACG
GTGACGGCGAGGAGGGTGTCCATCCCCAAGATCAGCAAGGACGACCTGGACATCGCCGGC
AGAGCCAGTCTCAGCATCGTCCGCGGAATTGAAGACGCCGAGAGGACCCTTCTCAATGAT
GGCAAGTTCGCGAAAAAACGAACAACAGCGTCGCTGCACGCCGCTTTCTTCGGGTCCAAG
CCCATCGTGCAGTCTCTTGGAAGGGACGGCCTCATCGGACTGGAAGCCACCAGGGAACTG
GCAAGAAAGTTCAACCTGGACTCGACGCAGGGCCGCGATGGCCTGCAGCGCTTCAACATC
CAGAACACGGTGATCTCGGACGCCTGTCCTCCCGTGCCGCGCTGCCCCTCGACCAAGTAC
CGGTCCCCCGACGGACGCTGCAACAACCTCCAGAACAGGGAGTGGGGCAAGTCCCTCACC
GCCTTCGAGAGGTTCATGCCGCCCAACTACGCCGACGGCCTGACCCTGCCCCGAGTGAGC
TTGGACGGCAACCCGCTGCCCAACCCCCGCACCATCTCGGTGGCCGCCGATCCCGACGGA
GACTTCCCCAACGAGCGGAACACGCTGATGCTCATGCAGTTCGCTCAGTTCGTCGACCAC
GACCTCACGCTCACCGGAGTCACCAGATTCCGCAACGGCTCGGCCATCACCTGCTGCGAC
GAGGAGTTCCTTACGAACCCGACCAAAAGGCACTTCGCCTGCATGCCCATCGACCTGGAT
GCCAACGATCACTTCTACTCAGAGTTCAACCTCAGGTGCATCGAGTTCGTACGCTCCGTG
CCGGCGCCCCGACCACAGTGCACGTTCGGACCTCGTGAACAGTTGAACCAGCTGACTGCT
TACATGGACTCCTCCAACATCTACGGCTCGACGGAAGAAGAAGCCAAGTCCCTGCGGAGC
TTCAGGGACGGTCGGCTGGCGTCCACGTTCTTCAGCAGGGACGAGCTGCTGCCTCGTCAA
ACAGATGGCACGCAGGAGTGCAACGAACAAGGGACCGACTTCGTTTGCTTCAGGGCAGGA
GACGAGCGCGTGAACGAGCAGGTGAGCCTGACGGCGATGCACACGCTGTGGCTACGAGAG
CACAACCGGGTCGCCGGAGAGCTGCACCGCCTCAACCCGGGCTGGAAGGACGAGATCCTG
TACCAGGAGGCCAGGAGGATCGTTGCCGCAGAGTTCCAGCACATCTGCTTCAACGAGTTT
CTGCCACTGCTCCTGGGTCGCAAAGTGATGGAACAGTTCGACCTGCTGCTTACGCCGTAC
GGATACAGCCATAGCTACGATCCCAATCTGAACGCCGGCATTGGCAACGTGTTCGCCACC
GCTGCATACAGATACGGGCACACCCTCGTGCAGGGCAACATCGAGCTGATCAACGAGGAC
GGATCCGTGCACAAGCGGATACCGCTGGCGGCCCAGTTCTTCAATCCGAACGAGATCTAT
CACCCGGGCAACCTGGATCGCTTCTTCAGGGGCCTGATCACGCAGCCTGCACAGACCTAC
GATCGCTTCGTCACGCAACAACTGACGAACCACCTGTACGAGCCCATCGGCCAGGGCTTC
GGTATGGACCTGGTGGCGTTGAACATCCAGCGGGGCCGAGACCACGGCATCCCCAGCTAC
AACGACTGGCGGGAACACTGCGGCATGTCCAGGATTACGGACTTCGCCCAGCTGGCCGAC
ATCATGACGCCGGAGTCCGCCAAGGCCTTCGCACAAGTCTACAAGTACCCCGACGACATC
GACTTGTTCCCGGCTGGCGTGAACGAGAAATCTGTGCCCGGCGGCACGCTAGGACCCACC
TTCGCCTGTCTCGTGGCCGAGCAGTTCCGACGGATGAAGAACGGCGACCGCTTCTGGTAC
GAGAACGGAGGGCTCGAGTCGTCCTTCAACGAAGTTCAAATCGAGGAAATTCGAAAGGCC
AGCCTGGCGCGGGTCATCTGCGACAACTCCAACCTGAACTACGTTCAGCCTCTGGTCATG
ATCAGAGAAGCGCAGTGGAACCCGAAGGTGGATTGCAATGGCGAAGACATTCCGAGAGTG
AGCTTGGACAACTGGCAGAACGAGCCGGTCTGGACGTAGGGCCACCGACTCTCGCGGGGG
GAACTTTTATTTGTTGTGTTCCCCAATATTCCCTGGCATTACTCCCCTACCCCACGATGT
TGTTCACTTGCCGCGTTTTTTCCTCGTTGCTTTCTTGTTGCACGCATGTGACGATGCCCC
GCGATATTCGTTTCATTAAAGGTCACACGAGCGCACGTGCGCTTCATTCTTTCATCGT
>AAY66814.1 selenium dependent salivary glutathione peroxidase
[Ixodes scapularis]
SEQ ID NO: 51
MLSALSLALLCAVGAGVGPVAQAAAPLQIDCVQDESTERLQNFTFKDVLEKDTIPLSRFKGYVAL
VVNVATYUGLTPTYLQLNALQARFGERNFTVLGFPCNQFGKQEPGTRQEILNGIRYVRPGNNYVP
NFPMFQKIEVNGENQHPLYTFLKGRCTSPNPVFSAKDKLFYSPQNNNDIRWNFEKFLVDRRGVPV
KRYEPRYSPDEVARDIEVLTRSS
SEQ ID NO: 52
>DQ066177.1 Ixodes scapularis isolate ISNU-L-ISN-78 selenium
dependent salivary glutathione peroxidase mRNA, complete cds
ATGTTGTCGGCGCTCTCGCTAGCGCTGCTCTGCGCGGTCGGGGCCGGCGTGGGGCCGGTCGCACA
GGCCGCGGCACCGCTCCAGATCGACTGCGTCCAAGACGAGTCCACCGAGCGTCTGCAAAACTTCA
CCTTCAAGGATGTCCTGGAGAAGGACACCATCCCCCTGAGCCGGTTCAAGGGCTACGTCGCTCTG
GTGGTGAACGTGGCCACCTACTGAGGCCTGACCCCGACCTACCTGCAACTGAATGCACTGCAGGC
CAGGTTCGGGGAAAGGAACTTCACCGTGCTCGGGTTTCCCTGCAACCAGTTCGGAAAGCAAGAGC
CCGGTACCCGCCAGGAGATTCTGAACGGGATCAGGTACGTGCGTCCGGGCAACAACTACGTGCCC
AACTTTCCCATGTTCCAGAAGATCGAGGTGAACGGGGAAAACCAGCACCCGCTCTACACTTTCCT
CAAGGGTCGCTGCACCTCGCCGAATCCGGTTTTCAGCGCCAAGGATAAACTCTTCTACTCGCCCC
AGAACAACAACGACATCCGTTGGAACTTCGAGAAGTTTCTGGTTGACAGGCGAGGAGTGCCGGTG
AAACGCTACGAGCCCCGCTACTCGCCCGACGAGGTCGCTCGCGACATCGAGGTCCTCACAAGATC
GAGCTAG
>AAY66626.1 putative secreted protein [Ixodes scapularis]
SEQ ID NO: 53
MKATLAVLCFLVAVACAIVVEEKMVSHPIDEEALDPRCVKPVDCPGNEKEVSFYDPRRGCQLIRL
GENCTHNDNYPTLDLCNEHCPPAPGTHPRRA
SEQ ID NO: 54
>DQ065989.1 Ixodes scapularis isolate IS-6-12L-5-58 putative
secreted protein mRNA, complete cds
ATGAAGGCAACCCTTGCAGTTCTTTGCTTTCTTGTAGCAGTAGCGTGCGCCATTGTGGTGGAGGA
GAAGATGGTGTCCCATCCCATTGACGAAGAGGCTCTCGATCCAAGATGCGTAAAGCCGGTGGACT
GCCCCGGAAATGAAAAGGAGGTTTCCTTTTACGACCCACGAAGGGGATGTCAACTAATTAGACTG
GGGGAAAACTGTACCCACAATGACAATTACCCAACACTTGATCTTTGCAATGAACATTGCCCACC
TGCTCCAGGGACGCACCCGAGACGCGCCTGA

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for inducing an immune response against at least one tick salivary antigen in a subject, the composition comprising at least one nucleoside-modified RNA molecule encoding at least one tick salivary antigen.

2. The composition of claim 1, wherein the at least one nucleoside-modified RNA molecule comprises pseudouridine.

3. The composition of claim 1, wherein the at least one nucleoside-modified RNA molecule comprises 1-methyl-pseudouridine.

4. The composition of claim 1, wherein the at least one tick salivary antigen is selected from the group consisting of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein.

5. The composition of claim 1, wherein the composition comprises a plurality of nucleoside-modified RNA molecules encoding at least two tick salivary antigens selected from the group consisting of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein.

6. The composition of claim 1, wherein the at least one tick salivary antigen comprises an amino acid sequence selected from the group consisting of: SEQ ID NO:1, SEQ ID NO:3, SEQ ID NO:5, SEQ ID NO:7, SEQ ID NO:9, SEQ ID NO:11, SEQ ID NO: 13, SEQ ID NO:15, SEQ ID NO:18, SEQ ID NO:20, SEQ ID NO:22, SEQ ID NO:24, SEQ ID NO:26, SEQ ID NO:29, SEQ ID NO:32, SEQ ID NO:34, SEQ ID NO:37, SEQ ID NO:40, SEQ ID NO:42, SEQ ID NO:45, SEQ ID NO:48, SEQ ID NO:51, and SEQ ID NO:53.

7. The composition of claim 1, wherein the at least one nucleoside-modified RNA molecule comprises a nucleotide sequence encoded by a DNA molecule comprising at least one nucleotide sequence selected from the group consisting of: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:17, SEQ ID NO:19, SEQ ID NO:21, SEQ ID NO:23, SEQ ID NO:25, SEQ ID NO:27, SEQ ID NO:28, SEQ ID NO:30, SEQ ID NO:31, SEQ ID NO:33, SEQ ID NO:35, SEQ ID NO:36, SEQ ID NO:38, SEQ ID NO:39, SEQ ID NO:41, SEQ ID NO:43, SEQ ID NO:44, SEQ ID NO:46, SEQ ID NO:47, SEQ ID NO:49, SEQ ID NO:50, SEQ ID NO:52 and SEQ ID NO:54.

8. The composition of claim 1, wherein the composition further comprises an adjuvant.

9. The composition of claim 1, wherein the at least one nucleoside-modified RNA further encodes at least one adjuvant.

10. The composition of claim 1, further comprising a lipid nanoparticle (LNP).

11. The composition of claim 12, wherein the at least one nucleoside-modified RNA is encapsulated within the LNP.

12. The composition of claim 1, wherein the composition is a vaccine.

13. A method of inducing an immune response against one or more tick salivary antigen in a subject comprising administering to the subject an effective amount of a composition comprising at least one nucleoside-modified RNA molecule encoding at least one tick salivary antigen.

14. The method of claim 13, wherein the at least one nucleoside-modified RNA molecule comprises pseudouridine.

15. The method of claim 13, wherein the at least one nucleoside-modified RNA molecule comprises 1-methyl-pseudouridine.

16. The method of claim 13, wherein the at least one tick salivary antigen is selected from the group consisting of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein.

17. The method of claim 13, comprising administering to the subject an effective amount of a composition comprising a plurality of nucleoside-modified RNA molecules encoding at least two tick salivary antigens selected from the group consisting of Salp10, Salp15, Salp25A, Salp25B, Salp25C, Salp25D, Salp14, TSLPI, Salp26A, P11, Salp16A, Salp17, TIX5, P32, Salp12, SG27, IsPDIA3, SG10, SG09, vitellogenin-6, peroxinectin, selenium dependent salivary glutathione peroxidase, and IS-6-12L-5-58 putative secreted protein.

18. The method of claim 13, wherein the at least one tick salivary antigen comprises an amino acid sequence selected from the group consisting of: SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:18, SEQ ID NO:20, SEQ ID NO:22, SEQ ID NO:24, SEQ ID NO:26, SEQ ID NO:29, SEQ ID NO:32, SEQ ID NO:34, SEQ ID NO:37, SEQ ID NO:40, SEQ ID NO:42, SEQ ID NO:45, SEQ ID NO:48, SEQ ID NO:51, and SEQ ID NO:53.

19. The method of claim 13, wherein the at least one nucleoside-modified RNA molecule comprises a nucleotide sequence encoded by a DNA molecule comprising at least one nucleotide sequence selected from the group consisting of: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:17, SEQ ID NO:19, SEQ ID NO:21, SEQ ID NO:23, SEQ ID NO:25, SEQ ID NO:27, SEQ ID NO:28, SEQ ID NO:30, SEQ ID NO:31, SEQ ID NO:33, SEQ ID NO:35, SEQ ID NO:36, SEQ ID NO:38, SEQ ID NO:39, SEQ ID NO:41, SEQ ID NO:43, SEQ ID NO:44, SEQ ID NO:46, SEQ ID NO:47, SEQ ID NO:49, SEQ ID NO:50, SEQ ID NO:52 and SEQ ID NO:54.

20. The method of claim 13, wherein the method further comprises administering to the subject an effective amount of an adjuvant.

21. The method of claim 13, wherein the at least one nucleoside-modified RNA further encodes at least one adjuvant.

22. The method of claim 13, wherein the composition further comprises a lipid nanoparticle (LNP).

23. The method of claim 13, wherein the at least one nucleoside-modified RNA is encapsulated within the LNP.

24. The method of claim 13, wherein the composition is a vaccine.

25. The method of claim 13, wherein the composition is administered by a delivery route selected from the group consisting of intradermal, subcutaneous, inhalation, intranasal, and intramuscular.

26. The method of claim 13, wherein the method comprises a single administration of the composition.

27. The method of claim 13, wherein the method comprises multiple administrations of the composition.

28. The method of claim 13, wherein the method treats or prevents a disease or disorder associated with a tick-borne pathogen in the subject.

29. The method of claim 28, wherein the disease or disorder associated with a tick-borne pathogen is Lyme disease.
